SEQUENCE LISTING (17 sequences)

SEQ ID NO: 1 - DNA, 21 bp, synthetic construct
  source 1..21: mol_type = other DNA
  misc_feature 1..19: note = RNA
  misc_feature 20..21: note = DNA
cctgatgaaagattacttttt

SEQ ID NO: 2 - DNA, 21 bp, synthetic construct
  source 1..21: mol_type = other DNA
  misc_feature 1..20: note = RNA
  misc_feature 21: note = DNA
aaagtaatctttcatcaggct

SEQ ID NO: 3 - RNA, 21 nt, synthetic construct
  source 1..21: mol_type = other RNA
cgaaatggaagatgggaaatt

SEQ ID NO: 4 - RNA, 21 nt, synthetic construct
  source 1..21: mol_type = other RNA
tttcccatcttccatttcgtt

SEQ ID NO: 5 - RNA, 21 nt, synthetic construct
  source 1..21: mol_type = other RNA
cgaaacaggaatagaagaatt

SEQ ID NO: 6 - RNA, 21 nt, synthetic construct
  source 1..21: mol_type = other RNA
ttcttctattcctgtttcgtt

SEQ ID NO: 7 - RNA, 21 nt, synthetic construct
  source 1..21: mol_type = other RNA
aggaagaggtcataaagaatt

SEQ ID NO: 8 - RNA, 21 nt, synthetic construct
  source 1..21: mol_type = other RNA
ttctttatgacctcttccttt

SEQ ID NO: 9 - RNA, 21 nt, synthetic construct
  source 1..21: mol_type = other RNA
gcaccaagatccagagcattt

SEQ ID NO: 10 - RNA, 21 nt, synthetic construct
  source 1..21: mol_type = other RNA
atgctctggatcttggtgctt

SEQ ID NO: 11 - RNA, 19 nt, synthetic construct
  source 1..19: mol_type = other RNA
cctgatgaaagattacttt

SEQ ID NO: 12 - RNA, 19 nt, synthetic construct
  source 1..19: mol_type = other RNA
cgaaatggaagatgggaaa

SEQ ID NO: 13 - RNA, 19 nt, synthetic construct
  source 1..19: mol_type = other RNA
cgaaacaggaatagaagaa

SEQ ID NO: 14 - RNA, 19 nt, synthetic construct
  source 1..19: mol_type = other RNA
aggaagaggtcataaagaa

SEQ ID NO: 15 - RNA, 19 nt, synthetic construct
  source 1..19: mol_type = other RNA
gcaccaagatccagagcat

SEQ ID NO: 16 - DNA, 7980 bp, Homo sapiens
  source 1..7980: mol_type = other DNA
agtgacagcgggcggggcaggggccggagatggtggcggcggcagctcttccgacatgaggcagcggccgcgggggacacggcggcgggcgcggcggcggccgcatctctagctcggggaggcggcccccggcgtgggcgcgggcgggcgggcgtcccggccggcgtcgcgggagcccggctctgcggcagcgtctgcaccgccgcccggcgaggggcccgcgtccgtggttccacgcgctcggtcccgggacagctgcggccgcggcgggagcggcggcgcactcggggggaagcaggccccactagacgggccgcgccgacgggctccgcgcactcgcagcccggggcgcccccacccccagcgcccgccgccgcgggcagggccggcccggagcgcggggggcggccggggagcgcgagccgggggcgcccgccgaagctcgctgctccgggtcggggtccgggcccggggccggccgcgcgccgcctttgacgcatcggagcgcggctcctgcaggatggagggctccgcgccgccagcggagttgtttgtgcgcaggcggctcgcggggctgggagcgctcaaggtctgaacttccctccggagccgcagctggaggaggcgagcgcgcgaggaggagaagccgcgcggcgcggaggccaccctcggggcgagaggcgcggaaggcgagcgagcaaagcggtcccggagccacggcggccacgcggcggggacccccgggcgttctaggtactggtgaccccggccggggcaggccccgggactcgacaacatggcttccccaccccaccagcagctgctgcatcaccacagcaccgaggtgagctgcgactccagcggggacagcaacagcgtgcgcgtcaagatcaaccccaagcagctgtcctccaacagccaccccaagcactgcaaatacagcatctcctctagctgcagcagctctggggactccgggggcgtcccccggcgagtgggcggcggaggccggctgcgcaggcagaagaagctgccccagctgttcgagagggcctccagccgctggtgggaccccaagttcgactcggtgaacctggaggaggcctgcctggagcgctgcttcccgcagacccagcgccggttccggtatgcgctcttctacatcggcttcgcctgccttctgtggagcatctattttgcggtccacatgagatccagactgatcgtcatggtcgcccccgcgctgtgcttcctcctggtgtgtgtgggcttctttctgtttaccttcaccaagctgtacgcccggcattacgcgtggacctcgctggctctcaccctgctggtgttcgccctgaccctggctgcgcagttccaggtcttgacgcctgtctcaggacgcggcgacagctccaaccttacggccacagcccggcccacagatacttgcttatctcaagtggggagcttctccatgtgcatcgaagtgctctttttgctctataccgtcatgcacttacctttgtacctgagtttgtgtctgggggtggcctactctgtccttttcgagacctttggctaccatttccgggatgaagcctgcttcccctcgcccggagccggggccctgcactgggagctgctgagcagggggctgctccacggctgcatccacgccatcggggtccacctgttcgtcatgtcccaggtgaggtccaggagcaccttcctcaaggtggggcaatccattatgcacgggaaggacctggaagtggaaaaagccctcaaagagaggatgattcattccgtgatgccaagaatcatagccgatgacttaatgaagcagggagatgaggagagtgagaattctgtcaagaggcatgccacctcgagccccaagaacaggaagaaaaagtcttccatccaaaaagctcctatagccttccgcccttttaagatgcagcagatcgaagaagtcagtattttatttgcagatatcgtgggcttcaccaagatgagtgccaacaagtctgcccacgccctggtgggtctcctgaacgatctgttcggtcgcttcgaccgcctgtgtgaggagaccaagtgtgagaaaatcagcaccctgggagactgttactactgcgtggcgggctgtcccgagccccgggccgaccatgcctactgctgcatcgagatgggcctgggcatgatcaaggccatcgagcagttctgccaggagaagaaggagatggtgaacatgagagtcggggtgcacacgggcaccgtcctttgcggcatcctgggcatgaggaggtttaaatttgacgtgtggtccaacgatgtgaacctggccaatctcatggagcagctgggagtggccggcaaagttcacatttctgaggccaccgcaaaatacttagatgaccggtacgaaatggaagatgggaaagttattgaacggctgggccagagcgtggttgctgaccagttgaaaggtttgaagacatacctgatatcgggtcagagagccaaggagtctcgctgcagctgtgcagaggccttgctttctggctttgaggtcattgacggctcacaggtgtcctcaggccctaggggacaggggacagcgtcatcagggaatgtcagtgacttggcgcagactgtcaaaacctttgataaccttaagacctgcccttcgtgcggaatcacatttgctcccaaatctgaagccggcgccgagggaggagcacctcaaaacggctgccaagacgagcataaaaacagcaccaaggcttctggaggacctaatcccaaaactcagaacgggctcctcagccctccccaagaggagaagctcaccaacagtcagacttctctgtgtgagatcttgcaggagaagggaaggtgggcaggggtgagcctggaccagtcggctctccttccgctgaggttcaagaacatccgggagaaaacggacgcccactttgtggacgttatcaaagaagacagcctgatgaaagattacttttttaagccgcccattaatcagttcagcctgaacttcctggatcaggagctggagcgatcctacaggaccagctatcaggaagaggtcataaagaactcccccgtgaagacgtttgctagtcccaccttcagctccctcctggatgtgtttctgtcgaccacagtgtttctgacgctgtccaccacctgcttcctgaagtacgaggcggccaccgtgcctcccccgcccgccgccctggcggtcttcagtgcagccctgctgctggaggtgctgtccctcgcggtgtccatcaggatggtgttcttcctggaggacgtcatggcctgcaccaagcgcctgctggagtggatcgccggctggctaccacgtcactgcatcggggccatcctggtgtcgcttcccgcactggccgtctactcccatgtcacctccgaatatgagaccaacatacacttcccagtgttcacaggctcggccgcgctgattgccgtcgtgcactactgtaacttctgccagctcagctcctggatgaggtcctccctcgccaccgtcgtgggggccgggccgctgctcctgctctacgtctccctgtgcccagacagttctgtattaacttcgccccttgacgcagtacagaatttcagttccgagaggaacccgtgcaatagttcggtgccgcgtgacctccggcggcccgccagcctcatcggccaggaggtggttctcgtcttctttctcctgctcttgttggtctggttcctgaatcgcgaatttgaagtcagctaccgcctccactaccacggagacgtggaagcggatcttcaccgcaccaagatccagagcatgcgggaccaggcagactggctgctgaggaacatcatcccctaccacgtggctgagcagctgaaggtgtcccagacctactccaagaaccatgacagcggaggggtgatcttcgccagcatcgtcaacttcagcgagttctacgaggagaactacgagggcggcaaggagtgctaccgggtcctcaacgagctcatcggggactttgacgagctcctaagcaagccggactacagcagcatcgagaagatcaagaccatcggagccacgtacatggcggcgtcagggctgaacaccgcgcaggcccaggacggcagccacccgcaggagcacctgcagatcctgttcgagttcgccaaggagatgatgcgcgtggtggacgacttcaacaacaacatgctgtggttcaacttcaagctccgcgtcggcttcaaccatgggcccctcacggccggggtcatcggcaccaccaagctgctgtacgacatctggggagacaccgtcaacatcgccagcaggatggacaccaccggcgtggagtgccgcatccaggtgagcgaagagagctaccgcgtcttgagcaagatgggctatgacttcgactacagagggaccgtgaatgtcaaggggaaaggccagatgaagacctacctgtacccaaagtgcacggatcacagggtcatcccacagcaccagctgtccatctccccagacatccgcgtccaggtggatggcagcatcggacggtctcccacagacgagattgccaacctggtgccttctgtccagtatgtggacaagacatctctgggttctgacagcagcacgcaggccaaggatgcccacctgtcccccaagagaccgtggaaggagcccgtcaaagccgaagaaaggggtcgatttggcaaagccatagagaaagacgactgtgacgaaacaggaatagaagaagccaacgaactcaccaagctcaacgtttcaaagagtgtgtgaggcggcgcccacccgctgcccgaggtgctctgtttgtcgaaacacagtaatatttgtatttggctgttgtgctttccaagcgccacagttgccctccccggacgtggtgttatgtggtcatttcagccctaacttctgtgtggatcacagttattcagggttcattttcatccattcttccctttcgctcccttccctggaaaccccgctgcctctgggtcatccgttcagcacgtggtggagaacaagtgccttcagggctggcctcggcctcgagtctcgggacagaggccgccagtggagatcatggctttgggtattatttgacttttagaacaaaagctgtggttaagatctcatttttattgctttttcccacgtcccacgagacactattttcggttctctggctaataccctgtttttgagtttattttgtttctgtctatgtcacagtgttcctctacgacccgacctctctatgtaagcacacatgcgcacacacacttgcattcatgaatctgatataaagtgccagtaatccgccaagagggggtgcgaagggggcatgtcacgacagctccgccaccccccattgcccacccgcactttcccgagcaccgcgccccgtgggctgtgggtgagccgcgctccctgcactgagcgggtttaggggctcgcccacatgcatgcaggccaagacagcaaatgccagccgggcacgacgcccgtgtgcccaggcctcgggggtctcagagccgcctctcacccccgaccctccacccaggggtctccccatcgggagtggaggtgttggtcctggaagctgactcatcggagagggaaataccaaataaacatccgaggttgcatttatttatttatttatttacccggaagcatgtgcgctcccgggaggggtgctgggtcctcagggtggggggtcatagccacaggggagggcagttcccaggaaggtaaccagatgtctcttttgtgttttggccacctggggccgcccccaacctccccaccccaagaggggtctgagtgggacctcaaagcatggccagtgcaattctgaggctaggggaataggagaagcccaccagctgcccctgccacccacgggggctcctccctgccggggtccagctcagaaccacgggggtctgtgccggcagcaccggtgccatcttggcttccacaaattccagaagtttcttggctgcctcccgctctcagatggcacagggcctatcagtggcaggatttaatctggggagcaaattccagctctgaaatggagttctcacaccgtatctagggacagaggcatctggatgcaaacgcggggtttgttaggggcggaatcccgagggtggtgtgggggagctccgtccgggcagttggctgctggtcctgtctgattatgttttgccatattgagtctgaagtttgtgtggaagtgagctcgcccgagttgtccggtcggagttgtagccagtctttcaggggaccccgggacgctgatgaggcccagggtgcactccttgtcagtcttgcccgttgtccctgtgcattttgctttccgctgtcaaatctcactgtctcgttttcacaccgcatcagtccccatcatcactactcctccccagagacatacagattgccctccccggcatgaagaagtcacaattctgacccgccacctctccaggggcggtgcctgggatggccacccatcaggacagccagagccagggtcccggcgtcctgagagtccagggcacctccccccattagctgtaaggtttcctgtcaggtgtgactgtcagtttgctggtcttccaataccgaagaaagggtggttgtgacaatacctcttgcttctaaagaatgtattataaaacaccgcagatttttttttttccttaaaaaacactacctgatgctttccttgttcgtggggattgtggtcacatgaagctctttctgcatcagtattaaggtgtatatttgaatgtcctcccctcccctttcccctccaggctgtgtagctttgaggggctgggcgtttgctcacgaccttgctgtctcgctcagaacatgctccgcaaagttctccgcacacacttcttccccatcaagcccatttccttccccaaccacaaaggtgtttgtgattcctcaccccgggaaaccaaggagctgcaaagtggagtctggttcagccccgtgcagactcacccagagcttaacgttgtctttcaaacaccctgagccttcctaaacagccagtgcagacgttctctctgggccacgaagcccctcgggtcctccccgtcccctgctccgatgcatacctcagtgcagaaccacagaatctctgcagcggaaacgccgtgcatctcttgtctgttggcagcgagcacatcgtgctggagacacgagtttctaagcagctggcacgagggctgctgacggcatgggtcgtgcttcagggtggcaatacctcttaggaacttagggcaggaagcaatacttcagcattgaatgtgtgtaaatagttgctttgagttgcaattgctattttcttctcagtcccagctcagatcgaattatatatccatatatatatatatatatatatatatatatatatatatatatatatggtaaacaagcacacacaattttatccaatgcaaacaaatgtagagcatcagttacaaaaccctcgaatagcttgagagccccacaggctctgccacacccgtgacttcatccacactgacgtcacccgcgggggctccccctgcacatttgcacacgatccggagagccgaaggccgcgtgcttcctgtcacatgggctgtaatcatttgtagtttccaaagacacgtctgcatttgaatttctagattttcgaggtaaggagttttttttaattggttgtttggaaaatcacatcatgcctagaatctgaaattgaattagcaagaaccgactgtttgcattttccatatatccttttatctgctctttttaaattgttaattctaataatttcaaaatgcattcactgaagaaatggacattaaaatattctaaaatttaaa

SEQ ID NO: 17 - DNA, 153537 bp, Homo sapiens
  source 1..153537: mol_type = genomic DNA
gcggccgcggcgggagcggcggcgcactcggggggaagcaggccccactagacgggccgcgccgacgggctccgcgcactcgcagcccggggcgcccccacccccagcgcccgccgccgcgggcagggccggcccggagcgcggggggcggccggggagcgcgagccgggggcgcccgccgaagctcgctgctccgggtcggggtccgggcccggggccggccgcgcgccgcctttgacgcatcggagcgcggctcctgcaggatggagggctccgcgccgccagcggagttgtttgtgcgcaggcggctcgcggggctgggagcgctcaaggtctgaacttccctccggagccgcagctggaggaggcgagcgcgcgaggaggagaagccgcgcggcgcggaggccaccctcggggcgagaggcgcggaaggcgagcgagcaaagcggtcccggagccacggcggccacgcggcggggacccccgggcgttctaggtccgtcccgagcggttctcgtgcgcctcgaaggtgggggcggtgggggcggtgggagcaccgctgagccgggaaccacaggaacagatggtgcctgtccaggtcgcgtgctggagccgccccttaggacaggagcaggtcccgggcctctaggcgtgcatgcctaggtgggagcgccgctaactctccccgttttgctggcaggtactggtgaccccggccggggcaggccccgggactcgacaacatggcttccccaccccaccagcagctgctgcatcaccacagcaccgaggtgagctgcgactccagcggggacagcaacagcgtgcgcgtcaagatcaaccccaagcagctgtcctccaacagccaccccaagcactgcaaatacagcatctcctctagctgcagcagctctggggactccgggggcgtcccccggcgagtgggcggcggaggccggctgcgcaggcagaagaagctgccccagctgttcgagagggcctccagccgctggtgggaccccaagttcgactcggtgaacctggaggaggcctgcctggagcgctgcttcccgcagacccagcgccggttccggtatgcgctcttctacatcggcttcgcctgccttctgtggagcatctattttgcggtccacatgagatccagactgatcgtcatggtcgcccccgcgctgtgcttcctcctggtgtgtgtgggcttctttctgtttaccttcaccaagctgtacgcccggcattacgcgtggacctcgctggctctcaccctgctggtgttcgccctgaccctggctgcgcagttccaggtcttgacgcctgtctcaggacgcggcgacagctccaaccttacggccacagcccggcccacagatacttgcttatctcaagtggggagcttctccatgtgcatcgaagtgctctttttgctctataccgtcatgcacttacctttgtacctgagtttgtgtctgggggtggcctactctgtccttttcgagacctttggctaccatttccgggatgaagcctgcttcccctcgcccggagccggggccctgcactgggagctgctgagcagggggctgctccacggctgcatccacgccatcggggtccacctgttcgtcatgtcccaggtgaggtccaggagcaccttcctcaaggtggggcaatccattatgcacgggaaggacctggaagtggaaaaagccctcaaagagaggatgattcattccgtgatgccaagaatcatagccgatgacttaatgaagcagggagatgaggagagtgagaattctgtcaagaggcatgccacctcgagccccaagaacaggaagaaaaagtcttccatccaaaaagctcctatagccttccgcccttttaagatgcagcagatcgaagaagtcagtattttatttgcagatatcgtgggcttcaccaagatgagtgccaacaagtctgcccacgccctggtgggtctcctgaacgatctgttcggtcgcttcgaccgcctgtgtgaggagaccaagtgtgagaaaatcagcaccctgggagactgttactactgcgtggcgggctgtcccgagccccgggccgaccatgcctactgctgcatcgagatgggcctgggcatgatcaaggccatcgagcagttctgccaggagaagaaggagatggtgaacatgagagtcggggtgcacacgggcaccgtcctttgcggcatcctgggcatgaggaggtttaaatttgacgtgtggtccaacgatgtgaacctggccaatctcatggagcagctgggagtggccggcaaagttcacatttctgaggccaccgcaaaatacttagatgaccggtacgaaatggaagatgggaaagttattgaacggctgggccagagcgtggttgctgaccagttgaaaggtatgtgcactgctttacctcggcatcccccctccctgatcctgattgtattaaattaaaaaaaaaagcttccagcctcagtgtctgcagacagctcaggtatctcagaatgccttaccatggggggatctcagggtcagtgtgacttttctttcagcgggtcaaatatcacagatctgttcacactgcagaaagggacgctgtgcagagatgacctagacgcgacctttagaaggaaggagaactttgaacgtgcataaccactggttggctgaaatgtgggggcacgtccggtgaatgtaagggtatggagcccttcagcattcagcactgtttagcacttaaggaaattgcctttgcattcatttggtcaccctgaagtcccatccaaatgtttctctctgccctttttgcgaaaacgtggagaataaaatctttctcctctctaggcctcactttgcttatcctcaaaatagggataggaagtcggcgcagtcctgcaggtctctggagaattagacggagttacttacctctgagtacacactgtcttttatctaggtctttttttttttttaaaaaaagtttgcattttcagctttcagccatcatttgtttaaggatattttattatcatagtatttgccttaagtgaagagcttcatattgagcttctccagccaccaggtgatttggaagggagaaacgtacctgccggagagctggtattcccctccctcatcctctcaggtttggatctggtgcctggtccctggtttgaagctccttactgctattcttgatgtttttactgggctgcttgttttttctaatataggacaaaagtctttgagatctggaaaatgatattaaaataagtaggacgaaaaagaaagattttaagtaatctgctacatttcttcccccacttcctcctttttaaacagtccttatgttttactaaaatcttcaggattgaggggaacatctgttgtggaagtatgtagagaaaataacactgattttttttaaaagcctgcttttgtgaagctgtattgtattttgtggtccccacacactgcatacttaaacagttaaaagcctcaggaaagggaatcctcggaagaagtaattaatagttccaatcagagatggcagaagtaatagtttcaatcagagacggcagaagtaatagtgtcagtcagagacgtccagtggttgaggtagggcctgcataggtcgttggtgtgagatcatcctatgtgtccagttggcattctctttctctggcttgtgatctgagctcctgtatcttttatgtatttgttctctggcaagggaacatcatctttcttctgatttttctttattttactaactttgctgttgaagattttatggatgtagacgaaaactattttcacccgagaattcccctaacctagagcactttaaggactatgttaacaaggtcagaaactacactttctgttcacttgaaagatgctgccgtgatgagtgtctttccgtttagggcggtggcactcgctcttgagtctgaaggtgtcagtggacggggctgtgggtgtaaccccctgcttgccaccttggcatttttagggcccaggtgttgtgagctctgtccttggagactgtgggacggtggagctgggcggcagtacgttaaattaaatatgttggcccagaggacaatgagaacgttttgtaagctgtactttgtatgctgtattactcgaatttcaaaaatgaagagggtgtaattattaaatatgtttggaaattgccaagtcattgaatacctattttgtgaatttatgagtagttttatttttaagtttgtagatcaggggtctcagacacagagccaaaaaaaaaaaatgctagagtgagtttgtgtttttttttttttaaacctcaaatagtgtaaatttggtaaattaacttttcttgacatttgtgtacattgtatatttataaaagttttagtactgtagggagtagggaagatagaaggcttagcttagctgctgattttttaaaagtgatattctttcctttagctctttctctgagaccacgattttgttttcttgagctcattttacaaacctttgtggataggactccaggaatagagtatgtctacacaacttcctggcccttttggaaactgtccttacaaagctaaattgagatgacagcacagtttgtagaagaccctttcacacacatagttaagcttatctacatgtaagatcgattttagcctctgcctttgggctggtggcagatgttttctctatacaatattgggtattacgggaggctgaggcaggagaattgtttgaacccgggaagagaaggttgtagtgagccgaggctgcgccactgcacaccagcctgggcgacagagcaactctgtcttaaaaaacaacaacaacaaaaacaaaaaaaaccccaatactgggtgtattagcctttccctcttacccccttttctgatcccagggttaaaaagcagccagtagcggccctgcaggaatgggcatccatagaatgagaaggggcttctagaactagcctgggagcacagaaggaagctcctctcattcaaatttatccgaacaggctttgcagaagggtagtttcatgatttctgcacattccttcactctgaaatctgtgtcgtgcccctggcactgactgtgcacctggcactgtggatgtggagcaggatccgtgatcgtggagcaggatccgtgatcgtgaggtaggaggtgggactcgactccagaggcggggttcagatgccagaccgaattgaggactagctgatacagggatggggcagaagcagcgttctgcaaggcacgcccaccagtgtgccttgttagtttactgtcgccatggccacacccggagttactgcctctttccatggccgtgacctgacaacccaaaagccaccacccttttcctagaagtgtctgcctaagcctacctttactctacaggaaattaaaagtaggtataaggtaaggctgggcgcagtggctcatgcctgtaatcccagcactttgggaggccaaggcgggtggatcacctgtggtcatgagttggagaccagtgtggccaatatagtgaaaccccgtctctactaaaaatacaaaaattagcctggtgtggtggtgtgcgcctgtaatcccaggtgctcgggaggctgaggcagaagaatcgcttgaacccaggaggtggaggttgcagtgagctgtgatcatgtccactgcactccagcctgggcgacagcctaggcgagactgcgtctcaaaaaaaaaaaaaaaaaaaaaaaaaagtaggtataagtatgattgcaaaactgccctgagctgctactttccgcctttgggaaagcccagctctgcaggagcagtcatggagccgtataacactgcctgcctcttcaataaaggtgttttctttcctgccaccggctcacccttgaattatttcctgggcaaagccaagaaccctcttgggctcggccccactttggggcgcgtctgacctgcgttaatagttcccaacctccgtgagctcacagttaaaaatgttgtggagtatgttcgggagtggggagcgctttagagggggctcctgctggagctgggtcctgagatcaggctgtggctctccaggtggacagaagagcaccagggcagagcggagaggtgggtatggctgggccctgtgcttctctgtgggggcggacgattgttagttgttcatcaggacaaggaaggccatgggtagcgtgggaaggagtttgcatttgaccccataggtgtcagggaaccactcaacagggggcttaggtgatctgacttgcagttttagaaagttcactctttcagagctagtgagtgtgggttggaaagagattaatgaggtgtcgagtaaatcccaggtaggggtgatggtggcccaatccaaagttacagtagcagtgatggaggtgatgggatctgagtggcattaggaagagcagcttgatgggactgcaagccagatttaaacaaacgactggcaggaggggaagggcttgaccatgtgaatgggaaatggcattttaagtttctggaagtgacttggcgacaatgtggagtcaggaatgggtgaggggcctttgaagttgctatggtatgaatggagaatgcaggatggacttgcagagtacagaggcccggaacccctggcttgcttactcattcagcaaatcatttactccgtgccaggcacggttctgggttctggcaatacagccgtaaacaaaacagacaacaattcctgctcttgtaataggggacaagaatttttaagtcaataatataggaggatcgctggagcccacgaggtcgaggttgcagagagccgtgattgcaccactgcattccagcctgggcgacagagtgagaccctgtctctaagaaacaaaaaccaggccttcaaataggggagatctaatagaggggacaacaggggagtcaaaccataaataagtaaaatagaaaaagtgttttgtgaacaaacataaagcaggaaggggatacagcagatggggtgtacgagtgtgagaaatcttatctagggtggccagtcactgggagaaggttgcaggaatttttagcatactgctaggattttggaaataatctaaaaagtgaggggccaggcatggtggctcatgcctgtaatctcaacactttgggaggccgaggtaggcggatcacctgaggtcaggagtttgagaccagcctggccaacgtggtgaaaccccatatttactaaaaatacaaaaattaaccaggtgtggtggcaggtacctgtaatcccagctactcgggatgctgaggcaggagaattgcttgaacctgggagacggaggttgcagtgagccaagattgcaccattgcactccagcctgggcgacaagaatgagaccctgtctcaaaaaaaaaaaaaaaagtgtgagggacagttattaaagatatggagaaatgggccgggcgtggtggctcatgcctgtaatcccagcactttgggagaccgaggtgggcagatcaggaggtcaggagtttgagaccagcctgaccaacatggtgaaaccctgtctctactgaaaatgcaaaaattggctgggtgtggtcgcaggcgcctttagtcccagccacttgggaagctgaggcaggagaatcgttcgaacccaggaggtggagtttgcagtgagccgagatcgtgccattgcactccagcctgggcgatagggcgagactttgtcgccaaaaaaaaaaaaaaaaaaaaaaaaaaaaaatgaagaaacggtctgattaaggtgagggaccttccacagcatttgggatgaaaccgctaggcctgagggaaccgtggctgttgtattctgccttgggccacaagcaaaaaatggtgagacgtgttatctcgagctgttggcttttcctgtcatagatacagggtcacaaagacagaaaatgaaactgttagaaaatatctacaggcatcttcttttcccgtgtaaatattaggaagacagaatgaagatgctcctccgctctcatccacacccatggtcatatctctgaagaggggaataccagggagtgatcacattgtcagatgcagaaattaaaaaagcttttttccccagccaaactttatgcttgtggagcaacttggggaggagacccgctggtatgcatttgggcagattgtcacctgttggccacctctgtgatcgagacctttgccttagaggagttcctatgtagaccctgtctggtgggccgacagaccgacagtggacggaggcattctgtatgcgttagcctctgcaaacaaacacctgtctcatgccggaaatctgggcgccccattaaccctgagcacgggcctgccagctgacagaaccctagccaggtgttggtttggggttcagttgcatgctcaggtgttccccttccttcccccgttagcttgtccccacctaagcctgcaacccttgccactgggatgctaacccgttagcagagctcagtctccttgcttggaaattctgtgttgacctgcactgcattcttttttttttttttttttttttttttttgagacagagtctggctgtgtcacccaggctggaatgccatggcgtgatctcagcttactgcaacctctgcctcccaggttcaagcggttctcctgcctcagcctcccaaatagctgggattacaggtgtgcgccaccacgcccggctaatttttgtatttttagtagagatggggtttcaccatgtaggccaggccggtcttgaactcctgacctcaggtgatccaccagcctcagcctcccaaagggctgggattacaggcgtgagccaccacgcccagccttgacctgcattcttgagcaggatctgttatgcacgtggaaatacagcctctcagatagcaaatggcagagatgagatgaggcacacagttcccctccacagctggggtgggagacagaaaacagcagtagagtcgacactcaagctgcccccagtgaccatttgttcaggctctttgagacctggcttacagtgacaaggacacagctgttaattctgaagacaagctttcgagtagaagtaacctctggtaaagcaagagaaggttaaggtttgctgaattgccaatgggatgaagaaggaagggagggctgtcttttcaggtagaatgcctaaggaagggaaacatggagaaggaacaattagagcaagtcaagaagtttcaagatttttgtcttctggccagcttggtgagatagtaattatgtttctgcatattggtcagcgttgcctatgtactttgctgtggaatagtaatcttggaaaaaaaccaaccaaccaacccacattaatctgtccatgtctgggcacagagtgtgcagaagcctgcgtgggccgtaagtctcgtctttgtcttggttggtgattttagtattggtgtttttgctggaattccaaaactgtaagagtgtaatcattcaccattagaattctgcaggtcccctcctgactctggactagctgagcttaggggcctgtcatccatctggccctgggcatatcatttgtactttgcctgtgacacacctcaagaccttttccaacaggaactagcatccctggagggaattttgtgaagagatgagagtttttgaaacaggaagggaactggcaagggaagtggccaggcaccggtgctgccgcggaacagaagggaagggactgggctgcatcagagggtggatgtggggccgccatccgtggtgtgggccaagggtagggtggtcctcccctgggaagactgcagacagcagtgatattcttgtagtcttgtggatgtacccagtgaaccacaccagccaagccaacccgtggctaaattttagtcaacttcagcgaagaagactgccccttgtaaaacttgtctgctcttggaaatcaggttttttgtctcattttccccagcctttgctagcattgcacttgtaaaaaattcctggcatcctttctgcggagttaagtgggaaggctcaatgttctgggataggtgggaacatcaaacccgctagaggctgctggtggcagtggaggcagaatgagttgtagcagtagtggtggcagtagtgtgacctagagggcagaagctgggaaacagggtgaaatgctgggccccgacagtcagggatagctgcacctagcttatatgccttcctctaatccagtagaaattgttgtttctgtaggttttttttcttttctttttgagacagggtcttcctctgttgcccaggctggagtacagtgtgcagtcacagttcactgcagcctcgacctcctgcactcagatgatccttccatgtcagcctcttcagtagctaggacacaggcgcatgccaccacatccaactaatttttttttttttttttttttgagacggagtctcactcttgcccaggctggagagcagtggtgtgatcttaacttactgcaacctctgcctcctgggtgcaaggaattctcgtgtctcagtctcccgtgtggctgggattacagatgcatgccaccatgcccggccaatttttttttttttgtatttttagtagaaatggtgtttgaccatgttggccaggctggtgtcaaaatcttggcctcaagtgatctgcccgcctcagcctctcaaagtgctgggattataggtgtgagccaccatgcccggcctaattttaaaaattatttgtagagatgacgtctccctatgtttcccaggcttatgtgcattatttatataggttgtctattgatatttacagtattagaaattgaaacaaactgagaagcttaaaaatcttttctaaatttatttcaaaatagtaattcatttcacccattgcatgttaatgtaagtaacatttttataaaagtaactatattttgtaaaatgaaaaaatttgatgagaagggtggcactgccatacatttttgcaaatctctttaatgtctggattaataaacggttgaagttttttgttttttttttggtggatttttttccctcttgttgcccacattggagtgcagtggcacgatctcggcttactgcaacctctgcctcctgggttcaagcgattctcctgcctcagcctctcgagtagctgggattacaggcacgcaccaccacacccggctaattttttgtatttttagtagaaacagggtttcagcatgttagccactgcgcccgacccacggctgaggttttttgagtgataaaatgtacacaacagaaatctaccgtgttaaaacacttttaagtgtgttcgctgaattttcttaaccgctttggcattcaatttgttgcaatatgttgaaaaatgtgtagaaaatctggctttagttggaagagcattttcatagcctttttatgtgattctggatatttttctttgattctgtagaaacattcaacaagtgagtagtagtttctttttctttttctgagatggggtcctactctgtcacccaggctggagtgcagtggtgctaccatagctcactccagccttgacctcctgggctcaagtgatccttctgccttggcctaccaagtagctgggactacaggcatgcaccaccacaccccgctgattttttattttgagtggtagtttcttaaatgttagttgcaatatggaatctgaaattttattatctttttttgtacactattatattaaatgctgttgggtttttacccaggtgtgattttataacgtcatgcactagtcatttggaaaatattggtctcttgaatgatgcagctcttccacatgttgacgtcagtaaagtctaatagtctgaagatgttgagttcatcacagtggctgatgtaagttttccaaaactctacttgaaaactggaattttttatcattggcggcaaatactgtcagttattttccttgaagcgacagactcattcatattcccaggaatggtctgcccgtactcaagcctgaacgaccgtggtttgtctgtaggtcacgtgtaaataatgtgctaccaaaaaggtggccagttcagcttgcaatccaaacagctgcacagctgcttttctccaggaccgctgtgctttgatgggcagcagcgtcgtcgtaggcgtgcttctcatttcatcactcagactattcagaagacacctactcaagcttgcgatttaaccaatcattcatgttgacagcttcatctgggacattcttttttttcttttttctgtttttgtgacagagtctcgctctgtcgcccgggctggagtgcagtggtgtgatcttggctcactgcaacctccgcctcctgggttcaagtgattctcctgcctcagcctcccaagtagctgggatttacagatgtgtgccaccacgcccggctaatttttgtctttatggtagagacggggtttcgccatattggccaggctggtcttgaactcctgacctcaggtgatctccctacctcggcctcacaaaatgctgggattacaggcgtgagccactatgcccggcccatcagggacattcttaagtgaagctgtctgcctttcctttcgtaagtgtggggtagtgaaggacaagctgagggctagtgcagtttggtttccctgcatcgggacctgctcaggcactagcagtttcacccatggcttcttctgcactgtcactgcaagtgtcaacactgaagaaggcaaatgctgtcttagtgtcattgtgcaaatagtttagactccacagatgccttgcaaaggcctcaaggacctctagggatctgcggatcacactttgagtactgcttcgctagctgggctgcaagatgggaagactgagggttttgtatgtgccacccaaacacgcattctcatgtgtgtgaaacaacgccaattggagttgtcacacccattacatattgcctgtggaaatagcaagtgaccttagttcatacattgaccttttcttacaaaatccttaaaaatcttttgtgattctggtaaatctttgttgctcatgttcaaagtgtgtgataaagaccatcaacttcgagctggccatggctcgtgcctgtaataccagcgctttgggaggccgaggtgggtggatcacctgaggtcaagagttggagaccagcctggccaacacggtgaagctccgtctctcctaaaaatactataattagccaggcgtggtggtgggcacctgtaatcccagctactcaggaggttgaggcaggagaatcacttgaacctgggaggcggaggctgcagtgagccgagatggcaccactgcattccagcctgggcgacagagcgagactctgcctcaaaaaacaaacacatggctgggcgcagtgagtcacgcctgtaatcccagtactttgggaggccgaggcgggcggatcacctgaggtcgggagttcgagaccagcctgaccaacatggagaaaccccgtctctactaaaaacacaaaattagccaggcatggtggcacatgcctgtggtcccagctactcgggcggctgaggtaggagaattgcttgaatccaggaggcggaggttgcggtgagctgagatcatgccactgctctctagcctgggcaacaagagcgaaactccatctcaaaaacaaacaaacaaacaaagaaacaaccatcaactttgtttctgtaaattttttttggaagggctcacccttgaatggttgggtgcttggattttgatttctgatacattcgggcttaattctcggttgtatggtttcctaactgtgggactttgggcaagctacctaggggcgctgcaatttataggaatgatggtgtctctgtcagagggtcgttgggagaatcaactgggataatgcaagaaaagcacttatttgcatagtgactcgatggtggacagccattccatcaataactaacgctccccgctggctgagtcacatttcgtattgcactaaagggtgtttctttcttcctgcgctcaatctggcgcacatcttgtgtcgttcagtaccgaaacctagaacacactagggactgtgattgtgtcagtttctcgtaataaaaagggcaagtgtccaaaatacaaaatactgatagctcgccctggttaaactctagctgccgaacttggcagcctctttactgaaaaccctcgggggttcagcaggctcgttcctaggctgcttcagttccccagagcctggaggttcttaagatgatgggcccctcgatggttgaggaattataagcagatcttcagctgtgcaactccagctattggccgtggtttgaactagagtggtttttttcaactgtactgtatcctactgtaaactcatttgtttattcattaaatattgaatgggttcctaccattggcaaggcgaggactaggttctggagatagaggggtggcccctgccctcttggggcatgacgcctagaaagggaagtagacagtgatcaaataatctgtacaggccaggtgcagtggtcatgtctataatcccagcactttggaaggccaaggtaggaggatcgcttgagtccaggagctcgagaccagtctgggcaacatagtgagaccctgtctctaaaaataataaataattagccgtgcatggtggcatgtgcctgtattccaagctacttggcagactgagatgggaggatcgtttgagcccaggtggtcgaggctgcagtgagctgtgattgcaccactgtactcagcctgggtgacagagcaagactctgtctcaaaaaaaaaaaaaaaaatcacctgaacaaatattgtactagacttcctgactgtattacagatagggttgagatgatctctattttatagatgagaagactgaagcacagagaggttaggtaacttgtgatgatcacacagccagaaagggatactaaatggtggagccagtatttaaacccaaaggctgtattagaaaacactagtacccatctcacagaattgctgcgaggattaaatgagaaaatacaagttcttagaaaagttcttggcacataatacctgtcagtagagaagagctagtgttactgaaagttggaataattttcttagcagtggcagcaacagccaaaacaacgatacctgtctttagctctctgcatttaccctgctgtcatgtaccctgccctcacgtatcctgctgtctcagccactgtgaaatcctaggatcctgggagccgtgtgtgtttagatgcttttatttgcttggttctttccctttctataccatttacagactgagtttgtttttttcttttctttttttttttttttgagacaagagtctcactcctttgcccaggttggagtgcagtggcataatctcggctcactgtagcttccacctgtatcagaccattctaacactgctataaagttgctacctgaggccaggtgcatggctcacgcctgtaatcccagcactttcggaggctgaggcgggcggatcacttgagctcaggagttcgagaccagcctgtccgacatggcaaaaccccgtgtctactaaaaatacaaaaaattagccaggcgtggtggcatacgcctctaatttcagctacttgggaggctgaggcaggagaatcgctggaacctagaaggtggaggttgcagtgagcccagatcgcgccactgtactccagcctgggcaacagagtgagaccccatctcagaaacaaacaaacaaaaaagatgctacctgagactgagtaatttataaagaaagcaggtttaattggcacagttctgcatggctggggaggcgtcaggaaacttacaatcatggcagaagatgaaggagaaacaaggcgcatcttaaatggtgtcaggagagagagagagtgagagagagagcatgcgaagggggaactgccacttttaaaccatcaggtctcgtgagaactccctcactctcacgagaacagcatgggggaaaccacccccatgatctagtcccctcccaccaggtccctccctcgacacatggggattacaattcaagatgagatttgggtggggacacagagctaaactatatcaacctcccagggtcaagtgctcctcgtacctcagcctccctgtgactgcacatgtgcaccaccacgcccagctgattttttcaaatttttgagagagacagggtcttgccatgttgcctaggctggtctcaaactcctgggctcaagccatcctcctgccttggcctcccgaagtgttgggattacaggcgtgagccaaggcacccagctgagatggtttcttaacgtcctccaaaggtgatttttttcaagaatcaatataaactcttggattttagcttatttgatgtatttcattccattacagatacatgatttttgatgctcagattgtcccatttggggcccatgggagcccctttaagttggctgctgagtcctcttcatgcaaccccagtggtctttgataactttcatacccttttagtaacctttttaaagagttgagaaattataggtattaaaatctcattaattctgtttgtctgatatgtttattcagagagactgagctaaagtttaccctcatcttttttgaaaagaaaatgaatagtatatgcatgtttctatgaggaaataatgccacccaagaaacggtttgtgcatcttcgagttccttggaagctttaatttgcattcctacaattgagagcaatgtagaatttctttctgtatcttcccactaatgcctgttgaggggcaggtgtctgtgaatgtaggtaaatcgtgataaaaaaacaataaaaaggccgggcgtggtggttcacgtctgtaatcccagcactttgggaagctgaggcgggtggatcacttgaggtcaggagtttgagaccagcctggccaacatggggaaaccctgtctgtactggaaatacaaaaattagccgggtgtcgtggcgcacacctgtaatcccagctactcgggaaactgaggcaggagaattgtttcaacctgggaggcagaggttgcagtgagctgagatcacaccactgcactccagcctgggtgacacggtgagaactctgtctccaaaaacaaaacaaagcaaaacaaaacaaaaccaaaacaataaaaaaaaatcccaaaccaaaatgtagatgacctgtttgcagactgaggagtcctgcagcaaactccatcccccaggacagctgcggaatgttctagaaggcccaactctaggaaatcccttagtaggagccttgctctcaacagtgcctcctggaacgatgggccggccactctcttttccatcttgcatcatatgtatttgacttattttgtttataatctgtttccacgccactggaatgtatcttaagagcaggaattttccatgttttacttcctgctgtgttcctagtgctgggaatatagccaggcatataatacatgcctgataatattggctgaatgaatgaatgaataaatgaatgaatattacaggccagacacagtggctcatgcctgtaatcccagcactttggaaggccgaggcgggcggatcacctgaggtcgggagtttgagaccaacctggccaacatggtgaaaccccatctctactaaaaatacaaaaattagccgggcttggtggcgtgcacctgtagtcccagctactcgggaaattgagacaggagaatcacttgaacccgggaggcagaggttgctgagattgcaccactacattccagcctgggtgacagagtgagattctgtctcaaaaaaaaaaaccaaaaacaaaaacaaaacaaaacaaaaaatcccaaaccaaaacgtagatgacatgtttgcagactgaggagtcctcccccagcccagcaaaccccatccctcaggaccgctgtggaacattccagaaggcccaactccaggaattcccttagtaggagccttgctttcaacagtgcctcctggagctatgggccagccactctcttttccagcttgcatcatatgtatttgacttaattttgtttataatctgtttccacgccactggaatgcatcttaagagcaggaattttccatgttttacttcctgctgtgttcctagcgctgggaatatagccaggcatataatatatgtgtgataatatttgctgataaatgaatgaataaataagtaaatataagcctggtatggtggctcatgcctgtaatcccagcactttgggaggccaaggcaggcagatcagctgaggtcaggagttcaagaacagcctggccaacgtggagaaatgccatctctattaaaaatacaaaaatcagctgggcatggtggtgggcacctgtaatcccagctactcgggaagctgaggcaggagaatctcttgaacccaggagatggaggttgctgagattgcgcccctgcactccagcctggatgacagagcaagactctgtcttaaaaaaaaaaaaaaaaaatatatatatatatatatatatatatgtacatatgtatttatacatatatatagtgtgtgtgtgtgtatatatatatatatatatatatatatatatatatgtaactccacatgtgagtaccctgttatgtgtgaaaccacaccccaaacatctacacagaacaagcctccaggtagaggaaaggcagtaaccgctttgctggaatgactgagtggttcattaaatagaggctccggagggtcttgataggctctagcaaaggtggtggccgagagggctgaccagcgcatggtgcctgggaatgtgaatgggcccaggggcgccagagtgtggacagcttgggactgtgggctccgaggtgctgcgctgagggcgtggggcgcagggcagaaacacagaggactgagtgcttaggggaggcccaggaaatgtggggttgaacggcctagttctggcaattgtttttaagctttgtaagatggagatttcaagaagtatatttatcttaataatattgtagctcctggtaattcttatttgattaccatttatacacatggaaaaccagcatgcctgtttctggcaaagctccagggtagtctttgtgcataatttccaggatttttatgtaatgctattaaaatctgttcttttttttttcccccttccaaagcggggaaaaagcagcttttgaatcatttattttcgtgtttgagttgtttgggaaatattaactacattttctaacactctagcctggagaaaggaagaaaccccacatgcgtgtgatttatttccttgtgtatggatattaaatgcaatataccattaaggctatgacactttaagtgaaagtgtttattgcatgttgattcagttagttcgtaccctattgtgttactatttaaagtgtggattgcatcgcattctggaacttcataaatgagttctttttcccataaggaaaggccactgcttctgatgttcattaatgtgatttttgtgtgtgtgaatgagctgcctaaaaactgggtgtggtgagggtggtttctgaaagggaagcacttttatagcatgatcttatttgaaaatttaatttgtttttgtaaattagcttttggtcttaatacatttcctccactaacaagcacttttgggcaaataatttaatctctctggttctgcttttttcatattggtaaaatataaagtttgagatagatatgcttaaaatctccaccattgtctaagtatagctgagattctcacgtgattgggacatagtgctcattcttaatttgaaaagtgtttttttctttgtcctcttgtttcaactacgaactctcatttagacacatttagcatagggaagctccgatttatagagccactaaattgcagggcgtgtgtgagatttgtaacttcataaaagattctttacttcctttaaatataaatacgtaagattgagttctagaagtataatttacacatggtttcctgaggatcacgtttcctgaataaagcagggtatcgttagaggaaccccatttgattttttttttttttttttttttgagatagagtcttgctctgtcacccagggtagagtgcagtggcgtgatctcagctcactgcaaactccgcctcctgggttcaagtgagtcttgtgcctcagcctctcgagtagctgggactatagtcatgcaacatcatgcccgtctaatttttgtgtattttgtagagatggggtttcatcatgttgcccaggctggtcttgaactcctgagctcaagcaatccagctgccttggcctcctaaagtgctgtgattacaggcgtgacccatggtgcctggtctgggaatcctgtttgattttgaaagtctgagaaacacttttgaaatggcttggagaaaatgtgaagtgaataattattatttttttaatgtgcttcagttcccatgtgaacattattttagagagagattggaaggttagttgccagtaaaatgaatttcactgtgttggcaagtttttcactcgtaggactgagtgaatcctggactgacagtgttcttaccgatgattctgttgaggaaggacttgcttgctcttgttgctcttgacggtgagcttctggatctactggccctatattgagtgtgcaaggggacgtgttagtcctctagttgtcccccacattaatgcatctggaaagtgtctaccttagatgttgtaattagaaattagagggctgggcgcggtggctcacgcctgtaatcgcagcactttgggaggctgaggcatgtggatcacctgagatcaggagttcgagatcagcctggccaacgtccgaaaccctgtctctactaaaaatacaaaaattagccgggcatggtggtgcgcacctgtaatcccagttactcagaggctgagacaggagaatcgcttgaacctggtaagtagaggttgcagtgagctgagattgcgccattgcactccagcctaggtgacagagactgtctcaaaaaaaaaaaaaaaaaaattaaatgtcatcaacattgaaaggaaactcttggattttgtcaaagatttataaaaagttttgaatgattttatttttttcattcagaagcataatttccctcttaaaatccctgatgtggatcatttggttccctgtcatttccattttctttgttcagagtgtctcttctggatctcctgagtttctgtgtgtgttttcttcctttttgctcaaatgatgagcacacatgaaagcttttcctcctttgcctgggtgaggcggtgttccaggtgtgatctgcagtgcctgattattatattgtattaatctcctcaattttattttctggcttgtctgcatttttccttttctttcttctcccttttttttttcttattagagatgggggtcttgctgtgttgcggggcctcgagcgatcgtcccactttggcctcccaaagtgctaggattacgggcgtgagccacagcgcctggcctccttttctttatagtaatttgaatgtggtgattttaatcctccatggttttcttatccttcctcaacaaacagcaaagaggctgctggaaagccacatgacatttgctgtgatttgcacggctgatgggaaaacactacttggttctgaaggacagggttgtcattggatacctttgccctctgacccgagtgagaatctcacctggacaaagacccgagttgtgaaatcacacagagaaagtgtgattccgaacctgctgctgtcctgtgcttcctgaggacaagtgagtgactgactccgtgtgccaggaactgcaggaggttaaaaactttcagaactaccatgcaaataagaaatttcaaaggacttttctctaacacacatttcatagtgaacgtctcagctggattaacataattaacgctgtgttctttctggctcctgaaagaaattgtgattaggaacactttcagggctgatgagtctaaggtatcaactttgtcccttctgccacttgaagttcaaatggaaatgtttgctttcatagctccagtcatgatgttactgtatttagtagctattaagtttccaattccatagactcctgggagaagttccagttcgcactaaggcttctgttgcatttagcaccactgcctatggtgtgtggagctttcctgaacaagtaatagcagcaaggttcttagcttagtaataattttaagctattctttgattttttaaacaattaattaattaattattttgagacagcgtctggctctgtcgcccaggctggagtgcagtgacgtgatctcagctcattgcagcctccgcctcctgggctcaagtgatattctccagcttcagcctcctgagtagctgggactgcaggcgcatgccaacacgactgactaatttttgtattttttgtagagacaaaaatactttttttatgtagagacaaaaaaaaagttgaatgataaaatcttttattacttaataatatactttgattgagatttagccatttcattttcactagtgtccagacgttttcctgaaaggcgtgattccatgagatttaatactgaattataatttgatgtaaatgttttatataatcagttgtttgtttttaaacagtagcctcttttctaggcctgctctaggttgattaaatattaagttttctgatcagccagatccttaatagagagcttcggggacagcaaatggttgaatatgaaaccaacctcatttgccatgttgttagatataaatgtcagtgatagcactttctggatggttgagttttctgaagagcagaggattttctcttttaatcaatcagataattttctgttttaaccaattcaaacatttgattttatgtttctgaaatagataaagcactttcataaaaatggactgtgccaaataagaggtaattttcttgatgactcagttatttttgcctcttgttatttggcttttactaatatagcaatttctctataaaaataggaatctttgcttttttaaaaaaactgagatcattctgtttttccagttcttacattttaaaaaataatttttttggcttcttttgctatccatgtatgtagctgttggtgtgctttcctgtatcacatttgctcctggtttttacttcgttacattaaaaaaccaaaccaacaagtaattaagctcgttcagcctgcagatatcgtttgatggaaagggctaaggagtcagcctttgtgcaggtttttagttgtcagctatagtctgagtaagtgaggttgctaggtacaggcgttcaggatgtgtaagatgttattacagttatttttagtgtattattaagggggaggaggagaaagagaaatttgaatcaaatttgtgtcttattaaataggacacgttttccataagaccaaaagggcatttcttttgcttatgtactagaaatagaaaatggaaattagatattagattgtataatcgaaagtcaattagaattaatgaacaattctataactagttatactgtaagctaaaatgtgaatgttaaattagggaagtattgatttttatgaaaatttaccagaatttcaggaaaggactttgcttactttcccaagaaacgtctggtaggagtcactgtagaaatttggtgtgttctgtacaacactgcccaggaggaactggccacatttcttcctacttggttcttttgtttgtttgttttttgagacagggtcttgctctgttgcctagaccggagtgcagtggcacgatctcagctcactgcaacctccgtttcctggattcaggcaattctcgtgcctcagcctcccaagtaccggtgattacaggcacccgccaccacacctgtctaattttttgtatttttagtagagacagggtttcaccatgttggccaggctggccttgtactcctggcttcagatgatctgcccgcctcggcctcccggagtgctgggattacaggcgtgagccacagtacccagcctgctttatttttaattgaagctaaattcacataacttaatgatttttaaggtgaacagttcagtggcatatcgcacattcacagtgttgtgcaaccatcccctgggtctaattccgaagcgttttcatcacccctagaaggaaaccctacacccactaagcagccacttcccagctcccaagccctagcacctggcagccgctgtgctgcactttgcatctgcaggatttaccgagtctgggcgtttcttatacttggaatcgtaggatacgtggctttgtgtgtctggattctctcgctcagcatgtttccaaggttcattcatgttgtggcatggagtagtacctcctttttgttatgggtgcataaaattccattgtatggataaaccacattttgtttttccattcttccattgatggacattagggttgtttctgcctcttggctattgtggatggtgctgctgggaacactcgtgtacacgtttttgtttgagtatctgttttctcagttctttggggcacatacctgggcatggagttgctgggtcacgcggtaactctgtttaactttttgacaaacctctgccacttttaaaatgaggaacttgggctgggtgcagtggctcacgcctgtaatcccaacactttgggaggctgaggcaggaggatcacttgaggtcaggagttcgagaccagcctggccaacagggtgaaaccccgtgtctactaaaaatagaaaaatcagctgcgcatcatggcgcgtgcttataatcccagctactcgggaggctgaggcaggagaatcgcttgaacctggaaggcagaggttgcagtgagccgaggtcatgccactgcgctccagcctgggtgacagagtgagactctgtctcaaaaaataataaaataaaataaaataataaaataggaacttatatgaagaccctgtaaaaagttacctgtggcagtccgatgttttggaaaacatctgaaagcttaggtcaatttggtcatagatgtggctggccattatgtttcttgattgaaataattactttgttcactaccattcggaatagttttgtgattaggataaaaattataatgtagatataagagcctagagactctttttatatgaatgcataagttttggctgttcccttcaaattggcatgaggcttggcaacactggaattgtgtgttagataaacagttggtatctagggcaagcttgaaaaaaaagtagtatttaaacattgaccaaaaaaaaaaaatctcaacaactctaggaacttccttgttcaaatgaatccacaccgtctaaatacagcgtgataaaattaggcttccctctggtttccgtggggtcgagtgagccctgagcgtctcgtgcccagccaggactctgcctgctggctggcttcggagcacagggaggtgggacggtttccacggccagcagtgttcccacagcacttccttaatatagaacgtttttaaaagcagcgttggccacagaaaaagctcaactcccaaacactgtcatcagttcatagttcatttttattttgttttcatgttgttaaaacaagaaatagcatgtaaacatgtctgcattaccgaacatcagtagtgtttaggatagaagaggaacacattgagtttctaagtagaaaaaaaatctgttcatccttaaaggcaaaggaaggggcaaggtccctccttgaacaactgggtgaggttggacgcctccagctgtatttgctaagttgggcttggaagtatccatagggcgtggggggcctgctccagggctgacctcagctggagtcccctccaagtcaggggtatcgctcggcacacatttgtatgagttcacctgaaaagtgcccttgttgggggagcttggaatggcctgtagggcagctgggtcacagtggctgccggggatgcctgccaagctgttaagcagccctcgggcccacggagtcccagcaagtgtggtttactgtttgttaggatgtaattcaagcctgtgggtaggtaaaactacccaggattaaggggggttggcttttccagggaacaaaaggatcccaccagggagaggactgttctgggaagtgccctggtcccctgtggccaggcttgatttgctctctctttggaagccaccttggcacgcaggcactggtggaaaattttggagtacctgctggaggttgaagatgtgagcacgaagaaaccatggtcttgtctcgggagcctcccagactttcccctttacggctggtgagaacgtgaaatggttcacttgctgcagaagacagtctggcaggtccccaaaaggttaagcacggagctaccgtagggcccagccatgccactcctgggtttgtgcctaagaggtgaaaaacacacatccacacaaaaacttataggaatgttcatagcaacattatccacaattgccaaaaagtggaaacagcccaaatgcctgtctgctgatgaacacataaacaaaacaagtaacacatcaacaaaacgtagcacctctgtaggatagcatacgattcagccttaaaaaggaatggagttctgatacatgtgacaacgtggacgaatactgaaaacatgcccagtgaaaaaagccagaccacaaaggccacgtttcgatcgcttgagcccaggagttcgagaccagcctgggcaacatgtcaaaatcctgtcaactcaaaaattagccaggcatggtggcatgcacctgtggtcccagcccctcgggaggctgaggcgggaggatcacctgagcccaggaggtggaggttgcagtgagctgagattgcctgggtgacagagcaatctctgtctcaaaacaaaacaaaacaaaaacccaaatgccacgttttatatgattccatttctaggaactctagaactacaggcaaatccatacacacaaagtagattagtgatggccagggctggggaaactgaagggaaatgggaggtgaccgccgatgggtatggatggggtttcctttcggggtgatgaaaatgttctcgaatgggctatggtgatggttgcacaactctgtgtgcgaaacctccctgaattataaactataaacgagagaattacacagtatatgaattctctctatataaagctgttatgaaaagcgaacctaccagaccaacattaacaattagagtatattgtagtacaagacagttgtagattggcaagataagattggacatagagattttgtagattgtggagcggagagggatgaggtggacggtgctgttacagcctcttaggagggacagagatcctgaggtgatcaccgagcttcaggatcttgttcataattttgagattaagaatcttggtttggaatcccatgcctgtcatcccagcacttcgggaggccaaggtgggcagatcgcttgagctcaggagttcaagaccagcctggttcacatggtgaaatcctgtcgctgcaaaatgcaaaaactagatgggaatggtggcacgtgcctgtagtcccaactactctggaggctgaggtgggagggtcgcttgagcctgggaagttgaggctacagtgagctgagatcatgctgctgcactccagcctgggcagcacagcaagaccctatctaaaacaaagacaaaaacaaaccagaatcttcatttccatcagtcttcagaactgtgtgagtttaggagttgtgattcatgctgaccatgagaccgatgtataaagcaggtgccagtgtattaagttactttggggaatatgatgtacttttccagtggctggaatattaatatctacttcccaaggcagttcctccatgagatcaaggtatctgtgaggcctcattaatagtaacaagttagccgggcatggtggcacatacctgtagtctcagctactggggaggttgaggcaggagcattgcttgagctcacactgctgcacaccagcctgggtgacagagtgagactctgtcgcaaaaaaacaaaaaaaaagaagaagaaaggaagaaagaaacgaaagaaagggagaaagagagggagagagcgagagagacagaaacaaagagaaacaaagaaaagagaaagaaggaaggaaggaagagagagagagaaagaaagaaaataaatagtaacaggattgcttcaggactgtagtatccgagcaaatagtaacaggattgcttcaggactgtagtatccgagcaaatagtaacaggattgcttcaggactgtagtatctgagcaaaggatctgatttgatccagcacatcccacgagggactgagtgtttatgcccacagactcagtgccctccctgaactgtgcagagtaaactctgccaaactcttagctgttgttgttcttcttttttttttttttttttttatctttttgagatggaatctcgctctgttgcccaggctggagtgcagcaatgtgatcatagctcactacagcatccacctcttgggttcaaacaattctcatgtctcggcctcccaagaagctgggattacaggcatgtgccaccacatgtggctaattttttgtatttttaatagagacagggttttgctgtgttggccaggctggtctcgaactcctggtctcaaccagtctgcctgcctcagcctcccaaagtgctgggatgacagacgtgagccaccgtgcccggccttccttgttcttaaagaaggagatgttacagaacaaacgaatctccatacaaaacaaaacctcaccataaaagaggaaactccctgcttcatgaggggtgcaggtaaatgtagaaacgctcaccaagaaagccagagtggatttcacattaatagaatgcaggtaaaagacataacttgttgggatgtagggcgcagtatcagaatggaatagggttcaggtgagctcctaggatggcaggatttgaagaaggagcagcaccttgattaggggagaatgaagaagctttggagggctgggcgtggtggctcacgcctgtaattccagcactctgggaggcctaggtgggcggatcacctgaggtcaggggtttgagatcagcttgggaaacatggtgaaaccccatctctactaaaaatacaaaaattagccgggcgtggtggcgcatgcctgtaattccagctactagagaggctgaggcaggaggatcgtttgaacccaggaggcagaggttgcagtgagccaagatcatgccattgcacttcagcctgggtgacagagtaagaccctgtcttaaaaaaattaataaaaataaaaaacacagtccaatttttttgagctaattatggaagtattaatggataaaatctacattttgtactggtgctgagagtgagctgtcccctaagtagattatgtttcatgattctgtgtcaactttctctgggctgatgggccctctttggacactgacgtctcctcacgtccatgatccctcctgggtccgtgccagctgcgcgtgtcatcagagcggagcgtgcgcttgtccttcatcacaggcctgggtgatgtgtggctgacatgcgtcacatgggataggctctccgatctcagggctcgggctgccacccctgccacgtagtacatggcatgtggctgattctccacttgcttgcaatggaccctctgtggaaggcaccggagaggcggtttccctgtctcttctcctcctgtgttgccctcctccccctcgatagatgtgacctcccttccagctttgtgccagtgtctggcagcacttgattttttttctccttgtccttcatctgttttcattgcgtcgccaccttccccacccccaagcagcatccttctgccccttggtgctctccgcgcggccgtctgcctacctgtgagtacgctgaccagggctggttgactgcctacacattagcggagtctttctcattttttagggcacagtagatactcagcgagtgttgttttttttttaataattatgcaataaatgaatggatgtccgcacaccaccctgaacaccagcacccactggtcatccctggacacgcatgggtggaaggaccacactggttgtccctggaagagtagctcactctcgagcctgcaccgatgtcatctgagtcaaaacacagacgtctgggccccaccctagagagtctggctcagcgggtccagggtggggcaagaatgtgcattcctgcaagttcccaggtgatactgaggcggctgctggggggataccatttcaagaagcactgcttggaggcaacgcagacctctccctcttcgacgaggtcctgttcacttccatgtaaccagtcagggttggaatcccacatggagttcccctcctttggtgaccctctgctgcagctcttgggagcctcccctttagatgagttcctccctgctctgaccaggagttgctttttctataccacctcagctgatggtgttcacacacacattcatttaacacacatttacggagggctcctgggggctgggtgcacacacttggtgcggctctcttttggaacctggtgccttctctctctccttcatttttgaggcagagtcttgctctgtcgcccaggctggagtgcagtggtgcgatctcggctcatggcaatctctgcgtcccaggcttaagtgattcttccacctcagcttcccaagtagctgggattataggtatgcgccaccatgccccgctaatttttgtatttttagtagagacagggtttggccatgttggccaggctggtctcgaactcctgacctcaagcgatctgcccaccttgacctcccaaagtgctgggattacaggcgtgagccactgcgtccagcttccagtgtcctccctttcagcctctgggtgttgccagccagtgccaccactcagcactgtttatacctaaggcaccaatgggtaaatggagagacaggcgtctgaagacttttaaaaatgtgtactttatttggctgtagctaaaccagttagaaggccgggagttgaagttgatatgagtatcataaatgcgcaagttaagtagttacttaagaagtcacaggaatatgggtcgttccttgttgggtttctgataactagcaggcggctcagtttggctatgttaataacagaaacacagagattgccactgattctatcacatttcctgtttgtttgtttttgtttttgtttttgagacagtctcactctgtcatccaggctggagtgcagtggtgcaatcatggctcactgaagcctcgaccacccaggctcaagtgatccttccacctcagcctcctgagtagctggagctagaggtgtgcaccacctaatttttaaactttctgaattttttgtagagatggggtctcactgtgttgtccagtctggtttcaatctcctggcctcaagcaatacccccaccttggcctcccagagtgttgagattacaggtgtgagccaccgtgcccagccaacatttcattttttaaaaaatgctttattgaggctgggcccggtggctcaggcctgtaaacccagcactttgggaggctgaggcaggcagatcacttgaggtcaggagttcgagaccagcctggccaacatggtaaaaccctgtctctactaaaaatataaaaattagctgggcgtggtggtgggtgcctgtaatcccagctacttgggaggctgaggcaggagaatcacttgaacccaggaggcggaggttgctgtgagccgagattgtgccactgcattccagcctgggtgacagggtgagaccctgtgtcaaaaacaaaaacaaaacaaaacaaacaaaagaaagctttattgagatgtaattcacctgtcacccagcctgggtgacagggtgagaccctgtgtcaaaaagaaaaacacaaaacaaacaaaagaaagctttattgagatgtaattcacatgtcacacaattcagcagtttaagtgtacagttcctatactttgacatccatcactacagacaatttggggacgttttcttcactcgacagagaagcccggcacacccgagctgccacccccagtgctcccagcccctggcagcccctaagctgctttctgtctctttgcatttgcctgttctggacatttcataaaaatggaatcacatcatatgtggcccttcatgactggcttctctcactgagcctaatgttttctgggttcacccatggtgtagcatgtattagtgctttcttccttttcatggctgaataatattccattgcgttttgtttatctttgttgattatccattcctcagttgatggatatttgggttgtttcactttttgactatgatgaatagtgtttctatgaatgttggcagacacgttttcctgtggacatatgttttcatttctcttgggtatgtacctaggagtggaattgctgggtcatatggtaactctgtttaactttttgaggaatgtctggagtttgttttaaagtggctatgccattttctcttcccaccagagtgtgtgcgggccctgatttccccacgtctccataaacacttgctgctgttcgtcttttttattatagccacactagtggaaagacacttttgtatcccacacaaacagaaaacttatgtataggtgatttttctaaaaaaaagactttatgttttgataaaataagcatgttatattggttaagatggtttttgttgtgagttaacgaaaacctaactaaaattacctaaaacaaacaaacaaaaaaacgcatttactatctcatgtaacaagaagtatggcagttccagggttgattaattcagaggtgtgatgacatttccaggaccccaggtgcttttcatctttgctctctgctgtctttgccacgtgggagatgtcttcccttgtggcacaaagtggctgcagccattccgaatgacacagtgatatctagtaaagaagaggggcttttccaatcctgcatgtcttattttactggcatggaaactaagcagacatctctcagctccctttggccaaggcggggccatgtgccacatcctccttagttactggaaaggggagggaagccccattggggtgaacttaccctgctccagctacttactgcgggtgggctgtgtgtgcgcatgcatggtgtgtgtgcgtgtgcgtccatgcgtgggtgtgcatgtgtgtgcgtgcatgcgtgggtgtgcatatatgtgtgcatgcatgcatgggtgtgcatgggtgtgcatatatgtgtgcgtgcatgcgtgggtgtgcctgcatgtgtgtgtgcgtgcttgcatgggtgtgcctgtgtgcatgcatgcatgtgtgtgtgtgcgtgtgtgcatctccttcctaagcaggtagaagggagataccccaagcagaatcaggcttctgctggtaaggaaggtaggtgtgtgggaacggggaggacatgaagaaagaaaatgattcttagggatgcaatgcattagtggctgtgactttagttaaaacatagaattttgtttgttttttttcttttgagacagggtcctgttctgttgccctggctggtgtgcagtggcaatagtggctcactgtagccttgatctcctgggctctagccatcctcctgcctcagcctcttgagtatctgggaatacagtcgtgtaccaccatgcctggctaattttcaattttttttttgtagagacagggtctcgctatgttgccgaggctggtctttaactcctggtgtcaagtgacactcttgcctcagtctcccaaagtgctgggattacaggtgtgagccactgcccaaccttcatgcagcattttaaaaaatgaaaatgtagctgcttgttctaggagaaacttgtgaaattggatcatttaaccaactctgcctgtgcgtgacttgccttcagttctccagcttggcctcaatccctttttcttgctgcttggaacccacatcaccaccagtgacctcacaaggcagtgcccctccgcacccctcccttctccaagactcccccgccgccccctttcttgctcttacccacacgccactgcagacacagcgccctcctcccctgctgcccccctcttgctcttacccacacgcccctgcagacacagcaccctccccccacccaccgttacaccctctcttgcttttacccacacgcccctgcagacacagcgccctcctcccctcctgcaccctgggggctccccgcatgtgctgtgatgttgaacatgtgggtgtcaggatgaaaggaagaaatctagatttgatgtggtgggaggtaaaagcactctcgaaggctttcgagctgggctatgactgtcaggatgaatgtggttttaatatgtggaagagatttatttctttaatagtcacctgttaagagtcttagatatcactgatgtgtcttgttaagaaaccttataacaaacgtatttcttaaacttacatctggagctctgcaaagcattagctgttctgcaggaatgacagagggtctcctgtgcggccagctcctctggagtcagggagccatggagccactgctggttctgaggatggtgacagcaggaggtggagatggcttcatgatctgctgtgagcaacgtgcaaccttctccctgctgttaagtgactagaattcccaggtgccgcctgctgattagttccagtttgtgttccacgagagcagatgtgtgtgaggtgcagggaatgccccgctgcgggagatggttttctcctgccaccgtccctccccaggccacaccatccacacacatgagcacgctcgtcttcctttctcataaagcacagacctagaaatatttctcggtttaaaaatagtgttttccctacctcgagtcaaggttcagttctcatctctctaattttagttccttgtttttatgaactttgaaatgttttccttaaccagaaaacagaaaaaagcaatcccagagacaagggaaatttcttgctccatttccctgtagtaaaggcatttctatgtgagattttctttttatcctgagcaggttatcagaaaccaaaattttgaattaggagtttaaagaagaggcaagggggaaaaagtgctctctacgggaccttggttgccatgtggaatgtttttggagcataaaattgatatttttacaaaaaaaaaaaaacgaaaaaaatgtttctggtagaggcctggagaacgccgtcatgattctgaaggtgtatatattttttattgtttctgtcaaactaattttatataagacattaatggaaaggtcaaaacacataattctctgaagcctattagaaaaatttcttccgttgggctgggaacggtgggtcatgcctgaaatcccagcactttgggaggttgaggcgggaggatcactcttgaggccaagagtttgagactagcctggccaacgtggcaaaaccccatttctatcaaaaaatgtatatatatatacaaaaattaatcgggtgtggtggcgcacgcctgtagtctcagctactcgggaggctaaggcaggagaatcacttgagcctgggagatggaggttgcagtgaaccaagagcatgccactgcactagagcctgggcaacagagcaagactgtctcaaaaaaaaagtaaagaaaagaaaaattacctctgttaacttctttaaattcttaggtacaggtggtaatttttttctttgttaaaattgatgttggtcattttgtgtttgagttttctgttgctatttcatggggattttcctgtgcatctacataatatagtatttctttgaattaatatgccatagtttaatcatttcctgtttgagctttttagattgcttgaaggtttttctttcctttgtaaatactgcagctttaaggaatgtgtgacttttgaagctctctgttttggcagatggcaaagggggtgaccatcttatttttattaattttttattataaaagtaaaacatgtttttggtgttttagaaaagacagataagcaaaaagaagattaaaaaaacatttaccaactagagatagccattttgaatgtttcaacatgaggtttttagactttttcagtgcatatacgatatatatcaaatatttattcaaaatcaaatatatatcaatgcatataaatatatatagatataatggtgtcatatgaaacaccattaacaccattaaacaccattaacaccattaaacaccattaacaccattaaacaccattcatggatcatgaatacctttccacttaaatctgtatgtacatctttgtttattttattttattttatttttttgagacagagtcttgctctgtcatccaggctgaagtgcaggggcatgatctcggctcactgcaacctctgcctcccgggttcaagagattctcctgcctcagactccccagtagctgggactacaggcgtccgccaccacacccagctaatttttgtatttttagtagagacggggtttcaacatgttggccaggatggtctccaactcctgacctcaggtgatccacccaccttggcctcccaaagtgctgggattacatgcgtgagccaccatgcctggccaagtctgtacatacatctgtatcatcattttaaatgacttagattttattctggggatgtactataatttattcaggaaatatctcttgttggaaatgtagattgtttccagctttgaatcattgtatatcacaatatgaaactgttaataatccagccatccctcacatcaggatgcattgtataatttaatggtatgtcatagtttaattggcagcactttgcattatattttaatagaacaaaaagtaatgaaacatcatcattggcatcttagactcgatgaaatataatattgtcaattataacagtgtgatgaatatccttatttgccgttgtctttgcccacgataattagtagtggggccaaatctctttttcatatgtgtgttgcccttttgtatttcttcatgagttgcttgtgaattggttgtcttttccttactgatttgtgaaagttctttatttatgtaagtatcttaaatctttgtcttccatatatgttgtaaaatgcctcccattttctaatttgtctttcaatcgtgatatttcttgatatgtggaaatttaaaatctttatgttggtcttatctatcaatcattgtttatggtttctacctttagtatcatggttagaaaagccttccccacctgaggactatgtaaaatattaacctatattttcttctaatacttttatgtttttgttttcttttttttttttctttttttgagacagggtctcactctaggcaggctggagtgcagtggcgtgatctcactgcagctttgacctcctaggcttaggtgatcttcccacctcagcctcccaagtagctgggactacaggtgtacaccaccacacctggctaatttttaagtattttgtagagatggaggtctcattatattgcccagtctggtctcaaacttctgagttcaagcaatcctcccaccttggcctcccaaagtgttgggataacaagcatgaaccattgtgcctggactggatataccaccattttaaaaaatcagtttatcagttgatgaatatttgagttgcttgcagttcttggctattatgaatagagttgctgtgaacattcacaagtcttccgtggatgtatatttttatttgtttcggttaaatgcataggagtggagtgactgggttgtatgatgagtgtgtttaactttataagaaactatgaagctgaacatgttatttttctttgtaactggacacttaattgaatgcttattgtgtctgaattttttttttttttctgaagcagagtctcactttattgcccaggctggagtgcagtggcaccatcttggctcactaccacctctgcctcctgggttcaagcaattctgccgaagactcccaagtactaggattacaggtgtgcaccaccacgcctggctaatttcttttgtatttttagtagagatggcgttttgccatgctggccaggctggtctcaaactcctgacctcaagtaatccacccacctcagccttccaaagtgttgggattacaggtgtgagtcaccgcgcctggcctgtgtctgatttttaaaaatcagatagccttagatttttcttggcagaaaatcatataaatttaaaatcccctccttttcagtttttatatctcattttaaaaaattggactgaaaggtaaaggccaaaaatatgcctattctctgcttaggagttacagtaacttttgatgtgtgtgtgtgcatatacctgtgtgcgtgtgtgtgggtgttttcccgctgtaagaaaagaaggcatgatgtaatgtgcaggaaataatcataataaagagaatgctggcgtacttcttgagtgctcattgttgctaaattggtcatttactcctaaaggacgttggggaagggtgtgtttgatctctctacagccagaaagaccacctgaaaaccctcctgggcctcgtgaatctgctcatctctccagatgtcacaggaagtgtcctcggggggggacaaggctgtgctgttccatgcaggcatggtctcaggctgttcccaattgtgagtgagttttgaacaaaactgtaacttccctgtatagggcttacagttggcctcatcttacaccctgtagtccagtgagtccgcatgctagctgtggaaagcgtgtacagaaaggaggctgattctttttttttttttttgagacgaagtctcaaccttgtcccccaggctggaatgcaatggcgtgatctcggctcactgcaacgtccacctctgaggttcaagcgattctccttcttcagccccctgagtagctgggattacaggtgcctgccaccatgcccggctaatttttgtatttttagtagagacggggtttcaccatgttggccaagctggtctcggactcctcacctcaggtgatcctcctcccttggcctcccaaagtgctgggattacaggcgtgagccaccgcccccagccaggaggctgattcttaattattgatgcactgcagcctgggcaacagagcaagaccttgtctaaaaaaaaaattattgatgccatttggttctctttgaaactaattaatggctccttaatttgtcagtgcgtctatcttctagcgatcacagatgctgtttgggtgctcttgtcagtttttggtaaccatttctggtattttcttttcaaatccccagcctcactttgtgctggttatcacaagatccacttagaccttagaggtgagagcttgggaatatttcatctgcaagaaaaatcattcgtctgtctttgtgtgggacgagttaggttatgtaagctgatggaacacgaaacagaaaactggagcctttttctttgggcacacaaaaatgcaaaaatctaaatctcttattttgttgaatttttaagtggaaggagttgtagatttgccaccatcagtcctggagctgacttgaatttttcttttaggctcttgttagcttttctaacagagatgtcacagattagcaactaggcataaacctgagaaattgggcactaattttgttttgcatttttgttttgtaagacgggggtcttgctgtgttgcacaggctgcagtacagtggctgttcacaggtgtgatggtagcgcactgcagcctcgaactcgtgggctcaagtgatcctcccacctcagcctcccaagtagctgggaccacaggcatgtgctacgccacccagctgttattttgcatttgctctgtcatcacgttgtgcttttgtgcctgccttttctcctcattaactagatatctgtcacttaaagaaataacagcgatgcatttcaggataatagaacccctatgaactcttccagacttaaaatttatctgtttctccaaaaaacactgcctgcatttttcccaaataagccagatactctcagccaggtatctgtgcaggttggacccctgcccaccttacaagatccagtagcatctccgttggaaagcttttcctgagccctcccgcctggcccccccgctgccccagccgccattccttgccaggtctctttttacctccttttgcgttaatgggtctttggagttgtgtctccccttcactgagctctccaggacacattttgcaacatttttgtgtccctggtgtatggtgtttagcagacgctcagtatttacatgttgacagattgcacacatgggaccgggtacatttcttgccagtacatccagttttcccttacctcttctgctgtaaattaatgttcctctggggtttaaaaaattaatttagtggttaatttagtaagagattaaaattaggcactggccaggcacggtggctcatgcctgtaatcccagcactttgggaggctgatgtggacagatcaccagaggtcaggagttcgagactagcctggccaacatggcaaaaccccatctctactaaaaatacaaaaattatctgggcgtggtggcacatgcctgtaatcccagctacttgggaggctgggacaggagaattgcttgaacccgggaggcggaggttgcagtgagccaagatggtgccactgcactccatatatatatagtaacaggtctcaccttgtcacccaggctggagtggagtggcatgatcacagctcactgtagcctcagcttcctgggctgaagtgatcctcctacctcagcttcctgagtagctgggactacaggcatgcaccaccatagctggctaatttttctatttttttgtggagacggggtctcaccatgttgcccaggctggccttgaactcctgggctcaagcgatcctcctgcctcagcctctcaaaagtattgggattacaggtgtgagccactatacccggccataaagtagtacttaaaagtgtaacaactttcaataaatctttgtgagattcagaacaaccttttgaattgtctatgtaaagaaataccgaactgcttttttttcccctcgatcactttttaaccttccactttgtagacaatttgctagtgtgttttctatttttttttttttttgccactgggtattgccctgtcatccagactggggtacaatggcatgatcgtagctcactgaagcctcgacctcctgggctcaagcgacccacccgcctctgtcgcccaagtagctgggaatataggcgtacaccaccatgcccggctaattgtttttttaatttaaattttgtttttttttttttgcggaggcagatcctcactatgttgcccagcgtggtcttgaactcctggcctcaagtgctcctcccacctctacctcccaaagtgctgggattacaggcatgagccactgtgcccagccagatttttgtcatgattctatttttatctcttctatcgatttatttcttatatattttttctcacttattttaagtcattgcccgaaatttacaaagtacattttttatcagtctgcctccaaatattataccatttattatataagaacctcgtaagaattcttcataactgtaagttactacaaggttctatgagaaccttgtaaatgtttatgatgctaatagtgtcataataatgtattttatatatattataaacctcataacatgttatttttacagaaaattaccttttagagcaatgctgtttgatgaaacttcctgcaatgatggaaatgttttatatctagactatacaatatggtagctactatccatgtgtgaccattgagtacctgaaaacaactgaattttaaataaactagtttaatttaagtaatttaaatttaagcggctgccatatagacagtgtagttttagaacaatggaagattttcaaaatacttttgaaaattattttgaaaatctttcttctttttaggccacttcaggaactcttcgtttctttgtgtagatctataggtttctgtctcatctcccgactgaaaaacttcctttaacattttttatgctacaggtcttctgggaataaatgatctcagcctttgtctgtctgaaaaagtttttatttccctcccattagaaaatgtttttaggccgggcacaatggctcatgcctgttattcccagcactttgggaagccgaggtaggtggatcacctgcagtcaggagttcaagaccagcctggccaacatggcaaaaccctgtctctactaaaaatacaaaaattagctggatatggtggtgagagcctgtcatcccagctacttgggaggctgaggcaggagagtcatttgaacccaggaggtggaggttgcagtgagctgagatcgcgccattgcattccagtctgggcaactgagtgagacttttgtctcaaaaaaaaaaaaaagtttttaaagagggctgggtgcggtgactcatgcttgtaattccagcactttgggaggctgaggagagttgatcacttgaggccaggagttggagacctgcctggctgacaaggcgaaaccccgtctctaccaaaaattagctgggcatggtggcgcatgcctgtagttccagctacttgggaggccgaggcaggagaattacttgaacccaggaggcggaggttgcagagagctgagatcacgtcactgcattccagcctgggcaatagagtgagactctttctgaaaaaaaagaaaatgtttaaagaaaaatttcataccaaggagatccaagagacactcatttttaagagtcggttttttcttttttttggttttgtttgtttcttttttttttttttttcttttaggatatgaaggtttgtgttgaaaaattttttttgcaacttattcttttagtactttaaagatatcacaccactgccttctggcttttgttgttttggtgagaagtctgctataattcttttctttgttcctctgaatgtaatatgtttcccaattcccactgtcaccccggcagcttttaagattttctctttatttttagttttcgacaatttggacatgatgttgttttggtatttctcttttttggagtcctctaaacttcttggatctgtgctttgatgtcttttattatgtttaaaaaattattggccattatctcttcaaatatttccttctatcctttctttcttcttcaagcattttcttttttcttagaaacatttaataggggcttatgcacagaagctatgtctgttctcaagcttctgttcattagtctgtgaactggggctgcagcgagaagagacagtggatccctgcaccattatcccctggtcccagggcttgtataccgtagggaaggggtgattcagagggatgtgtaggtcagtgtgagtacaataacatcaaagttgtttgacctaaggtcaggctttatggtaagttcttggtcttacacgaggaacagcgcataagctggaaatctcagagggtttcctggaactggggttaatcagaagccacatggcggagtagcatccacgatggagctacttcagcctccacagtccactcttgtaatccagctcttgtaatctcacatgccctcttcttccttcatggtccctgagcctttaaggagggtgcttgatactgtatagctttagcagcagtgccttggtcatggaaaacagatcgggcccagtgggattccaaacaagggagattcacaggcttttaaatcgtcttcagtcttcagaatgcagtggctttggttctgtcagaataggcaaaacaatgtgagatatataacatcgatcactcggatagtagaattctgtgcacacagagattgcaatagaaagagaatcgcctgtaatcccagctactcaggaggctgaggcgcgagaatcgcttgaacccgggaggtggaggttgcagggagccgagaccgcaccaccgcactcccgcctggatgacagtgagactctgtctcaaaaaccaaacaaaaaaacccagaattcgtgggccagaacaacaatgaaaaaagaacgtattctgttaagaagccaactaaaatgcatcatgatgaaaattaaaactcggttcttctttagagacttgttgcagccaggaaatggttcaggattagcccaaattgtaggcagataataaaaactcacaaacaatggttaaggctagaatctaaatttttctttctccagtttccccatttctaccagggataaattatatatataatagtaagaccaatttatttgcaaaacaaattttagttgctttatacttggcctgaagatttgcatcaaatgtagcaagaattgtgatccaccatacggaccctttttaagttggctttgcaggaacctttataaggaattttggatttgactttttaaaagcctcaatgccagatattgatctgttttcaacttttttgattttttttttttttttttttttttttttttttggagactgagtttcgcttttgttgcccaggctggagtgcaagtagcaccatctctgctcactgcaacctccgcttcccgggttcaattgattctcccgcctcagcctcccgagtagctaggattacagacatggatcaccatggctggctgatttttgtatttttaggagagatgggatattaccatattggccaggctggcctcgaactcctggcctcaagtgatctgcccacctgggcctcccaaagtgccgggattacaggtgtgagccaccacgcctggctactacttggttactttgtaaccccgcctctttgatgggtcaagaaaagttgtggttatattatttatcttggtttttattattattattattttattttttgagacggagactctctctgttgccaagactggagtgcagtggtctgatcatggctcactgaagccttgaccttctgggctcaaagtaatcctcccatctcagactcctgagtagctaggactacatgcatgcatcatcacacccagctcatttttatgtatttttgtgtgtagatggggtcttatgttactcaggctggtctggaactcctggggtcaagtagtcctcctgcctcggcctcccaaagtgataggattacaggtgtgagccactgcacttcgcttttttctttttaaagagaaaggatctctctatgttgcccaggctggacttgaactcctggactcaagcgatcctcctgccttggtcttcgaaagcactgggattacaggtgtgagccaccaggcccagccagttttaatttctgttaggtggaatgacattccaggtttctaaaaataacttctacttggttatttttcaaaagttttgttgagttcagtaattatataaggtttataacataatattatttgaaactttcatccacatcctattctttactggtagagaaaagccacatataagaaatacatgtgtgtttgtcttttataaatatatgttacccagttttccagttactagctgcaagttttttttccaggtcataatttggagaaaaatatatgcgtgaaaattataacgataaagcttattttctatacaacaaacagtgagtgatatggaaatgtatgtccttactagtaattttaaaagtacaaaagagaatgccttaaaagtacacacacacacacacacacacacatacacacacatacatatttttttgagacagagtctcactctgttgccgaggctggagtgcagtagcgcaatcttaactcactgcaacctttgcctcccgggttcaagcaattcttgtgcctcaccctcctgagtggctgggattacaggcatgtgccaccacgcctggctaatttttttgtacttttagtagagacagggtttcaccatcttgggcagactggtctcaaactcctgccctcaagtgatccacctgcctcagcctcccaaagtgctgggattacaagtgtgagccaccatgcctggccgcattatatactttttaaggcagagaaaattttctaaaacttgatgggtggcctgttgttgctggttcacttcttctaaagaacagaagtaagaactgtaacactgtccgaatcctgggagagtcgccttagaagatggtcctgtagatacaagagaataatttactcaatgatacgaatataattgagaatagcaaaaattgaacagcagccaaattggtgatgagtgaggtaactctggtaagtcaccctgatagaatatattccatttattcattgttcatagaatatattctatttattcgttgttcttccaacctttgttgaagccccactctgtcagactcggcaccaagtgtctgagatgccttcgctcattacatcttcacggctcccccgagaagtagatgttactttcattatctgcattttacaaccaaggaaccgggatattaaattacttctctaggtcatgcagctagtgtgcggtgggctaaatgtacaatccccctaataataataataataataataataataacttacaacaacagcatattgaaataagaaaaaaaatttgaaaaatctctttgattcgtaaagatgaaaaaattgattctagttgttggtggtgagaggaaagggaacgcaggccctctgaaacactgttggtgggactacgaattgcagaaacctttttgaggacattttgcatatcttcagaatttcacatgtcatgccatttgaccaagtcctttcacttttagggatttaccctatggcaatatttgcacaagtacaaaaatgtatgtatgagaatcttggttactgtctttcttgtaatagcagaaagtgggaaatcacttgaacatctattaggtggaaattatggtctgtccttatggtggaatagttgtaaccatttgtaagaatgagatagatctgcgtgtacccatatgtgcaaaagagccaccgtgaattgtttttttttttttttgagatggagtctcgctttgtcactcatgctggagtgcagtggctcgatcttggctcactgcaagctccgcctcctgggttcacaccattctcctgcctcagcctcctgagtagctgggactacaggcgcccgccaccatgcccagctaatttttttttgtatttttagtagagacggggtttcaccgtgttagccaggatggtctcgatctcctgacctcatgatccgctcatctaggcctcccaaagtgccgggattacaggtgtgagccaccgtgcccggccatgaactgtgaattgaaaaaagcaagcaactgtaccatatgtaaaacatgatctcattttccataaaatagcgtgtttaataatgtgggtctagagagataataatacatgcattgaaggatatatacaaatttatatatagcgcgtgttctttacagtgggattagaggaacttgtactaatattttttttgctttgttttttttttgcaacagatatgattttattattagaaaaaccagatttaaaaaaagccattatatgagttttcctagttggcaaaaattcattatatgagttatcctagttggcaaaaaaggcacttaagtgaggaaaggagatgcaaaatcatccttgactgtaaaggggtgcaggtgggatatgagcttgtctgggtatgagaatatcatgttctatgcattttttatttcctaaactgcttaaattataaatcattaatggtaattattaaaagactttgttttccctacaattagaaaaacttatttgaagacctcaaatcccaaaagtgaaagaggaaaaaaaaaattattgccacggtttttcccttaacccatttaattaaagaattaattaaattctatgttgcagtttttcaccaaaagtgcaacatagagttagttcagtcaccacaaaaagattcagagattggattttgctgacttagtgaagctactaagatacaaattcacaaagtaaatgtttgcacacattagctacagtggcaaagtgtttgggaggttgcagtccacctcccggtgagggagatccatccttttggtcagcaaatgccctgaattgaccctctcccgagggtcagctgcaggctgtgctgatctcggaacttctctgggggtccagttctgtttaggacagaccataaaatacatctctctggggtgggtagagctaccccagtacttaccctggaaatggactcagctgtccctggagggtgactcagttgaggcagaattgtcttaatttggtaactctcctaatttgaagcagaagtcctgggtttgacccagacccatcttggtctggagtggagttgtggtgtgtctaatgctgccagatcatgttcactgttagaagcctggggaagggaaggaacttgctcagccaactcagaagcaggcatagcgcaagagggaggattttaagaattagtattttactatccttgtgagctggtgttattgacaaacattatttcaatctgtttaacgattttaagaattagtattttactacccttgtgagctggtgttgtttctaaatattatttcaatctgtttaacaattccattttttaaaaaccctgcactttgggcaggagttcgtatttctaatttaggatgaattttttctcatctccccccattctgtgattctaattttttttaagttatttcagttcttaaaactctttcatagagctgggcatcagccgcaattttctctaaatctgtctcagatgagcacttgcagtctaattgtataaaagatcaggaaatttagtattgaagttgcacttgctttgatgtaatgatgaatgtttccttcatctgaagagcatttgaaatttatggaaaagtaaatctttttgaattatatgtaaatgaaaacgttgggatgaaccaggttatgtttaactgggcagttttgaaattctaatagctcctacaagcgggaatcttttaaagctaattttgtgatatgaattgtttattattctaaagaataatattcacttttttttcatggatctcatgtaaatgttactggacaatatgacccatctctttaaaaagtgaaattgttagtgggaagctgtggctcacacttataattccagcactttgggaggctgaggcgggaggatcacctgagcccaggcatttgagaccagcctgggcaacagcgagacctcatttctacaaataataaaaacattagctgggtgtggtgacatgcatctttggtccaagctactctggaggctgaggtaggaggattgtttgaacctgagaggtggagcctgcagtgagccctgatcgtgcccctgcactccagcctgggcaacagagtgagacctccatctctaaataaataggtaaaggtaaaatcactgctcagtcactagagagaggagagagaaaagtaaatatactcaaggagtgggaaaagctttgaagtggcatatcaaaatgagaaaaataagtgttttttagagagaaaagatttgtggagaattatatattagaactatttaagacttcttaaaacttcacgattatatggttttgtaaggtttttggggattttcataatgagatgtagatggaacgtccccattgcccatcatagactattcaggcaagttcatacccattcctagaatggagaaccgtctcctctgcggtgaagaagcgcccctgttccttaagaggcagcagcaggaggtggaaaacaacatcccgcttgctaaagtcatgggccttagtccaggcgcggaggctcacgcctgtaatcccagcactttgggaggccaaggcgggcagatcacctgaggtcaggagttcgagaccagcctggccaacatggtgagacccagtctctactaaaaatacaaaactgagccgggtgtggcagtgggcgcctgtaatcccaggactttgggaggccgaggcgggcagatcacctgaggtcaggagttcgagatcagcctggccaacatggcgaaaccccgcctctactaaaaatacaaaaattagccaggtgtggtggctcatgcctgtaatcccagctgttcgggaggctgaggcagaattgcttaaatccgggaggtggaggttgcagtgagccaaaatagcgccattgccctgcagcctgggtgacagagtgagactccgtctcaataaataaataaatacataaaaataaagccatgggcctcgatgggaggctgtacattgacgtgcgacattcgacttctgccattatttcagatttcgcacctgcccttctggcaacatggtgttgctgtgaggatcttgagaggagatgaatgatgctttccaagtagtaagtattattccattagggaaaataggaataaaattggaaagcatcaagtatttgaaaggaaacgaaaacagggcttactttccctttcggccagcgttagaagggtgcagtttggaattgaaagtgaaaggagcatgcacgtgttcggggcaggttgcggggagagtgaagatcattttaaagaccctggtgctttgatcttagagcaccactcatttgtactcagttcagatctaagtgaattactgtgcaaacgacatgacgagagaggggcattttccaatagtagcttctggtctggcacatgagccagttagagattgacacacgcagtcatgtagctgctataaattgaagcctggtgatggacgtactacaggcatggctgcagcgaatgcctatgacctttcacttctcctccctctgtcactctgtgtattcgctctctccattttcctccctcacctattcaccagccatgaatcaggagtggactgggcaaggatcagcctgtagggaaatgatgtgggccgagggtaaagagtgaggaataattaaggcagcccttgaattagttgtacatttttttcttttgtgtagtcttcttttttttttgtaagcaaacaaaaaccacgttgctggccaaatgtagtggctcatgcctgtgatcccagcactttgggaggtccaggtgggaggattgcttgagtccaggagtttgagaccagcctgggcaacatggtgagaccctgtctctataaacaaataaattaaaaaaattagctgggcatagtggtacccacctggagtcccagctaatggggagactgaggtgggaggattgcttgagcccaggaaactgaggctgcagtgagctctgattgtgccattgtactccagcccgagtgacagagtgagaccctgtctctcaaaaacaaaaaacaacactttattatggatgatttcaaacataaacaaaagtgaagagagtagtataatcaactctcaggtacacgttacccagcttcagttacccactcataacctttcttgttaatttctacaaaaagcttgctgggtcttgttgaatctgttgatcaatttgggaagaaatgccatcttaacaatactgagtccttcaatccaagaacctggtatctccttacatttatttagatattatttaatgtatcaacaaagttttgtaattttaacttactaattttgatgctattacaaatggaattatttcttaattttgtttttggattgttcattgctggtatacacaaaacagttgatttcctgatattgattttgtatcttatgaacatgttaaacttgcttattagttctagtagttttttgtagactcctgaagattttctatatatagagctagtgaatatgaagagttttacttgttttccaatctgtgtcttttaaatttctttttcctgccttattgttctggatagcacctccagtacaatgttgagtagaaatggtgagagtagacatccttgttttgtttctgaacaaagaaccaacttttggtttcatcagttttctctattactttccctaattcattgatttctggtctagtatttattattgccttccttctgctttttttgggatttagtttactctcctttttatagtttcttaaggtggaaactcaggtttgtgttttttttttttgagacatgatctcgccccgtggcccaggctggagtgcagtggtgcgatactggctcactgcagcttccaccttccaggctcaagagattctctcacctcagccccccaaggagctgaggctacaggtgtgtaccattgggccagctaatttttatttttattttttgtagacacagggtctcactatattaccaaggctcttcttcaactcctgggcttgagcgatcgtcccagcttggccttccaaagtgttgagattacaggtgtgagccactgttccctgctgatgctcagtttattaacttgagacttttcttttttttttcctgatacaggcttttaacattataaatttctccctaagtacttctcatagctgcatcctaaattttgatctgctgtgtttttcttttcatccagtttgaagtatttttaaacttcccttgtgatttcttctttgacctttgaatcctttagatatgttttctaatttctaagtagttggggagtccacaaatttatttctgttcttgatttttaaaaatttcattttggtagagaacatgctttatatgattttaattcttttaaatttattgatacttgttttatgacctagcccatgattcattctagacaatgcgatatgtacacttgaaaagaatatatattctatttgttgttgcttgacatggtctacagatgttagttacgtcaggttggttgacaatgctgttcaaagtctgtatatccttgctgattttctgtttaactattatgtcaattattgagagtgggatattgaagtctccaaacattattgtgaattgcctatttctccctttggttcagtcagtgtttgctttattgtattttgtgctctgctattaagtgcttatgtatttatgtttttatatattcctcatgagttgacccttttattgttacaaaatttctttttctcttataacttttttgtcttaaagtttattcagtctgtgggatttttttgtgtttttgtttgtttgttttgagacagggtatcactctgtcacccaggctggagtgcagtggcacaatcatagctcactataaccttgaattcctgtactcaagcaatccacctgccacagcctcccatacttactttgatattattatagccactgcaactttcttatggttttgtttgcatagtacatttttctccatctgcatctcttgcagacagcacatggttagaacttctaaaaaatttaatctgacagtcttcaccttttattggagtgtttaatccatttacatttaatgcaattattgatatgcttggattacatctaacattttgctatgttttctctgtctcacatctttttgttctgtttctccttactgttttcttttgtgttaaatagatattttctagtgttctgttttcatctgtttggtgaggtttttaatattttttggttatatttttagtggttactctaaggattgcaatatgcatctcatcacagtctactttttaaagatctatatatagcttttaaaaagtagagatggggttttgccatgttgcccaggctggtttcaaactcctggattcaagtgatccttccatctcagcctcccaaagtgctgggattacaggcatgagccactgtgcctagagctcacagtctactgtagattaatgctaattcattttcaataaaatatagaaactttgttccattaaatgtctatgtctatctacctcctctgtggtattatgtcatgtatatgatatctttgtatgtcataagcccagtagtaaaagtctgtagttattgttatatgtacttgtcttttaaatcagttaagtgtaaaaaagagactatttataaatctgctgtatatttgcctatgtaattagtattactagcattctttatttcttcatgtggattcaggatataatctggtgtttttttctttcatcttgaagggcttcctttagtatttcttgtaagtagggtctgccagtaacaaattttctgtctttgtttatctaggaatatctttatttctacttgttagttttggggtatggttttgatggatatggaattcttgattggaagtttttctttcagtgctttgaatatgtcattttactgttttctggtctctgttaatttctaacgtgaagtcagctgttaatcttatggtagtgctccttttcatgaagaaccattttgtcttcatgcagtcaagattttctctttgtttttggcttccagcagtttggctatgatgtgtcatggtggctcactttattgagttggagttttttgagcttactttgagtttgttggatgtgaacattaattttttaaaatcaaatgtgagaagtttatgccattagtttttcaaaaaattttttctgcttccttttgcttcgttttcttttgaaacttcctttatatatatgttggcatgcttgatggtatccctgtgtttctgaggttctttttatttttctttatgcttttttatttcttcttcagattggataattttaagttcactgattctttcttctcccatgtcaactctgttgttgagcccctctagtgaacttttcattacagttattatacttttgaactctagaatttctgtttggtttctttttataatttctgtctctttattagtattctctatttgatgagtcattgttacatactttcctttagttctttaaacatgaattccttcagttccattcttacagtagtttcttataactttgaaatctttgttaagtccaatatctgggcccccttagagtttccagttatgactcttgcctgagtataattcacaatactttatgtttctttgcatgtcttatattttatttttttaagctctgtattttagttaatacgttgtagcaactctggattatgatcacctttggcaaaacgcaggtttgttgtttatttctttttgtttgtgacttgcctggattaattctgtgaagtctgtctctgtagcagctgctgatgatcctgctcattttttttttcttatttttaaaatgtgtttaagcctggctttgcaggggttaacccaggtcagcatagcttagtggtcaggcaatgattattcagaggtgtgctgaaacaccttgagctactaatgcttttcacagtttgcggtggatatgtgtgtacgtttagggacatgttcagaattcaggcatatattatacgagcctgtcttggtttttcctttttgccaggccttttctggcctcctttgtgcgtggggtaggcctcactttagccaaggatgagtggataaccagggccctcttcagtctttcctgcctgtgtgcagccttccagagcacgaagaatgtgtgggacttcagggactttaccactgcccgtgatagctcccttattctctgtatcttccggctaggtttttggctggtctcctgatctgttggttgccacaaccggcattgcaggctcagggtggctgagatgttgaccttctgtacatttcctgttgtttacaggaaatcaccgtggtttatgtcagtgcctttgggcatgtgctctcctccaaatcaagtcaacccctccagcagtggagccaccaattttcacagctcgtcccaccctggtagatgtagcagcatgggtgagctgaggggaaggaggggagcccctcaagccaaagcatcacagaacaccaccgctctaactcaagatttggtcatttttcttcttcattttattttttaattcttgagatgaggccttcctctgtcactcaagctggcgtgcagtggcacaatcatagttcactgtaacctcaagctcccaggctcaagagattttcccacctcagcctcctgagtggctgagactacaggtgtgcatcaccacgccaggctgacttttttgtttttcgttgagacggggtcttgatatgttgcccaggctggtctctgactcccagcctcaagcaatcctcctgcctcggcctcccaaagtgctacaattgcaggtgggagccaccatgcccagctgcgtagtttttcttgaataaacatttttcaattttttgtatgtctttggttgatttccaaccctgtggtggttgtttttgaccgacttgtctagttttgttgttactttttgaggagaggttttgccaagcttctcacttcattattccagaagttcttcccctctccccaggtcatctttttggttttttcttctaactttctttctttctttttttttctttttttttttttttcgatggattctcgctgtgtcgcccaggctggagtgcagtggcatgatctcggctcactgcagcctctgccttccaggttctagcacttctcctgcctcagcctcccaggttgctgggattacaggcacccgccaccagctaatttttgtattttttagtagagatggagtttcatcatgttggccaggctggtctcgaactcctgacctcaggtgatctgcccacctcggcctcccagagtgctgggattaaaggcgtgagccatcgtgcacggccggtttttcttccaactttctaagtactataagcttgttgtaaaatttggcaatatagaaatgtataaaatggaaatgaaaatctttactaatcccatcccttaaagatagccactgttaataagttagtgacttgctttccaagctttaatcatctttctttaatcatcatatttttgattatatcctcttaccaaagttcctttacccctcaccacaaaggtttctaattcagagacttcctttagctcccttgattcaaggagataaagttaattagcaatcaggagatgaggagccctttcctggcaaggacagaattctaagacggagggatttttagctcctcggtggactcagttccgctgcaccttttgcagcatcttgcctgctgggtctcctctctgcagttttcttgttccctggccttttatcctcctgatcacacataccttcccaggttctccaggaaagtagtggatcagtcacttccccctgcaacgtcattgtaggcatgattacatcttggagtgttctctcaacaagagtcagtgtagcatggggttccgggcctgagctctggtgtcacattgcttggagttcaaatttcaattcttcttcttttttttttttttttcttggagacaagagtttctctgtttcccaggctggagtgcagtggggcgatcttggctcactgcaacctctgcctcccaggttcaagtgattctcctgcctcagcctcctgagtagctggtactacaggtgcacgccaccatgcctggctaatttttgtatttttagtagagacgggggttttaccgtgttggccagggtggttttgaacccctgacctcaagtgatcttgccaccttggcctcccaaagtgttgggattacaggcatgagccactgcgcccagcctcaactctctttttttttttttttagacagagtctcactctgtcactcaggctggagtgcagtggcatgatctcggctcactgcaacctccacctcctgggttcaagcgattcttctgcctcgacctcccaagtagctgggattacaggtgcccaccaccacgcctggctaatttttgtatttttagtagagatggggtttcactatgttggccaggctggtctcgaactcttgacctgaagcgatctgcccaccttggcctcccaaagtgctgggattactcaactctccttcttagagtcctttgtgaccttgggttccctgggccttggttttctcatctctgaagtggggctgggagcaactcttgcagcactgggttggaacgtgctggccacagtgctcacacatcgagaacacgccgtaaatgctccccgggcctccgcttcctggtgtcgtcatccttattcagaatgaacaagaaatcactccttcagagtaatgtttcagacagaggagagagctttgtgggggccctctggcgtgctcacagaagctttggtattcgtcaaccactgtaggagccaagcatttcctacagaatgcagggagcagggctggcggggagatagtgcgtgaaaagagaggctggtgttgggaaagaaacagagacagaacggtggccggcggacaaacgacaataagccaggatgatggtgtcactgcagccgggtgcggagtttctttctggggtgatgaaagtgttctaggttgatggtggtgatggttgcacagccctgtgtgaatatgctagacaccactgaattgtaccctggaaacagatgaattgtacggtatgtgaattatatctcaataaagctgttattaaaaaaaagatggacagtcccagctactcaggaagctgaggcaggagtattgcttgaggccaggggttggaggctgcagtgagccgagattgcaccacggcactccagcctaggcaacagaaccagacgcttctctttaaacaaaaacctaaacaattgtagatttttttaaaatgaggctttttgttttttttatgccaaaacccttcatagacatgaaataacagaagagtaagattggcggggggggggggggcggtttcatcagtacaggttacctgaaagaactgagttgttggtctgacctctgctaggactcgccatgtgtcatggcaggcctggcacctgaaggagagctttagcaatttgcactcacatcatcctggacctcagcctgggtgatggtcactgtcttgagacagcttcagaacaggcaacctgggtgtggccgccttgtcaaccagagataataaaaacacaggtaaacctctgcacctccatcgtcatgccgaattataacaacagcacatatttgttgaagtcttactatgcactaggcacagtgctgagtgcccatgatgcagaggggaatttgagagtgtcagtcatttctgccccttcggatcacgcactgctttccagtgttcccaggagaccagttttgaggttagaattctgcttcttacagtgaggactgattctgactcctaggaggacgaaagccccttttaaatttactttggctgggtgtggtggctcacggctgtaatcccagcactctgggaggccgaggtgggtggatcacttgaggtcagcattttcagaccagcctggccaacatggtgaaacaccatctctactaaaaatacaaaaattacctaagtgtggtggtgcacgcctatcgtcccagctacttgggaggctgaggcaggagaatccctggagcccgggagcggaggttacagtgagctgagattgcatcactgcactccagcctggatgacagagtgagactcgtctcaaaaacaaaacaaaacacaacaaaacattactttgaagccacagcattttgcacaggtaggaaaactgctgggcctggagatcttctataaatatgttgagagctgatgttgaagagattattgaagaatcctgatgtcacagaattttctatagacaacctccttgcggggagggcgcagctttcgggttctgtatcgcacaacaccggctgggagctccctgcatgtgtgtttccagcactgcctgtctatgggagctaaggaagtggcttcaaaaccgcttgccagagacgcgcagtcacccagtcgttgcagcgcttgtacttgtgggagctgtcttgcaggctcaggcatctttgataaggaattcctcatacttagaagtggcacctttgttctgctggggaattgttcccaaaaggtatctttttttttttttgaaacggatcctcgctctgtcgcccacgctggagtgcagtggcaccatctcggctcactgcaagctctgccttctgggttcatgccgttctcctgcctcagcctcccaagtagctgggactacaggcgcccggcgccacgcctggctaatttttttgtatttttagtagagacggggtttcaccgtgttagccgggatggtctcgatctcctgacctcgtgatccccccaccccggcctcccaaagtgctgggattgtaggtgtgagccaccgcgtctggcctttttttttttcctttttgagacagagtcttgctctgctgcccaggctggagtacaatggcctgatcttgactcactgcaacctctacctcctgggttcaagcaattctcctgtctcagcctcccgagtagctgggattataggtgtgtgccactatgcccagctaattttttgtatgtttagtagagacagggtttcaccatgttggccaggttggtcttgaactcctgacctcaagtgatctgctcgcctcagcctcccaaagtactgggattacaggtgtaagccaccacgcctgaccccaaaagacatctaaaaaggctttttcacttgtgaaatctgctgaatagagctggcttcctgaggcctgggggcccttcccggtgtggaagaatggctctgctgttggctaccacagtgctggggcctgtactcccacctcctctgcctctgtctgccttgtgtgcctccccttccgcgggggccctgctctctgcctctttctgaccttccagagtttttgaggtgtgagtgtgttgttgaatttcatttacataataattaaagagtcagggtttcatgatctactgatgtaaaacccgcaaaaaccctaaaatctcgagtggtgttgtacagcatgtgggattcttttgtataacttgatgattttcaaaatatccatttcggctgggcacagtggctcacacctgtaatcccagcactttgggaggtcgaggcaggtggatcacgaggtcaggagatttagaccatcctggctaacacagtgaaacccgtctctactaacaatacaaaattagccgggcatggtggcgggcacctgtagtcccagctactcgggaggctgaagcaggagaattgcttgaacccgggaggcgtagcttgcagtgagccgagattgtgccactgcactccagcctgggtgacagagcgagagtccgtctcaaaaaaaaaaaaagaaaaaaatccatttaatcagagccatacttgttgattactacaggtgcttcccgtatctaatacctccagcaagaaggtttgcgaaatgggcagagagtaataggaagagtttcaggaagatggtttgagtgaaaatcatttcagaaccgagccctgcgaagtcttggtttctgcaatccaccacagtttgggaaaatcgggaattttgtttagtccatttctatttttgtgcaccacaaatatgtaaacaaagctgggatcttcgtcgggggcggagggaagggcgacagaaccctgacatctaatactggtcatcggtatttccatgtgtttcctgatcccattgtctctgctatttaaattaattaattccacaagtatgcactcagtaaatattgtgagccaggcactattctcggctctgtggttgcatcagtgaacaaaacagacgctaatcctgctttcgtgagccatctataatagaatgggcgagtcaagaaattgacacatgcattagaaaaatatgtagcgtgtcgctggtaagtgcttgagagctcatcagggaagggcaggagtttgtggagtgggggtgagggatgtggggtggggcggggcaagggtggcaagaggcctggggctgctggttgcaactgtaaccagagtggccagggaaatccttgctcctctgggtaatgccctatccagaagacccttgcaaatttctaaaatgctgtgttttattaccgagaaagagtcgtctgggagctctccgaggcaaataacttttgaagttggcatgagcaggatattgtgcaatggggctcactggcttcttcccaggcctccgcatctgatgtgaaccacagctttgcaatcgggtccccgtgccatccaaaaatattttgtgaccttgccggttggcagttttttaaaaatgttattttcagtctccaaaaaagggaaacatcaaagtgcaggctgtccactcctgcccttcttgatagacacagacagctgggccacacaatcagccattgtggcgacaagtgcccaactgttataaatagacggcgggctcgcacgtgaagcgctccagaggaagggcaagtgctctggggacactgccaactgctgtttccgtcgagagctcttctcaaagatcaatattattttatgtctcagtgactgaatttgaaaaaacgactgtgaaagcacctggtaacttaactgtctccaggattccattccaaacgcatcttctctcttcactgttaatcttagatgtttcccatgcttatttagtaatcattgcctgacggcccacttcactgcgatttacagtctcttgcggctcacttgaaatggtagagttatggataaccgcgctggtgtatgtgcgtgtgatgcgtggccggaagcgcatgcccattgaataatcacgttcgtcccacatcttagcccctcttccaagacatgtttactatcgtttacgtgtgacatgtcaaaacatcttaaaactatgccacgcatgtaataggctctcagtgattaaaaaatcagtttgtctcttaaggagaaaagaataacagcatgtgactcttgtgagtccctgacaaagaatctgttgttccccgtgcacttgaagagccgctcatgtcaagaagcaacttgtgattatgcaatgtgtgacttaaggcattgtcagatgcgttcggctgaaacgggaccccgccccagcagtttgccttactaagttaacatggtaggtcagattttagtattgtagaaaaagccacattcaatattctttgagtagcatatttatttcctagggctactgtaccaaagtacctcaaaccaggtggcttcaaacaacagagttacacgctagttgatatctagtgattcaaagaacagaattccacactagttgttatctaggaatgcggagtgagtgatagagggggttttggtggcgttatattctaattttgaagtaagtttaaatttacagagatgctcccaaacagtataaagaattcccaaatactcttcacccagattccctgaattaacattccgacatgtttgtctgtctgtctctccacatacacacatcccttttttccggaataatttgagaatagactgcagagagtgcctctagattctcctgtgtgggtttcctaaaaacaaggacgctgtcccggatagcctgcaccgacctgcccaaatcaggacgtgggcactgtcacaccatgccaccaaatgcagaggccccggagcccgcccaggaccacaggcgccgtctgtgtcaatggctctcgagtctccttcaatctggggctgttccttcgtctctgtctttcacgaccttgacatttttttaagagaacaggccagttactttgtagaatgtccctcggtttgggctggtctcatattccttcatgatgagaatcaggtggtaggttcctggcaggagagtccctgcagggttgtcacgacaggaacgcacacaggtctgatcagtcccatttctggagaagttaacttatcatttggttgaggtggtgtctaccaggtttctccactgtaaaattaatttgttggtattttgtaattatcagttaaaaagtactgtttctggtggggagattttttggggactcggaaaatatcctgtttctcattaagctttttgcccatttgttttagcatcctgggatgattcttgcctgagggaattagtatgagcattgccaaatggtgatgtttctagttgcattgtttcttttgcattgattagttggcattccactgcaagctggagcttgcctgctgtctttctctccttcctatcaatacactcatagactcctattttacccattgggttataattcattaccattatcatttgcttcgatacccagattgtcccagatttggccaaaagcagcgcctccaagctggcccctgtgttcttttgatgtgttcatgtcattctttgagcacatctttacttcttcttcttttttttttttttttagatggagtcttgctctgttgcccaggtgggagttcagtggcgtgatctctgctcattgcaagctccgcctcccgggttcacgccattctcccgcctcagtctcccgagtagctgggactacaggcgcccgccaccacgcccggctaatttttgtatttttagtagagacggggtttcactgtgttaaccaggatggtctcaatcccctgacctcgtgatctgcctgcctcagcctcccaaagtgttgggattacaggtgagagccaccatgcctggccaggttttttcgtttttgtttttgttttttttgagatggaatctcactctgttgccccggctggagtgcaatggcgtgatcatggctcactgaaacctctgcctcctgggtccatgcaattctcctgcctcagcctcccatgtagctgggattacaggcgtgcgccaccacacccagctaatttttgtatatttagtagagatggggtttcaccatgttggccaggctgatctccaacttgtaacctcaggtgatctgcctgcctcgtcctcccaaagtgctgggatgacaggtgtgagccactgctcccggccacatgtttagttcttggttctaggctcacccggaactttccttgctccagccctggaatcagccatttctccaataagccctggttccttttagtagaaagtggtttttagaaaccaggatctaagtattaggtgtgttcacgctactgggctattacggctacaaggacttctcaagcagcgtagttaggaattttttttttttttttttttttttgagacggagtttcactcttgttgccaagactggcatgcagtggcaggaacttggctcaccgcaacctctgcctcccggttcaagcagttctcctgcctcagcctccctagtagctgggattacaggcatgcgccaccacgtccggctaattttgtatttttagtagagacggggtttcaccatgttggtcaggctgggtctcaaactcctgacctcaggtgacccatccatcttggcttcccagagtgctgggattacaggcatgagccaccgcgcctggcatagctaggaaattaaacatatgcatgtacttccctacacgtacacatatgtttgtacatacacacacgggcacacatgcgtttgtagttctttgtatccatctgtcccgctcgtatctatgtatccattcatctcatataaaaatcatgataccatccaagtcaaatggtttgtttcagctttctcccctttccttggctgtgcccctcttttctaacaataacagtaacagctcttatcatattaactgtttttatttattgacccttgtatgtcaccagtcttctgacatgcaggccatccagtccatgttcctggcccaggaacatggcagccaagggtccccacccccacttcggcccccaacagtggactctcgggatagttctttttttttttttgagacacagtctcactctgtcacccaggctggagtatagtggtgtgatctcggctcactgcagccactaactcctgggttcaagcagtcctcccaccctagcttcctgagtagcagggactacaggtgtacaccatcatgcccgtctacttttgtattttttgtagaaacagggtcggtcttaccttgttgcccaggctggtggatagttctttatagcagtagcctaaacagaaaaacatatacaacataagctaagaaaaaggcctcccttatcgatatttctgggttaatctgaaagattttgaagtctggaagaatcagctggttgttggtaaagggacggtggacatgaaggatgcggaagggatgggttggaggcgggaggtgagtttcatgtggtgtttttgactcagagggtcacttttattgttctgccttgtagtcagaagaaaaatcttttcacgtggtttcggctcagaagtatgttagaaagtagactacagagactgaaggtgacattccagagactttttaaggataatctggccaaaggtgtttactatttgattagccagcatccttgggctcggctagggtcttggtctggtctgagaacatgttgggggattgtgccagtgtcggtggtgggtgcctgtgcctggtcatctatgggcagctaggggtgggtgctgccccttccgactttctctccactgcagagacgaagccttcagcagcatttccctggaccctcttctctctggtcccaggtcaggttctgtacagcaatggaaagcaggggagaggatgcccacgctgtgcgtggccgtttggagcagactcagggaccagtgggacctcctcttctgggtgagcttggcaaccactcttttaggaattccagtttgtaatgccttccgggtgagccctgtgagtcgcttgctcttagtagtgtcctttgctgccccagccttctgcttccctgtatttaaaacctttctgagccaggattacgatttcctgataccatgactgacccttggtcgttatgagtcctgatggtcccaggagcttttccttcatttgcttgggttttgagttgtgagctggagaccaggggttttgcttgatggggacattcctggctcttctcctggggagtgaccatgggccctgcctaggtgagatcctctggattgtgaggagggacttgagcagccctactagagtgccgtctatgggaaccctcagcatcgcctgtccaagccagcatctagtggacgctcacgctgtgtgagacactgagcttatccagcaagggcagaaatggcagctcttgggaaagaagggagtggggcatcgctgtgtctcctattatttgccattgtggcctccctctccaggtgcaccgaggggcctatctgtgtgattcttgcaggggtactgacgtccgtttcatctcacccgtgtccctctacaagtgcaggagttgtagggctatgttattcttaggtagcttaatggatccattttcataataaactccattttattttgtttgccattgtactgccatttgcggaacaaagagaagttatataaaccacagaagggggagaatgtgacccaggaataaactgttgtgtttcacagtaagtgtatggaactaatttagaatgatttttcattgtcaaacaaagaacaaatggaaatttgaacaatgcatggttacttttaacaaagacttcattttaatgttattagtgttttctcattttaaaatggcagtgcaaatataaccttaaaatagggtttatgactaattacatttcttttttgtatgtctgacaatttccaattattgggtgtttgtgttccccagcccaccctcccccaaaaatgacagggaacagaaaccagtgaaaagatctctctttggcgcccttcccagactaggacctgttttgttcagggtgagcacagaggagaactgggtcctcttgtcaggctgaaggggacgtcacagaagccccaagtgggtcacggcagagcgtcagccagaacctccagggctcactcccaggcctcagtcaccagatgcttcgtggctacctgggtaagggagggttcagggcaagttagggactgcaatgagaaaacaacacactctggccggatgcggtggctcacgcctgtaatcccagcactttgggaggccgaggcgggcggatcacctgaggtcaggagttcgagatcagcctggccaacgtggatttcaaccaaggacctgcttggtcatagatttcaaccgaaggacctgattggtcatggatttcaaccaaaggacctgattggtcatggatttcaaccaaaggacctgattggtcatgcagacagaactttgaaggctgtcatgggttgtgtccatgttgtttggttttccatcttgaacgaataccagtgtgcgtctaacagctcacacagaagcccattggttacaaaggaattgcaattgtcaatccaattgccccccagggagaactgaagatttttttaaataatgtcttcgtaacagctgtgtaagtcccccaggcccttaactagagaaaccacatcacagaattttatttactttataaattctaatattttagaattaagataatttgtagtttagatgatttttgctaagctagctctctctttctttttatctcttcctttctctcttttctttctttctttgatggagtctcaaaaaaaggagaagggtgtccaaacatcctggatgaaaatggctccatgctgtcatagaattttatttactttataaatcctaatattttagaacttaaaaaagatactttgtagtttagatggttatagttaagctatttcattttttttaaaaaacgaagaaattagcattcaatgtctgcctagaaacaatcttatatacagtccttatgtacaaatcttttttgtggaatatgttttcgtagttttttgttttttgttttagattgggtcttgctctgttgaccaggctggagtccagtggcgtacttatagcttactgcagcctcgacctccagagctcagtcagtcctcctacctcagcctcccaagtagctgggactgcaggtgcatgccagcacacttggataatttaaaaaaaaaactttttttttgttgttttgttgagacggggtctcacttatgttgcccaggctggtctcgaacttctgggctctagcgaagctctttcattttagaaaaggatcatgtcacctcgatatgtgaggtctgtggaaaatcttgctttaagatgatctgcgtgaactagtagcttagattattgagtcttattttcttcagaactctgataggcacaattactagatttaagttaaagggctgtaaaacagcctcagtactttttcatgaagtagacaagtttcacagtgctttggaaatgactactaagctactcccagcctgctcattgagaactcacgatttcctctggatgagtgggaatgttgttggttctgcatttgtcatcagtgcttaggacacacacacacaagcaacgtgtgctggcccatgcagtttttccttatttacaaagctcatgtgagaattgtgactctgcccattcgtggatctgcacaacatatggtgacgaatataagttcgggggcgtggggggaatgcgcatgtgctatggttcagtgctgggcactgggtgttgtggttcagtgctgggcactgtgtgccgtggttcagtgctgggcactgtgctgattgcttctgcgactccggacagatacttaaggctcagctcaggttcgtcattaacacgaatcacaaattcagacctggtgccgtggctcacacctgtgatcccagcactttgggaggccgaggcaggagtatcacttgagcccaggatttggaggccagcccgggaaacataggaagacccccaactctacaaaaaatatgaaaaaaagtttagccaggtgtggtggtatgcaactgtagtcccagctactggggaggctgaaatgggaggatcgcttgagcctaggagttcaagaccagcctgggcaacatgatgaaacctcatctctacaaaaaaaaaaaaaaaaaaaaagaaagaaaaaaagcatggtggtgcatgtctgtagtcccagctactcgggcggctgaggtgaaaggatcacttgagccaaggaggtcgaggctgcagtgagccaagatcgcgccactgtactgcagcctgggcaacagagtgagaccctatctcaaaaaattcacaagggttttcaaagctagttactgcctgagaccatcttcccctcttggatggatgcagcaaattgggaggcaaaattatttggctctttttttttttttttttttttttttaaggcagagtcttgctctgttgcccaggctggagtgcagtggtccgatctcagctcactgcaacctcagcctcctggattcaagcaattctcctgcctcagcctcctgagtagctgggattacaggtacacgccaccacacctggctaatttttgtatttttagtagagacagggtttcaccatgttagtcaggctggtcgcgaactcctgaccttgtgatctgcccgtcttggcctctgaaagtgctgggattacaggcgtgaaccaccgtgcccagccgcgtttggctccttcctaactaaacagagattaaactgtttattgttacctgggctttcattttttcatagcagtcactttattttcatctctttgtcctcagcttcccctcaaatatttgtcaagtgtctaccaaggctgggaatgatagtaaaaggagtttaaaaaacaaaagaggctctaaaaatggtactagactcttccattcaggactttgtccaaagcctcactttataatgcttttgtgatttatcctcattcccataaactcagatgatacgaatttatacgagtttgcattaataaatatttcttgcatgcttgtcttatcaaccataccgagccagcacagcctcatctcgtaaagagttggcagtcacgtgcgggagccgggagcccgttgctgtgaggccgggaagtggattgttggtctggggaaagatttattttctgggggcaaaagaggcggggtggagagtccctccgcctgcctggagtgctgatgtggcctcagagctaggtcctgaaaaattgctgggatttagatgcaggctctacatgtagagagattgtttaaatgtataatggttcagaagtgacactctcttgctatgtttctctgaaaatagatcagccttctctgttgggatctatgaagctccagagataaaactgatatacattaaagtggctcgtggttaaaggaagtatgtttgttaatattaaaccgaggtttctctggtaactagattttaaatgatttgattccataggtttataatggagaaatccagatctccaaaatgtaagttttactatatgggaagcctttttttgatttcctttgctgtgtttggtaaatagcctggaagaaaatggctccgtcctatcacagaattttattatttacttcataaacctagtgttttagaattttttttttgtttttttgtttttttgttttttttgagatggagtctcactctgttgctcaggatggagtgcagtggtgcgatctcagcccactgcaacctccgcctcctgggttcaagcgattctcctgcttcagcctccggagtagctgggattacaggcacacaccaccacacctggctaatttttgtatttttagtagagatggggttgcaccatgttggctagactggtgtcgaactcctgatctcaggtgatccgcccacctgggtctcccaaagtgctgggattacaggcatgagccaccgtgcctggcctagaattttaaaaagattctttgtagtttagatggttctagttaagctatttcatttttttaaaaaggaagaaactagcattcaatgtctgcctagaaaacaaaaataaaaaaacaacaaaaaaggaagaaactgaggtctgtggcacttaagtgccccactcaaggttgcagagttcctagtgatgttattttttaatgaacttttcttctccttctcttttttgttaaattttggcagactgttttaccaaagcctaactgtacttttcttaattaactttggaatgaattctggatatactcattccagcaatgcacagaaaattctaggcaaagaataaaagactccacattgtttattattattatttttgagatgtagtcttgctctgttgctcaggttggaatgcagtggcggaatcttggttcactgcaacctctgcctctcatgttcaagtgagtctcctgcctcggcctcccaagtagatgagattacgggcagctgagattacaggcacgtgccaccatgcctggctaatttttgtatttttagtagagatggggtttcaccatgttggccaggctggtctcgaactcctgacctcaagtgatcctcctgcctcggcttcccaaagtgctgggattataggtgtgagccaccgcgccgggccagactccacattgtgaaaaaacgagggaaccgcttctgattttgctccagtggtgtattcctctgtcttgttataatgctttatgtttacatataacacattatacgatttttaaagcaaccccacagggaagagttttaattttttgtttgtttgtttttgagacagagtcttgctctgtcgcccaggctggagtgcagtggcgccatctgggctcactgcaagctccgcctcccgggttcacgccattctcctgcctcagcctcccaagtagctgggacttcagtcgcccgccaccacgcccggctaattttttgtatttttagtagagatgaggtttcagcatgttagtcaggatggtctcgatctcctgacctcgtgatctgcccgcctcggcctcccaaagtgctgggattacaggcgtgagccactgcgcccggccgagttttaatatttttagtcctgttttacaaaggggagaaacgaagcacagagaagtttaggaacttgcccaaatcatacagttgataagttagcagaccaaaaccggaatcgtccaaacggagtggcagccagaaagcattttataggtaaagtcacatcctacactgtaaataaaaacctcttggaacacagagttcgccaggtggctgttggattttcatgtcagcgctggtctagctccgttttgcaggattttttgttgttgttatcacagtatattctaaaaataactatatgttcatttccactctttcagataattaatatttataaatcctagtcactaggaaatatagttcaaccaacaattgaatggaatgcaggtcctctgggaacttatctaaaattaaatcctattttaacagaattagctagattaaggtcatggtgtaaaaactgatttttcagaaggaaataaaaatgcaaaatatcctaagaattcattttgtaaataattgaatcaaataacaatggggcggggaagaagccatatatttttcctctctttttagaaatgaattgaaggccagacatgctggttcacatgtaatcccagcactttgagaggccaagcgggagaattgcttaagcccaagagtttgagaccagcctgtgcaacataggaaaaccctgtctctacaaaagaatttaaaaacagctgagtcagctgggcgccgtggctcacgcttgtaatcccagcactttcggaggctgaggtggatggatcataaggtcaggagtttgagaccagcctggccaacatggtgaaacccccgtctctgctaaaaaaacaaaaattagccaggagtggtggcggacgcctgtagtcccagctactcaggaggatgaggcaggagatctgcttgaacccgggaggcagaggttgcagtgagtcgaaatcacaccagtacacttcagcctgggcgacagagcaagactccgtctcaaaaaaaaaaaaaaaaaaaaaacttagctgggggcagtggcacatccctgaggtcccagctacttgggaggccgaggtgggaagatggcttgggcccaggtgatcgaggctttagtgagctgtgattactccaccgtactccagcatgggcaacacggtgaaaccttgtctcaaaaaaaaaaaaaacaaaaaaaatcagataaataaaaacaaaaaattgtttagactcatgaatcatcaactttttctttttctttgcccattttcttttttaagtattagcttggcattaaagcagtagtatatgaagatggaatttgctagtactttatttgtgattccagagttaaatgctactgcaatcaagtcttccttgaatttagcaatagatttaaaagtttagaaagagcctcttttttttttttttttttttttggttttaatacggggccttgctttgtcgcccttgctcgatcgcccagtgatctcagctcactgcaacttctgcctcctgggtttaagtgatcctcctgcttcaggctcccaagtagctgggatgacaggcgtaaaccactacgcccagctacattttgtgtttttagtagagacggaggtgtcaccatattggccaggctggtctcaaactcctgacctcaggtgatccacctgccttggcctcccacagtgctgggattacaggcgtgagccaccatgcccagcccaagtctagctctttagaaggaaaaaataagggtagaaattcagagaaggatgagtgtggagtggcttccttgctctatttctagaatatgccatttcttgggaagtcttatgaccctgctggtctttaatgtcccctatatgattttcctccctgggctttgattctctgctttatctcctgtggtcttttccatccagaaattttgaaggggtatttaccttcaacttaaaactgactactacaggtaagtagtggccctgctgtttccaccctcccatgtctactggaagcagaattgtttcacctgtctcgttggaaaatcaaatgaggtaatggaaatgaaaagtactacacaagtgtgattattaaagacagttcagtcaataggtgaaaagactcgtgtagatactttatttaacctggaatcaatgtcttatactcagtagacactccttagatgtgagtttaaagatgtcattactcctataataccagcactgtgggaggccaaggcgggtggatcacctgaggtcaggagttcgagaccagtctggccaacatggtgaaatctcgtctctactaaaaatacaaaaattaaccaggtgtggtggcggctgcctgtaatcctagctactcgggaggccgaagtgggaggatcgcatgaacccaggaggtggagattgcagtgagccgagattgcgccactgcactctagcttgggtgacagagcgagactctatctcaaaaaaaaaaaaaaaattaagaagatgtcattttaatgagcagttctttgttaagtaaagaagaagaggaaaccaggaaatatgatagtgcttggccacctcaccaagtgtttgtcttaaaagaagctttcttgattaagtaaaaatttaaaaggaaggaaagaaaaacccaacctcaccgcaacaaactaccctagtctgaggtaattgtgtagtgtaatgtccctacacctgaatttttcatgcacctgcagatttaggggctctgcctatctttgagtataaatgtccactgaattattagcaaaatgatttgttctcagaaaccaaaattttagcctagatgaatgccgccattggagttatttatatgaaaaggcgtgttaagctcaatggtcttctcttctcttctcttgttttcttttcttttggatttgagacagggttttgctctgccacccagcctggagtgcagtggtgcaatcatgggtcaccgcagcccctacctcctgggctcaggtgatcctcccacttcagcctcccaagtagctgggactacaggtgtgcaccatcatgcccagctaatttttgcatttttcgtagagatgggatctcactatgttacccaggctggtcttgaacacccgagctcaaacgatccgcctgcctcgacctcccaaagggctgggattacaggcgtgagtccctgcacctgacctcaattgttcctttcagcaaaagtaaaattttaactttcaagggtgcaatcctgcttttttttttttttttttttttgagacagagtttcactcttgttgcccaggctggaatgaaatggtgcgatctcggctcactgcaacctctgcctcccaggttcaagtgattctcctatctcagcgtcccgagtagttgagattacagatacatgccaccgtgcctggctaatttttgtatttttagtagagatggagtttcatcttattggtcaggctggcctcgaactcctgacctcagaacatctgcctgcctcagcctcccaaagtgctgggattacaggcgtgagccaccgcacctggcctgcaatcctgcatttaaagcatttttaaagtgtcgttggaacacttaaaaagacaagaattaaaacagtagcagaacacctaaaatggcaaggaaacggcatgagggcgtgtaacacagaaaggggtgggtgtttgagaaacagcaccgcaagtacgtggggaagcggcaaagatggaggacatgggagatgggggtggaaaggggcttggggcctggtagtgaaagactttgcatgccacgttagagtctctgctctgttctgttagcaggagtttgtagtagtacctattcacagactggtgcgggttaactgtgagggtaatgcgtgtaaggtgcttagaacagtgtttgacacataatccgtattctctaaatcttagttgtaggagatgttgttgctattatttttataaaagggcaagcattacgagtttcagagcaaggcttatggggtgataatataatggaggatgaattggaagaggagaaactggaagcctggagaatggtttggaagctctttgctggggcccgagagacattcagggttcaggcagtgggagtggaaggaaagtagtggattccacagcgagacttggcagggcttgatggtgagggaaaggaaggagaagaaagtgacctggaggcttctggtttggatggttggggagtgatgaccccgagagaagagaggagagaaacagggttgtgaggatgagacccaactactctgttcctcttttctcaactgaaaccatgctgacttcatactgcaaagagtgtacagcgacacggtatctcagtccgttttactgctgcaacaaaatacctgcaactgggtgatttccaaacaatagaaatttatttcttacagttctggaggcagttctggaggccgagaagtccaggatcgaggccttggcattgggtgtctggtgccatgtcctcacatgacagaagagcaaaggctacggactcgttccctcaggcctttcatgcggttgctcacccctaagccctctcgtattagggtcttcatcccatttgagaaggtggagcactcatgacttcttccttacctcctaaagaccccactgcctttttttttttttttgagacagaatcgtactctgtcgcccaagctggagtgcaggggtgcaatcacggctcactgcagcctggacctcctgggctcaggcgatccccccacctcagcctcccttgtagctgagaccacaggcgtgtgccaccacgtctggctcattaaaaacaattttgtttttgtagagacggagtcttcgctgtgttgcctaggctggtcttggactcctggactcaagtgattcttccacctcagcctcccaaactgttgggattacaggtgttgagccactgtgccagtaagaccccacttaataccatcccttcgtgatgaagtttaaacacgtcaattttgggggacatttagaccatagcacataggtttcaaaaatgagtcaaagccttaaatggattgtctcttttctagtaccagctgataggcctcacgtctgttcactgccttctgatgctcctccttaaccctgtgtaggactctgtagtacaggtctccgctcaaagacagttgtaaacgtggcacagttagtcaagagcattacaataaaaaatgctcagcatcactcatcacagagaaatgcaaatcaaaaccagaaggaaatatcaccttatgccagtcagaatggctattagaattatatgagaataatattaagaagtggggtcttactgggcacaatggctattattaatcccactactagatatctacccagaggaaaagaaatcgatctatcaaagggatacctgcacttgtatgttcatcacagcaatgttggcaatagcaaaggtatggaatcaatgtggccatcatcgaatggatgggtaaagaaactgtggtatatgtatatgcagtgggatactgtccagccatcaaggggaacaaaaccctgttatttacaataacatggatggaactgaaggtcattattattatgattattttttatttttattttttagagaagagtcttgctctgttgcccaggttagagtgcagtggcacaatcatagctcactgcagccttgacctcctgggctcaaatgatcctcccacctcagccacacgagtagttgggaccacaggcatgtaccaccatgcctgtctaattttgtttattttttgtagacatggggtctcactatgttgcccaggctggtcttgaactcctggggctcaagcgattgtcccaccttggcctctcgaagttctggagttacaggcatgagccaccatgcccagcttggaagtcattaagtgaaggaagccagacacaaaagacatattgcatgttcttatatgtgggagctaaaaggtttgatcacatggaggtagagagtggcaagaagggtaacagaggctgggaagggtgagtggcggggaggagaagggtcaagagaagtcggttaaagggtaccatcatgcagtaacgtagaaggaataagttcaaggcttgagagcagagtaatattattctaatattatatatattctcacctaacaaaaaatgtattgtggctgggcatggtggctcccgcctataatcccagcactttgggaggccaagacaggcagatcacctgaggtcaggagttcgagaccagcctggccaacatggcgaaaccctttctctactaaaaatacaaaaattagccgggtggttggtgggtgcctgtaatcccagctactctggaggctgaggcaggagaattgcttgaacctgggaggcggaggttgcagtgagccgagattgcaccactgcactccagcctgggtgacagagcgagattgtctcaaaaaaaaaaaaaaaaaaaacaaacaaaccccaaaaccaaaatgtattgtatttgagtgatgggcaccctgaatgccctgacttcatcactgcgcattatatacatgccataaaatttctcacataccctccaagtttgtataaataaaaagggggtgttgaatacagactttcaaagaaaaagcattctgctcaaaactcataaggggacgtggtgtgtgtgtgagttctgaaagaagtttgaaggaccagggttgaccacatgatcacgggaaacagcagctcctgggttgcttcaaggtgctgtggctgttggtggatgaaatcagcctactaggagataggaagatacaaggagcttctccagtgtactggaagatcacagctctggcatcatgggatctaaggagctggcttgtctttagtgaagggaggggggctggggccccttggaatctttttttttttttttttttttttttttgagatggagtttcactcatttgcccaggctggagtgcaatggcacgatttgggctcaccacaacctccgccttccgggttcaagtgattctccaccctcagcctcccaagtagctgggattacaatcatgtgccaccacgcctgtctaattttgtatttttagtagagacggggtttttccatgttggtcaggctggtctcaaaactcccgacctcaggtgatccgcctgcctcggcatcccaaagtgctgggattccaggtgtgagccaccgtgcccggcccccttggaatctttaaggccaagacctgggaggctggaggtcgagcatgaaacaaagcgaaatttccctgagaaggaagaaagaagtgaggattccctgaaaccaagaagcagctctgtgttcccatctttcctcgtgtgtgtcattggcttcccatgcatataggctttgcgaggcaatctaagcatgtgtgcacgtgtgttttgtgtttttgtgtataattgagtatggctgacttttgagtgggcacccataatcactctgtatggcaattacacttatattaactttaatccatttcagagtaatgactcataatatttaatacccacatatcattgtgtattatatattaagtaattattaagatataatagctgtcctgtggttctttctgcagttctgcaatgcataagtaaatatatatctacttagcacctggcagaattctgacactggttccctggagtgagggctgttgtggtagcaaaggccaagtggaagctgccagaactgcctctgtctaccaaaatagtcaacccaaagcaattatcacattcctgggaggatggcagagattagtgccaccatcagggacttgaaagatgcaagggtggtgatcgtgtcacatctccgtttaatccatatttggcttgtctagaagacggatggatcttagagaatgacagcaggttgtcataaacttaaactaggcggtgactgcagttacagctgctgtgccagatgtgtttttgttgctggaacaaatcaacacatttcctggtacctggtatgcggtgagtgtcttctccttctctacctggtagtaaagatgaccagaagggtcttgcttccagccggcaaggtccacagcccgccttcatagttacacttcaggggtctgtcagctaccagccttatgttatcacttcagtctccttcttcattccatggggtgccaacggataaccactcagatagaatgtaaaaggactttgggccaggtgcagtggctcatgcctgtaatcccagcgctttgggaggctgaggtaggcagatcacttgaggtcaggagtttgacaccagcctggccaatatggtgaaaccccatctaataaacccctactaataatacaaaaattagctgggcctggtggcaggtgcctgtaatcccagctactcaggaggctgaggcaggagaaactcttgaacctgggaggcggaggttgcagtgagccgaaatcgtaccagtgcactccagcctgggtgacagagtgagaccttgtctcaaaaaaagttcataaaaggactttggctcctcttctggggagagggttagcatggttttggttgtaagacggatagtagcattatatggcatggaagcctagttgctatttacttgatttggaagttaagtacggttgaaaagaggagtagatatggacaccaagttgacaaggggtacgtgtagtgagttcgttttgcgtcaccttggctaagttggagctctgtatgtaacttgagccacaagagaccatttgtgtaagaccgagaaggcagaggggacacagcagccacatgcactgtggctcatgcacgtggtctctgatctgcaggctcaccttgttggcgcacggcagccatcaggcctgtacctcttctgaaacccaccaaatctttcgttgttcccctcatctgcgtatgtggcagacagtttttctatgttttcaggcaattgttgagcaaataataatagaatttttgaacaggccagcatcggtgctaatatgtgccaggcacagatctgagccctttagagtatttactcatttaatcctcaaaaccctgtaaagtcactgatgttgccataatctgtcagactcagaaactgaggcacaaagaggttaagtaattgcttaagccttcacaggaattgggatctgaacccaggcattttggtgtcgagtccatgaactgacgaagaacatagataactgttgtcagcttgtcaaggctcatgcctgtaatcccagcactttgggaggccgaggcgggtggatcacaaggtcaggagattgagaccatcctggccaccatggtgaaaccctatctctactaaatcataaaaaattagcgtctgtagtcccagctactcaggaggctgaggcaggggagtcgcttgaacctcggtggtggaagttgcagtgagccaagattacaccactgcactctggcctggcgacagagcaagactccgtctcaaaaaaaaaaaaaaaaaaaattacttcaaggagagactgctgagggttcctctgtctcctgtgtggatggaatttggcctccatttggaatcgggaatcttgcatatttagttctgcagtattacgtgaatctacggaagcaactcatttggaattagattccaagccgttataaataataaattttttgtctctgtcatttttgtagtaacagcaagagaaaagaatttaggaccaaaacttatgagagttttaaatattttttgctaagtgcttatatgtgatttatgggaaaatcataatttttaccctggaaggcagccttactcataaagaatggccatcttgtagcacctccaggacatcagtctgaagtctaagttaggtgtgtttgcacagcaaccaaatggtgagagcatttagcagctgggggacggcggcattgttgtgattgggttgccacgtggtggggcgggtgagggacggcagcagagccttggcagagcggagctgtgtggccgacggcttcctctttcttcctccagcgtgcgattataacttgttgctcatgcaccggcatgcagctctgtttgctcagtctgtttttctgtaaagtctctttttaaaaaagtatatgatacaggctgggcacggtggctcatgcctgtaatccgagcactttgggaagccaaggcaggtggatcacttgaggtcaggagttcaagaccagcctggccaacatggtgaaacctcacctgtactaaaaatacaaaaaattagccgggcatggtggtgggcacctgtaatcccagctacatgggaggctgaggcacgagaatctcttgaaccagggaggcagaggttgcagtgagccgagatctcaccattgcactccagcctgggcaacaagagcaaaactccatctcaaaaaaaaaaaaaaattgtgtgatatagatggaagaccacgtaaaataaatgtacaatctgattgttataaggtgaccgcctttgcatatctaccaggcaggttgcaggagagcacagccagccacccagcagccgtcctgccctccacctgccgcagggggccagatcttgccattcctggtggtcccgtctctgctcttcttggttttctttctttcgttctttctttttttttttgagatggagtcttgctctgttgcccaggctggagtgcagtggtgccatctctgctcaccacaacctccacctgctgggttcaagcaattctattgcctcagcctcccgagtagctgggattacaggcacgggccaccatgcctggctaatttttgtatttttagtagagatggggtttcaccatgttggccaggctggtctcgaactcctgaccctgtgatctgcccggctcggcctcccaaagtgctggggaggtgtgagccactgcgcccggactttttggttttcttatccaactgtgagtccttcaacacttggcgtcgggtgtttggcttgtctgttttttggcttgcttttgcttttttctccaacttttaatttaattttaattaattaattttatttatttattgagacagggtctcagtctgtcactcaggctggagtgtagtggcacgatgatggctcacctctgcctcgacctcccaggctcagatgatcctctcacctctgccccctgaggaactggaactacaggtgtgcaccaccatgcctgattaatttttgtattagccagtggtttcgccatgttacccaggctggtctcaaactcctgggctcaaacaatccacccgcctcggcctcccaaagtgctgcgattccaggtgtgagctactgtgcctggccttcgcaacttttcattttgataaatttcaaacctacagaaaagttgaaataatagtataatgaacactaatatcccacttcacctgattgacccgtggttaacattttgccaaatttctttggctgtctcagtgtaggtgtgtatatatgtatgtgtgaatataattatacacacatttgtgcaagaaccctgaatgggatataatacagtacacacatatgtatgtgcatatgtgtatgtgtgtgtatctatatttgtcctcttttttctttgctgagccattgaaccgttaactgactaaattgcagacattgtggccctgcacccttaaattcttcagcatgtatctcctatgaacaaggacattctcctgtacagacaaaacaaattatcctactcaagaaatataacattgacagaatactcttataaaatatatagttcatgttcaaatttccccagcatctaacagtatcctttagagcttttatggtgttcatccaggatccaatcagggatgatcttttcctaaaagaggacactcaagatctagatctagaacagttccgcacatttttttttctttactgctattggcatttttgaagagtttggtttcctgtttcttaaaataaattttgcattgaagtgtaatatgcatacataaaatgcataagtgtacagtttgaagaattatcacaaaaggaacacatccactaaaccagcacccagatgaaacagaatatcccagaagcctccctcgtgtccctcccaacgctacccacttcctccttctaaaggtaaccatgtcctgacttctggcaccataaattgcgtttgcctgtgtttgaatttcgtacaaatggaacctgcagtgtgtattctttttttttttttgagaccgagttttgctgttgttgcccaggctggagcgcgatgccatgacctcgactcactgcaacctccacctcccaggttcaagtgattctcctgcctcagcttcctgaggagctgggactacaggcacccaccaccatgcctggctgattttttgtatttttagtagagacggggtttcaccatgttggccaggttggtctcaaactcctgacctcaggtaatccacccgcctaggcctcccaaagtgctgggattacggacttgagccactgcccctggtcagtatatattcttttgtggctgcctctgtcactcagtgtaaagtttgtgagcttcattcctgttgcctgttgcttacgctggggttccttcatcgtcactgctgttgtgccttctcggtcatatcacaacgtatccatcgatcctcctcttgatggatacatgggttgtttctgggctttgacttttacaataatccctgtgaatacttacgtactgtatcctggtgcacacttgcatgcatttctttggccgaaatcaatgtctagaaggggaattattgagccatagagtgtgagtgtcttcaacttccatcggtaataccgacttgttttacaaatggtgataacaaatggtgataacattgttatcacctcatacccgtcagcagagtatgagggttcctgtgggtggctctgcatcctcaccagcgcttgtcactctgttgaattgcagccactctggtgggtgggtgcagtttcttttggtaattttcatttgtgattctgtatttgatttgcagttccctggctgagtgtcttttcatatgtctgctggccacctgggttggctcttcagtgaagtgcctgttcagctttcttacacatttttctactgagtggtctggttttgttttcttattgttgggcttgttttgttttgttttgtattgtcttgagacagagtctcactctgtcgcccaggctggagtgcagtggcacaatctcggctcattgcaacctccacctcccgggttcaacattcttttgcctcagtctccccagtagctgggattacaggcacgtgccacctcacccagctaatttttgtatttttagtagagatggggtttcaccatgttggccaggctgctcttcaactcccgacctcgggtggtccaccaaccttggcctcctaaagtgctgagattacatgtgtgagcccccgggcctggcctcttactgattttgaggagttctttatattcaggctaccagccctttgtcagttatatgtgaggcaaataccttctcccactctagttttgcctttttactacgaaattgccatttttgtagatcgaaatggtcaaatattggcaattggcatatggttcagcctaccataaaattgatccactttttttctttgttaggtgaggactttttttgtcctatttctctacttctgaggtcatgaaactataatattatttcctagatactctattgtttcacttttcagatttatgtctataatccacgtgaaatgtgaactgatattttttgtgtacactatgaggtagggatcaagttttactttaaaaaatatatagctaggccgggcgcagtggctcacgtctgtaatcccagcactttgggaggccgaggagggtggatcacgaggtgaggagttcgagaccagcctgaccaagatggtgaaaccccgtctctactaaaaatacgaaaattagccgcgtgcagtggcaggtgcctgtagtcctagctactcaagagactgaggcaggagaattgcttgaacccgggaggcagaggttgcagtgagccgagatcgcgccactgcactctagcttgggtgacagagcaagactccatctcaaagaaaaaaaaaagaaaaaaagaaaatagataacgaattgagcactttttattaaagacagttttactaaggtatcatttgcacactatgaagctcagctgtgaaaagtatacaattttatttttagaaaatttagggtatcatctggttttagaacatttccatcatcccatgaggtccatcatatctggtttcaggcaatacctgcttccagtcccaaaccacaaatctactttctgtgtctatagatttgctttttctagacatttcatataattggaatcatgcactattgtagtcttttgtagctgacttctcaattagcttattgtttttgaggttcttctgtattgtagcacagatcaagtatttaggtcctttttcgtgagttaaattcggtggtgtggatatatcacagtgtgtccatccattcaccagctgctggacacgtggattattcccacgtcttggctgtaataaacagagctgctctgaacatcgcttgcaagtcttagttgtttccatttctcgtgagtagaggcttggattggaattgcaagattgtatggtaacgtataattttattttgctaattatcatttgtgaactgacttttttctcttgctatacggttctaaggatcataacacatgtataggttttgtagccaccagtacaatcaggatacagaacagtttaatcaccccccaaaaatctttcccaagggttgaccttttaatatgatttggctgtgtccccacccaaatctcatcttgaattgtgactcccacaattcccacatgttgtgggagggacccagtgggaggtaattgaatcgtgggggcaggtcattcccatgctgttctcgtgatagtgaataagtctcacgagatctgatgggttttttttcttttttttttttttgaaacggaattttgctcttgtcacccaggctggagtgcaatggcatgatcttggctcaccacaacctctgcctcctgggttcaagcgattctcctgcctcagcctcccaagtagctgggattacaggcatggaccaccacacccagctaatttttgttttactagtagagatggggtttcaccatgttggccaggctggtctcgaactcttgagctcaggtgatatgcccacctcggcctcccacagtgctgggattacaggcgtgagccaccacgtccagttgatctgatggttttataaagaggagttcccctgcacacactctctctcttccctgccgtcatggaagacgtgacttgctcctccttgccctctgccatgattgtgaggcctccctagccatggggaactgcaagccagttaaacctttccttcgtaattacccagtctcggataattataaagtctttatgaacagtgtgaaaacagactaatacaccctttatgggtgttaccagtggagggtcttgactacaagttgtctgggttcttaaccagattcttggcattttgaacaaagaattggacaaaatgcacaaagaaagcaacgagaaaacgaagcaagggaagcacagatttactgaaatgaaagtacactccacggagtgggagcaggctcgagcaggagcaagtggctgattagagaattttctggagccttaataccctgtagaggtttaccattggttacttggttacaccctatgtaagtgaaggagtggcctgtgaccagtctgattggttgcaggaggcgaccaatcagaggttgaagtgaagttgcaacgttacacctgaagacctggcctgggaccagtctggttggttgcgggagggaaccaatcagaggtactttccatttttcatctgccacgaggtgcaaagggagtagtagcctctgatccttttgttacctgggtatggttttccttttgattcagttctaaaaagtcagggtgaatcggccttaagttccctgcctccaggcccctgccgacccagcaacaactatccattccctgtagttttgcctttgggagcctgtcattaaaatggaaccacacagcatgtaccctttgaagactggtttcttccacttagtctagagctattgagattcgtcctggatgctgcatgggtctctagttcattccttgtgatgctgagtagtgccccattgtggggagggaccacagtgtgtttatccattcatcctgggaaggacattaggttggtttccaggttttggcaattatgaatagtgccgctacaaacatttgcgtacagcgttttttgtaaacataaggtttgatttctctagggtaaatatccatgattggggtttctgggtgtatggtcattgcaggattaactttttttttttttttttttttgagacggagtcttgctgttgttggctcaggctggagtgcaatggcgtgatctcagctcactgcaatgtccgcctcccgggttcaagtagttctcctgtctcagcctcccgagtagctgggactacaggtgacgccaccatgcccagctaatttttgtatttttagtagagacagggtttcaccatgttgcctacgctggtctcagactcctgacctcaggtgatccacctgccttgacctcccaaagtgctgggattataggcgtgatccaccgcgcccagcctctactttagtttcataatgggctttaatttgtgaattaacttcttgatgtatctgctcatccatccattcattcattcatttctatattcacccaacaaatagttaagtgccttacttctgtctttgctatttctttgcctttggagcactgttttgaaagagccagcgcccactgtcagctgtccagctggtagcactgtcatctggaagattttagatactttctcatgagtgattctacaccagcctgaatctcacactacctgacatcctctcaatcgtagccacacccaagggtcctactttgctgtccccactgccaccagaccaaacacacccccttccctgaaagtctgggagtcggtttcttcttcacaatagaagttggcctctgctggtcatgccccaaaccttctggagctgcgggttctgccttctctgcagctgccttcccgtgggctgccctggtcagtgcaggacatggcacaggacctccgccctgccacacccccgggccatttcctcggcaggcctcgtgccagacttctccaaaggactgaccgcgtgtgccttctccaccttgcacacactcgcacaccaacctgccagcctggcctgtgctcttcgccgcccaaacgcacctctccctggtgtggtcactgacagtctttgcgtggccagtggccaaacccaggctggctttgctagtggcctcatcttctctctgcagcgttcacagtgatccacttctttttttttttttttggagacagagtcttgctctgttgccctggctggagtgcagtggcaccaatgtccgctcactgcaacctccacctcccgggttcaaggcaattctcctgcctcagcctcctgagtagctgggattacagtcatacaccaccatgcctggctaatttttgtatatatatattttttaatagagacgggggtttcaccatgttagccaggctggtctcaaactcctgagctcaggtgatccacccgcctcggcctctcaaagtgctgggattacaggctttagccatcacacccagcccccctccctacccctttgtccctcctcagagccctgctggcccacttccctggccgttcaaaacattgcagctccaaggctgcttcccaggcctccccacctgctgtcttctccctggtgatattatggacagtggatctctggccctgagctcccttctgatttcatggtatggccacctccttgtccctatcgcttgagcgtgtcaccagcatcttcagcttgggaggtccagaactgagctcctgattttgccccctaaacttttacttcttctggtctcactcatccgtccacgtactgaaggcagaacccaggaacccaggaagtgtccttgttccctttgttcacatcctgtcggttctgcctagaccttaaatcatgaacctgacctaaggccactccagcttttgcctggaccacagcaaaaggccaggagtgagcccccgcttctactcctgacacctccggagccattcgccacccagagctgtgtaggctttaaaaagagccagccagccgcgtgggatcctggatgggatcctggaacataaatcaggtgaaaaattggtgaaactcaaatcaagttcatcgtctagttaatttcctgttcttgattatcaagctgtgcttgacgtcgtacaataagacattcacgttaggagaagtggagcgagggatatatgagaactccctgtaccgtatatgtaactttcctatatgattaacattagtttgaattaaaaagttttaaaaaattggatgggtgcagtggcttatgcctataatcccggcactttgggaggctgaggtggccggatcatgaggtcaggactttgagacgagcctgaccaacatggtgaaaccccgtctctactaaaaatacaaaaattagccaggcatggtggttggtgcctgtaatcccagctactcaggaggctgaggcaggagaatcactcgaacccaggaggcggaggttgcagtgagccgagattgtgtcattgcactccagcctgggcaacagagcaagactcagtctcaagagaaaaaaaaaaagttaaaaaattaaaatgcaaaataagaaagaatcgaactgtgtgctgtcttacttaaaacactgcagagctcttgtgtgtgcctagcatgtaagtcagatgcctcttgtcgttttgagacagagtctcctaccgtcccccaggctggagtgcagtggcgtaatctcggcccactgcagcctccacctcctgggttcaagcgatgctcgtgcctcagcctcccaagtagctgggatcacaggcatgtaccccgcctggctaatttttgtatttttagtagacacggggtttcatcatgctagccaggatggtctcgaactccttacctcaagtgatccgcctacctcggcctcccaaagtgctgggattacaggtgtgaatcactgcacctggccaggcagaaacctcttaatggctcccagggcctggcccacctccccagcctcctctcatgccacactttcttccctcagtctgcatcgggaacagttgccacctttcagccacgtgagcatgctgggaccgtcctttccatgctgatccctgcatccctcgtgccttccccagacctacaccaccgcaattgcctccatctcactgtgatatctccacttcagtctcatttcttcagaaaggacgtccctgactctgtccaaaccgcatgttgggtattctctattgcaggaagcttttgctttcttcctggctcttaatcacagctggtgactcgggtccttgcttgtggattctccctgccactggttcctaccactgagtcaggtgatggacgccatgttcacagttttatcaccagcgtctagcgtggcctgcagcgggtgctcccatagaacccataaacaaacaacttcttgtttcttattatgtgaagaaagttggtctgtgtaattcaccctccagagaaatgacagcactttcagtactttgttctgcccttccttgatctccacgctgtttgcagatggaccaccgttgtagagatgtgatttgattcttgacgtttctgagtgtccacggtgctgcgccttcccccaggtactacagtactgagattaataagacagagtcaatgtaaggtgttagtaggatttagaataggtctaatctcattcttaacggatttcaggagcttattatgtgttgaaagaatgaataggataaaagttgttaagaaattttctccgtttaatttttatttattaatttatattttagagacagggtcttactctgatacccaggctggagtgcaatggtgcagtcacagctcactgaggctttgaattcctgggttcaagcaatccttctgcttcagcctcccaaattgcggggattacaagcatgagccactggctctggcctattttctccttttaaaaaaaattgctgcaggtatacacatatgcacggatatacacatacaaatatgtaccttatacatatatgtatataataaatacatgcatatgcggaattgtggcatagtcttagtcttttttttttttttttttttttttgagacggagtctcgctctggtgcccaggctggagtgcagtggtgcaatctcggctcactgcaagctccgcttcccgggttcacgccattctcatgcctcagcctcctgaggaactgtgattacagcatgggccaccaggcctggctaatttttgtgtttttagtagagatggggtttcaccatgttggccaggctggtctcaaactcatgacctcaagtgatcctcccacctcggcctcccaaattgctgggattacaagcatgagccaccatgcctggttgtatttagtctttaacctgtcctcaggctgtttctctctgggcagggagaagcatggaaagggggatgggccctggtggccacctttgaagcatggaggctccggtgccaccctgcccttaggctgggcacaaaggtggctgagggaaggctagtcagctgctggaggtgtcctttatggtctcagtgattaaaggttatgttgtccatgaatttaattttatttcatgatggaaaagaggacattgcaaattaagtatgtcacagaaatgtaaggagccatggggtctgatagggctgagggtgagtggcttgccgtcgagtttccagaaggcgttattatggaccctaaaggggatttcatgcgtttcaaagactctaacagaagtgttctgatgaagtggagtctggtgctgggacctgccacttcagcctggaattgtcctcgtgtgaagcgcagtaaaaacccagaatggatgtcggcaataagggaatttctgggatatgatgggggaacgaatattgtcacctccatttgtgtcaggaagtctcagtatgcagatgagtcagctgcagggggtggcctcccaggctgctggtggagcccagaagcagggggcaggcgtttggcctctgatactctcctagctctgagttgggacggagctcgtgtatctacagtgtgctcacacagacttggtgccggtagatggtgaggggataggttggcaggtgggtagggggcctcccgaggcagtagggacctggaggaggctggtagaaagtggagattggccaaagtgacctgaattagattctacctggttcatgaatttgcctgaggctaaggcttcagcgcaagggatggtggagtaatacctttcctgaaacacctgctgccggcgtggccttgtctgtctctgccagatcttgttagcactgtggcttcaggcctagttaaacattcaagcccaatgcggttggagagcacccatggcacgcccattgtgcagcagtgtgtttaatacatgtgctgcttttaatgggtgtgaaagatcagagagcttgggtgtgtgttttaccaaccactttttaaactcacatagtaaaatttatagtatataaaaagcagaaaatatggtgtatcttctaaatattcacatttaaaacattttttagaaataatgtatgtcaagcaaggtcaagctaatgtgtttatataggctctaaattagagttgtgaaccagtatctcatcccacttatagggttttttttgaaaacgttcaaatgtttccagctcttttgtttctttattctaaaacattatctgctattttgttcgatagagtaactgtttctattccactttctttttttttttttgagacggagtctcgctctgtcgcccaggctggagtgcagtggcgcgatcttagctcactgcaagctccgcctcctgggttcacgccattctcctgcctcagcctcccaagcagctgggactacagacgtgtgccaccaagcccagctaattttttgtatattttttagtagagaggggttttcaccttgttggccaggctggtctcgaactcctggcctcaagtgatccacccgcctcagcctctcaaagtgctgggattacagacgtgagccaccacgcctggcttattccactttgtaattggctatccctgctattaatatacagggtactctccctctcgtatttgcgtctaaatgtcatctataataaataacagtgaatacttactggtttcatatttagttttacttttatcccacccattttcttttagtagttcttatgacttcacgtttatttaataaaggtaaaaaagaactttctgtttccaattcattcctttcaccaataaggaaggatgtggatcttcactgcttcctacatagctgccagcctcatgtagctgtttaaaatttaaattataattgaaatcattaaaaaattaattccttgttcatactagctacatttcgagtggtcagtagccacatgtgcctggaggccacagaactggacggctacaggatgtacccatcatcacgggaagttcatagaaagttctttttctttttctttttctttttttgagatggagtctgctgtgttgcccaggctggagtgcagtggcgcgatctcagctcactgcagccttcacccactgggttcaagtgattcttatgcctcagcctcccgagtagctgggattacaggctcccgccaccacgcccggttaatttttgtatttttagtggagacagggtttcaccatgttggccaggctggtctcaaactcttgacctcacgtgatccttccaccttggcctcccaaagtgctgggattataggcatgagctaccatgcctggcatgtatctcttcttctctaagtactcagctgtcttgtttgaggctagtacctccactagtctggaaccctccttcctgggctttctgcccacttccatctctcctttgacaacagtggtttttccagggtctgtgcagagctcagctttctgccataagacaggggagtttctgaagactcctcaccccttttgggagtgagggctgctgactgaagctccaggtcactgttttatgagctagctctccgtttgttgatggccggggacccagcctttgtcccctcctctcgtgcttgggcactcactactcactgcctcggttttgctttcagttttgattgtgcattgagtccacaacttcaatgggattttcttaaggcataaatgacagttgcatggtgtgcagtgggagaacaaatcgcccagcctgggacatccctgggtggcagagtggacctgctctcaacttatggaatgtgcagcaacaccttgaggttattaataaatacctgagtctgggcatggtggttcatgcctgtaattccagcactttggcaggctgaggccggaggatcacttcaggccaggagttcaagcccagcctgggcaacatagtgagattccatctttaaaaaaaaatttttttttaaaagaaggtgggcttggtggtgcacacctgtcatcccagctacttgggaagatgaggtgggaggatcacttgagcccaggacgtcgaggctgcagtgagccgtgatcatgccactgcactgcagcctgggtgacagagaaagaccttttctcttaaaaaaacaaaacaaaacaaaacacacacacacacacacaactgcgattcttccagaaaagttcagaaaaaggaagtccgaaatacatagtccatagtatttcagccaccagtgctgctgaagtgaaattagagatagcagagatatctcaggagaaaatggaggtttttgggtttctccgttagatgactcgaacaagtctgcatgtgctctgtaaacaagaaaacacagtctgggaaactgtcagcctctgccccagccagtgtgcaaaatactcttcaaaagagagaaaactagaaaacaagttgaaaaccgggtgatctgacaagtgtggaaattcacagcaagcatgtgcctgtgctgcagatacgatctgggagagagccagcagcctggatccacatttcccagaagcaacaggtggtgaaggcagcggcatgactgctcccgcggaaggttagtcacgcatgttcagcatcagccgggcgcggtggctcacgcctgtaattccagcactttgggaggccgaggcgggtggatcacttgaggtcaggagttcaagaccagcctggccaacatggtgaaaccccaactgtactaaaaacacaaaaattagccgggcatggtggcaggtgcctgtaatcccagctactcaggaggctgtggaagagaattgcttgaacccgggaggcagaggttgcagtgagccgagatcatgccactgcactcctgcctgggcaacagagcgagactccatctcaaaaaaaaaaaaaaaaagagaagaatattccgcaacttagagcttttcctttgcatgatatgatgtttacaggcgaggtggctccgcagcctcggtggcctttcttccagctgaagatgcccacaggaggtgtcagtgcaggtataaccacctgtgggtcagaagaaaatgcctttgcaagtaaatctcctcctggatgaaaaggcccatctgtgttccaggcaaggacagtggccgcttctgcgtttctgtggccaagcgtctggcgtgcgggtgtggctgggctcaccatgctgtgcgggctacacctgctgcaggagaagtgtgtttgtgctcgaagaagacaatgtgggtgtcggcccggcgtttttaggttggactacaatagtgagagctgcaaattggatgattcttggagctatttcatgaaggtcaaattttcttttgttttgttttatttaatctctcccagacttctgaacttttgctatgttgaatttttagttcttcaaggttatttggccttattcttgacttgcatgtttttctgccctagctctaccacattatctttttttttttttttttgagaaggagtttcactctgctgcccaggctggagtgcaatggtgcgatcttggctcactgcaacctctgcctcctgggttcaagtgatcctcctgcctcagcctcccaagtagctgggattacaggcgtgcatcaccacgcctggctaattttcgtattttcagtagagacagggttttaccatgttggtcaggctggtcttgaactcctgacctcaagcaaccccccgcctcggcctcccaaagtgctgggattacaggtgtgagccaccatgcctggcccacattatctatttctaagcctttaatatttttcatttacctgctactgctcactctaccttgtattctcagggagggtgtatttcttcccagctttattttatttcttctctaaatctcagaattattgcaccttgttgatgttcacattactgtatttagtttattttattgtattattaatattattattatatttgagacagggtctcaccctatcacctaggctggagcatagtgacacagtcatagctcactgcagccttcatttcctgggctcaagcagtcctcctgcctcagcctcccaagtagctgagatcacagaaatgcaccaccacacccagcttccttttctttttcaaaaaaacagctttattgacgtataattcacatatgatgcaattcaccctctcaagtgtatgatttggtggtttttagtatattcatggtgttgtgcaaccatcaccacaacctaattttacattttcgtcaccccaaaaagtaaccctgcatccgttagcagttacaccctctctctcctaaacccatggcagccactcatctgctttctgtgtctacagatctccctatgccagacatttcatataaaggggatcacctagttcgtggccttttgggactggcttctttaactaaacatcatacagctttatttccattttcagtgtattccctttttttgttttcaagtatttagtggctacctagtctcagattttaaatttagtttttcctttctcttctttctataatctatttttcatcatctacactgtgtgcttgaatcagcacctcttagaaacctacggatgagctgttagctctctagggacaagaacacggtctgggacccccgtccctgctgcaggtgtgacctgaccccctccctctgccgacgtgacctatccccctccctcccgtctgtggcatctggtgcatttggtgctgtcatttcacaaataaaaggtgatcagctggtggtctgtttcagttctggtatctttccccagctctcttgtttcatagcctcttcccattatttaaactctgctctgttacaggattcttaatagttattgcagaccttcatttccattttgttgtctgcaactcattgttgaagtgccatttgttgcttctgaacagcccatagacacttggacttgaatgcatatgactctcttcttgtgggtccagatcgtcactcactgactgtcctcacattcacagctgttctcaggacaagcaccgtctttccctttttcggatgtccctgtttacctgaatcaactcatttaatcctcacaacagccctgtgctatgatatgaccatcatatcacagcattttcgttttctttttcttttatttttttgagacagggtctcattctgtcacccaggctagagtgcagtggcgtgatctcggctatctgcaacctctgcctcccgggttcgagagattttcctgccttagcctcccgagtagctgggacttacaggcacctgccaccacagctggctaatttttgtagttttagtagagacagggttttgccatgttggccaagctggtcttgaactcccgacctcaagtgatctgcccacctcggcctctcaaagtcctgggattataggcgtgagtcaccgcgccggccagcatttccattatctagacaagggaactgaggtcaagcattggagtgaaagtcacctaggtggctttttttcttttttaacctatttatttctttcaacccttagcccagatcctaaccttcggtaagtactcagtaaacatttagtctgctgggtgcatgaacgagcgaatgagcaaatgaatgacagcgatagcacctggtgggctgttcgctggtgtgctgggtgtgctgggtgtgctgggtgtcgtctctcagagactcatttctacagtctttgtgaaggctcgtgctagtaaaccctaattcaagaggtttcttagaggtgggaggcgaaggtctctgaagtgtgtggactcctggactctccctatgttagcattctggaatgtagcatttagcagtggggcatttattaactcggaatacagaagataagaagccaagccaggcacggtcgctcacgcctgtaatcccagcactttgggaggctgaggtgggcggatcatgaggtcaggagatcgagaccatcctggctaacacggtgaaaccccatctctactaaaaatacaaaaaaaattagccaggcatagtggtgggtgcctgtagtcccagctgctcgggaggctgaggcaggagaatggcgtgaacccgggaggcagagcttgcagtgagctgagggagccactgtactccagcctgggccacacagcgagactctgtctcaaaaaaaaaaaaaaaaaaaaggccaaaacaggggaatgtggtcactcaggggatgcctgtcattcccatttggctgtgatggcagctgtttagccattactgggagagagagggaagctgggtatagaaggtacctgtttagaagcaggggtggggaagcgggagggagagagagagagaatgcgtgcacatgctctaaaattttattttctggctgggtgtggtggctcacgcctttaatcccagcactttgggagacagaggcaggcggatcatttgaggtcaggagttcgagaccagcctgtccagcatggcaaaaccccctctctgctaaaaacacaaaaattagccaggcatggtggcacacacctgtaattccagctactcaggaggctgaggcaggagtattccttgaacccgggaggcggaggttgcagtgagctgagatcgcgccactacgctccagcctgggcaacagagcaagactctgtctcaaaaaaaaacaaaaaaatttttttttggttaagcagaagatcaaattaaacaggcaaggtgcctaccctttgtttctttcactttcttccataaatccatctcatattttattatttgggacttctttttttttttttttcttttttgagacaaagtctcactctgtcacccaggctggagtgcaatggtgcgatctcggctgactgcaacctccgcctcctgggttcaagcaattctcctgcctcaacctcctgagtagctgggattacaggcatgtgccaccacgcccagttaatttttgtatttttagtagagacgggatttcgccatgttggccaggctggtctcaaactcctgagcacaactgatgctcccgtctcggcctccctaagtgctgggattacaggagtgagctaccgcgcctgacctgggacttcatttgttaagaagaggttaagtttcaccagcagccatttgtagtaggaacttgagatttatcatcaaatattgggacaacaagaacaaatcagttcttatgattatttatggtgcatatgagtaggaaggacttacaaccctccggagagcattttaatgttaacatatgaaagatattctctcttttgtaccaaaacttttgttcttcttacgcagaaagaatagataattataaaatatatctatgactccataggctttggctatttggtagattaagagtatttttatctttatttttcttattggctgattacagtttagtttaaataactctcatgccgcttggtcgtttccgacctagtcgtgaggaagaatgcaatgcaaagtgaaagacgtttttggaatgtttctgaaccagcccaagctgattgcctcttttctgtttattgttttaagtcataagtgacgtaagcttctaggcatttgttatgctatgtggctggctcccaaagtcatgtcttaaaacattctttccttcctgattattcttctctttcttgtttttcttttcttctttttcttttaatttttttttcttttttgagacggagtttcactcttgttgcccaggctgaagtgcaatggcacgatctcagctcactgcaacctccgcctctcaagtttaagcgattctcctgcctcagcctcccaagtagctgggatttacaggcgtgtgccgccacgcctggcttgtttttctttttctttttttttttgagacagggtctcactctcttgcccaggctggagtgcaacggcgcactcacagctcactgcagcctcaacctcccgggctcaagtgatcctcccatcttagcctcctgggtagctgggaccacaggtgccaccacacccagctgatttttaaattttttatagagatgggatctcgctatgttgtccaggctggtctcaaactcctgggctcaagtgatcctcctgccgcggcctccgagagtgccggggtcacagctgtgatccccgtgcctcgctgttgttctctttctgttgagttggcagtcaggtccttgattcttggtggctgagggtatctcagtccaaggcaagaatcccacagaaagagactgggacaaatgaggccagaaatgggcctcgcccatcactcttttccaaaatgggctttgtcagtcactcttcctatacttgggtcaccaaacctgatcaccactatgtgcaaagtgcagttttagttttcaaagaagtgacactatacttaagaatacttaagaacgttaactgcgttaacctccacgcacacagccatagacaaattcccccgtcagacaggagcccattaggaaatgcccacccctctctgtagcctttgtaaaattaagctgataaaagacatcgctgttgttgaggtttaaaatcaatggataaccaggaaagaatataagaaatcatattaattattctcctgtttacactacacttaaatcatttagctgtcttttttttttttttggaccttctgctgcaaaagagccattctaatgaacttggaccccagggtccatgtgtgtggttattcattaaatcatgtcgcctgcagagtctcctgacttaagactttcgctgggcttgaactcagtaacctgtcctgatgtatttctcaggcgtttgtgattaggcagctgggctctgccttacctgtcagggtgataacccttctggttctgacaaaaggtttcactttaaatttagctttccatattgaagcataaataaaaagataatttacttcccgattttgctcgttgtggacacggaggtgtgtttgaccttcgtaaagctagtaggatcatgttatcactatgggttggtcattcagcgcttcctaaagaaaaactcaagtcagaaacaaatcaggagcaattattgatttaacagttttgcaaatgcttttgtatgtagaaggtgcatgaaaaagattccagtgatacgcagatcaaacactgcgtcgataatcacagctaatgtcgagtgcttgctgcgtgcgtgttgatcatttaagcctcacatgcgagctgtgtggtaagccctcctcctgccctcgccttagagatgaggaaactgagaggcagcgatgaagcagctcactagccctgctcctgtcctccccttagagatgaggaaactgagaggcagcgatgaaacagctcactagccctgctcctgtcctccccttagagatgaggaaactgagaggcagcgatgaagcagctcactagctctgctcctgccctccccttagagatgaggaaactgagaggcagagatgaagcagctcacttagatcacgggattagggggcggacctggacccgactccgaggccccctccagcagccaggccctcgcccatcacgcttggttgcttgtaatgggagccttgcctctcgccagcagtttgtggggagtaggtttcccgtttccactgttatttcctgactcccgtgcctcccaacttcccacccgctgtggctggcatttccaccggctccctggcttgtcctacgcctcatgcctcctatgtggctcttctttctcctcccgcccccctgctgcccgtggccccagaatggaggcttcccaggatggcccacacagtgccctgcacaggtgctgtggctcagccctggccattccctccacacgttctctgctccagccacactgagctccaagccatcctttagctatgcctgctgcaggccacctctgtagagggcctttcatgacctgttctagtaacccccctgcctggtgtcagccacccccgcccctggcctggtttgctattgccgggctgacctcgccccgtctttcagttctccgtgtggagggtgctgatccgtccgtctccatctgcttgtgaagcaataacagaacagcataggctgggtaatctatacagcacggaaatttctgtcctcatgcttcaggaggctgggaagtccaagatcaaggcaccagcaggttcaggtgtctggggagggctgcatcttctggaagggagaaggaaactggagggcaagcgagagagccccttacagcaagcccctttatgcaggcgcctgatcccatcgcaggggaggagccctcctggcctcgtcacctcttaaagccctgcttccttaagactctcactccacaacgcctgaactttggaggggacacgttcgagccacagccccttccttcctgaggcttctcaagatgccccctcctgtgcttcctggagcacccgtccttccttgtcctgggacttggcacgcctgaccatagtcatacctgcttgtcctcttggcccctcttcaggtaatgggctccacacaggtcacgatctcggccaaaatagtcacacatcgtccctgcacccatcagtgtgtagtaaacaggaagtgcttaataaatgtttgatgaattaagagtgactcagtaatcccagtacaaaaaagcaacagcagacgttggagatgtgcttgaggcacgtgcccagcttgtgagcagaggccgccgctgccactccgtgctccccgtggactgccgtgctgagcgctgtgaatgtgctggctgtcccgtcgtgctccagctcttcaggcctccctgctcagcctctctcagcttcagttgccacgcacttctaggagaggccatcttgactttctcttgcagtcccggagccacacagggagcaggtgcgcagtcacctgggtgagggccccttcctgggtttcagaggcagcccagttcatgctcccagggcagcccttgtgcatggggagccgtagcctcaggcaggggcaccctgagccaacagcaccttgcagggccagccacagacataatggaaaacctcatccttcagccgcaacttttcctacctcccatcaaaaaaatgtccctcctctctcagtatgtggggactgcggtctccagagtggccatccacagtcacactggctgtcagtggctcctgtcaccaccctgccacctggtgacttgtgggccacactcaggagtcatgggaaggagacccttagagcagccccaggacaggcctccttgcgtgtacttctttgatgaaggcgggtataagccctggcagctcattgtcctctcttgagtccacataggttgcattttttaactctggcaaagtgaagttacaaagtcagtctggtgtcgcctgggacaggtccgagaaattagtataaagatggcttcactcctgcgtcgtattccagttcacgtttccatttcagtgtatacatgttacagtcgtgattgatcctgtgcataaattgggatgtgggtttttgttggagaaaaaaatgcacaaaagttcaggagctaagtaagagtgaaagattttcattggaccaggtgtggtggctcacacctgcaatcccagcactttgggaggccaagatgggcggatcacttgagtctaggagttcgagaccagcctggccaacacagtgagacccccatctcaaaaaagaaagaaagaaatagttggacatagtggcggacgtttgtagtcccagctactcaggaggctgaggctataggaggtgggggctgtagggagccatggttgcaccactgcgctccagcctgggcaacagagcaagaccctatctctaaaggaaaaacaaaacaaaacaaaacaaaacaaaacatgaagagtttcttggggccagttggcatctaaaaatatctaagtcttttgattctcagctatgacctggctacgttctaggctgtctttacgtgtttctaggactcttggcaaattctcagaacagttgacttaaaaagattagccacgtagtatttttacaaggagagagggaggtgaagtgatgagatggggccatacttataaaatgattcgggattattgatgaagtagtccctaggtttctgattaattaattttaaagcgctgaccaaatatgtacccagcgtaagaagccagagtcgatcagtgttgtagacacgtcgcctctcgatggcatctttccatgccacacccttcacgacctgaagctggagcttaaacccttcaagaagggtccctgcgaccgggtgcagtggctcacacctgtcatctttgggaggccaaggtgggcagatcactaggagttctaggccagtctggccgacatggagaaaccctgtctctactaaaaatacaaaaattagccaggcgtggtggtggacacctgcctgctcacaggctgagtcaggagaattgcttgaacccaggaggtggaggctgcagtgagctgagatcgcaccattgcactccatcctgggtgagagagtgagactccatctcaattaaaaaaaaaaaaaagaagggaccctgctttgttcatgattttattctcggtgtgtggcatacactaggcctgaaataaatgttatttaaatgttaaattacattgtccctccttccgatacatttgccatctggttaaattccagtgaagtcaaaggatggatttctttaaataaagtttgcttctttttcaaaaattccttgggctctttctagaaaagctgctcctttgccatcttcccagcagattcccggggcaggatccacccacctgttcacagctccttccagcgcagtgtgggtccggctgttagctcagcctcaaaggagaattgtaaagatacaggtcaagtgtccttttgaccaccttttttttttttttctatatgcgggattttgtgagacaataaaatgcatgtggacctcagcttaacaaaagctgaatgttcctggggtgagtgtttgaatccaacaggaaatagatgctcaacagctctgagtctggggaggttttaatggcctcttggtccctggtagggtcttgtttttcggggtagttttgcagcctctgacacaggtaagatggtcagtaccggaagctgccatgacgtctagactgaccgttaggaaggtactcactggactggtattcacagatggaaataaaccaaagcagtgtctagggtcaaccaatctgctttgctgccttcacggggaggtcctgggaccctttcatggaggaactttttaatgcttattttaaccctgaaggtaaaacatgaaacatgcagccattactccaagaaaacgtaacgtgggtgagtggtgtgtctcgggggcccacggtctccctgaggccgcatctctctggagagatcgtggacgtccgtcgtaaactatgacctattcacattttcactttactctcaactttccaggagtgagtcctctccagagagcaagacggagctgcgtttcaacagacggtgctttcaatctgtgctgactgtaacttttatccatcataggtttgaagacatacctgatatcgggtcagagagccaaggagtctcgctgcagctgtgcagaggccttgctttctggctttgaggtcattgacggctcacaggtgtcctcaggccctaggggacaggggacagcgtcatcagggaatgtcagtgacttggcgcagactgtcaaaacctttgataaccttaaggttagtttttttgttttttttttcctacttctaaaactttctattaattctgcagtagagcacgtgaaataataagcagcctccaggtctgaggcaaacaacttggaatgaccttcgatttggttttcacgattttaatatttagtctaaagttagtagcattctgcatttttaacatccttgctttttgacatatgacgcacctccattgcctttccattgcacaaggatgcttctcatttgaattattagttattttctcttcctgggacctgtggtgccttttgggatttctagaagctggtgataaatgacagtaaaggtggctttccttggcaccttgggtggagagctagttgataatctgatttcagagaccactttctcaaatacttctacgtctcttgaaatccactattttatgattacttgaaatgatgttggtgtgggtgaatcctaggacacagctcactgagaggacaggctgccgagaacattgtagtatttcattattggaaccgggatctcctggtagctgcctagaagtgcccgtttctggactgacctgtcatcataaactaccagggaattggcgcggtcgaccttgggtgtgtgaaatcatttcagtttatgagctgttcctttgctttcacatttgatttagtttccccactggtaccttctgtttatttttatagtttaaacatctactgtctccattcttaaaatgagacagagaatcaaaacagctctcctatgctgctttgtgttctcagataacacagtggggtatgtgtgaggatggcggccagtggttacagcttcacccaatacgtccctgcctctccacattcattcacaggtttgaaagccgctattggtatttacgaggaacagtcttcattatgtggcagtcagctggggtatgcagattacttactgggtcagaatacatagtgcctgtcgcccagttacacgtaatgattgcactcgggttctgtcaactccgctcgtggatcgcggtcgttgggtgctttgttagtgtaatgcagggcttctcaccttggcaccgctggcattttggaccaggtagtcatgtgttgtggggctgtcctgtgcactgtcagatggctggcagcatccctagccgccacctgccagatggcagcatacccacccgtaccctactccccagctgtgccaaccaaaaatgtctccagacatggcccagtgtcccctgggggtgcagagtcacccctgggtgaaaaccaccgggacagtggtgttggaccctctcttccctgtactcttggcacatgtgtaaagctcatgtatctgagattccctggatgcctgcaaactttagagacagccgccttgcctgggctgacgttgtgcaggagtcagaaaatggagctctgggctcctagacagcggacccgggcagggaggggctggcgtggggtggaatcgggaatggttttgctatttggggaagaattgtttcctccagcctgcaaactgagctgtatttaagtcgtgaaagacagccccgagccaaaaatatataattcgttgtgtttgccattttagcttccacatatgtgtgtttctgtccgttaggggatgcggtcgcagagttcaccatatcacatgctcacgggtttagtgaccacggttttagcaatggcgattctacactacattgcactctagtcatatatatttgttttggtcggaatgagcaaaaaccatttagcgtatcttcttggcatgtggagattttccaaatattgttgaaagtttctgagaatcttgtgatctatcaagttgacagaaggccgggcatggtggctcacgcctgtaatcctggtgctgtgggcggccgaggtgggagggatgcctgaggctaggatttccagaccagcctggtcatcgtagcaagacatcatctctacaaaaaaattttaaaaattaactgggcttggtggcctgtgcctgcggtcccagctacttgggaggctgagccaagaggatcacttgagcccaggaatttaggctgcatgagctatgattttgccactgcactccagcttaggccacagagtgagaccttgtcctgtctccaaaaaaagggaggagggtgctcagtggtaaagtaaattcgggaagcttgggatattcaacacagaaagggtttctttactgcaggacttctcggagccttcactaggctagtagacagtagctaggccttcagaggaggacgcggtatatacagcattctcaggcctctttgagcacaattgttttttttcaagctgcaccttgcagggctaatgtggtcacaccttttgggggaatgctcatgtagtaaaaggagtttcatgccaaggaccaatacattagagaaaaggtgttcaggaaacgttccctgaaggagcatgcgtagattttcacgtgtgtcctgttcctgctgctgctcacttgagatactcatggcttctctttcccctgaactcctgtctgcattcaccgttcagctccaccacgccttcccgtgcctccgcagctgataacgctgttccctcgtctccaaattctcagtcccttcagtctgtcccacttggctgtaacatacacgatcttaaatctttgtttcttatgcgtcttcctgggagctctttgcattcagagactgagtgttaaataacttaaatatttcccaagtgtcgagcatggtgcctggcatgtagtaattaacagtttctttttggctttttaattctttaattaaacacatggagcgatgcagctctgcagagccaggcgaaggactgaagtcagaagcccgagcagaactgtgtagaaccagggcagcgcccacgaagggtttatttcctgcccttagcatgtgtgtcctctgtgaccttaagtcatctcctaggtcatagctggaaaattcttatctaatgtaacataacctgtgtactgtaacgacctaccaggaaatgatagtgatgtcaaaggccagggctttggtcctgaagtcatagctcaaagggaaccctttctacacccaagtgttggcttcttttttttttttttttttttttaaagagatgggatctcgctctgttgcccaggctagagtaccagtggtatgatcttagctcactgcagctcgaaactctcaggctcaagtgatcctcccaagtcaacctgctgagtagctaggaccacaggcatgtgccactatgcctagctaatttttaaatttttttttttttgctatattgcccaggatggttgccaactcctggggttataccatcctcctgccttggcctcttaaaatgctgggattacagtcatgagccactgtgcctggccttgggttcttgaaatgaccagcagtgagaggtcctcatcctaagggccctgacattgcataatgtgggggacagatactgcccgagtgaaggaaggagaggtcaagaaggaaaccaaggagtaagcattagacatgaggacgccactgtcagcgtcgcggaggggaggcggaccacggcgggcaggggcgcaacttgaagcaagacccttctcaagccttggctttgcgactgtgaagaaactgcagccaggtgcagtggctcatgcctgtagtcctagccactcagacagctgaggcaggaagatcgcttgagcccaggagttagaggctgcagtgagctgtgatgatgccactgcactccaaccagggtgacagaatgagacactgtctcaaaaaaaaaaaaaaaaaaagaaaagaaagagcaaacacacacttaattcctctaagtgagagttttcctcttcagtgcggtggtgataaggaagagacctctcgggattgctgggagatgagccggaatggtgcctgtggacgccagggctgagaagaagcctcgctcacactcggcacgcacgccttcactccacgcagagcagggacagtgaccacagacggagccatggaggactgggaggggccgagagacggtcccagcttgcctgcaggaagtcactgactctctggggaaggtcccatcttctccacggttccgattcgagggagattctcacagtgattgcggctgagcattgctgtgggttggatgctctcggccgtaaccaagtcacttgtatgacacgggagcatgtgtgccttggcctgttgaatgtgacgtcagcgctccagcaggcatttgctttccattttctcatgaaccctgtgggtgatgagtggatctagatgccatatatcaataaaaacagcttttccaacatcttcatgaagaaaaagaaaacccaactgggctcagtggctcatgcctgtaatcccaacactttgggaggccgaggcagagagatcacctgcggtcaggagttcaaggccagcctggccaacatggtgaaaccccatctctactaaaaatacaaaaattagccaggtgtggtggcatatgcctgtagtcctaagccactcaggaggctgaggcaggaggatcccttgaacctagtaggtgaaggttgcagtgagctgagaccacgccactgcacttcagactgggcaatagagtgagactccatctcgaaaagaaaagaaaacccaaaattgaattacttgtatcacattacttctgatcagggaaccatttgagctgtgaaagcatcttagtacatctctgaaaggcctccctggagcgcacctgcgtggctggtcttcctgagtttaccttcatctattattcagaaccccgggctgcttggccactgggtgtttctacatggaccagtcagtaatgaagctagtgatgtagatttacccatacggggatgattcagagaggacctgtcctgctttcaaaaggtttctccttcccctttaaggccactcacctcaggaaccatccttttgtgacagtgaattttgaaaatgaccctaaatgataacttcttttatctacttgttttctatgtgtcaagtcattatgagtaggctcagttggcccaatttgaggaagtaaggggcttttcattcgttatagaaattgaaaaaatgatttcatattttatagacacatacatatataaatatatatagcttagaaattaaataggtatttattataagtggtagtctgaattgatagtggtgatgcttgcacatccctgtgaatatgctataaaccctggagttatatactttaaatgggtgcattatatgctttgcaaatgacatcttaagaaagctgttgtaaaactaaagggtagtctccgccaggtgaggtggtcacacctgtaattccagcactttgggaggccaaggcagatggatcacctgaggtcaagagttcgagaccagcctggccaacatggtgaaaccccatgtctactaaaaatttaaaaattagccgggtgtggtggctggtgcctgtagtcccagctacttgggagactgaggcaggagaatctcttgaacctggaggcggaggttgcagtgagctgagatcatgtcactgccctctagcctgggcaacagagtgagattccatctcaaaaaaaaaaaacaataaaaaaaaaaaaccatctctacaaaaactataaaagttagccaggcatgttggtatccacctgtaatcccagctacctgggaggctgaggtgggaggatcgcttgagtctgggaggcagaggctgcagtgagccaagattgcaccattgcactctagcctgggcactagaatgagaccccatctcaaaaaagaaaggtggggggcggggtaggaataggaacttcacagtggacaaaccgggcagatgccaccttcaccaaatgatccaggtgagcctccctgctcactgccatccggtaccctagtgtggtgtgctgagaagggcatgtcacctctgcggcattctttccgcaaaccccaaacctcagtctgctctgagaaagcaccaggcaggtccagatctggacatttgacaaaatgcctggccgggacttcaaacgcagcagggccagaagagacagagaaagacagaggaacccccataggctgggggtgagcagggggcatgagggctaaatgcaacacgggacctagaggagacccgaacagcaaagggacggtgcggaaactggggacatcagaataaagccagcagcttagttaacggtatcgcgccagcatgagttgctttggttttgataactgtacggtgagtatgtacggaggatatatgcagagtccttgaactgtctttcccactctctgcaagtctaaaattatttcaaagtaagaaattatatatgtgtgtgtgtgtgtgtgtgtgtgtgtgtgtgtgtgtgtgtagagagagggatggaaatgtatgtgcggaatcattgaactggcattgccagagagtgaaatattgcatttccatgaaatttccccagatgccaaatattttcttatcttgacttcatgatggactgtttccacagctggtaacagtcttcctgcctgacagtctgctgagtatattttcatctattcttgactcggatcgccatcaccagcctggttattgagcccagtggtcctttagggctaagggattgagtgagaggtccagggacttttcgactccgccccgtgtggctctgagattgtgtgtgtgagttttacacgtgtgtgttcatgtggtgacttgtctagtattctcaggccatctgctgtctgttcttactgctttggggatgtaagaaaatttctttttctgtgataattactgcttatcttttcttctaatattctgcttctatttttctctaatgttgcattagcagaaatactctattttgtacttatgcccgtacctaacctgttagcttaaacagcaatgccagggacatagagttagccccatggctctgccgcactgacaggctgccccgttcacctggagatcgttggggccaggagctctgctgcctgggctcaccgcctctttacgcggttccgaccgtaccgtgtggctctgggccagtgcttgctctgtctcagtgtcctcatctgtaaaatggaaccgatcatagtacctgcatcgtggttgcttacgagggttaagcagatacaaggagtttcaaagaacacctcaggaaattgtgtttgtggttattgtggttgcaatatcatgactattcctgctcaccatgttaacatcattttgagtttgggagcaaaggatgcttctgataacacgacatcttgcaaaacagtcgtgttgagaagacgaagcaggcaaagccagggctcttctactggtgtatcctcctcatttctgagaaaagaggtttccccactccctcccttgacagttttgaaaagggacagctgttggcttagaaatgatttatcttgtgatgagaaataaccttgaagaaaaaacgattttatagctttgtgccatatattttgaccggaatgtagggtgcacgtcctgaaatgtgttaaaaacaaatgcaaatgtgcccaagtgtgttagagaaatggctacactcttccctccaaagcccacgtccacatgggggaaggagcaaagataatccggaaaaatgcagagtttgctcttgggcgttctccagggaatctcaggttacccaggcaacagccagcaaactccctggcccgccaggcgtgctgctgggaggaccttagaagtcttgagacttcccttgatgccggagggcccagggtgctgcttggaagaaccaggaggacaggcatagctgctcaggtcactctagggaaacaaggcccaggagagcaaggtgccaggattattcctgggtcctccttgtgccaggaaagggggccagagccatatgacggtggtccgggtcccagggccctggagtctgtggccgtctgcggaatgatcattgctgcctcacagtgtggctgcaaaggacgagatcatacagtcgagactgcagatattgcgtggatgggggcccgacttgttcacgggtaggatcttggcccatagacattcctaagtcatagtagctactgtcattatgacactgctttttctctaatgtgcctgcccgtgtggctccctgtgagccagctgctagggttgtctcccaaggcagtgaggtgcttccatgcctgcagcttcctggagtcagaaatactcttcccagggagggcttctcagcaggggtggagatgcctgcagctgtgcagggggagacgctgtttagccggctgggaaacatttcacagaggcatctgtacttggcaaaaaacatgttgtgaaatgtcttaatccagattaaaattaagctaatgaatcgctcaaggaaaattgccatgggggttggggggtaggaggtcaggggagggaaacaaacccacccaccattgcatggtgggaccctcctggtacagaaggggcctctaactacagctcagaaggggatgttgagatgtgagaatctctccattgggtagatggatttctctttttttcttttcttttcttttcttttcttttcttttcttttcttttcttttctttctttctttcttttctttttttttttttttttttttttttttgagacagagttttgctcttttgcccgggctggagtgcagtggcaccatctcggctcactggaacctccacctcccgggttcaagagattcccctgcctcagcctccctagtagctgaaactacaggtgtgcaccaccacgccaggctaattttttgtattttagtagagacgggctttcaccatcttggccaggatggtctcaatctcctgaccttgtgatccacccatgttggcctcccaaagtgctgggattacaggcatgagccactgcgcctggcttcttttttcttttttaaagagacaaggtcttgctctatgacccagtctgcagtacagtggcatgaccatagctcatggcagcctctccctcctgggctcaagcgaccctcccacttcagcctcctgagtagctgggactacaagcacacaccactgtgcccagctaatttctaaaatttttaaaaagcttttgtagagatggggtcttgatacattgcccaggttgatctcaaactcctgggctcaagcaagccaccacacccagctctaagatttcttttgtttcatttgtttgaaagtttttatctagaaaattatccctcaaaaggaaggtagctgaggaatcttgctctgtcgcccaggctggagtgcagtggcacaatctgggctcactgcaacctccgcctcccgggtccaagcgactctcctgcctcagtctccccagtagctgggattacaggtgtgtgccaccacacccagctaatttttgtatttttaatagagacagggttttgccatgttgaccaggctggtcttaaactcctgacctcaggtgatccacccgcctcagcctcccaaagggctgggattacagggatgagccactgcgcccagccaaaatccgcattttcaaagtgcagtaaaatgatggctgctgggtccgggtgacacaggtctgtgggaccatgccacccagcgcggcctggagtctgcacctggctctccccccacctcccatactcccacttcccggacaaacctgctgggaatggaataggcagtgagcactgtcccggccctcaggaggcccgagcgtgggtgtctacagctggccatgtgacccagacagcccccttccttctgcaagcgcagcctcatttgccatttgtgggcggctctgccagacctcacttctgtatcagaacttcttatcactgcagggtgcaggcctcggcctctgttcaaggggatgtaagcaaagcactgagttccccacctgtggagaccactgccccaggcccccagagctcagcctcatcccagtgggcatgcccgggctgcaccggccttccagcccctctgagggaagcccttgtggagggcatggagccccctgtcctcagtgtgccctctggtgccaagttggtttgactgtcacggtgacacattcatggctctaaatgcttagaaaatctcataaagccagtgtgccgtggggggcactgaagaagcaggcggcctcgtcttaaagcacaggagggttggccgggcacggtggctcacgcctgtaatcccagcactttgggagggcaaggcaggcagatcacttgaggccaggagttcgagaccagcctgaccaacgtggtgaaaccccatctctactgaaaatacaaaaataagccaggcgtggtggtgggcgcctgtaatcccagctactcaggaggttgaggcagcagaattgcttgaacccaggaggtggaggttgcagtgagccaagatctccccactgcactccagcctgggtgacagagcgagactcagtcccaaaaaaacaaacaaacaaaaaaagagagaaaagcacagcagatgaacttgaggcaagaaaaatacctgaaacagactgacgccaggagattcctgtgactaaatggctacagaagaatagataccaagggctgttctgtctgtaatcaaggacattgaattaccagatgaactttaatgaccagagagcatagttaacagaagttttcatattcactaccagcatttactgagcacgcacttgcacttcgggctttggctgagccaagtgcagcagacgcttccacacctccagcttcctttcctggtctaccggttgggccctgaaatggctggtgtgccccatcaagcaccaagccccccaccttagccacaccagagagttccctcaggcatcttttaggcatgggtatcaggcacacctattttattcttctcttggtgttagatttttttacatttaataagagcagtggtctcattggcaaatatatacatatttatacatatatatatgtattttgagaatgggtttgctctgtcacccacgctggagtgcagtggcacaatcacagtgcactgcagccttgacttccttcgctgaagcaatcctcctgcctcagcctcccgagtggctgggaccacaggcacgtgccatcaaggtcagctaattggttttatttttagtagagacaaggtctccctatgttgcccaggctggtcttgaactcctggccctaagcagtcctcctgcttcggcctcccaaagttctgggattacaggtgtgagccagcacaccagcaaaatgtttagaagtttaactctgccagtcaataccaagaggtgctttcaaatagactcactgtatgttattgataactagaacattttacctctgttgataatgtcagtttttaaagagctgtaagcaaatgttatctagatgccattatttgctcagatatgttaattaatgatttcgctagctgttttggcatgtatattttcattttttgaatgcatacaaatgttcaaatttagcttgtatgttacatcagattcccaaactatacaaccctgtagaatctttgtttcttttatttaatatagtacaggtctgcagtctaactttaaatacaacctgggtcatatctttgcccttaggtagtgacacttactttaaatttccaaaataatatctggcctcccataatttatttcaatgttttacacttcttgctgcaaggaaattgcttttgagtccaaaaaaaaaaaaaaatatatttcttgccacggtttgtcttcttcattggaagctgttagttgctttcttagatggttcctaggttacctggtcacattaataataataataagtttattacgggtatactgtatgccaggaactgtgctaggctatatatatacttatttcatttaattattatttttttaagacagggtctttctcttttgccccaggctggagtgcagtggcaccatcaaagctcactgcggccttgacctcccagactcaagcagccctcccacctcagccccacaagtagctgggactacaggcacatgccaccaggccaggctaatttttatgctttttgttgaggcagggttttgccatgttgcccaggctggcctcaaattcctggactcaagccatcctcccaccccagcttcccaaagtgctgggattacagccgccacacccagccaatttcatttaattcttagcaacatatcttcactctttgtttatagatgagggaactgaggtaaagtgatttgaacagagtctccaaactagtaagtacttgactagggtttgaatcctagtttatgagaaataaaggctttgctctttttagggtacctgtattaaatcttgccttctcttttctaggtccattaattcctataaacttttaagtacgcctagatctggaaatcgtgtacctttgattattcagcccatcttatgagactagctcaccatcacacggagcttaagaaccttggatttgtgtcctcgcatagttggtggccagtttctttacttaataacagctttgttgagatataattcacatactataaaatcgatctttttggccaggcacagtggctcatgcttgtaatcccagcactttgggaggccaagacgggtggatcacctgaggtcaggagttcaagaccagcctggccaacatggcgaaaccccgtttctactaaaaacacaaaacaagtagccgggcgtggtggcaggcacctgtaatcgcagctactcaggaggctgaggcaggagaatcgcttgaacccaggaggtggacgttgcagtgagccaagatcgtgacactgtactccagcctgggtgacaagagtgaaactccatctcaaaaaatatttaaaaatcgatctttttaaagtgtacagttcagtggtttttagtatatttagcaaggttgtgcaaccatcaccgtgatcaatttcagaacatttttatcatcccaaagagaaaccctgttgaaataccacaaaccaggtggcttttaaacaacagaaatgtatttctcagggttctggaggctgggaagtccgagatcaaggtcccggaagattcagtgtccagtgaggacccgttcctgcttcacagatggtgccttctcccagggcgaagggacgagggattcctctggggcctcttttatgagggcactaatcctattttgaaggcttgtcccccatgacctaattacctcccaaggctttgccccccaatgccatccccttgggaattaggatttcagcgtctgacttttgagggggcacaaacattcgaatcgtagcaaaccccacgccccttagcagccactcccccttcctgcccccggtcccgcctggcagccaggaatctgccttctccctccaaggattcaccttttctgaatatttcacatagacgggatcacgtaatatctgtgctgtgtctggcttccctccccgagcataatgttttcgaggttcaccatgctgtggcaggcgtcagtgcttccgtctttttcgtggctgagtaatatcccattgttcgaatagaccacagtttgttgatccattgttcagtcagtgaacattcccggttccatttttgggtcctgttgcgttcgtgcccgggtttctgtgtgtttccactgtcactttgcccgttggctttcagctcatccccgagcggttctgtgtgccttccccttgctcaccaccatgctgcgtgacggcagatggcaacagcggacctgcctgaattcagggtggcagtcgcggtttctgggtgtgtccccacagtctgtgtttttcttctagacctgcccttcgtgcggaatcacatttgctcccaaatctgaagccggcgccgagggaggagcacctcaaaacggctgccaagacgagcataaaaacagcaccaaggtaacaagctcatggctgttgttcctgtcagttcctctcctggcgcatccacccaaggtgacattcctgtcttgtcgacttcttagttctgagagtaactcctcttgggtaacagcactccgcacccgcgggtcctccgggtgttgaagagaccggcagggagctcgagggagggtcagcagcaacccacgtggaaaccagcgctcaggggctgagctgggcgggattcctttaaaggccaataagggtttaaaatgcgtggattcgagtgccgggaggggatttgtggtactggaagctggactcagtttatgagagaatatagacgggcagagacaagagggggtcttcacggcgagaaaggctgggtgaagagagaggttgggagttagggccggtggcaggaaggctttcaggaaaaccagacttggacaccagggccaggactcagcccacccgcgagagcctgtcggcgggggctcatgcaggcgactcacctggggcgcgggctctcgtcctccatctgctctccatctgccaaaggattgcaggtgcaggtcatcccatgccttcccacagccacgggggacacccacgacacgagcccgtttccatccaccttgcctgtgccctgacagccctggacccctcgtcatacttctcctggacccccgacccatgcaccctgctcccagaaccgtggttctggcctaagcagactctgggggcctcggctcctcactttcctcccttcccctccacctccgcaccgactcttcccatgtctcaatgtcctggcagcctgaacacccagcgttaccactgggcactgttgggtaacaggaaggagcagacgggcagtcagggctgggattggagcaggtctgtaggcgcaggggggtgggggttcctcgcaggtcagaaagtcccacgcccagtgcagcagcgccccgcagcggaggtcggggtgggacaggcgtgtgtgcccagtgcccggtgccacttcacttcccgtgtctgcgtcctctttgtctcgaggcaggcttctggaggacctaatcccaaaactcagaacgggctcctcagccctccccaagaggagaagctcaccaacagtcagacttctctgtgtgagatcttgcaggagaagggaaggtgggcaggggtgagcctggaccagtcggctctccttccgctgaggttcaagaacatccgggagaaaacggacgcccactttgtggacgttatcaaagaagacaggtacgactggggctgtctgggaaaaagcaaaggttgcaggcagaagcctctctgcctttttcttttcttttctttaagactgagtctcgctctgtcatccaggctgcagtgcagtggcgcgatcttggctcactgcagcctctatctcccaggttcaaacgatgctcctgcctcagcctcccaagtagctggggttactggcacccaccaccatgcctggctaggttttgtatttttagtagagatggagtttcatcatgttggccaggctggtctccatactcctgacctcgggtgatccacctaccccaccctcccagagtgctgggattataggcgtgagccaccacgcctggctttttttttttttttttttttttttataaggactaggtcttgctgtgttgcccaggctggagtgcagtggctcaatctcggctcactgcaacctccacctcccaggttcaagtaattctcctgcctaggtctctcaagtagctgggactacaggcatacacaaccatgcccagctaatttttgtgtttttagtagagatgggtttttaacatgttggccaggcgggtctcgaactcttggcctcaggtgatctgcccgcctcggcctcctaaagtgctgggattacaggtgtgagcctgtgcctggcctaatggtaacatttttgaaaaatacaggtccagtccttttatcaaatattcctgaagttggattggttagattcaggctctccctctggccaggcagagactgcagaagggatgccgtgtcctcctcagggtgtcctctccagggacacgcagtgcccgtctgttcctcattagtgatactaattttgattaattaggcaaggtggtgccgggttgtctcatctagggttattatattcctcttatgactaataagcaatctgtgagaaaatacttgaagatcttgtacatatttcattccccatcagaattccctagatttaacattcattgatgattcttgcctctgtcttcactataatgcctcaaagggtgattttcagccaggcgctgtggctcacgcctataatcccaacactttgggaggccgaggcaggtagatcacctgaagtcaggggttcaagaccagcctggccaacaaggtgaaaccccgtctctactaaaagtacaaaaattagctgggcgtggtggcgggtgcctgtcatcccagctactcaggaggctgaggtaggagaatcgcttgaacccaggaggcagaggctgcaatgagctgagatcgcagcactgcaactccagcctgggcaacagagcgagtctccatctcaaaaaacaaataagaaataaatggagcagcacagctaatagcagtgtcgcttctgccacttgactgctaaaatgtggccttgtgcacatcctctctgcagctgcgtggttgctcttccctcagtccacactccctgagtcaggggaggagagagggtcagtgtcctgggacagaattatttctcttctgagtctgagaatgttggaagttactggatacctgtcacagaaaggagagtgtccgccgtgctgcaaggacaggaaggaacacacgtgatgagccgtactttgtgtggggggcacatgaggcacttgacatatttcacctgatttactctttttttttttttttgagacatagtcttgctgtgtcacccaggctgtaatgcagtggcgccatcttggctcattgcaacctccatctcccaggttcaagtgattctcctgccttagccacccgagtagctgggattacaggcacctgccaccaggcctggctaatttttgtattttttgtagaaacgggggttcaccatgttggccaggctggtcccgagctcctgacctcaggtgatccgcttgcctcggcctcccaaaggtctggggattacaaggatgagccaccgcgcccagcctcgtctaatttacttctgatagtcactgtatgagggagagaggctaacacagctatatttgctttctgaaatcatgttaacgttcccttagtttctagtaattttctgcagtatagaaaggatattactatttgctggcaattcataaatgaatgaaagagtcatggaaagcggggttgatttccctgctattaaaatcactgagctttatcattctgcaggtttcacatgcaatctgcatatgtgacaatagcaccaaaattacgctaactataaacacaattttgtgataacaacctgttagcttttacggtggcaattactgtcagtgctctgaatgacaggaccgccggttctgaattatgttttcgtttgaagcctccaaatccatgtaagtactctcagcacagtgatctgcgtgttcttcacaaaagattgtttcaaagaacagaaaagctgccttcatcttctcctaatcctagacattaggaatgtgttttcaaaaacaagcctcagcagctcacgctgtcatcccagcactttgggaggccgagatgggtggatctcttgagcccaggagttcgagaccagcctgaccaacatggtgaaaccccgcgtctgctaaaaaatacaaaaattagctgggtgcggtggcaggcgcctgtaatcccagctactcaggaggctgaggcaggagaattgcttgaacctgggaggcggagattgcagtgagccaagatcgctgcgccattgcactccagcctgggcgacagagtgagaccttgcctcaaaaaaaaaaaaccccaaaaaaccaaggttctgatgatgtgtgctataatcatggtggatggtcattctttaacgaaggtgaaacacaaaatatgattatgctttgaggtgcgaaggactttccacagatctgttttaacaaggggcagctgagtggcgtctgttccacaggcgcttttagacgcagctgttgtaatatacctaatgagcagcgttttggtagctaattatgatctgaaaacattggcacagttatggtgctgggtattgatcgactgcatttgttgtgtcttctagcctgatgaaagattacttttttaagccgcccattaatcagttcagcctgaacttcctggatcaggagctggagcgatcctacaggaccagctatcaggaagaggtgcgttcatttctccttgcgcttttactaaagaatgtgtttgttgttctcacatggtcattcactgttagttttgtgctttaccaagggatgggaacacgaaggcgaacattgatttcccacactctgtaaaccctaagtggcaactgtacatgaaatgtctctttgcaaaggaggaaaaaaaccctttctttagtcctttgtcttaaagcgcagcctcgccattactagcccctttcctctgccgtggcatctccaggctatccccagccttggaaggggcccccccaccacaacctgtcctgaaaggaaacccccaacctgccctggaaggaaacccccccacctgccttggaaggggcccccacacctgccctggtaaaggaaccccccccacctgccatggaaggggtcccccacctgccctggaaaggaacccctccccacctgccctggaaaggaaccccgacacctgccctggaagggaaccccccacacctgccctggaagggaatcccccacacctgccctggaagggaaccccccacctgccctggaagggaactccccaccgacctgccctggaagagcccccgtcccacctgtcctggagtcttgggtgagactcttctctgttgccctccctgctttcttcccctgagaagggcccaggaggccttcacctctctctctaactccggctccatgttccctctcccgcccccaccgccttaaccccttgtacttgacttctcagcccatcacccaatcccacttcctccaagaagctctccccgactgcagactggattgtacttcccttcatacacttccttctccgtgtgtgccccattttggaatctttccgtgtgtggttctggattcgtgttcctccctccctagatgctcagctctgtgaggtaggacccccgtctctctcctccagcccctccttacagcctccagcacagtcccaacacccatctctctcctccagcccctcctcacagcctccagcagagtcccagcgctcacaagtgctccttagggggttgctggaaggaataatacaagaacctagagcctggtttgaatcctggcggcttccagactgcattctctcctctgggcccaaacctaacctctgagtgttcccaagtcaagagtggatgccctccccacccggaacaaaagggatttctcactctgattcccttggttcctccagcaggtcttcattaaactccgtggtgtcctggcttgggctctagagagaccctggtgagcccagtagagccagttcatgcctcgtggggtgggaggtgcccccttccgtcagcctccaagggacagtgaatatccgttaatgtcccagacatggctcccggagtggacttgcaagttggcagttggctggttggtggcccgtaaggcaaaatatcgtctcatttaatgctcatagcaatgctgtagggaagctgatgattatcctccttttaccaaagagctgacgctgagcaaggctgaacaggtggctgtggtcccatagccggggtcggacacgagtggtctggctgccaaaccaggcgtccgggccgcttctcgccctgcactgctgagcctcccaaacagcagacacctttgccatgccttcctccaggcaccaaatgtacgctcagcctctgctctgccagggactgttcttggtgctggggaagggggaaagacgtggcctgatggggagcaggaattagaaacaacatccggagctggaggtctgcgagtgccctcccagcacacggcgaggcaccaagcccaggctgggggctccaaggggggaactcctgagcctgcctcagtggggggactaggaattaatggaatgtgggatgagggaacattctggagagtggggccagcacccacaaacgttagagataccaaagagcctggttcgtccagggagttgcaggcgcatccggggcagggtggggcaggagtggcaggacgtggcaggggccaggtcacggctcccatgccatggactttatgccacaggccatggggagctgttgaaggatctggaacaggttgtgttacaaagatcacacagctcacacctgtaatcccaattactcaggaagctgaggcaggaggatcacttgaggccagtagatagaccagcctgggcaacatagtgagaccctgtctcttccaacaaaaagaagaaaaacatggccgggcgcggtggctcatgcctgtaatcccagcactttgggaggctgaggcgggcagatcacctgaggtcaggagttcgagaccagcctggccaacatagtgaaaccccgtctctactaaaaatacaaaacttagccaggtatggtggcgggcgcctgtaatcccagctacttgggaagctgagacaggaaaatcgcctgaacccgggaggcggaggttgcagtgagccgagaccatgccgttgcactattgcactccggcctgggcaagaagagtgaaactccgtctcaaaaaaagaaaaaaaacaaacatgtatcagctgagtttcggagggtgataagagtgtggacaagacaggaggccacatgacaggtgggaggctgttaccgaaactcagggcaggtggccacagggtcccggtggggaggaaaggagagatgccaggacaagggagcctcccagctggcccgcgtttggggactcattgtgctgggggctccacgtgggtgcgttcttagccgagcatcccaccgcccacaaggtgcaagtactggtttatccctggttttggataaagagactgaggcatggacgcgtgggtatcatagctaatcatggtggagtggccttgagcccagacagcccggctgcagagcctgtgctctgatgatggtgggacggccgcacccggaggccctgggatgtgggtagtgagcagggcggagcctggcccacggtgccctggcagccagtggcactgtccctgggtttcgcccatggggagagagggcgaaagagtgtcatgatgtgggggagcgtcgttctcagtgggactgggacaggtggcactcccgggatgcacaggggtggggcagatgggcctgggagcctctggcatcagggccaggggtggcccagagggggaggctgtggcagggacaggtgtctctggcaagtagcagggcaggcaggagagactgcaagggaggtggagggaggagcggggcctctggcagcgtccatggatggggcagttctacagaggagatggcagccccaatcaggaaaggccctgtacccgagcccttggcttggaaggtgggaggtgtgggctctcagaaggacggcagcccacgtggaaaccttccggcctcacatgtgggcaaggcagtgatgtctcagggacatcacatgcatgcacccggccctgactcgaacccctgacgggcactagatggagccccacagaaaggggacgtgcgtgggcctctgccaaggtgagtgggcatgggcagtgtcgcccctgaaattcctacacctggccgggtacagtggctcaggcctgtaatcccagcactttgggaggccgaggcaggcagatcacgaggtcgagatagagaccatcctagccaacttggtgaaatcccatctctactaatcatacaaaaattagctgggcgtggtggcgggtgcctgtcctcccagctactccagaggctgaggcaggagaattgcttgaacctgggaggcggaggttgcagtgagccaagatcgcgccactgcactccagcctggcgacagccagatcccatctcaaaaaaaaaaaaaaaaaaaaggaaaagaaaagaaaaatgaaaaaaaagaaattcccacaccttctgaggggattccagggctgggacctgacttgcccttttgggcactacagagcctccagcttccttggagagaatttggaaccaaaccctaacccccaggttgccctggcagggtccagcctggcatcagggctgactggcagccttgctcccaccgtccaagtgcccctcctgccctcccacctagttcctgggagtggtcgaggcacccggagctggtttgtcccaaagaacgcacaccgcacccagggctgccaggagacctttccgctccgtgcgtctccagctcctgccctcagccccgagcctggccgggatgctgagtggacacccatggcctctcaggggttgccagaaggttgcaaggccaaatggaaagcagctgaactggctacatcccttcctctcccaaccacagcctctgcctttggatcctacaccttcctctaggttagggacacactcaggagtcaaccggtccatgccaagtcctggcctggctccttgttcgctgtgtgccctaggggtagcccctggccttggcctcccaatctgtacaccgagatgggtggtagttcctgcctgtggggtgccgtgagcgtcaatggagatcgcacctccccagagccttgcagggcctgggcctggtcacctcttggacgtgtcagcatcgttatttctccccctcaattgccgaacactagtgtgtgcctctctagtgtactggcccccagcacagagccaggccccgtaggttcccaggcgatgatgtgccacttaaatccccgtcggccacttccgccacgaagccccgctttcgcacctgctgaacctaggactccgccatgcagcaggaccgccctgcacattgctctcgtccaactgccttacagatgcagggacctcacctgcgccagtcagtaactcatcttgcagctaaatatataatgtgtctatgagaatgtgctgggtcataatcagttgctctgtgaccctggcagggttttttttgtgtttttttgttttcggaaacggtataattctgcagtggtgtgatcatagttcactgcggcttcaaactcctgggctcaagcaatcctcccacctcagatcccaagtacctgggactacaggcacaaaccactatgcccagctaattattttattttttggtagacggggtctcgctatgttgcccaggctggagtgcagtggcatgattatagctcactgcggcctgaaactcctgggctcacacaatcctcctgcgtcatcctcctgagtagctgggaccacactcacgcactgccatgcctggctaattttaaagttttttgtagagatggagtctcactgtgttacgctggccgggaactcctgggctccagcgaccctcctacgtcagcctcccaaagtgtcgggattacaggcgtgagccaccgtgcccagccccctgggcaagtttaacttctctgtgccttggactccccagtgttaaatggggtagtagagggagacagccctccgagagccttcctgccctgtgcctggcccagagcagcagtgctccgcacgtgttgcctgtttctccatctgaactgcgctcctggccgcccatggcctcccagtcattctgctccactcctgctccacacaggtcataaagaactcccccgtgaagacgtttgctagtcccaccttcagctccctcctggatgtgtttctgtcgaccacagtgtttctgacgctgtccaccacctgcttcctgaagtacgaggcggccaccgtgcctcccccgcccgccgccctggcggtcttcagtgcagccctgctgctggaggtgctgtccctcgcggtgtccatcaggtaagcgccgcgtggactgggtctccagctctgagccgttagctccctcttctggctccatggcttatggctgagcggttggactggtccccagccagctgtccctgctggctgatttctggagatgggtccccgtgtcctccccgagccctgtgccccagcatcctccctccctgacgttggcccagatgcggggggaccacctcgctggagggggcggtgcctccacagccccaaccgctgccagcttgtccccagatttccaggttcagagactgcaaaggagaaggtgagaagataggaaacaatctggaggaaggaaaggaaaagacatgagcgggggtagtgggcctgggacagcaggcgcctcttcagagtaccggagccaaacagcatctccacgcacttgcctcaggcagtggtctcttggatgtgacgccaacagaagaaagaatagacaagcccaacttcctcagaagttaagctgttgcgctacaaagaacaccatcaagaaagagaaagaacccacagaacgggagggaacacttgcagatcaggcctcggacaaggtctaacatctagaatggataaagagcacacatagcctgataataaaaagacagctcattttaaaggggacagaggctccgaatagaccacaggtgcgtcttgggaaagctgggccctcgggaccagcctggctccctgagggggctcgcagcggctctgtattcctgagcaggaaggtggccctgtgtggtccccgtcctgggcaccacaagccccttgggtggtcctgtcaccctcaacaggatggctgccggcctagcagagtcccactagcaacggtgccccggagctgggcttcaaacagacatggggaggcagccctgaccttgagcgtctgctgcccttatccaacatcccagggcagaaatgaatggctttgagtctggcgtgaggcgccggaatgctggcttctcccatctggggtgggcaggctgggattcctgccccctccttcccacctgcactgtacccaggaccctttcagcttcctccgaggccgccatgggccatgctgtctgtggcgtgtgctcagtgtttcccagaactccctgtgggctcatgccccatcacccccagcgagcagccctcttcctggcacagcctcccctcctgcctcagtgccctcctgtgcttctggggacccctcccaaaggagccgtcgcctgcaaaccctttcagggtccacttccccagaacccaaaccagacatcctcaggcccctggttggtttgtttttcctctttaaaaagatcagctttaggctgggcatggtggctcatgcctgtaatcccagcactttgggaggccaaggcaggtggatcacctgaggctgggagttcgagaccagcctggacagcatggtgaaacctcatctctactaaaaaataccaaaaaaaaaaaaatagccattcttggttgtgggcacgtgtaaccccagctactcgggaggctgaggcaggagaatcaacttgaacccaggaggtggagattgcagtgagctgagatcgcaccattgcactccattctgggtgacgagcaaaactccatctcaaaaaagaaaaaaaagaagatctgctttattgtgacatgatttatatacaataaactcattcaagtggacaactcagtgatttttagcaaatttgaagaattgtatgaatatcaccactatgtaactatcgggcacctccatcaccccaaaaagaaacccatcctcatgggcagtcactcccccaggtctgccttccctggtccacccccaggttttcctctaatgtgggtagctgtagcgtcttgagggctctgaggccagcattgtctccattcctttttactttcaccctttagctattattccctgctccttcagacccaattctaaagcattgagggcaaccccggtgtagactgccgagggctggggtgaggtgcgctggaactgcctgcacagacatactccccaaggatctggtgaaggtggtggctgctgcacccctgaacaggcctgtggtgggatttggttccatttaccaggaatcgtcaagcctgggagaggcccagaccctgaaaggcttccctggcccctctgagggccgtgttgaggaccccaggttgacgggaacctgcgctgtggggtatacgggcaccccatccctttcctgtacaggaggaaagccaggcgcctcctcagtgactggcccaggccccgcccgttgcctccaggaggcttcctgcatcaccctctctgctctgccccagcaggcgtgaggtctcattttcagatccctgcagtctcagggaccagcatgaaggccccctccaacctcacatctttcccgctgctgactgggaacttggttccctctgtcccccctcctagggcctctgtattctgcatatttgtggttattcttggaagtattccaaagtcttcattatcctttaaatatcaggaagccagggctgtccaggaccctaaaagggtgaggctgtgcctgcgtccatgtggggaatggctggagagtgagcgttccctcgcctggccacaccacgcagccaagagggagaggcatggtaggtgttcaggcccgaaggctgggggcggggggctctgtgccagaggcttttttgctgttcagtcacaacagcggcccccgcccagcctctcctgaggcagggacagcaggcctatccccagctccctggcagcaagcttgagagaaggactgtctccccacgaggaccccacaccttctcttcacctccccatgtggccccaccatggcccaggggtccttggactcagccaagacctgttcctcctcctcacagtggttcttggcccgcacactcagctgcttctttatacagaaacttttattgtgtcaacatcacactcctgaaacgaagcccgctagacacacgcctgcaaaaggagtcagtgaatacagtcgctctgcttagaaggacaaagtcttttaaaatgaaatacgtggtgctggacctcacgtacaacagagccccgctgtctggagagtcctgaagccgccagctagaggtgaacatgcacgatgaacatgacagcccagatgcagacggcacgcgtgtgcgggtttggtggtctaagcgccacgagccaggttcagcctgtggtgtgattttctgaaacactgatgatgatgccccaaacagcagagaccactgtcactcagtttccacaggagctgcgctccaagaaaatccagggtgtgtcagcaccaccacagagcaccctgtttgtagctgggacacagcctctgggctaagatgctgtcagcagccttctgcctttgcaggtgcccaggggcttggctgctgtgtggggtgcagggcagaacctccctgggtggcacctgcccacatgcaggacagctctgtccctggcgcctgcccaccagatgccaggaccgcctcttctgctgcgacaactgcacgggctccacagaattccaggacgcctccaaagctgcccccgtcaagacctcctgcctcagaagctgatgggagccggtgacggcaacgtggtggcccccaggagactctcctcagggcctgtgatactctcacgtgttctgttaggcgagcgaagcatgtgcttttcacaagcatattttatcacaacgcaacatgtgaccctagaatcaggtacaatgagctaagcctggcttccttgccttctaccaaaagtgagggaggtagactcccaccccacgcctgctctcacgcccatcccaggggcaacacgggagagagcagagggcggcgctggctgcggcctcgcacctgtctcctggccgatgacccaccccgggccccgtcgctcctgctaacccccatgcctctcgcaggatggtgttcttcctggaggacgtcatggcctgcaccaagcgcctgctggagtggatcgccggctggctaccacgtcactgcatcggggccatcctggtgtcgcttcccgcactggccgtctactcccatgtcacctccgaatatgagaccaacatacacgtaagtaaaggctcagccgttttatttccacgctctcctggactccctaatctctcttttctttaaatcacttgggatagcaatttaaaaataaaccctttggccaggcatggtggctcacgcctgtaatcccagcactgtgggaggtggaggtgggcagatcacaaggtcaagagatcgagaccatcctggccaacatggtgaaaccccgtctctactaaaaatacaattagctggtcatggtggcaggcgcctttaatcccagctacttgggaggctgaggcaggagaatcgcttgaacctgggaggcagaggttgcagtgagctgaggttgcaccaccacactgcagcctgggtgatggagcgagactgcgtcacaaaataaaataaaataaataaaataaaataaacccttgaagaggcacgaactcttttactctcctgagccagcttttttagaagccgacttagcattggcctgcccagatgctcacgcccgggcagtggccgggctggggagtccccggtgggcacgagtccctgccagtgtggcttcttcttcatcaagttccaaatcagttattttccttcacattttacaaagaagcgtctgtcccacctttcctaaccatgcgagaagcatacctggcacacagaattgacttgtgcccgggtcggctgctgctgatcaggaagggaactgtgctaaactccgtggagagggaaccagcacggccgtgggagatgatccttctggtgcaagtgagcatgagcttccgggcgggctgatggtgtttgacactttagccccaaatcacatctcactgggccccaaggaaaagtgttttgaaggaaactgatcacacagcgtttctcaaacagggaggcctccctttcctttccacgaagacacgtgccatttcccacacctgcagaagagtgtgatgccatgtgaacagggagcctcacgccgggtagggaacctagaggcttgatctgtccttgacactgcccactagcaaaagtaagactgactgcagatagtgagcaattagctggtatcggtgatatttttaggttacacagaaacatggattaggccgggagcggtggctcatacctgtaatcccaggcactttgagaggccgaggcaggtggatcacctgaggtcaggagttcgagaccagcctggccaacatggtgaaacccagtctttactaaaaatacaaaaattagcgaggtgtggtgccgggagcctgtcatcccagctactcatgaggctgaggtaggagaatcacttgaacccggaaggcagaggttgcagtgagctgagattgcaccactgcactccagcctgggcgacagagtgagactcagtcaaaaaaaaaaggaaacatggattagtggcgaatgttttggcggccgggtatagcaggggtggcggggtggccctgggcggctgtcctaacccttcgcctcttgcagttcccagtgttcacaggctcggccgcgctgattgccgtcgtgcactactgtaacttctgccagctcagctcctggatgaggtcctccctcgccaccgtcgtgggggccgggccgctgctcctgctctacgtctccctgtgcccagacaggtacgcggccagcactgcttgcgggcaccagggtgcgtggccaccttgcccgaggttgcgtagggcctggctgcatttgcacattgcttctcacgccccgatagagatatttcccatcatgacccaaaatagctgtcagcacacatcataccaaagagctaatgagatgccctcagagtaatatgcaaaagaaagaaaaagaatatcatttagaacagcactctgggtagttgaatgtagacatcctggaaaacataatgctgccattgcattataccgaggtgacttctgatgaatgggctggaatctaagaaagtaggacctgaaggatttcatagaagatggtcaaggccatgaaagagcggagctgcagatggatgtgaaggtggagaggaagctggtgctgactttccttgagtggggtagggttcatgaaccgggacaggtcacagcctggaaggaggaggtgaagacgtaggatgatcttgcaagtcacctggggcttatttcaaaatattatgtcaggctgggcacagtggctcatgcctgtaatcccagcactttgggaggccaaggcaggcagatcacctgaggtcaggagttcaagaccagcctggccaacatggtgaaaccccgtctctacgaaaaatacaaaaattagtcaggcatgggggcgtgcacctgtaatcgcagctacttgggaggctgaggtaggagaaccgcttgtacccgggaggcggaggttgcagtgagctgagattgtgccattgcactccagcctgggtgacaagagtgagattccatctcaaaaacaaaaaataataaataggtcaaaacagttccccatatgatgtcatgagacgtaggattaatgaccagtcactgaacataggtcttttattttgaaatcctctgcatactgaggaggacgctggccttcagaaccgtatcactggtaggtgctgaggaatgggatggcgcagacacagaaacaggagtcagggaggccggtagacctctggggacaagcccagttctcatcattgtactacaaaaaatgtaaaaacttacaggatggcagtgataggtgctatatttaaaatatttgccattagcgacatccagtaagatccctatctttcatgtgttgcactaactgaataattcttgatttgttaaaaataagtcccagccggtcttggtgctcacgcctgtaatcctaccacttttgggaggctgaagcgggtggattgtctgagctcaggagttcaagacagcctgggcaacacggtgaaaccctgtctctactaaaaatacaaaaaattagccaggcatggcggtgtgcacctgtagtcccagctactcaggaggctgaggcaggagaattggttgaccctgggaggcggaggttgcagtgaaccgagatcgcgccactgcactccagcctgggtgacagagcaagactcagtctccaaaaataaaaataaaaaataagtctcaaaattgcctattacacatttgctactggtttcagggtattttaagccttttcaactaaatgtgttgttggaaatacagtaataccttgtataacagaatgtaagcagtgaacaatttattcaaatatttcgtgatatcttaaacccattaagcttccattcatgtaataaatttctcccaaatattgggcttttcctataactgcttataaaaatgtttcactattagtgtgtattgaagaaccgcctgtttacaattacagagggtttaggtgcgcagaatgttttaaagtcacatgggacatttcttccttgtccttgcaggagatctaacattctggctccctgagttattaagagaaccagagggacccctggcatttccgaaatgccccgttggagtggcattgctgttgccaaggacctctgcttgaattcattcaaaagcacaaaatgattctatttgccgcagtattgtcagtatcagacggagtaggcacggggtgtctttggttattagcaaagcaacagtccagccatgggcagtgtgagcatccgtcctcagtttacttaagaaaagaatcacaatacaaaagacatttaaattccctgcaaattaagatcaaaaagggaatttaaaataggactgtcattcagtacttgaaaggaaactttaatgaatagattaagcaaaataaagcagacttggatttttctgaaggtatggccatctagaacgatggtcacgatgagctctttctcgagagtatgaaatccagcaaactgctttttaccttttaaatccatcacaggtccctcatcgggcattcaccgtggcctttagtcatggctgcggggcttttctacctggagctggagagccacagtgatgggggacccctctgccgcctttgtgcccagagttcaaatagcagatctggggtagggatgatggttgaagttccactcggcccagaactcctccatccacttgaggcctcagagcccctaatcccatcacggttgctaagcctcagcaggatagagaggatgcgggatggtaaacaaagacgtaaactcagctttaccaagggaactgcccccagcattgggagacacagatgtgaagaaagaggtgggagcaggtggctttatgtggatgtacaaccacgtctaaaaaaggaacatagtttgttcaagctcttctcaggttgccttgtggaatgccttctttgctctttatgataatatttttctgcattttaatttcagttctgtattaacttcgccccttgacgcagtacagaatttcaggtaagcttttaatattgcacttctgtctgaataaacgagagtgactgtattaccctgcccatttcgaagcttcctgaaaacaatgggatcttgaataaaaggaacaatacgtgcattcttagaatagaaagtggaaaatatctcctcgttgaactgtatcatagttataatgtctaatggggaaaataaaattagttttattattttaataagacattacagtcatgtaattttatgtaagactttcgcatgcaacaaaggaaatctcgcaaatcaaagaaaaaagttattttgtaaaatattttaaaataaccgacttgaaatcagccaggcacggtggctcacgcctgtaatcccagcattttgggaggccgaggcgggcggatcacgaggtcaggagatggagaccatcctggctaacacggtgaaaccccgtctctactaaacaaaatacaaaaaattagctgggcatggtggcgggcgcctgtagtcccagctactcaggaggctgaggcgggagaatggcgtgaacccgggaggcggagcttgcagtgagccaagattgtgccactgcactccagcctgggtgacagaatgagactgtgtctcaaaaagtaataaaataactgacttgaaatccaaaatactaaatgtttgacccttgctttactataaacagcagaataaattctaaataaattgaagagtcggaaataaaaattaaactatataaaatctagaaaaagtaaaattcaatagttttcaagcctctaaaactgagataactttccaggcttacaaataataaatagaaccacaacgaaaagatggacagatttgagaggaagggagagaaaatcaaagcacacagaaaaggaccgggctcggtgactcccgcctgtaatcccagcactttgggaggccaaggcaggaagattacttgaggcctggagttctagaccagcctgggcaacatagcgaaatcctgttttggcaaaaaattgtaaaaattagccaggtatggtggtatgcacctgtagtcccagctactcgggaagctaaggtgggcttgaggccgggaatttgaggctgcattgagctgtgatggtgtccttgcaccccagcctggttgtcacagcaagactctgtctcagaaaaaaaaggccaggcacgttggctcatgcctgtaattccagcactttgggaggccaaggcaggtggatcacctgagatcaggagttcaagaccagcctggccaacatggcaaaaccccgtctctactaaaaatacaaaaattagctggggtggtggtgggcacctgtaatcctagccacttgggaggctgaggcaggagaattgcttgaacctgggaagcggaggttgcagtgagccaagatcggccactgcactccagcctgggcgacagagtgagactctgtctcaaaaaataaaataaaataaaaaaaagattcattagcaactgtagttgtcaataataaataaattaactgattatacatttactcaaataatacaactgttaaaatgcagtaaatatggaaaacatttaagattttcatttactaggtgaaactatatatgtactataggtacaactaggtatttatatatagtaaaaatatgtacaagatttatacttaggatataatgaatgagtgatgattacagctttagtacttaaaagtaaagcacctagcatctttttttaaccttttattgatgtgtatgtcagtatgcacagaagagtacctgatcataaaaggacagctcagtgcattttcaccagagaacacacccatttaactaccacccagaccaagaagtagagcattaccactaccccagaagccctccaaccccaccgctgctgaccccctctcccccgacctgaccacctccctaggggcagattttaaatggcactgaaatataccaaagtgtcaaccgaagccacgtttggctggttaggactgcaagtgatgtttgctttctatttttttcagtttttcaatccctcttctccatgataacggattacactgtattttactctaacttccatttatgacttataaattaactcctgcatttagtcacatgaaatgcctctttttgagccaggtgtggtggctcacgcctgtaatcccaacactttgggaggctgaggcaggtggatcacctgaggtcaggagttcgagaccagcctgaccaacatgtttgtgaaaccccatctctactaaaaatacaaaaattagctggacgtggtggcgggcacctgtaatcccagctactcgggaggctgaggcaggagaattgctagaacccgggaggcagaggttgcagtgagcctagatcgcaccactgcactccagcctgggggacagagcaagactccatctcaaaaaaaaaaaaagaaaagaaaaagaaattcttctttttgaagtggatctttgctatttgaatttaggaataggatcggctcctttgttcctttggtattaaaataggaaacacacattctagctgggaagctgtcctcgtacctgtttcttacagatgccttctgttgcgaggcagcccccttggtttctgagtgcacatgctgtgtgtgatggcttttcttccttggaagcaaaggtggaaagcaaacgctgtgctctgccttcctctccctctggcactagcccatgtgctgctgtccctcctctgcctggaggggcctggcctgcgcccgtctcgagtcagctgtgctgctggctccggctcccgcagattagagtgagctgtacaagcgataactgattgagaagcccagatacagtctcccatttccaccacagagactggaaacaccccctgcacataatggggatgttagaccttggtgacacctgctcctaaaggggactgttcttggtgtcaccactttgagaaaccaaaagaaaggtcaactcctaacccaacgagcggggctcacaaaaccttcccccagtccatattctcttcctgctctatttacaatcaaccagagggctcatggagctggacagaaatatgtttgagtcatcatcgtaacaaatggttattttgcttagtacttgagatgtaggatatgtaatgtcatctgtttcaatagcagttcacgaaatgagtcagtgaagcacagggtgtcttcccactgaggactcttggttcctggtcataggcgttgagtcacaaagcccgtatcagggctcccaagaccagccccaggtggactcaggactccgcaatctgctctctcaccgctgtgattacagcgaaaagacataaggcagaatcagcaaagggaagggtgtgtgagttgacgtttggaggaagccggcaccagcttccaagagtcctctcccggtagagtcacacgggatgggcttaattcctccagcagtgagtggtgacaacacttgtaacatgctgcctaccagggaagctcattaaagtctcggtgtccaggcttgtattaggggctgttcaggtctcccgcctctgcccagcacgtgcctaaattccagacttccagcaggaaagcgggtattcagcgtaaacctccttatttagacagtgtaggcagtgtgagccgctcttatcaaggaatggcaggaaccctccccaaatcccagctccctggtgccagccaccacctttgcaggcaggtctttccaaggatggcagcctcaggcctttcctgcacggaaccttaggctggaagggacgtagccatgctccagtccatcttcacagcttacaagcgaggaaactgagacccagaaagatggcatggcttacccaagattctgaaactcccccgcggtccagccaggatggggcccaggggttctgttccacccacggccagggcttttggcggttgtgcttcgcagagggccgtgatggccacacaggtcaagctcattgttttttaatatcccaagaacagttagtgaaacttattagacactttgattaaaggactgagcttttggccgggcatggtggctcgtgcctgtaatcccagcactttgggaggccaaggcgggtggatcacctgaggtcaggagttccagaccaacctggtcaacatggtgaaaccctgtctctactaaaaaatacaaaaattagccaagtatggtggcgcacacctgtagtcccagctactcgggaggctgaggcaagagaatcacttgaacctggaagcggaggttgcagtgagccaaggtcgcgccattgcactccagccagggcaacagagcaagaaagaaaaaagaagaattgagttttggccaggcgtggtggctcatccctgtaatcccagcacttggggaggctaaggcaggcagattggttgagctcaggagttcaagaccagcctgggcaagatggcgagaccctgtctctaaaagaaaaagaattgagctttccagtgtgatggagctgtctaaactgtggatccgccagtcgcatatataagtatatagtgaccaagcccattattgcatcaaatcaactggcctactctgtgcgcgggaggaatgcatcagggtacagcatttacatgaacagaacagtcacggtgttgtaaaaaagaaaaagactagaatgcagttcaacaaatgttaatagtgattgcctctggatggcaggacgatggacaaatcttttttgtctatttttttctacattttctatgatgagtatgtagtatttttaaatgggaaaaaatgtattccagcacactggggaggcagaagcagaacagtttgaacccaggagtttgagaccagcctgggcaacatagtaagacctcatctctacaaaaagttcaaaaaattagccaggtgtggtggtgtgtgcctgtagtcccagctaatctggaggctgaggcgggaggatcgctttagcctgggaggtcaaagctgcagtgagctaggatcgcaccactgcagtccagcttggatgacagagacagaccctgtctcttcttaaaaaaaaaaaaaaatacatatatatatatatatatatatatatatatatatatatatttcaaacaaactttaagaaattgtgctactcagccaggtgtggtggctcacacctgtaatcccagcactttgggaggtcgaggcgggcagatcactttaggtcaggagtttgagaccagcccagccaacatggtgaaaccccgtttctaccaaaaaaaaaatacaaaaattagctgggcctggtggcgcacactactcaggaggctgggccgcaagaatcgttgagcccaggaggtggaggttgcagtgagctgagatcacaccactgcatgccagcccgggctagagagtgagactctgtctcaaaaaagaaaaaagaaaaagaaaaaaagaaattgtgctactcaccactcatctgcagccccctgaccatggctgtctccatcagtgccactgaagtggagacagccccgctgaaagcaggctcagttatcaaagggagcagagactgggcgcggtggctcacgcctgtgatcccagcactttgggaggccgaggcgggtggatcacgaggtcacgagtttgagaccaacctgtctctagtaaaagtacaaaaataatctgggcatggtggcgtgcacctgtagtccgagctacacagaaggctgaagcaggagaattgcttgaacccaggaggcggagtttgtggtgaggcgagatcacgccactgcactttaacctgggcaacaaagcgagactccttctcaaaaaaaagaaaaaaaaaagcagaaactggttcagccttgtttagaagggcaatttggaaatttctaaggaaattctaaatgtgcaatatttaaaatgactcagcacttccatacgcacaaagataggggtaggggaatattccctgtgacactgtttgtggtaagaaaagacagactcgggcccagttccatcagggggaatctggctaaattacagaatttccgagtgctggaacactgcacagccatgagaaaataatggcactggttcagatgttagcattgtcctgagtgatttcggcaaaaagaactaacccagaatcaggggagatggcgtcttgtcaaacttttttatacaggtagagagtatagaactcataaaagctatttgtaaaatgataagtttattggattagaaatctaagaaccaaggccaggcgcggtggctcacacctgtaatcccagcactttgggaggctgaggtgggcggatcacctgaggtcgggagtttgagacaagcctgaccaacatggagaaaccccgtctctactaaaaatacaaaattagctgggtgtggtggcacatgtctgtgaacccagctactggggaggctgaggcaggagaatcacttgaacccgggaggcggaggttgtagtgagccaagatctcaccagcaacaagagcaaaactccgtctcaaaaaaaaagaaatctaagaaccaaatatatcagagaatgttaaatttcttgggtatgatgatgatattgtagttacatagggaaatatccttattcttaggagatacatgctgaaatatttagagctgaagtgccatgtcagcaacttactttcaaaaagttcattcagcaaaaagccgggtgcagtggctcacacctgtaatcccagcactttaaggcaagctgatcactttgagatcaggagtttgagaccagcctggccaacatgatgaaaccctgtctctactaaaaatacaaaaattagccaggcatggtggcacacacctatagtcccagctattcaggaggctgaggcaggaggatcacttgaactcaggaagcagagggtgcagtgagccaagatcgcaccactgcactctagcctgggcaacagagcaagactcaatcttaaaaaaaaaaaaaaaaaaagatgttaggccaagcacagttcaagcctgtaatcccagcactttggaaggccaaggtgggaggatggcttgaagttacgagtttaagaccagccttggcaaccagcaagaccctgtctcttcaaaaaaaaaattttttaattagctaggtgtggtggtacatgcgtgtaatcccagctacttaggaggctgggcaggaggatcgtttgagcccaggagtttaaggctgcagtgagccatgattgtgccattgcactccagcctggacaacaaccatgtctctaaaaaaacaaaggagagagagaaaagaaaagatttttgaaagaagctaaagacttctaaaaaaaaagaaaaagaaaaagaaaagaaaaacgattaccaagcacttactaaagatgttagttacagggctgggcacagcagctcacgcctgtaattccagcactttgggaggccaaggcaggaaaattgcttgagcccaggagtgcaagaccagcctgggcaacacagtgagaccccacctctatttaacaaaatgaaatcaatcagtttcaaaagttagtctttatccctccataccttcagccagcccaaaccaggaatgggcagcttgacagcttcaggttgttgaaggcctggactcaacaaagctttgcgtagctagcggggctgttctgagcttggaagcgccgagcagtaacctctccctttcccgtgtctttgctctccagttccgagaggaacccgtgcaatagttcggtgccgcgtgacctccggcggcccgccagcctcatcggccaggaggtggttctcgtcttctttctcctgctcttgttggtctggttcctgaatcgcgaatttgaagtcagctaccgcctccactaccacggagacgtggaagcggatcttcaccgcaccaagatccagagcatgcgggaccaggcagactggctgctgaggaacatcatcccctaccacgtggctgagcagctgaaggtgtcccagacctactccaagaaccatgacagcggaggggtgatcttcgccagcatcgtcaacttcagcgagttctacgaggagaactacgagggcggcaaggagtgctaccgggtcctcaacgagctcatcggggactttgacgagctcctaagcaagccggactacagcagcatcgagaagatcaagaccatcggagccacgtacatggcggcgtcagggctgaacaccgcgcaggcccaggacggcagccacccgcaggagcacctgcagatcctgttcgagttcgccaaggagatgatgcgcgtggtggacgacttcaacaacaacatgctgtggttcaacttcaagctccgcgtcggcttcaaccatgggcccctcacggccggggtcatcggcaccaccaagctgctgtacgacatctggggagacaccgtcaacatcgccagcaggatggacaccaccggcgtggagtgccgcatccaggtgagcgaagagagctaccgcgtcttgagcaagatgggctatgacttcgactacagagggaccgtgaatgtcaaggggaaaggccagatgaagacctacctgtacccaaagtgcacggatcacagggtcatcccacagcaccagctgtccatctccccagacatccgcgtccaggtggatggcagcatcggacggtctcccacagacgagattgccaacctggtgccttctgtccagtatgtggacaagacatctctgggttctgacagcagcacgcaggccaaggatgcccacctgtcccccaagagaccgtggaaggagcccgtcaaagccgaagaaaggggtcgatttggcaaagccatagagaaagacgactgtgacgaaacaggaatagaagaagccaacgaactcaccaagctcaacgtttcaaagagtgtgtgaggcggcgcccacccgctgcccgaggtgctctgtttgtcgaaacacagtaatatttgtatttggctgttgtgctttccaagcgccacagttgccctccccggacgtggtgttatgtggtcatttcagccctaacttctgtgtggatcacagttattcagggttcattttcatccattcttccctttcgctcccttccctggaaaccccgctgcctctgggtcatccgttcagcacgtggtggagaacaagtgccttcagggctggcctcggcctcgagtctcgggacagaggccgccagtggagatcatggctttgggtattatttgacttttagaacaaaagctgtggttaagatctcatttttattgctttttcccacgtcccacgagacactattttcggttctctggctaataccctgtttttgagtttattttgtttctgtctatgtcacagtgttcctctacgacccgacctctctatgtaagcacacatgcgcacacacacttgcattcatgaatctgatataaagtgccagtaatccgccaagagggggtgcgaagggggcatgtcacgacagctccgccaccccccattgcccacccgcactttcccgagcaccgcgccccgtgggctgtgggtgagccgcgctccctgcactgagcgggtttaggggctcgcccacatgcatgcaggccaagacagcaaatgccagccgggcacgacgcccgtgtgcccaggcctcgggggtctcagagccgcctctcacccccgaccctccacccaggggtctccccatcgggagtggaggtgttggtcctggaagctgactcatcggagagggaaataccaaataaacatccgaggttgcatttatttatttatttatttacccggaagcatgtgcgctcccgggaggggtgctgggtcctcagggtggggggtcatagccacaggggagggcagttcccaggaaggtaaccagatgtctcttttgtgttttggccacctggggccgcccccaacctccccaccccaagaggggtctgagtgggacctcaaagcatggccagtgcaattctgaggctaggggaataggagaagcccaccagctgcccctgccacccacgggggctcctccctgccggggtccagctcagaaccacgggggtctgtgccggcagcaccggtgccatcttggcttccacaaattccagaagtttcttggctgcctcccgctctcagatggcacagggcctatcagtggcaggatttaatctggggagcaaattccagctctgaaatggagttctcacaccgtatctagggacagaggcatctggatgcaaacgcggggtttgttaggggcggaatcccgagggtggtgtgggggagctccgtccgggcagttggctgctggtcctgtctgattatgttttgccatattgagtctgaagtttgtgtggaagtgagctcgcccgagttgtccggtcggagttgtagccagtctttcaggggaccccgggacgctgatgaggcccagggtgcactccttgtcagtcttgcccgttgtccctgtgcattttgctttccgctgtcaaatctcactgtctcgttttcacaccgcatcagtccccatcatcactactcctccccagagacatacagattgccctccccggcatgaagaagtcacaattctgacccgccacctctccaggggcggtgcctgggatggccacccatcaggacagccagagccagggtcccggcgtcctgagagtccagggcacctccccccattagctgtaaggtttcctgtcaggtgtgactgtcagtttgctggtcttccaataccgaagaaagggtggttgtgacaatacctcttgcttctaaagaatgtattataaaacaccgcagatttttttttttccttaaaaaacactacctgatgctttccttgttcgtggggattgtggtcacatgaagctctttctgcatcagtattaaggtgtatatttgaatgtcctcccctcccctttcccctccaggctgtgtagctttgaggggctgggcgtttgctcacgaccttgctgtctcgctcagaacatgctccgcaaagttctccgcacacacttcttccccatcaagcccatttccttccccaaccacaaaggtgtttgtgattcctcaccccgggaaaccaaggagctgcaaagtggagtctggttcagccccgtgcagactcacccagagcttaacgttgtctttcaaacaccctgagccttcctaaacagccagtgcagacgttctctctgggccacgaagcccctcgggtcctccccgtcccctgctccgatgcatacctcagtgcagaaccacagaatctctgcagcggaaacgccgtgcatctcttgtctgttggcagcgagcacatcgtgctggagacacgagtttctaagcagctggcacgagggctgctgacggcatgggtcgtgcttcagggtggcaatacctcttaggaacttagggcaggaagcaatacttcagcattgaatgtgtgtaaatagttgctttgagttgcaattgctattttcttctcagtcccagctcagatcgaattatatatccatatatatatatatatatatatatatatatatatatatatatatatggtaaacaagcacacacaattttatccaatgcaaacaaatgtagagcatcagttacaaaaccctcgaatagcttgagagccccacaggctctgccacacccgtgacttcatccacactgacgtcacccgcgggggctccccctgcacatttgcacacgatccggagagccgaaggccgcgtgcttcctgtcacatgggctgtaatcatttgtagtttccaaagacacgtctgcatttgaatttctagattttcgaggtaaggagttttttttaattggttgtttggaaaatcacatcatgcctagaatctgaaattgaattagcaagaaccgactgtttgcattttccatatatccttttatctgctctttttaaattgttaattctaataatttcaaaatgcattcactgaagaaatggacattaaaatattctaaaatttaaatata

(19) United States
(12) Patent Application Publication
(10) Pub. No.: US 2026/0043031 A1
(43) Pub. Date: Feb. 12, 2026

(54) COMPOSITIONS AND METHODS FOR INHIBITING ADENYLATE CYCLASE 9 (AC9)

(71) Applicant: INSTITUT DE CARDIOLOGIE DE MONTRÉAL, Montréal (CA)

(72) Inventors: Mathieu BRODEUR, Montréal (CA); David RHAINDS, Montréal (CA); Jean-Claude TARDIF, Laval (CA)

(21) Appl. No.: 19/101,314

(22) PCT Filed: Aug. 3, 2023
(86) PCT No.: PCT/CA2023/051037

§ 371 Date: Feb. 5, 2025

Related U.S. Application Data
(60) Provisional application No. 63/395,371, filed on Aug. 5, 2022.

(51) Int. Cl. C12N 15/113

(52) U.S. Cl. CPC C12N 15/1137; C12Y 406/01001; C12N 2310/14

(57) ABSTRACT

Disclosed are methods of decreasing serum low density lipoprotein (LDL) level in a subject. The methods include inhibiting expression or function of an adenylate cyclase in the subject, wherein the inhibiting includes administration of an inhibitory nucleic acid molecule, such as an siRNA, to the subject.

### TECHNICAL FIELD

This disclosure relates to inhibitory nucleic acid molecules and compositions and methods thereof for decreasing low density lipoprotein (LDL) and increasing LDL receptors (LDLr) in a subject.

### BACKGROUND

Adenylate cyclases (AC), also referred as adenyl cyclase and adenylyl cyclase, are regulatory enzymes that modulate signaling pathways and physiological responses in cells by converting adenosine triphosphate (ATP) to 3′,5′-cyclic AMP (cAMP), a key secondary messenger. Adenylate cyclase type 9 (AC9) is an atypical member of the membrane-bound AC family that is weakly activated by forskolin (Ostrom et al. (2022) Physiol. Rev. 102: 815-857), auto-inhibited by its C-terminal cytosolic domain (C2b) (Pelvolgyi et al. (2018) Cell Signal. 51:266-275), and endocytosed after stimulation by G-protein-coupled receptors (GPCRs) (Lazar et al. (2020) eLife 9: e58039). Like other ACs, AC9 can form heterodimers with AC5 and AC6 (Baldwin et al. (2019) Mol. Pharmacol. 9: 349-360). Notably, expression of full length AC9 blocks endogenous GPCR-associated stimulation of AC and cAMP production while a C-terminally-truncated AC9 does not (Pelvolgyi et al. (2018) Cell Signal. 51:266-275).

Throughout the body, low density lipoprotein (LDL) transports fat molecules to cells via the bloodstream. LDL receptors (LDLr) on the surface of receiving cells can bind to and endocytose LDL, thereby internalizing LDL and lowering LDL levels in the blood. Excess LDL in the blood is associated with increased cardiovascular complications, such as coronary heart disease, resulting in one-fourth of all deaths in industrialized countries (Goldstein et al. (2015) Cell 161:161-172). Regulating circulating LDL by modulating LDLr is, therefore, of therapeutic interest.

Several mechanisms are implicated in the regulation of LDLr expression. Down regulation of LDLr protein can be mediated by inducible degrader of LDLr (IDOL), which stimulates proteasomal degradation of LDLr (Zelcer et al. (2009) Science 325:100-104). Additionally, lysosomal degradation of LDLr protein can be stimulated by the proprotein convertase subtilisin/kexin type 9 (PCSK9) (Park et al. (2004) J Biol Chem. 48:50630-50638). On the contrary, transcriptional upregulation of LDLr can be mediate by sterol regulatory element-binding protein-2 (SREBP-2), which, when intracellular cholesterol is low, can translocate to the nucleus of a cell and stimulate LDLr gene expression (Goldstein et al. (2015) Cell 161:161-172). AC-cAMP and protein kinase A (PKA) can further influence the upregulation of LDLr. For example, phosphodiesterase (PDE) inhibitors can induce SREBP2 nuclear translocation by a PKA-dependent mechanism (Shimizu-Albergine et al. (2013) Proc Natl Acad Sci., 113:E5685-5693), thereby increasing LDLr transcription. Furthermore, a functional cAMP-responsive element (CRE) present in the LDLr promotor can stimulate LDLr transcription (Liu et al. (2000) J Biol Chem. 275:5214-5221).

There remains a need for compositions and methods that can decrease LDL and/or increase LDLr in a subject.

### SUMMARY OF THE INVENTION

The invention provides compositions and methods of decreasing low density lipoprotein (LDL) in the serum of a subject. Furthermore, the invention provides compositions and methods of increasing LDL receptors (LDLr) in a subjects.

In a first aspect, the invention provides a method of decreasing serum low density lipoprotein (LDL) level in a subject, the method including inhibiting expression or function of an adenylate cyclase in the subject, wherein the inhibiting includes administration of an inhibitory nucleic acid molecule to the subject.

In a second aspect, the invention provides a method of increasing LDL receptor expression in a subject, the method including inhibiting expression or function of an adenylate cyclase in the subject, wherein the inhibition includes administration of an inhibitory nucleic acid molecule to the subject.

In some aspects, the adenylate cyclase is adenylate cyclase type 9 (AC9).

In some aspects, the AC9 contains an mRNA sequence of SEQ ID NO: 16; and/or a DNA sequence of SEQ ID NO: 17.

In some aspects, the inhibitory nucleic acid molecule is an anti-sense oligonucleotide (ASO), a small interfering RNA (siRNA), a short hairpin RNA (shRNA), a double-stranded RNA (dsRNA), or a microRNA (miRNA).

In some aspects, the inhibitory nucleic acid molecule is an siRNA.

In some aspects, the siRNA includes a sequence complementary to at least 15 contiguous nucleotides set forth within any one of SEQ ID NOs: 1-10, 16, and 17.

In some aspects, the siRNA includes a sequence complementary to at least 19 contiguous nucleotides set forth within any one of SEQ ID NOs: 1-10, 16, and 17.

In some aspects, the siRNA includes a sequence complementary to at least 21 contiguous nucleotides set forth within any one of SEQ ID NOs: 1-10, 16, and 17.

In some aspects, the siRNA includes a sequence complementary to at least 25 contiguous nucleotides set forth within any one of SEQ ID NOs: 16 and 17.

In some aspects, the siRNA molecule contains 3′ overhangs, such as: a single uracil overhang at one or more 3′ ends of the siRNA; a double uracil overhang at one or more 3′ ends of the siRNA; a single thymine overhang at one or more 3′ ends of the siRNA; a double thymine overhang at one or more 3′ ends of the siRNA; or a single cytosine and single thymine overhang at one or more 3′ ends of the siRNA.

In some aspects, the siRNA includes a nucleotide sequence of any one or more of SEQ ID NOs: 1-10.

In some aspects, the siRNA includes a sense strand including the sequence of SEQ ID NO: 1 and an antisense strand including the sequence of SEQ ID NO: 2. In some aspects, the siRNA includes a sense strand including the sequence of SEQ ID NO: 3 and an antisense strand including the sequence of SEQ ID NO: 4. In some aspects, the siRNA includes a sense strand including the sequence of SEQ ID NO: 5 and an antisense strand including the sequence of SEQ ID NO: 6. In some aspects, the siRNA includes a sense strand including the sequence of SEQ ID NO: 7 and an antisense strand including the sequence of SEQ ID NO: 8. In some aspects, the siRNA includes a sense strand including the sequence of SEQ ID NO: 9 and an antisense strand including the sequence of SEQ ID NO: 10.

In some aspects, the siRNA includes a non-natural or modified nucleoside or nucleotide.

In some aspects, the modification is chosen from a 2′-O-methyl (2′-O-Me) modified nucleoside, a phosphorothioate (PS) bond between nucleosides, and a 2′-fluoro (2′-F) modified nucleoside.

In some aspects, the siRNA molecule targets the sequence of any one of SEQ ID NOs: 11-15.

In some aspects, the method of any of the foregoing aspects further includes administering a second therapeutic agent to the subject.

In some aspects, the second therapeutic agent is selected from the group including of a statin, a proprotein convertase subtilisin/kexin type 9 (PCSK9) inhibitor, an ATP Citrate Lyase (ACL) inhibitor, a lipoprotein(a) (Lp(a)) inhibitor, an angiopoietin-like 3 (ANGPTL3) inhibitor, a cholesterylester transfer protein (CETP) inhibitor, a microsomal triglyceride transfer protein (MTP) inhibitor, an apolipoprotein B (ApoB) inhibitor, a bile acid binding resin, and colchicine.

In some aspects, statin is atorvastatin.

In some aspects, the PCSK9 inhibitor is an siRNA molecule or a monoclonal antibody targeting PCSK9.

In some aspects, the ACL inhibitor is bempedoic acid.

In some aspects, the Lp(a) inhibitor is an siRNA molecule targeting Lp(a).

In some aspects, the MTP inhibitor is lomitapide.

In some aspects, the ApoB inhibitor is mipomersen.

In a third aspect, the invention provides a siRNA molecule including: a sense strand including the sequence of SEQ ID NO: 3 and an antisense strand including the sequence of SEQ ID NO: 4; a sense strand including the sequence of SEQ ID NO: 5 and an antisense strand including the sequence of SEQ ID NO: 6; a sense strand including the sequence of SEQ ID NO: 7 and an antisense strand including the sequence of SEQ ID NO: 8; or a sense strand including the sequence of SEQ ID NO: 9 and an antisense strand including the sequence of SEQ ID NO: 10.

In some aspects, the siRNA includes a non-natural or modified nucleoside or nucleotide.

In some aspects, the modification is chosen from a 2′-O-methyl (2′-O-Me) modified nucleoside, a phosphorothioate (PS) bond between nucleosides, and a 2′-fluoro (2′-F) modified nucleoside.

In some aspects, the siRNA molecule targets the sequence of any one of SEQ ID NOs: 11-15.

### BRIEF DESCRIPTION OF THE DRAWINGS

The accompanying drawings are included to illustrate embodiments of the disclosure and further an understanding of its implementations.

FIG. 1A shows a Western blot of AC9, LDLr, and ABCA1 protein levels after siRNA-mediated knockdown of AC9 in HepG2 cells, relative to an siScramble control. Actin is shown as a loading control.

FIG. 1B is a graph showing quantification of AC9, LDLr, and ABCA1 protein levels after siRNA-mediated knockdown of AC9 in HepG2 cells (left bar for each AC9, LDLr, and ABCA1), relative to an siScramble control (right bar for each AC9, LDLr, and ABCA1). In brief, densitometry data from the Western blot in FIG. 1A was calculated for AC9, LDLr, and ABCA1 and normalized to the densitometry data for the actin loading control. Protein expression is represented as a percentage of the control, siScramble. Error bars represent the mean±standard deviation. n=4-6. Paired t-test: * 10=p≤0.05; **=p≤0.01; and ***=p≤0.001, relative to siScramble.

FIG. 1C is a graph showing quantification of ³H-CE-LDL association after siRNA-mediated knockdown of AC9 in HepG2 cells, relative to an siScramble control. In brief, human LDL labeled with ³H-cholesteryl oleate (CE) were incubated with HepG2 cells at 20 μg protein/ml for 4 h. At the end, cells were solubilized in 0.2N NaOH and radioactivity was estimated with a beta counter, while being normalized to cell proteins estimated by Lowry assay. Error bars represent the mean±standard deviation. n=5. Paired t-test: *=p≤0.05, relative to siScramble.

FIG. 1D is a graph showing quantification of ³H-CE-LDL cholesterol efflux after siRNA-mediated knockdown of AC9 in HepG2 cells. In brief, transfected HepG2 cells were loaded for 24 h with ³H-cholesterol and subjected to an 18 h equilibration. Then, cholesterol efflux (4 h) toward apo A-I at 10 μg/ml was conducted. This dose of apo A-I is considered saturating for the ABCA1 transporter. At the end, media was harvested, cells were solubilized in 0.2N NaOH and radioactivity was estimated with a beta counter. Cholesterol efflux percent was calculated by dividing the radioactivity in media by the radioactivity measured in cells and media. Error bars represent the mean±standard deviation. n=4. Paired t-test: *=p≤0.05, relative to siScramble.

FIG. 2A is a graph showing quantification of AC9, LDLr, SREBP2, and ABCA1 protein level by untargeted relative proteomics after siRNA-mediated knockdown of AC9 in HepG2 cells, relative to an siScramble control. Error bars represent the mean±standard deviation. n=4. Paired t-test: *=p 0.05; and ***=p≤0.001, relative to siScramble.

FIG. 2B is a graph showing quantification of PRKAR1A, PRKAR1B, and AKAP12 protein level by untargeted relative proteomics after siRNA-mediated knockdown of AC9 in HepG2 cells, relative to an siScramble control. Error bars represent the mean±standard deviation. n=4. Paired t-test: *=p 0.05; and ***=p≤0.001, relative to siScramble.

FIG. 3 is a graph showing quantification of ³H-CE-LDL association in HepG2 cells transfected with an AC9 siRNA (siAC9) and treated or not with 5 μM of atorvastatin (Ato) 24 h before cholesterol association assays. In brief, human LDL labeled with ³H-cholesteryl oleate (CE) were incubated with HepG2 cells at 20 μg protein/ml for 4 h. At the end, cells were solubilized in 0.2N NaOH and radioactivity was estimated with a beta counter, while being normalized to cell proteins estimated by Lowry assay. Left bar is siScramble and the right bar is siAC9 for both the control and Ato. These results illustrate the additive effects of AC9 siRNA and atorvastatin on LDL cholesterol uptake by LDLr. Error bars represent the mean±standard deviation. n=5. Repeated measures ANOVA with uncorrected Fisher's LSD: *=p≤0.05, relative to siScramble; a=p≤0.05, relative to control.

FIG. 4 is a graph showing quantification of LDLr protein in HepG2 cells treated with 300 μM of exogenous cAMP, 5 μM of atorvastatin (Ato), or both cAMP and Ato. In brief, densitometry data from the Western blot was calculated and normalized to the densitometry data for the actin loading control. Protein expression is represented as a percentage of the control without Ato. The left bar is the control and the right bar is cAMP for both without Ato and with Ato. These results illustrate the combined effected of exogenous cAMP and atorvastatin to increase LDLr protein. Error bars represent the mean±standard deviation. n=5. Paired t-test: *=p≤0.05, relative to control; a=p≤0.05, relative to “without Ato”.

FIG. 5A is a graph showing quantification of LDLr protein expression in HepG2 cells transfected with an AC9 siRNA (siAC9) and treated or not with 2 μM of H89 (a PKA inhibitor) 24 h before cellular proteins extraction. In brief, densitometry data from the Western blot was calculated and normalized to the densitometry data for the actin loading control. Protein expression is represented as a percentage of the siScramble control. The left bar is the control and the right bar is H89 for both siScramble and siAC9. These results show that LDLr increases due to AC9 siRNA are mostly PKA-dependent. Error bars represent the mean±standard deviation. n=6. Paired t-test: a=p≤0.05, relative to siScramble-control; b=p≤0.05, relative to siAC9-Control.

FIG. 5B is a graph showing quantification of ABCA1 protein expression in HepG2 cells transfected with an AC9 siRNA (siAC9) and treated or not with 2 μM of H89 (a PKA inhibitor) 24 h before cellular proteins extraction. In brief, densitometry data from the Western blot was calculated and normalized to the densitometry data for the actin loading control. Protein expression is represented as a percentage of the siScramble control. The left bar is the control and the right bar is H89 for both siScrmable and siAC9. These results show that ABCA1 increases due to AC9 siRNA are mostly PKA-dependent. Error bars represent the mean±standard deviation. n=6. Paired t-test: a=p≤0.05, relative to siScramble-Control.

FIG. 5C is a graph showing quantification of ³H-CE-LDL association in HepG2 cells transfected with an AC9 siRNA (siAC9) and treated or not with 2 μM of H89 (a PKA inhibitor) 24 h before cholesterol association assays. In brief, human LDL labeled with ³H-cholesteryl oleate (CE) were incubated with HepG2 cells at 20 μg protein/ml for 4 h. This dose of ³H-CE-LDL is considered saturating for the LDLr transporter. At the end, cells were solubilized in 0.2N NaOH and radioactivity was estimated with a beta counter, while being normalized to cell proteins estimated by Lowry assay. The left bar is the control and the right bar is H89 for both siScramble and siAC9. These results show that the increase of LDL uptake due to AC9 siRNA is mostly PKA-dependent. Error bars represent the mean±standard deviation. n=3. Paired t-test: a=p≤0.05, relative to siScramble-Control.

FIG. 5D is a graph showing quantification of cholesterol efflux in HepG2 cells transfected with an AC9 siRNA (siAC9) and treated or not with 2 μM of H89 (a PKA inhibitor) 24 h before cholesterol efflux assays. In brief, treated HepG2 cells were loaded with ³H-cholesterol, equilibrated and used for cholesterol efflux (4 h) toward apo A-I at 10 μg/ml. The left bar is the control and the right bar is H89 for both siScramble and siAC9. This dose of apo A-I is considered saturating for the ABCA1 transporter. At the end, media was harvested, cells were solubilized in 0.2N NaOH and radioactivity was estimated with a beta counter. Cholesterol efflux percent was calculated by dividing the radioactivity in media by the radioactivity measured in cells and media. Error bars represent the mean±standard deviation. n=4. Paired t-test: a=p≤0.05, relative to siScramble-Control.

FIG. 6 is a graph showing quantification of AC9, SREBP2, PCSK9, and LDLr mRNA expression by quantitative PCR in HepG2 cells 48-hours post-transfection with an AC9 siRNA (siAC9), relative to an siScramble control. These results illustrate that AC9 siRNA increases mRNA expression of SREBP2 and LDLR, but not PCSK9. The left bar is siScramble and the right bar is siAC9 for AC9, SREBP2, LDLr, and PCSK9. Error bars represent the mean±standard deviation. n=6. Paired t-test: *=p≤0.05 and ***=p≤0.001, relative to siScramble.

FIG. 7 is a graph showing quantification of SREBP2 transcriptional activity in HepG2 cells 24- and 48-hours post treatment with an AC9 siRNA (siAC9), relative to an siScramble control. Briefly, SREBP-2 transcriptional activity was estimated by using a kit in which the SREBP-2 contained in a nuclear extract, binds specifically to immobilized SREBP-response element and is detected by addition of a specific primary antibody against SREBP-2. The left bar is siScramble and the right bar is siAC9 at each time point. These results show that AC9 siRNA increases SREBP2 transcriptional activity. Error bars represent the mean±standard deviation. n=5. Paired t-test: *=p≤0.05 and **=p≤0.01, relative to siScramble.

FIG. 8 is a graph showing quantification of LDLr protein expression in HepG2 cells treated with an AC9 siRNA (siAC9), a SREBP2 siRNA (siSREBP2), or both siAC9 and siSREBP2, relative to an siScramble control. In brief, densitometry data from the Western blot was calculated and normalized to the densitometry data for the actin loading control. Protein expression is represented as a percentage of the siScramble control. These results indicate the SREBP2 siRNA can block the increase of LDLr protein due to AC9 siRNA. Error bars represent the mean±standard deviation. n=6. Paired t-test: a=p≤0.05, relative to siScramble; b=p≤0.05, relative to siAC9.

FIG. 9A is a graph showing quantification of cholesterol efflux to apo A-I in HepG2 cells treated with an AC9 siRNA (siAC9), a SREBP2 siRNA (siSREBP2), or both siAC9 and siSREBP2, with siScramble serving as a control. In brief, transfected HepG2 cells were loaded with ³H-cholesterol, equilibrated and used for cholesterol efflux (4 h) toward apo A-I at 10 μg/ml. This dose of apo A-I is considered saturating for the ABCA1 transporter. At the end, media was harvested, cells were solubilized in 0.2N NaOH and radioactivity was estimated with a beta counter. Cholesterol efflux percent was calculated by dividing the radioactivity in media by the radioactivity measured in cells and media. These results indicate that SREBP2 is involved in the effects of AC9 siRNA on cholesterol efflux via ABCA1. Error bars represent the mean±standard deviation. n=6. Repeated measures ANOVA with uncorrected Fisher's LSD: a=p≤0.05, relative to siScramble; b=p≤0.05, relative to siAC9.

FIG. 9B is a graph showing quantification of cholesterol efflux to apo A-I in HepG2 cells transfected with an AC9 siRNA (siAC9) and treated or not with 5 μM of GSK-2033 (a LXR inhibitor), 24 h before cholesterol efflux assays, with siScramble serving as a control. In brief, transfected HepG2 cells were loaded with ³H-cholesterol, equilibrated and used for cholesterol efflux (4 h) toward apo A-I at 10 μg/ml. This dose of apo A-I is considered saturating for the ABCA1 transporter. At the end, media was harvested, cells were solubilized in 0.2N NaOH and radioactivity was estimated with a beta counter. Cholesterol efflux percent was calculated by dividing the radioactivity in media by the radioactivity measured in cells and media. The left bar is siScramble and the right bar is siAC9 for both the control and GSK-2033. These results indicate that LXR is involved in the effects of AC9 siRNA on cholesterol efflux via ABCA1. Error bars represent the mean±standard deviation. n=6. Repeated measures ANOVA with uncorrected Fisher's LSD: a=p≤0.05, relative to siScramble-Control; b=p≤0.05, relative to siAC9-Control.

FIG. 10A is a graph showing quantification of ABCA1 protein expression in HepG2 cells treated with an AC9 siRNA (siAC9), an LDLr siRNA (siLDLr), or both siAC9 and siLDLr, relative to an siScramble control. In brief, densitometry data from the Western blot was calculated and normalized to the densitometry data for the actin loading control. Protein expression is represented as a percentage of the siScramble. These results indicate that LDLr siRNA blocks the increase of ABCA1 protein due to AC9 siRNA. Error bars represent the mean±standard deviation. n=4. Repeated measures ANOVA with uncorrected Fisher's LSD: a=p≤0.05, relative to siScramble; b=p≤0.05, relative to siAC9.

FIG. 10B is a graph showing quantification of cholesterol efflux to apo A-I in HepG2 cells treated with an AC9 siRNA (siAC9), an LDLr siRNA (siLDLr), or both siAC9 and siLDLr, with siScramble serving as a control. In brief, transfected HepG2 cells were loaded with ³H-cholesterol, equilibrated and used for cholesterol efflux (4 h) toward apo A-I at 10 μg/ml. This dose of apo A-I is considered saturating for the ABCA1 transporter. At the end, media was harvested, cells were solubilized in 0.2N NaOH and radioactivity was estimated with a beta counter. Cholesterol efflux percent was calculated by dividing the radioactivity in media by the radioactivity measured in cells and media. These results indicate that LDLr siRNA blocks the increase of cholesterol efflux due to AC9 siRNA. Error bars represent the mean±standard deviation. n=13. Repeated measures ANOVA with uncorrected Fisher's LSD: a=p≤0.05, relative to siScramble; b=p≤0.05, relative to siAC9.

FIG. 11 is a schematic illustrating the mechanism of AC9 knock-down effect on LDLr and ABCA1 expression and function. Briefly, the reduction of AC9 expression in protein leads to increased cellular cAMP levels, due to removal of known inhibitory properties of AC9 on other members of the family. This in turn leads to PKA activation and target phosphorylation. One of these is SREBP-2, which activity is increased through increased expression and PKA-mediated phosphorylation. SREBP-2 is a key transcription factor that leads to increased LDLr mRNA expression. This results in higher LDLr protein levels associated with enhanced uptake of LDL particles. As LDL particles are degraded in lysosomes, cellular cholesterol is enriched and leads to activation of LXR activity. Both SREBP-2 and LXR can increase ABCA1 mRNA expression, while PKA activation is linked to ABCA1 phosphorylation. Overall, this leads to higher ACBA1 protein levels and cholesterol efflux towards apoA-I as acceptor, a homeostatic physiological response to the higher levels of cellular cholesterol. Thus, AC9 knock-down results in higher LDL particle uptake that could translate into lower LDL-cholesterol levels in the blood.

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “about,” as applied to one or more values of interest, refers to a value that falls within 10%, 9%, 8%, 7%, 6%, 5%, 4%, 3%, 2%, 1%, or less in either direction (greater than or less than) of a stated reference value, unless otherwise stated or otherwise evident from the context (except where such number would exceed 100% of a possible value).

As used herein, “administration” refers to providing or giving a subject a therapeutic agent by any effective route. Exemplary routes of administration are described herein below.

As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

As used herein, the term “auxiliary moiety” refers to any moiety, including, but not limited to, a small molecule, a peptide, a carbohydrate, a neutral organic polymer, a positively charged polymer, a therapeutic agent, a targeting moiety, an endosomal escape moiety, and any combination thereof, which can be conjugated to a nucleic acid molecule. In some embodiments, an “auxiliary moiety” is linked to an inhibitory nucleic acid molecule disclosed herein by forming one or more covalent or non-covalent bonds with one or more conjugating groups attached to a phosphate linkage, a phosphorothioate linkage, a 5′ positions of a nucleotide sugar, or any portion of a nucleobase. One skilled in the art will readily understand appropriate points of attachment of a particular auxiliary moiety to a nucleic acid molecule.

As used herein, “delivery vehicle” refers to any substance (e.g., molecule, peptide, conjugate, and construct) that facilitates, at least in part, the in vivo delivery of a nucleic acid molecule to targeted cells.

As used herein, the terms “effective amount,” “therapeutically effective amount,” and a “sufficient amount” of a composition described herein refer to a quantity sufficient to, when administered to the subject, effect beneficial or desired results, as such, an “effective amount” or synonym thereto depends upon the context in which it is being applied. For example, in the context of decreasing low density lipoprotein (LDL), it is an amount of the composition sufficient to achieve a treatment response as compared to the response obtained without administration of the composition. The amount of a given composition described herein that will correspond to such an amount will vary depending upon various factors, such as the given agent, the pharmaceutical compositions, the route of administration, the type of disease or disorder, the identity of the subject (e.g., age, sex, weight) or host being treated, and the like, but can nevertheless be routinely determined by one skilled in the art.

As used herein, a “formulation” includes at least an inhibitory nucleic acid molecule and a delivery vehicle.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “inhibitory nucleic acid molecule” refers to a nucleic acid molecule that has sufficient complementarity to bind to a target nucleic acid molecule to inhibit expression of protein encoded by the target nucleic acid molecule. Exemplary inhibitory nucleic acid molecules are anti-sense oligonucleotides (ASOs), small interfering RNA (siRNAs), short hairpin RNA (shRNAs), double stranded RNAs (dsRNAs), and microRNA (miRNAs). Inhibitory nucleic acid molecules may reduce target protein expression by 10% or more (e.g., 10%, 20%, 30%, 40%, 50%, 60%, 70%, 80%, 90%, 95%, 98%, 99% or more). In one embodiment, the target nucleic acid molecule encodes AC9.

As used herein “modified” refers to a changed state or structure of a nucleic acid molecule described herein. Molecules may be modified in many ways including chemically, structurally, and functionally. In one embodiment, the inhibitory nucleic acid molecules of the present invention are modified by the introduction of non-natural nucleosides and/or nucleotides. In other embodiments, the inhibitory nucleic acid molecules of the present invention are modified by conjugation of an auxiliary moiety.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions, and/or dosage forms which are, within the scope of sound medical judgment, suitable for use in contact with the tissues a subject, such as a mammal (e.g., a human) without excessive toxicity, irritation, allergic response, or other problem or complication, commensurate with a reasonable benefit/risk ratio.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “therapeutic agent” refers to any agent that, when administered to a subject, has a therapeutic, diagnostic, and/or prophylactic effect and/or elicits a desired biological and/or pharmacological effect.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” is considered a replicon, such as plasmid, phage, viral construct or cosmid, to which another nucleic acid (e.g., DNA or RNA) segment may be attached. Vectors are used to transduce and express the nucleic acid segment in cells.

### DETAILED DESCRIPTION

Described herein are compositions (e.g., inhibitory nucleic acid molecules) and methods thereof for decreasing low density lipoprotein (LDL) in the serum of a subject. Furthermore, the invention provides compositions (e.g., inhibitory nucleic acid molecules) and methods thereof for increasing LDL receptor (LDLr) expression in a subject.

The inhibitory nucleic acid molecules (e.g., a small interfering RNA (siRNA), a double-stranded RNA (dsRNA), an anti-sense oligonucleotide (ASO), a microRNA (miRNA), or a short hairpin RNA (shRNA)), or compositions thereof, described herein may be used in methods for reducing expression of adenylate cyclase 9 (AC9). Advantageously, the methods of the present disclosure provide for effective mechanisms to decrease LDL and/or increase LDLr in a subject. In doing so, the present methods are useful for reducing LDL concentrations in the blood (e.g., serum) of a subject.

### Inhibitory Nucleic Acid Molecules

Exemplary inhibitory nucleic acid molecules of the disclosure are siRNAs, dsRNAs, ASOs, miRNAs, and shRNAs; however any nucleic acid molecule capable of reducing AC9 mRNA and/or protein expression is envisioned for use of the methods described herein. In some instances, the inhibitory nucleic acid molecules of the disclosure may be referred as RNA inhibitory (RNAi) molecules.

For any of the inhibitory nucleic acid molecules described herein (e.g., siRNA, dsRNA, ASO, miRNA, shRNA, or other inhibitory nucleic acid molecules capable of reducing expression of a target gene) the inhibitory nucleic acid molecule contains at least some sequence complementarity to the nucleotide sequence of SEQ ID NO: 16. In some embodiments, the inhibitory nucleic acid molecule comprises or consists of sequence complementary to at least 15 contiguous nucleotides set forth within SEQ ID NO: 16. In some embodiments, the inhibitory nucleic acid molecule comprises or consists of sequence complementary to at least 16 contiguous nucleotides set forth within SEQ ID NO: 16. In some embodiments, the inhibitory nucleic acid molecule comprises or consists of sequence complementary to at least 17 contiguous nucleotides set forth within SEQ ID NO: 16. In some embodiments, the inhibitory nucleic acid molecule comprises or consists of sequence complementary to at least 18 contiguous nucleotides set forth within SEQ ID NO: 16. In some embodiments, the inhibitory nucleic acid molecule comprises or consists of sequence complementary to at least 19 contiguous nucleotides set forth within SEQ ID NO: 16. In some embodiments, the inhibitory nucleic acid molecule comprises or consists of sequence complementary to at least 20 contiguous nucleotides set forth within SEQ ID NO: 16. In some embodiments, the inhibitory nucleic acid molecule comprises or consists of sequence complementary to at least 21 contiguous nucleotides set forth within SEQ ID NO: 16. In some embodiments, the inhibitory nucleic acid molecule comprises or consists of sequence complementary to at least 22 contiguous nucleotides set forth within SEQ ID NO: 16. In some embodiments, the inhibitory nucleic acid molecule comprises or consists of sequence complementary to at least 23 contiguous nucleotides set forth within SEQ ID NO: 16. In some embodiments, the inhibitory nucleic acid molecule comprises or consists of sequence complementary to at least 24 contiguous nucleotides set forth within SEQ ID NO: 16. In some embodiments, the inhibitory nucleic acid molecule comprises or consists of sequence complementary to at least 25 contiguous nucleotides set forth within SEQ ID NOs: 16. In some embodiments, the inhibitory nucleic acid molecule comprises or consists of sequence complementary to at least 26 contiguous nucleotides set forth within SEQ ID NO: 16. In some embodiments, the inhibitory nucleic acid molecule comprises or consists of sequence complementary to at least 27 contiguous nucleotides set forth within SEQ ID NO: 16. In some embodiments, the inhibitory nucleic acid molecule comprises or consists of sequence complementary to at least 28 contiguous nucleotides set forth within SEQ ID NO: 16. In some embodiments, the inhibitory nucleic acid molecule comprises or consists of sequence complementary to at least 29 contiguous nucleotides set forth within SEQ ID NO: 16. In some embodiments, the inhibitory nucleic acid molecule comprises or consists of sequence complementary to at least 30 contiguous nucleotides set forth within SEQ ID NO: 16.

In some embodiments, the inhibitory nucleic acid is an siRNA targeting AC9. In some embodiments, the inhibitory nucleic acid is an dsRNA targeting AC9. In some embodiments, the inhibitory nucleic acid is an ASO targeting AC9. In some embodiments, the inhibitory nucleic acid is an miRNA targeting AC9. In some embodiments, the inhibitory nucleic acid is an shRNA targeting AC9. Each of these modalities is described further below.

Small Interfering RNA (siRNA)

siRNAs of the disclosure are single-stranded (ss) or double-stranded (ds) nucleic acid molecules made of DNA, RNA, or both DNA and RNA (e.g., a chimeric) that are complementary to a target gene of interest and prevent translation of the target's mRNA into a protein. Once an siRNA molecule enters a cell, it is incorporated into an RNA-induced silencing complex (RISC). Upon siRNA hybridization to a target mRNA, the RISC complex will cleave the target mRNA, thereby inactivating the target mRNA, resulting in reduced mRNA and protein levels of the target.

In some embodiments, siRNAs of the disclosure may include a nucleotide sequence of about 10 to about 30 nucleotides in length (e.g., 9, about 10, about 11, about 12, about 13, about 14, about 15, about 16, about 17, about 18, about 19, about 20, about 21, about 22, about 23, about 24, about 25, about 26, about 27, about 28, about 29, about 30, or 31 nucleotides in length).

In some embodiments, siRNAs of the disclosure may include a nucleotide sequence of 10 to 30 nucleotides in length (e.g., 10, 11, 12, 13, 14, 15, 16, 17, 18, 19, 20, 21, 22, 23, 24, 25, 26, 27, 28, 29, or 30 nucleotides in length).

It is within the scope of the disclosure that any length, known and previously unknown in the art, may be employed for the current invention.

In some embodiments, the siRNA contains an antisense strand. In some embodiments, lengths for an antisense strand of the siRNA molecules of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 17 nucleotides. In some embodiments, the antisense strand is 18 nucleotides. In some embodiments, the antisense strand is 19 nucleotides. In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the siRNA contains a sense strand. In some embodiments, the sense strand of the siRNA molecules of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

In some embodiments, the sense and antisense strands of an siRNA molecule of the disclosure are completely complementary. In some embodiments, the sense and antisense strands of an siRNA molecule of the disclosure are completely complementary to the extent that their lengths overlap with one another. Depending on the sequence of the first and second strand, complementarity need not be complete or perfect, which means that the first and second strand are not 100% base-paired due to mismatches. One or more mismatches may be present within the ds siRNA without impacting the siRNA's ability to reduced expression of a target gene of interest.

The nucleotide sequence of an siRNA of the disclosure may contain sufficient complementary to a portion of a target gene of interest (e.g., AC9 mRNA) such that the siRNA can hybridize with the target gene of interest. In some embodiments, the siRNA is at least 70%, at least 75%, at least 80%, at least 85%, at least 90%, at least 95%, at least 96%, at least 97%, at least 98%, or at least 99% complementary to the target gene of interest (e.g., AC9 mRNA), or a portion thereof. In some embodiments, the siRNA is complementary to the target gene of interest (e.g., AC9 mRNA), or a portion thereof.

In some embodiments, the nucleotide sequence of the siRNA may contain sufficient complementary to an exon sequence of a target gene of interest (e.g., an exon of AC9). In some embodiments, the nucleotide sequence of the siRNA may contain sufficient complementary to an intron sequence of a target gene of interest (e.g., an intron of AC9). In some embodiments, the siRNA of the disclosure may contain sufficient complementarity to a pre-mRNA transcript or an mRNA transcript encoding AC9. The target sequence of interest may be any one of SEQ ID NOs: 11-15 (e.g., see table 2). The target gene of interest (e.g., AC9) may be any one of SEQ ID NOs: 16-17 (e.g., see Table 3).

In some embodiments, the siRNAs described herein have 0-7 nucleotide 3′ overhangs or 0-4 nucleotide 5′ overhangs. In some embodiments, the siRNA molecule has a single uracil (e.g., U) overhang at each 3′ end of the siRNA. In some embodiments, the siRNA molecule has a double uracil (e.g., UU) overhang at each 3′ end of the siRNA. In some embodiments, the siRNA molecule has a single thymine (e.g., T) overhang at each 3′ end of the siRNA. In some embodiments, the siRNA molecule has a double thymine (e.g., TT) overhang at each 3′ end of the siRNA. In some embodiments, the siRNA molecule has a cytosine and thymine (e.g., CT) overhang at each 3′ end of the siRNA.

Different siRNAs can be combined for decreasing the protein expression of a target gene of interest (e.g., AC9). A combination of two siRNAs may be used in a method of the invention, such as two different siRNAs, three different siRNAs, four different siRNAs, or five different siRNAs targeting the same gene of interest (e.g., AC9, or variants thereof). In some embodiments, the siRNA sequence may contain at least 85%, 86%, 87%, 88%, 89%, 90%, 91%, 92%, 93%, 94%, 95%, 96%, 97%, 98%, 99% sequence identity to any one or more of SEQ ID NOs: 1-10 (e.g., see Table 1), or a complementary sequence thereof. In some embodiments, the siRNA sequence may contain the sequence of any one or more of SEQ ID NOs: 1-10 (e.g., see Table 1), or a complementary sequence thereof.

In some embodiments, the siRNA contains at least 15 contiguous nucleotides set forth within any one of SEQ ID NOs: 1-10 (e.g., see Table 1). In some embodiments, the siRNA contains at least 16 contiguous nucleotides set forth within any one of SEQ ID NOs: 1-10 (e.g., see Table 1). In some embodiments, the siRNA contains at least 17 contiguous nucleotides set forth within any one of SEQ ID NOs: 1-10 (e.g., see Table 1). In some embodiments, the siRNA contains at least 18 contiguous nucleotides set forth within any one of SEQ ID NOs: 1-10 (e.g., see Table 1). In some embodiments, the siRNA contains at least 19 contiguous nucleotides set forth within any one of SEQ ID NOs: 1-10 (e.g., see Table 1). In some embodiments, the siRNA contains at least 20 contiguous nucleotides set forth within any one of SEQ ID NOs: 1-10 (e.g., see Table 1). In some embodiments, the siRNA contains 21 contiguous nucleotides set forth within any one of SEQ ID NOs: 1-10 (e.g., see Table 1).

TABLE 1
EXEMPLARY siRNA SEQUENCES

| | STRAND | | STRAND |
|---|---|---|---|
| NAME | NAME | SEQ ID NO | SEQUENCE (5' to 3') |
| ADCY9_ID1039 | Sense 1 | SEQ ID NO: 1 | CCUGAUGAAAGAUUACUUUTT |
| | Antisense 1 | SEQ ID NO: 2 | AAAGUAAUCUUUCAUCAGGCT |
| ADCY9-1 | Sense 2 | SEQ ID NO: 3 | CGAAAUGGAAGAUGGGAAAUU |
| | Antisense 2 | SEQ ID NO: 4 | UUUCCCAUCUUCCAUUUCGUU |
| ADCY9-2 | Sense 3 | SEQ ID NO: 5 | CGAAACAGGAAUAGAAGAAUU |
| | Antisense 3 | SEQ ID NO: 6 | UUCUUCUAUUCCUGUUUCGUU |
| ADCY9-3 | Sense 4 | SEQ ID NO: 7 | AGGAAGAGGUCAUAAAGAAUU |
| | Antisense 4 | SEQ ID NO: 8 | UUCUUUAUGACCUCUUCCUUU |
| ADCY9-4 | Sense 5 | SEQ ID NO: 9 | GCACCAAGAUCCAGAGCAUUU |
| | Antisense 5 | SEQ ID NO: | AUGCUCUGGAUCUUGGUGCUU |
| | | 10 | |

A = adenine;
C = cytosine;
G = guanine;
T = thymine;
U = uracil.
Note:
the RNA sequence of SEQ ID NO: 1 contains thymine nucleotides at positions 20-21, when reading from 5' to 3'; the RNA sequence of SEQ ID NO: 2 contains a thymine nucleotide at position 21, when reading from 5' to 3'.

In some embodiments, the siRNA of the disclosure may target a nucleotide sequence of any one of SEQ ID NOs: 11-15 (e.g., see Table 2), or a complementary sequence thereof, or variant thereof with at least 85%, 86%, 87%, 88%, 89%, 90%, 91%, 92%, 93%, 94%, 95%, 96%, 97%, 98%, 99% thereto.

TABLE 2
TARGET SEQUENCES

| SEQ ID NO | mRNA SEQUENCE (5' to 3') |
|---|---|
| SEQ ID NO: 11 | CCUGAUGAAAGAUUACUUU |
| SEQ ID NO: 12 | CGAAAUGGAAGAUGGGAAA |
| SEQ ID NO: 13 | CGAAACAGGAAUAGAAGAA |
| SEQ ID NO: 14 | AGGAAGAGGUCAUAAAGAA |
| SEQ ID NO: 15 | GCACCAAGAUCCAGAGCAU |

In some embodiments, the siRNA comprises sequence complementary to at least 15 contiguous nucleotides set forth within SEQ ID NO: 16. In some embodiments, the siRNA comprises sequence complementary to at least 16 contiguous nucleotides set forth within SEQ ID NO: 16. In some embodiments, the siRNA comprises sequence complementary to at least 17 contiguous nucleotides set forth within SEQ ID NO: 16. In some embodiments, the siRNA comprises sequence complementary to at least 18 contiguous nucleotides set forth within SEQ ID NO: 16. In some embodiments, the siRNA comprises sequence complementary to at least 19 contiguous nucleotides set forth within SEQ ID NO: 16. In some embodiments, the siRNA comprises sequence complementary to at least 20 contiguous nucleotides set forth within SEQ ID NO: 16. In some embodiments, the siRNA comprises sequence complementary to at least 21 contiguous nucleotides set forth within SEQ ID NO: 16. In some embodiments, the siRNA comprises sequence complementary to at least 22 contiguous nucleotides set forth within SEQ ID NO: 16. In some embodiments, the siRNA comprises sequence complementary to at least 23 contiguous nucleotides set forth within SEQ ID NO: 16. In some embodiments, the siRNA comprises sequence complementary to at least 24 contiguous nucleotides set forth within SEQ ID NO: 16. In some embodiments, the siRNA comprises sequence complementary to at least 25 contiguous nucleotides set forth within SEQ ID NO: 16. In some embodiments, the siRNA comprises sequence complementary to at least 26 contiguous nucleotides set forth within SEQ ID NO: 16. In some embodiments, the siRNA comprises sequence complementary to at least 27 contiguous nucleotides set forth within SEQ ID NO: 16. In some embodiments, the siRNA comprises sequence complementary to at least 28 contiguous nucleotides set forth within SEQ ID NO: 16. In some embodiments, the siRNA comprises sequence complementary to at least 29 contiguous nucleotides set forth within SEQ ID NO: 16. In some embodiments, the siRNA comprises sequence complementary to at least 30 contiguous nucleotides set forth within SEQ ID NO: 16. The nucleotide sequence of SEQ ID NO: 16 is set forth in Table 3.

In some embodiments, the siRNA comprises sequence complementary to at least 15 contiguous nucleotides set forth within SEQ ID NO: 17. In some embodiments, the siRNA comprises sequence complementary to at least 17 contiguous nucleotides set forth within SEQ ID NO: 17. In some embodiments, the siRNA comprises sequence complementary to at least 17 contiguous nucleotides set forth within SEQ ID NO: 17. In some embodiments, the siRNA comprises sequence complementary to at least 18 contiguous nucleotides set forth within SEQ ID NO: 17. In some embodiments, the siRNA comprises sequence complementary to at least 19 contiguous nucleotides set forth within SEQ ID NO: 17. In some embodiments, the siRNA comprises sequence complementary to at least 20 contiguous nucleotides set forth within SEQ ID NO: 17. In some embodiments, the siRNA comprises sequence complementary to at least 21 contiguous nucleotides set forth within SEQ ID NO: 17. In some embodiments, the siRNA comprises sequence complementary to at least 22 contiguous nucleotides set forth within SEQ ID NO: 17. In some embodiments, the siRNA comprises sequence complementary to at least 23 contiguous nucleotides set forth within SEQ ID NO: 17. In some embodiments, the siRNA comprises sequence complementary to at least 24 contiguous nucleotides set forth within SEQ ID NO: 17. In some embodiments, the siRNA comprises sequence complementary to at least 25 contiguous nucleotides set forth within SEQ ID NO: 17. In some embodiments, the siRNA comprises sequence complementary to at least 26 contiguous nucleotides set forth within SEQ ID NO: 17. In some embodiments, the siRNA comprises sequence complementary to at least 27 contiguous nucleotides set forth within SEQ ID NO: 17. In some embodiments, the siRNA comprises sequence complementary to at least 28 contiguous nucleotides set forth within SEQ ID NO: 17. In some embodiments, the siRNA comprises sequence complementary to at least 29 contiguous nucleotides set forth within SEQ ID NO: 17. In some embodiments, the siRNA comprises sequence complementary to at least 30 contiguous nucleotides set forth within SEQ ID NO: 17. The nucleotide sequence of SEQ ID NO: 17 is set forth in Table 3.

TABLE 3
ADENYLATE CYCLASE 9 SEQUENCE

| | SEQ ID | |
|---|---|---|
| NAME | NO | SEQUENCE (5′ to 3′) |
| Homo sapiens | SEQ ID | AGTGACAGCGGGCGGGGCAGGGGCCGGAGATGGTGGCGGCGGCAGCTCTTCC |
| adenylate | NO: 16 | GACATGAGGCAGCGGCCGCGGGGGACACGGCGGCGGGCGCGGCGGCGGCCGC |
| cyclase 9 | | ATCTCTAGCTCGGGGAGGCGGCCCCCGGCGTGGGCGCGGGCGGGCGGGCGTC |
| (ADCY9), | | CCGGCCGGCGTCGCGGGAGCCCGGCTCTGCGGCAGCGTCTGCACCGCCGCCC |
| mRNA | | GGCGAGGGGCCCGCGTCCGTGGTTCCACGCGCTCGGTCCCGGGACAGCTGCG |
| NCBI Reference | | GCCGCGGCGGGAGCGGCGGCGCACTCGGGGGGAAGCAGGCCCCACTAGACGG |
| Sequence: | | GCCGCGCCGACGGGCTCCGCGCACTCGCAGCCCGGGGCGCCCCCACCCCCAG |
| NM_001116.4 | | CGCCCGCCGCCGCGGGCAGGGCCGGCCCGGAGCGCGGGGGGCGGCCGGGGAG |
| | | CGCGAGCCGGGGGCGCCCGCCGAAGCTCGCTGCTCCGGGTCGGGGTCCGGGC |
| | | CCGGGGCCGGCCGCGCGCCGCCTTTGACGCATCGGAGCGCGGCTCCTGCAGG |
| | | ATGGAGGGCTCCGCGCCGCCAGCGGAGTTGTTTGTGCGCAGGCGGCTCGCGG |
| | | GGCTGGGAGCGCTCAAGGTCTGAACTTCCCTCCGGAGCCGCAGCTGGAGGAG |
| | | GCGAGCGCGCGAGGAGGAGAAGCCGCGCGGCGCGGAGGCCACCCTCGGGGCG |
| | | AGAGGCGCGGAAGGCGAGCGAGCAAAGCGGTCCCGGAGCCACGGCGGCCACG |
| | | CGGCGGGGACCCCCGGGCGTTCTAGGTACTGGTGACCCCGGCCGGGGCAGGC |
| | | CCCGGGACTCGACAACATGGCTTCCCCACCCCACCAGCAGCTGCTGCATCAC |
| | | CACAGCACCGAGGTGAGCTGCGACTCCAGCGGGGACAGCAACAGCGTGCGCG |
| | | TCAAGATCAACCCCAAGCAGCTGTCCTCCAACAGCCACCCCAAGCACTGCAA |
| | | ATACAGCATCTCCTCTAGCTGCAGCAGCTCTGGGGACTCCGGGGGCGTCCCC |
| | | CGGCGAGTGGGCGGCGGAGGCCGGCTGCGCAGGCAGAAGAAGCTGCCCCAGC |
| | | TGTTCGAGAGGGCCTCCAGCCGCTGGTGGGACCCCAAGTTCGACTCGGTGAA |
| | | CCTGGAGGAGGCCTGCCTGGAGCGCTGCTTCCCGCAGACCCAGCGCCGGTTC |
| | | CGGTATGCGCTCTTCTACATCGGCTTCGCCTGCCTTCTGTGGAGCATCTATT |
| | | TTGCGGTCCACATGAGATCCAGACTGATCGTCATGGTCGCCCCCGCGCTGTG |
| | | CTTCCTCCTGGTGTGTGTGGGCTTCTTTCTGTTTACCTTCACCAAGCTGTAC |
| | | GCCCGGCATTACGCGTGGACCTCGCTGGCTCTCACCCTGCTGGTGTTCGCCC |
| | | TGACCCTGGCTGCGCAGTTCCAGGTCTTGACGCCTGTCTCAGGACGCGGCGA |
| | | CAGCTCCAACCTTACGGCCACAGCCCGGCCCACAGATACTTGCTTATCTCAA |
| | | GTGGGGAGCTTCTCCATGTGCATCGAAGTGCTCTTTTTGCTCTATACCGTCA |
| | | TGCACTTACCTTTGTACCTGAGTTTGTGTCTGGGGGTGGCCTACTCTGTCCT |
| | | TTTCGAGACCTTTGGCTACCATTTCCGGGATGAAGCCTGCTTCCCCTCGCCC |
| | | GGAGCCGGGGCCCTGCACTGGGAGCTGCTGAGCAGGGGGCTGCTCCACGGCT |
| | | GCATCCACGCCATCGGGGTCCACCTGTTCGTCATGTCCCAGGTGAGGTCCAG |
| | | GAGCACCTTCCTCAAGGTGGGGCAATCCATTATGCACGGGAAGGACCTGGAA |
| | | GTGGAAAAAGCCCTCAAAGAGAGGATGATTCATTCCGTGATGCCAAGAATCA |
| | | TAGCCGATGACTTAATGAAGCAGGGAGATGAGGAGAGTGAGAATTCTGTCAA |
| | | GAGGCATGCCACCTCGAGCCCCAAGAACAGGAAGAAAAAGTCTTCCATCCAA |
| | | AAAGCTCCTATAGCCTTCCGCCCTTTTAAGATGCAGCAGATCGAAGAAGTCA |
| | | GTATTTTATTTGCAGATATCGTGGGCTTCACCAAGATGAGTGCCAACAAGTC |
| | | TGCCCACGCCCTGGTGGGTCTCCTGAACGATCTGTTCGGTCGCTTCGACCGC |
| | | CTGTGTGAGGAGACCAAGTGTGAGAAAATCAGCACCCTGGGAGACTGTTACT |
| | | ACTGCGTGGCGGGCTGTCCCGAGCCCCGGGCCGACCATGCCTACTGCTGCAT |
| | | CGAGATGGGCCTGGGCATGATCAAGGCCATCGAGCAGTTCTGCCAGGAGAAG |
| | | AAGGAGATGGTGAACATGAGAGTCGGGGTGCACACGGGCACCGTCCTTTGCG |
| | | GCATCCTGGGCATGAGGAGGTTTAAATTTGACGTGTGGTCCAACGATGTGAA |
| | | CCTGGCCAATCTCATGGAGCAGCTGGGAGTGGCCGGCAAAGTTCACATTTCT |
| | | GAGGCCACCGCAAAATACTTAGATGACCGGTACGAAATGGAAGATGGGAAAG |
| | | TTATTGAACGGCTGGGCCAGAGCGTGGTTGCTGACCAGTTGAAAGGTTTGAA |
| | | GACATACCTGATATCGGGTCAGAGAGCCAAGGAGTCTCGCTGCAGCTGTGCA |
| | | GAGGCCTTGCTTTCTGGCTTTGAGGTCATTGACGGCTCACAGGTGTCCTCAG |
| | | GCCCTAGGGGACAGGGGACAGCGTCATCAGGGAATGTCAGTGACTTGGCGCA |
| | | GACTGTCAAAACCTTTGATAACCTTAAGACCTGCCCTTCGTGCGGAATCACA |
| | | TTTGCTCCCAAATCTGAAGCCGGCGCCGAGGGAGGAGCACCTCAAAACGGCT |
| | | GCCAAGACGAGCATAAAAACAGCACCAAGGCTTCTGGAGGACCTAATCCCAA |
| | | AACTCAGAACGGGCTCCTCAGCCCTCCCCAAGAGGAGAAGCTCACCAACAGT |
| | | CAGACTTCTCTGTGTGAGATCTTGCAGGAGAAGGGAAGGTGGGCAGGGGTGA |
| | | GCCTGGACCAGTCGGCTCTCCTTCCGCTGAGGTTCAAGAACATCCGGGAGAA |
| | | AACGGACGCCCACTTTGTGGACGTTATCAAAGAAGACAGCCTGATGAAAGAT |
| | | TACTTTTTTAAGCCGCCCATTAATCAGTTCAGCCTGAACTTCCTGGATCAGG |
| | | AGCTGGAGCGATCCTACAGGACCAGCTATCAGGAAGAGGTCATAAAGAACTC |
| | | CCCCGTGAAGACGTTTGCTAGTCCCACCTTCAGCTCCCTCCTGGATGTGTTT |
| | | CTGTCGACCACAGTGTTTCTGACGCTGTCCACCACCTGCTTCCTGAAGTACG |
| | | AGGCGGCCACCGTGCCTCCCCCGCCCGCCGCCCTGGCGGTCTTCAGTGCAGC |
| | | CCTGCTGCTGGAGGTGCTGTCCCTCGCGGTGTCCATCAGGATGGTGTTCTTC |
| | | CTGGAGGACGTCATGGCCTGCACCAAGCGCCTGCTGGAGTGGATCGCCGGCT |
| | | GGCTACCACGTCACTGCATCGGGGCCATCCTGGTGTCGCTTCCCGCACTGGC |
| | | CGTCTACTCCCATGTCACCTCCGAATATGAGACCAACATACACTTCCCAGTG |
| | | TTCACAGGCTCGGCCGCGCTGATTGCCGTCGTGCACTACTGTAACTTCTGCC |
| | | AGCTCAGCTCCTGGATGAGGTCCTCCCTCGCCACCGTCGTGGGGGCCGGGCC |
| | | GCTGCTCCTGCTCTACGTCTCCCTGTGCCCAGACAGTTCTGTATTAACTTCG |
| | | CCCCTTGACGCAGTACAGAATTTCAGTTCCGAGAGGAACCCGTGCAATAGTT |
| | | CGGTGCCGCGTGACCTCCGGCGGCCCGCCAGCCTCATCGGCCAGGAGGTGGT |
| | | TCTCGTCTTCTTTCTCCTGCTCTTGTTGGTCTGGTTCCTGAATCGCGAATTT |
| | | GAAGTCAGCTACCGCCTCCACTACCACGGAGACGTGGAAGCGGATCTTCACC |
| | | GCACCAAGATCCAGAGCATGCGGGACCAGGCAGACTGGCTGCTGAGGAACAT |
| | | CATCCCCTACCACGTGGCTGAGCAGCTGAAGGTGTCCCAGACCTACTCCAAG |
| | | AACCATGACAGCGGAGGGGTGATCTTCGCCAGCATCGTCAACTTCAGCGAGT |
| | | TCTACGAGGAGAACTACGAGGGCGGCAAGGAGTGCTACCGGGTCCTCAACGA |
| | | GCTCATCGGGGACTTTGACGAGCTCCTAAGCAAGCCGGACTACAGCAGCATC |
| | | GAGAAGATCAAGACCATCGGAGCCACGTACATGGCGGCGTCAGGGCTGAACA |
| | | CCGCGCAGGCCCAGGACGGCAGCCACCCGCAGGAGCACCTGCAGATCCTGTT |
| | | CGAGTTCGCCAAGGAGATGATGCGCGTGGTGGACGACTTCAACAACAACATG |
| | | CTGTGGTTCAACTTCAAGCTCCGCGTCGGCTTCAACCATGGGCCCCTCACGG |
| | | CCGGGGTCATCGGCACCACCAAGCTGCTGTACGACATCTGGGGAGACACCGT |
| | | CAACATCGCCAGCAGGATGGACACCACCGGCGTGGAGTGCCGCATCCAGGTG |
| | | AGCGAAGAGAGCTACCGCGTCTTGAGCAAGATGGGCTATGACTTCGACTACA |
| | | GAGGGACCGTGAATGTCAAGGGGAAAGGCCAGATGAAGACCTACCTGTACCC |
| | | AAAGTGCACGGATCACAGGGTCATCCCACAGCACCAGCTGTCCATCTCCCCA |
| | | GACATCCGCGTCCAGGTGGATGGCAGCATCGGACGGTCTCCCACAGACGAGA |
| | | TTGCCAACCTGGTGCCTTCTGTCCAGTATGTGGACAAGACATCTCTGGGTTC |
| | | TGACAGCAGCACGCAGGCCAAGGATGCCCACCTGTCCCCCAAGAGACCGTGG |
| | | AAGGAGCCCGTCAAAGCCGAAGAAAGGGGTCGATTTGGCAAAGCCATAGAGA |
| | | AAGACGACTGTGACGAAACAGGAATAGAAGAAGCCAACGAACTCACCAAGCT |
| | | CAACGTTTCAAAGAGTGTGTGAGGCGGCGCCCACCCGCTGCCCGAGGTGCTC |
| | | TGTTTGTCGAAACACAGTAATATTTGTATTTGGCTGTTGTGCTTTCCAAGCG |
| | | CCACAGTTGCCCTCCCCGGACGTGGTGTTATGTGGTCATTTCAGCCCTAACT |
| | | TCTGTGTGGATCACAGTTATTCAGGGTTCATTTTCATCCATTCTTCCCTTTC |
| | | GCTCCCTTCCCTGGAAACCCCGCTGCCTCTGGGTCATCCGTTCAGCACGTGG |
| | | TGGAGAACAAGTGCCTTCAGGGCTGGCCTCGGCCTCGAGTCTCGGGACAGAG |
| | | GCCGCCAGTGGAGATCATGGCTTTGGGTATTATTTGACTTTTAGAACAAAAG |
| | | CTGTGGTTAAGATCTCATTTTTATTGCTTTTTCCCACGTCCCACGAGACACT |
| | | ATTTTCGGTTCTCTGGCTAATACCCTGTTTTTGAGTTTATTTTGTTTCTGTC |
| | | TATGTCACAGTGTTCCTCTACGACCCGACCTCTCTATGTAAGCACACATGCG |
| | | CACACACACTTGCATTCATGAATCTGATATAAAGTGCCAGTAATCCGCCAAG |
| | | AGGGGGTGCGAAGGGGGCATGTCACGACAGCTCCGCCACCCCCCATTGCCCA |
| | | CCCGCACTTTCCCGAGCACCGCGCCCCGTGGGCTGTGGGTGAGCCGCGCTCC |
| | | CTGCACTGAGCGGGTTTAGGGGCTCGCCCACATGCATGCAGGCCAAGACAGC |
| | | AAATGCCAGCCGGGCACGACGCCCGTGTGCCCAGGCCTCGGGGGTCTCAGAG |
| | | CCGCCTCTCACCCCCGACCCTCCACCCAGGGGTCTCCCCATCGGGAGTGGAG |
| | | GTGTTGGTCCTGGAAGCTGACTCATCGGAGAGGGAAATACCAAATAAACATC |
| | | CGAGGTTGCATTTATTTATTTATTTATTTACCCGGAAGCATGTGCGCTCCCG |
| | | GGAGGGGTGCTGGGTCCTCAGGGTGGGGGGTCATAGCCACAGGGGAGGGCAG |
| | | TTCCCAGGAAGGTAACCAGATGTCTCTTTTGTGTTTTGGCCACCTGGGGCCG |
| | | CCCCCAACCTCCCCACCCCAAGAGGGGTCTGAGTGGGACCTCAAAGCATGGC |
| | | CAGTGCAATTCTGAGGCTAGGGGAATAGGAGAAGCCCACCAGCTGCCCCTGC |
| | | CACCCACGGGGGCTCCTCCCTGCCGGGGTCCAGCTCAGAACCACGGGGGTCT |
| | | GTGCCGGCAGCACCGGTGCCATCTTGGCTTCCACAAATTCCAGAAGTTTCTT |
| | | GGCTGCCTCCCGCTCTCAGATGGCACAGGGCCTATCAGTGGCAGGATTTAAT |
| | | CTGGGGAGCAAATTCCAGCTCTGAAATGGAGTTCTCACACCGTATCTAGGGA |
| | | CAGAGGCATCTGGATGCAAACGCGGGGTTTGTTAGGGGCGGAATCCCGAGGG |
| | | TGGTGTGGGGGAGCTCCGTCCGGGCAGTTGGCTGCTGGTCCTGTCTGATTAT |
| | | GTTTTGCCATATTGAGTCTGAAGTTTGTGTGGAAGTGAGCTCGCCCGAGTTG |
| | | TCCGGTCGGAGTTGTAGCCAGTCTTTCAGGGGACCCCGGGACGCTGATGAGG |
| | | CCCAGGGTGCACTCCTTGTCAGTCTTGCCCGTTGTCCCTGTGCATTTTGCTT |
| | | TCCGCTGTCAAATCTCACTGTCTCGTTTTCACACCGCATCAGTCCCCATCAT |
| | | CACTACTCCTCCCCAGAGACATACAGATTGCCCTCCCCGGCATGAAGAAGTC |
| | | ACAATTCTGACCCGCCACCTCTCCAGGGGCGGTGCCTGGGATGGCCACCCAT |
| | | CAGGACAGCCAGAGCCAGGGTCCCGGCGTCCTGAGAGTCCAGGGCACCTCCC |
| | | CCCATTAGCTGTAAGGTTTCCTGTCAGGTGTGACTGTCAGTTTGCTGGTCTT |
| | | CCAATACCGAAGAAAGGGTGGTTGTGACAATACCTCTTGCTTCTAAAGAATG |
| | | TATTATAAAACACCGCAGATTTTTTTTTTTCCTTAAAAAACACTACCTGATG |
| | | CTTTCCTTGTTCGTGGGGATTGTGGTCACATGAAGCTCTTTCTGCATCAGTA |
| | | TTAAGGTGTATATTTGAATGTCCTCCCCTCCCCTTTCCCCTCCAGGCTGTGT |
| | | AGCTTTGAGGGGCTGGGCGTTTGCTCACGACCTTGCTGTCTCGCTCAGAACA |
| | | TGCTCCGCAAAGTTCTCCGCACACACTTCTTCCCCATCAAGCCCATTTCCTT |
| | | CCCCAACCACAAAGGTGTTTGTGATTCCTCACCCCGGGAAACCAAGGAGCTG |
| | | CAAAGTGGAGTCTGGTTCAGCCCCGTGCAGACTCACCCAGAGCTTAACGTTG |
| | | TCTTTCAAACACCCTGAGCCTTCCTAAACAGCCAGTGCAGACGTTCTCTCTG |
| | | GGCCACGAAGCCCCTCGGGTCCTCCCCGTCCCCTGCTCCGATGCATACCTCA |
| | | GTGCAGAACCACAGAATCTCTGCAGCGGAAACGCCGTGCATCTCTTGTCTGT |
| | | TGGCAGCGAGCACATCGTGCTGGAGACACGAGTTTCTAAGCAGCTGGCACGA |
| | | GGGCTGCTGACGGCATGGGTCGTGCTTCAGGGTGGCAATACCTCTTAGGAAC |
| | | TTAGGGCAGGAAGCAATACTTCAGCATTGAATGTGTGTAAATAGTTGCTTTG |
| | | AGTTGCAATTGCTATTTTCTTCTCAGTCCCAGCTCAGATCGAATTATATATC |
| | | CATATATATATATATATATATATATATATATATATATATATATATATGGTAA |
| | | ACAAGCACACACAATTTTATCCAATGCAAACAAATGTAGAGCATCAGTTACA |
| | | AAACCCTCGAATAGCTTGAGAGCCCCACAGGCTCTGCCACACCCGTGACTTC |
| | | ATCCACACTGACGTCACCCGCGGGGGCTCCCCCTGCACATTTGCACACGATC |
| | | CGGAGAGCCGAAGGCCGCGTGCTTCCTGTCACATGGGCTGTAATCATTTGTA |
| | | GTTTCCAAAGACACGTCTGCATTTGAATTTCTAGATTTTCGAGGTAAGGAGT |
| | | TTTTTTTAATTGGTTGTTTGGAAAATCACATCATGCCTAGAATCTGAAATTG |
| | | AATTAGCAAGAACCGACTGTTTGCATTTTCCATATATCCTTTTATCTGCTCT |
| | | TTTTAAATTGTTAATTCTAATAATTTCAAAATGCATTCACTGAAGAAATGGA |
| | | CATTAAAATATTCTAAAATTTAAA |
| Homo sapiens | SEQ ID | GCGGCCGCGGCGGGAGCGGCGGCGCACTCGGGGGGAAGCAGGCCCCACTA |
| adenylate | NO: 17 | GACGGGCCGCGCCGACGGGCTCCGCGCACTCGCAGCCCGGGGCGCCCCCA |
| cyclase 9 | | CCCCCAGCGCCCGCCGCCGCGGGCAGGGCCGGCCCGGAGCGCGGGGGGCG |
| (ADCY9), DNA | | GCCGGGGAGCGCGAGCCGGGGGCGCCCGCCGAAGCTCGCTGCTCCGGGTC |
| chr16: 4012650- | | GGGGTCCGGGCCCGGGGCCGGCCGCGCGCCGCCTTTGACGCATCGGAGCG |
| 4166186 | | CGGCTCCTGCAGGATGGAGGGCTCCGCGCCGCCAGCGGAGTTGTTTGTGC |
| | | GCAGGCGGCTCGCGGGGCTGGGAGCGCTCAAGGTCTGAACTTCCCTCCGG |
| | | AGCCGCAGCTGGAGGAGGCGAGCGCGCGAGGAGGAGAAGCCGCGCGGCGC |
| | | GGAGGCCACCCTCGGGGCGAGAGGCGCGGAAGGCGAGCGAGCAAAGCGGT |
| | | CCCGGAGCCACGGCGGCCACGCGGCGGGGACCCCCGGGCGTTCTAGgtcc |
| | | gtcccgagcggttctcgtgcgcctcgaaggtgggggcggtgggggcggtg |
| | | ggagcaccgctgagccgggaaccacaggaacagatggtgcctgtccaggt |
| | | cgcgtgctggagccgccccttaggacaggagcaggtcccgggcctctagg |
| | | cgtgcatgcctaggtgggagcgccgctaactctccccgttttgctggcag |
| | | GTACTGGTGACCCCGGCCGGGGCAGGCCCCGGGACTCGACAACATGGCTT |
| | | CCCCACCCCACCAGCAGCTGCTGCATCACCACAGCACCGAGGTGAGCTGC |
| | | GACTCCAGCGGGGACAGCAACAGCGTGCGCGTCAAGATCAACCCCAAGCA |
| | | GCTGTCCTCCAACAGCCACCCCAAGCACTGCAAATACAGCATCTCCTCTA |
| | | GCTGCAGCAGCTCTGGGGACTCCGGGGGCGTCCCCCGGCGAGTGGGCGGC |
| | | GGAGGCCGGCTGCGCAGGCAGAAGAAGCTGCCCCAGCTGTTCGAGAGGGC |
| | | CTCCAGCCGCTGGTGGGACCCCAAGTTCGACTCGGTGAACCTGGAGGAGG |
| | | CCTGCCTGGAGCGCTGCTTCCCGCAGACCCAGCGCCGGTTCCGGTATGCG |
| | | CTCTTCTACATCGGCTTCGCCTGCCTTCTGTGGAGCATCTATTTTGCGGT |
| | | CCACATGAGATCCAGACTGATCGTCATGGTCGCCCCCGCGCTGTGCTTCC |
| | | TCCTGGTGTGTGTGGGCTTCTTTCTGTTTACCTTCACCAAGCTGTACGCC |
| | | CGGCATTACGCGTGGACCTCGCTGGCTCTCACCCTGCTGGTGTTCGCCCT |
| | | GACCCTGGCTGCGCAGTTCCAGGTCTTGACGCCTGTCTCAGGACGCGGCG |
| | | ACAGCTCCAACCTTACGGCCACAGCCCGGCCCACAGATACTTGCTTATCT |
| | | CAAGTGGGGAGCTTCTCCATGTGCATCGAAGTGCTCTTTTTGCTCTATAC |
| | | CGTCATGCACTTACCTTTGTACCTGAGTTTGTGTCTGGGGGTGGCCTACT |
| | | CTGTCCTTTTCGAGACCTTTGGCTACCATTTCCGGGATGAAGCCTGCTTC |
| | | CCCTCGCCCGGAGCCGGGGCCCTGCACTGGGAGCTGCTGAGCAGGGGGCT |
| | | GCTCCACGGCTGCATCCACGCCATCGGGGTCCACCTGTTCGTCATGTCCC |
| | | AGGTGAGGTCCAGGAGCACCTTCCTCAAGGTGGGGCAATCCATTATGCAC |
| | | GGGAAGGACCTGGAAGTGGAAAAAGCCCTCAAAGAGAGGATGATTCATTC |
| | | CGTGATGCCAAGAATCATAGCCGATGACTTAATGAAGCAGGGAGATGAGG |
| | | AGAGTGAGAATTCTGTCAAGAGGCATGCCACCTCGAGCCCCAAGAACAGG |
| | | AAGAAAAAGTCTTCCATCCAAAAAGCTCCTATAGCCTTCCGCCCTTTTAA |
| | | GATGCAGCAGATCGAAGAAGTCAGTATTTTATTTGCAGATATCGTGGGCT |
| | | TCACCAAGATGAGTGCCAACAAGTCTGCCCACGCCCTGGTGGGTCTCCTG |
| | | AACGATCTGTTCGGTCGCTTCGACCGCCTGTGTGAGGAGACCAAGTGTGA |
| | | GAAAATCAGCACCCTGGGAGACTGTTACTACTGCGTGGCGGGCTGTCCCG |
| | | AGCCCCGGGCCGACCATGCCTACTGCTGCATCGAGATGGGCCTGGGCATG |
| | | ATCAAGGCCATCGAGCAGTTCTGCCAGGAGAAGAAGGAGATGGTGAACAT |
| | | GAGAGTCGGGGTGCACACGGGCACCGTCCTTTGCGGCATCCTGGGCATGA |
| | | GGAGGTTTAAATTTGACGTGTGGTCCAACGATGTGAACCTGGCCAATCTC |
| | | ATGGAGCAGCTGGGAGTGGCCGGCAAAGTTCACATTTCTGAGGCCACCGC |
| | | AAAATACTTAGATGACCGGTACGAAATGGAAGATGGGAAAGTTATTGAAC |
| | | GGCTGGGCCAGAGCGTGGTTGCTGACCAGTTGAAAGgtatgtgcactgct |
| | | ttacctcggcatcccccctccctgatcctgattgtattaaattaaaaaaa |
| | | aaagcttccagcctcagtgtctgcagacagctcaggtatctcagaatgcc |
| | | ttaccatggggggatctcagggtcagtgtgacttttctttcagcgggtca |
| | | aatatcacagatctgttcacactgcagaaagggacgctgtgcagagatga |
| | | cctagacgcgacctttagaaggaaggagaactttgaacgtgcataaccac |
| | | tggttggctgaaatgtgggggcacgtccggtgaatgtaagggtatggagc |
| | | ccttcagcattcagcactgtttagcacttaaggaaattgcctttgcattc |
| | | atttggtcaccctgaagtcccatccaaatgtttctctctgccctttttgc |
| | | gaaaacgtggagaataaaatctttctcctctctaggcctcactttgctta |
| | | tcctcaaaatagggataggaagtcggcgcagtcctgcaggtctctggaga |
| | | attagacggagttacttacctctgagtacacactgtcttttatctaggtc |
| | | tttttttttttttaaaaaaagtttgcattttcagctttcagccatcattt |
| | | gtttaaggatattttattatcatagtatttgccttaagtgaagagcttca |
| | | tattgagcttctccagccaccaggtgatttggaagggagaaacgtacctg |
| | | ccggagagctggtattcccctccctcatcctctcaggtttggatctggtg |
| | | cctggtccctggtttgaagctccttactgctattcttgatgtttttactg |
| | | ggctgcttgttttttctaatataggacaaaagtctttgagatctggaaaa |
| | | tgatattaaaataagtaggacgaaaaagaaagattttaagtaatctgcta |
| | | catttcttcccccacttcctcctttttaaacagtccttatgttttactaa |
| | | aatcttcaggattgaggggaacatctgttgtggaagtatgtagagaaaat |
| | | aacactgattttttttaaaagcctgcttttgtgaagctgtattgtatttt |
| | | gtggtccccacacactgcatacttaaacagttaaaagcctcaggaaaggg |
| | | aatcctcggaagaagtaattaatagttccaatcagagatggcagaagtaa |
| | | tagtttcaatcagagacggcagaagtaatagtgtcagtcagagacgtcca |
| | | gtggttgaggtagggcctgcataggtcgttggtgtgagatcatcctatgt |
| | | gtccagttggcattctctttctctggcttgtgatctgagctcctgtatct |
| | | tttatgtatttgttctctggcaagggaacatcatctttcttctgattttt |
| | | ctttattttactaactttgctgttgaagattttatggatgtagacgaaaa |
| | | ctattttcacccgagaattcccctaacctagagcactttaaggactatgt |
| | | taacaaggtcagaaactacactttctgttcacttgaaagatgctgccgtg |
| | | atgagtgtctttccgtttagggcggtggcactcgctcttgagtctgaagg |
| | | tgtcagtggacggggctgtgggtgtaaccccctgcttgccaccttggcat |
| | | ttttagggcccaggtgttgtgagctctgtccttggagactgtgggacggt |
| | | ggagctgggcggcagtacgttaaattaaatatgttggcccagaggacaat |
| | | gagaacgttttgtaagctgtactttgtatgctgtattactcgaatttcaa |
| | | aaatgaagagggtgtaattattaaatatgtttggaaattgccaagtcatt |
| | | gaatacctattttgtgaatttatgagtagttttatttttaagtttgtaga |
| | | tcaggggtctcagacacagagccaaaaaaaaaaaatgctagagtgagttt |
| | | gtgtttttttttttttaaacctcaaatagtgtaaatttggtaaattaact |
| | | tttcttgacatttgtgtacattgtatatttataaaagttttagtactgta |
| | | gggagtagggaagatagaaggcttagcttagctgctgattttttaaaagt |
| | | gatattctttcctttagctctttctctgagaccacgattttgttttcttg |
| | | agctcattttacaaacctttgtggataggactccaggaatagagtatgtc |
| | | tacacaacttcctggcccttttggaaactgtccttacaaagctaaattga |
| | | gatgacagcacagtttgtagaagaccctttcacacacatagttaagctta |
| | | tctacatgtaagatcgattttagcctctgcctttgggctggtggcagatg |
| | | ttttctctatacaatattgggtattacgggaggctgaggcaggagaattg |
| | | tttgaacccgggaagagaaggttgtagtgagccgaggctgcgccactgca |
| | | caccagcctgggcgacagagcaactctgtcttaaaaaacaacaacaacaa |
| | | aaacaaaaaaaaccccaatactgggtgtattagcctttccctcttacccc |
| | | cttttctgatcccagggttaaaaagcagccagtagcggccctgcaggaat |
| | | gggcatccatagaatgagaaggggcttctagaactagcctgggagcacag |
| | | aaggaagctcctctcattcaaatttatccgaacaggctttgcagaagggt |
| | | agtttcatgatttctgcacattccttcactctgaaatctgtgtcgtgccc |
| | | ctggcactgactgtgcacctggcactgtggatgtggagcaggatccgtga |
| | | tcgtggagcaggatccgtgatcgtgaggtaggaggtgggactcgactcca |
| | | gaggcggggttcagatgccagaccgaattgaggactagctgatacaggga |
| | | tggggcagaagcagcgttctgcaaggcacgcccaccagtgtgccttgtta |
| | | gtttactgtcgccatggccacacccggagttactgcctctttccatggcc |
| | | gtgacctgacaacccaaaagccaccacccttttcctagaagtgtctgcct |
| | | aagcctacctttactctacaggaaattaaaagtaggtataaggtaaggct |
| | | gggcgcagtggctcatgcctgtaatcccagcactttgggaggccaaggcg |
| | | ggtggatcacctgtggtcatgagttggagaccagtgtggccaatatagtg |
| | | aaaccccgtctctactaaaaatacaaaaattagcctggtgtggtggtgtg |
| | | cgcctgtaatcccaggtgctcgggaggctgaggcagaagaatcgcttgaa |
| | | cccaggaggtggaggttgcagtgagctgtgatcatgtccactgcactcca |
| | | gcctgggcgacagcctaggcgagactgcgtctcaaaaaaaaaaaaaaaaa |
| | | aaaaaaaaagtaggtataagtatgattgcaaaactgccctgagctgctac |
| | | tttccgcctttgggaaagcccagctctgcaggagcagtcatggagccgta |
| | | taacactgcctgcctcttcaataaaggtgttttctttcctgccaccggct |
| | | cacccttgaattatttcctgggcaaagccaagaaccctcttgggctcggc |
| | | cccactttggggcgcgtctgacctgcgttaatagttcccaacctccgtga |
| | | gctcacagttaaaaatgttgtggagtatgttcgggagtggggagcgcttt |
| | | agagggggctcctgctggagctgggtcctgagatcaggctgtggctctcc |
| | | aggtggacagaagagcaccagggcagagcggagaggtgggtatggctggg |
| | | ccctgtgcttctctgtgggggcggacgattgttagttgttcatcaggaca |
| | | aggaaggccatgggtagcgtgggaaggagtttgcatttgaccccataggt |
| | | gtcagggaaccactcaacagggggcttaggtgatctgacttgcagtttta |
| | | gaaagttcactctttcagagctagtgagtgtgggttggaaagagattaat |
| | | gaggtgtcgagtaaatcccaggtaggggtgatggtggcccaatccaaagt |
| | | tacagtagcagtgatggaggtgatgggatctgagtggcattaggaagagc |
| | | agcttgatgggactgcaagccagatttaaacaaacgactggcaggagggg |
| | | aagggcttgaccatgtgaatgggaaatggcattttaagtttctggaagtg |
| | | acttggcgacaatgtggagtcaggaatgggtgaggggcctttgaagttgc |
| | | tatggtatgaatggagaatgcaggatggacttgcagagtacagaggcccg |
| | | gaacccctggcttgcttactcattcagcaaatcatttactccgtgccagg |
| | | cacggttctgggttctggcaatacagccgtaaacaaaacagacaacaatt |
| | | cctgctcttgtaataggggacaagaatttttaagtcaataatataggagg |
| | | atcgctggagcccacgaggtcgaggttgcagagagccgtgattgcaccac |
| | | tgcattccagcctgggcgacagagtgagaccctgtctctaagaaacaaaa |
| | | accaggccttcaaataggggagatctaatagaggggacaacaggggagtc |
| | | aaaccataaataagtaaaatagaaaaagtgttttgtgaacaaacataaag |
| | | caggaaggggatacagcagatggggtgtacgagtgtgagaaatcttatct |
| | | agggtggccagtcactgggagaaggttgcaggaatttttagcatactgct |
| | | aggattttggaaataatctaaaaagtgaggggccaggcatggtggctcat |
| | | gcctgtaatctcaacactttgggaggccgaggtaggcggatcacctgagg |
| | | tcaggagtttgagaccagcctggccaacgtggtgaaaccccatatttact |
| | | aaaaatacaaaaattaaccaggtgtggtggcaggtacctgtaatcccagc |
| | | tactcgggatgctgaggcaggagaattgcttgaacctgggagacggaggt |
| | | tgcagtgagccaagattgcaccattgcactccagcctgggcgacaagaat |
| | | gagaccctgtctcaaaaaaaaaaaaaaaagtgtgagggacagttattaaa |
| | | gatatggagaaatgggccgggcgtggtggctcatgcctgtaatcccagca |
| | | ctttgggagaccgaggtgggcagatcaggaggtcaggagtttgagaccag |
| | | cctgaccaacatggtgaaaccctgtctctactgaaaatgcaaaaattggc |
| | | tgggtgtggtcgcaggcgcctttagtcccagccacttgggaagctgaggc |
| | | aggagaatcgttcgaacccaggaggtggagtttgcagtgagccgagatcg |
| | | tgccattgcactccagcctgggcgatagggcgagactttgtcgccaaaaa |
| | | aaaaaaaaaaaaaaaaaaaaaaaaatgaagaaacggtctgattaaggtga |
| | | gggaccttccacagcatttgggatgaaaccgctaggcctgagggaaccgt |
| | | ggctgttgtattctgccttgggccacaagcaaaaaatggtgagacgtgtt |
| | | atctcgagctgttggcttttcctgtcatagatacagggtcacaaagacag |
| | | aaaatgaaactgttagaaaatatctacaggcatcttcttttcccgtgtaa |
| | | atattaggaagacagaatgaagatgctcctccgctctcatccacacccat |
| | | ggtcatatctctgaagaggggaataccagggagtgatcacattgtcagat |
| | | gcagaaattaaaaaagcttttttccccagccaaactttatgcttgtggag |
| | | caacttggggaggagacccgctggtatgcatttgggcagattgtcacctg |
| | | ttggccacctctgtgatcgagacctttgccttagaggagttcctatgtag |
| | | accctgtctggtgggccgacagaccgacagtggacggaggcattctgtat |
| | | gcgttagcctctgcaaacaaacacctgtctcatgccggaaatctgggcgc |
| | | cccattaaccctgagcacgggcctgccagctgacagaaccctagccaggt |
| | | gttggtttggggttcagttgcatgctcaggtgttccccttccttcccccg |
| | | ttagcttgtccccacctaagcctgcaacccttgccactgggatgctaacc |
| | | cgttagcagagctcagtctccttgcttggaaattctgtgttgacctgcac |
| | | tgcattcttttttttttttttttttttttttttttgagacagagtctggc |
| | | tgtgtcacccaggctggaatgccatggcgtgatctcagcttactgcaacc |
| | | tctgcctcccaggttcaagcggttctcctgcctcagcctcccaaatagct |
| | | gggattacaggtgtgcgccaccacgcccggctaatttttgtatttttagt |
| | | agagatggggtttcaccatgtaggccaggccggtcttgaactcctgacct |
| | | caggtgatccaccagcctcagcctcccaaagggctgggattacaggcgtg |
| | | agccaccacgcccagccttgacctgcattcttgagcaggatctgttatgc |
| | | acgtggaaatacagcctctcagatagcaaatggcagagatgagatgaggc |
| | | acacagttcccctccacagctggggtgggagacagaaaacagcagtagag |
| | | tcgacactcaagctgcccccagtgaccatttgttcaggctctttgagacc |
| | | tggcttacagtgacaaggacacagctgttaattctgaagacaagctttcg |
| | | agtagaagtaacctctggtaaagcaagagaaggttaaggtttgctgaatt |
| | | gccaatgggatgaagaaggaagggagggctgtcttttcaggtagaatgcc |
| | | taaggaagggaaacatggagaaggaacaattagagcaagtcaagaagttt |
| | | caagatttttgtcttctggccagcttggtgagatagtaattatgtttctg |
| | | catattggtcagcgttgcctatgtactttgctgtggaatagtaatcttgg |
| | | aaaaaaaccaaccaaccaacccacattaatctgtccatgtctgggcacag |
| | | agtgtgcagaagcctgcgtgggccgtaagtctcgtctttgtcttggttgg |
| | | tgattttagtattggtgtttttgctggaattccaaaactgtaagagtgta |
| | | atcattcaccattagaattctgcaggtcccctcctgactctggactagct |
| | | gagcttaggggcctgtcatccatctggccctgggcatatcatttgtactt |
| | | tgcctgtgacacacctcaagaccttttccaacaggaactagcatccctgg |
| | | agggaattttgtgaagagatgagagtttttgaaacaggaagggaactggc |
| | | aagggaagtggccaggcaccggtgctgccgcggaacagaagggaagggac |
| | | tgggctgcatcagagggtggatgtggggccgccatccgtggtgtgggcca |
| | | agggtagggtggtcctcccctgggaagactgcagacagcagtgatattct |
| | | tgtagtcttgtggatgtacccagtgaaccacaccagccaagccaacccgt |
| | | ggctaaattttagtcaacttcagcgaagaagactgccccttgtaaaactt |
| | | gtctgctcttggaaatcaggttttttgtctcattttccccagcctttgct |
| | | agcattgcacttgtaaaaaattcctggcatcctttctgcggagttaagtg |
| | | ggaaggctcaatgttctgggataggtgggaacatcaaacccgctagaggc |
| | | tgctggtggcagtggaggcagaatgagttgtagcagtagtggtggcagta |
| | | gtgtgacctagagggcagaagctgggaaacagggtgaaatgctgggcccc |
| | | gacagtcagggatagctgcacctagcttatatgccttcctctaatccagt |
| | | agaaattgttgtttctgtaggttttttttcttttctttttgagacagggt |
| | | cttcctctgttgcccaggctggagtacagtgtgcagtcacagttcactgc |
| | | agcctcgacctcctgcactcagatgatccttccatgtcagcctcttcagt |
| | | agctaggacacaggcgcatgccaccacatccaactaattttttttttttt |
| | | ttttttttgagacggagtctcactcttgcccaggctggagagcagtggtg |
| | | tgatcttaacttactgcaacctctgcctcctgggtgcaaggaattctcgt |
| | | gtctcagtctcccgtgtggctgggattacagatgcatgccaccatgcccg |
| | | gccaatttttttttttttgtatttttagtagaaatggtgtttgaccatgt |
| | | tggccaggctggtgtcaaaatcttggcctcaagtgatctgcccgcctcag |
| | | cctctcaaagtgctgggattataggtgtgagccaccatgcccggcctaat |
| | | tttaaaaattatttgtagagatgacgtctccctatgtttcccaggcttat |
| | | gtgcattatttatataggttgtctattgatatttacagtattagaaattg |
| | | aaacaaactgagaagcttaaaaatcttttctaaatttatttcaaaatagt |
| | | aattcatttcacccattgcatgttaatgtaagtaacatttttataaaagt |
| | | aactatattttgtaaaatgaaaaaatttgatgagaagggtggcactgcca |
| | | tacatttttgcaaatctctttaatgtctggattaataaacggttgaagtt |
| | | ttttgttttttttttggtggatttttttccctcttgttgcccacattgga |
| | | gtgcagtggcacgatctcggcttactgcaacctctgcctcctgggttcaa |
| | | gcgattctcctgcctcagcctctcgagtagctgggattacaggcacgcac |
| | | caccacacccggctaattttttgtatttttagtagaaacagggtttcagc |
| | | atgttagccactgcgcccgacccacggctgaggttttttgagtgataaaa |
| | | tgtacacaacagaaatctaccgtgttaaaacacttttaagtgtgttcgct |
| | | gaattttcttaaccgctttggcattcaatttgttgcaatatgttgaaaaa |
| | | tgtgtagaaaatctggctttagttggaagagcattttcatagccttttta |
| | | tgtgattctggatatttttctttgattctgtagaaacattcaacaagtga |
| | | gtagtagtttctttttctttttctgagatggggtcctactctgtcaccca |
| | | ggctggagtgcagtggtgctaccatagctcactccagccttgacctcctg |
| | | ggctcaagtgatccttctgccttggcctaccaagtagctgggactacagg |
| | | catgcaccaccacaccccgctgattttttattttgagtggtagtttctta |
| | | aatgttagttgcaatatggaatctgaaattttattatctttttttgtaca |
| | | ctattatattaaatgctgttgggtttttacccaggtgtgattttataacg |
| | | tcatgcactagtcatttggaaaatattggtctcttgaatgatgcagctct |
| | | tccacatgttgacgtcagtaaagtctaatagtctgaagatgttgagttca |
| | | tcacagtggctgatgtaagttttccaaaactctacttgaaaactggaatt |
| | | ttttatcattggcggcaaatactgtcagttattttccttgaagcgacaga |
| | | ctcattcatattcccaggaatggtctgcccgtactcaagcctgaacgacc |
| | | gtggtttgtctgtaggtcacgtgtaaataatgtgctaccaaaaaggtggc |
| | | cagttcagcttgcaatccaaacagctgcacagctgcttttctccaggacc |
| | | gctgtgctttgatgggcagcagcgtcgtcgtaggcgtgcttctcatttca |
| | | tcactcagactattcagaagacacctactcaagcttgcgatttaaccaat |
| | | cattcatgttgacagcttcatctgggacattcttttttttcttttttctg |
| | | tttttgtgacagagtctcgctctgtcgcccgggctggagtgcagtggtgt |
| | | gatcttggctcactgcaacctccgcctcctgggttcaagtgattctcctg |
| | | cctcagcctcccaagtagctgggatttacagatgtgtgccaccacgcccg |
| | | gctaatttttgtctttatggtagagacggggtttcgccatattggccagg |
| | | ctggtcttgaactcctgacctcaggtgatctccctacctcggcctcacaa |
| | | aatgctgggattacaggcgtgagccactatgcccggcccatcagggacat |
| | | tcttaagtgaagctgtctgcctttcctttcgtaagtgtggggtagtgaag |
| | | gacaagctgagggctagtgcagtttggtttccctgcatcgggacctgctc |
| | | aggcactagcagtttcacccatggcttcttctgcactgtcactgcaagtg |
| | | tcaacactgaagaaggcaaatgctgtcttagtgtcattgtgcaaatagtt |
| | | tagactccacagatgccttgcaaaggcctcaaggacctctagggatctgc |
| | | ggatcacactttgagtactgcttcgctagctgggctgcaagatgggaaga |
| | | ctgagggttttgtatgtgccacccaaacacgcattctcatgtgtgtgaaa |
| | | caacgccaattggagttgtcacacccattacatattgcctgtggaaatag |
| | | caagtgaccttagttcatacattgaccttttcttacaaaatccttaaaaa |
| | | tcttttgtgattctggtaaatctttgttgctcatgttcaaagtgtgtgat |
| | | aaagaccatcaacttcgagctggccatggctcgtgcctgtaataccagcg |
| | | ctttgggaggccgaggtgggtggatcacctgaggtcaagagttggagacc |
| | | agcctggccaacacggtgaagctccgtctctcctaaaaatactataatta |
| | | gccaggcgtggtggtgggcacctgtaatcccagctactcaggaggttgag |
| | | gcaggagaatcacttgaacctgggaggcggaggctgcagtgagccgagat |
| | | ggcaccactgcattccagcctgggcgacagagcgagactctgcctcaaaa |
| | | aacaaacacatggctgggcgcagtgagtcacgcctgtaatcccagtactt |
| | | tgggaggccgaggcgggcggatcacctgaggtcgggagttcgagaccagc |
| | | ctgaccaacatggagaaaccccgtctctactaaaaacacaaaattagcca |
| | | ggcatggtggcacatgcctgtggtcccagctactcgggcggctgaggtag |
| | | gagaattgcttgaatccaggaggcggaggttgcggtgagctgagatcatg |
| | | ccactgctctctagcctgggcaacaagagcgaaactccatctcaaaaaca |
| | | aacaaacaaacaaagaaacaaccatcaactttgtttctgtaaattttttt |
| | | tggaagggctcacccttgaatggttgggtgcttggattttgatttctgat |
| | | acattcgggcttaattctcggttgtatggtttcctaactgtgggactttg |
| | | ggcaagctacctaggggcgctgcaatttataggaatgatggtgtctctgt |
| | | cagagggtcgttgggagaatcaactgggataatgcaagaaaagcacttat |
| | | ttgcatagtgactcgatggtggacagccattccatcaataactaacgctc |
| | | cccgctggctgagtcacatttcgtattgcactaaagggtgtttctttctt |
| | | cctgcgctcaatctggcgcacatcttgtgtcgttcagtaccgaaacctag |
| | | aacacactagggactgtgattgtgtcagtttctcgtaataaaaagggcaa |
| | | gtgtccaaaatacaaaatactgatagctcgccctggttaaactctagctg |
| | | ccgaacttggcagcctctttactgaaaaccctcgggggttcagcaggctc |
| | | gttcctaggctgcttcagttccccagagcctggaggttcttaagatgatg |
| | | ggcccctcgatggttgaggaattataagcagatcttcagctgtgcaactc |
| | | cagctattggccgtggtttgaactagagtggtttttttcaactgtactgt |
| | | atcctactgtaaactcatttgtttattcattaaatattgaatgggttcct |
| | | accattggcaaggcgaggactaggttctggagatagaggggtggcccctg |
| | | ccctcttggggcatgacgcctagaaagggaagtagacagtgatcaaataa |
| | | tctgtacaggccaggtgcagtggtcatgtctataatcccagcactttgga |
| | | aggccaaggtaggaggatcgcttgagtccaggagctcgagaccagtctgg |
| | | gcaacatagtgagaccctgtctctaaaaataataaataattagccgtgca |
| | | tggtggcatgtgcctgtattccaagctacttggcagactgagatgggagg |
| | | atcgtttgagcccaggtggtcgaggctgcagtgagctgtgattgcaccac |
| | | tgtactcagcctgggtgacagagcaagactctgtctcaaaaaaaaaaaaa |
| | | aaaatcacctgaacaaatattgtactagacttcctgactgtattacagat |
| | | agggttgagatgatctctattttatagatgagaagactgaagcacagaga |
| | | ggttaggtaacttgtgatgatcacacagccagaaagggatactaaatggt |
| | | ggagccagtatttaaacccaaaggctgtattagaaaacactagtacccat |
| | | ctcacagaattgctgcgaggattaaatgagaaaatacaagttcttagaaa |
| | | agttcttggcacataatacctgtcagtagagaagagctagtgttactgaa |
| | | agttggaataattttcttagcagtggcagcaacagccaaaacaacgatac |
| | | ctgtctttagctctctgcatttaccctgctgtcatgtaccctgccctcac |
| | | gtatcctgctgtctcagccactgtgaaatcctaggatcctgggagccgtg |
| | | tgtgtttagatgcttttatttgcttggttctttccctttctataccattt |
| | | acagactgagtttgtttttttcttttctttttttttttttttgagacaag |
| | | agtctcactcctttgcccaggttggagtgcagtggcataatctcggctca |
| | | ctgtagcttccacctgtatcagaccattctaacactgctataaagttgct |
| | | acctgaggccaggtgcatggctcacgcctgtaatcccagcactttcggag |
| | | gctgaggcgggcggatcacttgagctcaggagttcgagaccagcctgtcc |
| | | gacatggcaaaaccccgtgtctactaaaaatacaaaaaattagccaggcg |
| | | tggtggcatacgcctctaatttcagctacttgggaggctgaggcaggaga |
| | | atcgctggaacctagaaggtggaggttgcagtgagcccagatcgcgccac |
| | | tgtactccagcctgggcaacagagtgagaccccatctcagaaacaaacaa |
| | | acaaaaaagatgctacctgagactgagtaatttataaagaaagcaggttt |
| | | aattggcacagttctgcatggctggggaggcgtcaggaaacttacaatca |
| | | tggcagaagatgaaggagaaacaaggcgcatcttaaatggtgtcaggaga |
| | | gagagagagtgagagagagagcatgcgaagggggaactgccacttttaaa |
| | | ccatcaggtctcgtgagaactccctcactctcacgagaacagcatggggg |
| | | aaaccacccccatgatctagtcccctcccaccaggtccctccctcgacac |
| | | atggggattacaattcaagatgagatttgggtggggacacagagctaaac |
| | | tatatcaacctcccagggtcaagtgctcctcgtacctcagcctccctgtg |
| | | actgcacatgtgcaccaccacgcccagctgattttttcaaatttttgaga |
| | | gagacagggtcttgccatgttgcctaggctggtctcaaactcctgggctc |
| | | aagccatcctcctgccttggcctcccgaagtgttgggattacaggcgtga |
| | | gccaaggcacccagctgagatggtttcttaacgtcctccaaaggtgattt |
| | | ttttcaagaatcaatataaactcttggattttagcttatttgatgtattt |
| | | cattccattacagatacatgatttttgatgctcagattgtcccatttggg |
| | | gcccatgggagcccctttaagttggctgctgagtcctcttcatgcaaccc |
| | | cagtggtctttgataactttcatacccttttagtaacctttttaaagagt |
| | | tgagaaattataggtattaaaatctcattaattctgtttgtctgatatgt |
| | | ttattcagagagactgagctaaagtttaccctcatcttttttgaaaagaa |
| | | aatgaatagtatatgcatgtttctatgaggaaataatgccacccaagaaa |
| | | cggtttgtgcatcttcgagttccttggaagctttaatttgcattcctaca |
| | | attgagagcaatgtagaatttctttctgtatcttcccactaatgcctgtt |
| | | gaggggcaggtgtctgtgaatgtaggtaaatcgtgataaaaaaacaataa |
| | | aaaggccgggcgtggtggttcacgtctgtaatcccagcactttgggaagc |
| | | tgaggcgggtggatcacttgaggtcaggagtttgagaccagcctggccaa |
| | | catggggaaaccctgtctgtactggaaatacaaaaattagccgggtgtcg |
| | | tggcgcacacctgtaatcccagctactcgggaaactgaggcaggagaatt |
| | | gtttcaacctgggaggcagaggttgcagtgagctgagatcacaccactgc |
| | | actccagcctgggtgacacggtgagaactctgtctccaaaaacaaaacaa |
| | | agcaaaacaaaacaaaaccaaaacaataaaaaaaaatcccaaaccaaaat |
| | | gtagatgacctgtttgcagactgaggagtcctgcagcaaactccatcccc |
| | | caggacagctgcggaatgttctagaaggcccaactctaggaaatccctta |
| | | gtaggagccttgctctcaacagtgcctcctggaacgatgggccggccact |
| | | ctcttttccatcttgcatcatatgtatttgacttattttgtttataatct |
| | | gtttccacgccactggaatgtatcttaagagcaggaattttccatgtttt |
| | | acttcctgctgtgttcctagtgctgggaatatagccaggcatataataca |
| | | tgcctgataatattggctgaatgaatgaatgaataaatgaatgaatatta |
| | | caggccagacacagtggctcatgcctgtaatcccagcactttggaaggcc |
| | | gaggcgggcggatcacctgaggtcgggagtttgagaccaacctggccaac |
| | | atggtgaaaccccatctctactaaaaatacaaaaattagccgggcttggt |
| | | ggcgtgcacctgtagtcccagctactcgggaaattgagacaggagaatca |
| | | cttgaacccgggaggcagaggttgctgagattgcaccactacattccagc |
| | | ctgggtgacagagtgagattctgtctcaaaaaaaaaaaccaaaaacaaaa |
| | | acaaaacaaaacaaaaaatcccaaaccaaaacgtagatgacatgtttgca |
| | | gactgaggagtcctcccccagcccagcaaaccccatccctcaggaccgct |
| | | gtggaacattccagaaggcccaactccaggaattcccttagtaggagcct |
| | | tgctttcaacagtgcctcctggagctatgggccagccactctcttttcca |
| | | gcttgcatcatatgtatttgacttaattttgtttataatctgtttccacg |
| | | ccactggaatgcatcttaagagcaggaattttccatgttttacttcctgc |
| | | tgtgttcctagcgctgggaatatagccaggcatataatatatgtgtgata |
| | | atatttgctgataaatgaatgaataaataagtaaatataagcctggtatg |
| | | gtggctcatgcctgtaatcccagcactttgggaggccaaggcaggcagat |
| | | cagctgaggtcaggagttcaagaacagcctggccaacgtggagaaatgcc |
| | | atctctattaaaaatacaaaaatcagctgggcatggtggtgggcacctgt |
| | | aatcccagctactcgggaagctgaggcaggagaatctcttgaacccagga |
| | | gatggaggttgctgagattgcgcccctgcactccagcctggatgacagag |
| | | caagactctgtcttaaaaaaaaaaaaaaaaaatatatatatatatatata |
| | | tatatatgtacatatgtatttatacatatatatagtgtgtgtgtgtgtat |
| | | atatatatatatatatatatatatatatatatgtaactccacatgtgagt |
| | | accctgttatgtgtgaaaccacaccccaaacatctacacagaacaagcct |
| | | ccaggtagaggaaaggcagtaaccgctttgctggaatgactgagtggttc |
| | | attaaatagaggctccggagggtcttgataggctctagcaaaggtggtgg |
| | | ccgagagggctgaccagcgcatggtgcctgggaatgtgaatgggcccagg |
| | | ggcgccagagtgtggacagcttgggactgtgggctccgaggtgctgcgct |
| | | gagggcgtggggcgcagggcagaaacacagaggactgagtgcttagggga |
| | | ggcccaggaaatgtggggttgaacggcctagttctggcaattgtttttaa |
| | | gctttgtaagatggagatttcaagaagtatatttatcttaataatattgt |
| | | agctcctggtaattcttatttgattaccatttatacacatggaaaaccag |
| | | catgcctgtttctggcaaagctccagggtagtctttgtgcataatttcca |
| | | ggatttttatgtaatgctattaaaatctgttcttttttttttcccccttc |
| | | caaagcggggaaaaagcagcttttgaatcatttattttcgtgtttgagtt |
| | | gtttgggaaatattaactacattttctaacactctagcctggagaaagga |
| | | agaaaccccacatgcgtgtgatttatttccttgtgtatggatattaaatg |
| | | caatataccattaaggctatgacactttaagtgaaagtgtttattgcatg |
| | | ttgattcagttagttcgtaccctattgtgttactatttaaagtgtggatt |
| | | gcatcgcattctggaacttcataaatgagttctttttcccataaggaaag |
| | | gccactgcttctgatgttcattaatgtgatttttgtgtgtgtgaatgagc |
| | | tgcctaaaaactgggtgtggtgagggtggtttctgaaagggaagcacttt |
| | | tatagcatgatcttatttgaaaatttaatttgtttttgtaaattagcttt |
| | | tggtcttaatacatttcctccactaacaagcacttttgggcaaataattt |
| | | aatctctctggttctgcttttttcatattggtaaaatataaagtttgaga |
| | | tagatatgcttaaaatctccaccattgtctaagtatagctgagattctca |
| | | cgtgattgggacatagtgctcattcttaatttgaaaagtgtttttttctt |
| | | tgtcctcttgtttcaactacgaactctcatttagacacatttagcatagg |
| | | gaagctccgatttatagagccactaaattgcagggcgtgtgtgagatttg |
| | | taacttcataaaagattctttacttcctttaaatataaatacgtaagatt |
| | | gagttctagaagtataatttacacatggtttcctgaggatcacgtttcct |
| | | gaataaagcagggtatcgttagaggaaccccatttgattttttttttttt |
| | | tttttttttgagatagagtcttgctctgtcacccagggtagagtgcagtg |
| | | gcgtgatctcagctcactgcaaactccgcctcctgggttcaagtgagtct |
| | | tgtgcctcagcctctcgagtagctgggactatagtcatgcaacatcatgc |
| | | ccgtctaatttttgtgtattttgtagagatggggtttcatcatgttgccc |
| | | aggctggtcttgaactcctgagctcaagcaatccagctgccttggcctcc |
| | | taaagtgctgtgattacaggcgtgacccatggtgcctggtctgggaatcc |
| | | tgtttgattttgaaagtctgagaaacacttttgaaatggcttggagaaaa |
| | | tgtgaagtgaataattattatttttttaatgtgcttcagttcccatgtga |
| | | acattattttagagagagattggaaggttagttgccagtaaaatgaattt |
| | | cactgtgttggcaagtttttcactcgtaggactgagtgaatcctggactg |
| | | acagtgttcttaccgatgattctgttgaggaaggacttgcttgctcttgt |
| | | tgctcttgacggtgagcttctggatctactggccctatattgagtgtgca |
| | | aggggacgtgttagtcctctagttgtcccccacattaatgcatctggaaa |
| | | gtgtctaccttagatgttgtaattagaaattagagggctgggcgcggtgg |
| | | ctcacgcctgtaatcgcagcactttgggaggctgaggcatgtggatcacc |
| | | tgagatcaggagttcgagatcagcctggccaacgtccgaaaccctgtctc |
| | | tactaaaaatacaaaaattagccgggcatggtggtgcgcacctgtaatcc |
| | | cagttactcagaggctgagacaggagaatcgcttgaacctggtaagtaga |
| | | ggttgcagtgagctgagattgcgccattgcactccagcctaggtgacaga |
| | | gactgtctcaaaaaaaaaaaaaaaaaaattaaatgtcatcaacattgaaa |
| | | ggaaactcttggattttgtcaaagatttataaaaagttttgaatgatttt |
| | | atttttttcattcagaagcataatttccctcttaaaatccctgatgtgga |
| | | tcatttggttccctgtcatttccattttctttgttcagagtgtctcttct |
| | | ggatctcctgagtttctgtgtgtgttttcttcctttttgctcaaatgatg |
| | | agcacacatgaaagcttttcctcctttgcctgggtgaggcggtgttccag |
| | | gtgtgatctgcagtgcctgattattatattgtattaatctcctcaatttt |
| | | attttctggcttgtctgcatttttccttttctttcttctccctttttttt |
| | | ttcttattagagatgggggtcttgctgtgttgcggggcctcgagcgatcg |
| | | tcccactttggcctcccaaagtgctaggattacgggcgtgagccacagcg |
| | | cctggcctccttttctttatagtaatttgaatgtggtgattttaatcctc |
| | | catggttttcttatccttcctcaacaaacagcaaagaggctgctggaaag |
| | | ccacatgacatttgctgtgatttgcacggctgatgggaaaacactacttg |
| | | gttctgaaggacagggttgtcattggatacctttgccctctgacccgagt |
| | | gagaatctcacctggacaaagacccgagttgtgaaatcacacagagaaag |
| | | tgtgattccgaacctgctgctgtcctgtgcttcctgaggacaagtgagtg |
| | | actgactccgtgtgccaggaactgcaggaggttaaaaactttcagaacta |
| | | ccatgcaaataagaaatttcaaaggacttttctctaacacacatttcata |
| | | gtgaacgtctcagctggattaacataattaacgctgtgttctttctggct |
| | | cctgaaagaaattgtgattaggaacactttcagggctgatgagtctaagg |
| | | tatcaactttgtcccttctgccacttgaagttcaaatggaaatgtttgct |
| | | ttcatagctccagtcatgatgttactgtatttagtagctattaagtttcc |
| | | aattccatagactcctgggagaagttccagttcgcactaaggcttctgtt |
| | | gcatttagcaccactgcctatggtgtgtggagctttcctgaacaagtaat |
| | | agcagcaaggttcttagcttagtaataattttaagctattctttgatttt |
| | | ttaaacaattaattaattaattattttgagacagcgtctggctctgtcgc |
| | | ccaggctggagtgcagtgacgtgatctcagctcattgcagcctccgcctc |
| | | ctgggctcaagtgatattctccagcttcagcctcctgagtagctgggact |
| | | gcaggcgcatgccaacacgactgactaatttttgtattttttgtagagac |
| | | aaaaatactttttttatgtagagacaaaaaaaaagttgaatgataaaatc |
| | | ttttattacttaataatatactttgattgagatttagccatttcattttc |
| | | actagtgtccagacgttttcctgaaaggcgtgattccatgagatttaata |
| | | ctgaattataatttgatgtaaatgttttatataatcagttgtttgttttt |
| | | aaacagtagcctcttttctaggcctgctctaggttgattaaatattaagt |
| | | tttctgatcagccagatccttaatagagagcttcggggacagcaaatggt |
| | | tgaatatgaaaccaacctcatttgccatgttgttagatataaatgtcagt |
| | | gatagcactttctggatggttgagttttctgaagagcagaggattttctc |
| | | ttttaatcaatcagataattttctgttttaaccaattcaaacatttgatt |
| | | ttatgtttctgaaatagataaagcactttcataaaaatggactgtgccaa |
| | | ataagaggtaattttcttgatgactcagttatttttgcctcttgttattt |
| | | ggcttttactaatatagcaatttctctataaaaataggaatctttgcttt |
| | | tttaaaaaaactgagatcattctgtttttccagttcttacattttaaaaa |
| | | ataatttttttggcttcttttgctatccatgtatgtagctgttggtgtgc |
| | | tttcctgtatcacatttgctcctggtttttacttcgttacattaaaaaac |
| | | caaaccaacaagtaattaagctcgttcagcctgcagatatcgtttgatgg |
| | | aaagggctaaggagtcagcctttgtgcaggtttttagttgtcagctatag |
| | | tctgagtaagtgaggttgctaggtacaggcgttcaggatgtgtaagatgt |
| | | tattacagttatttttagtgtattattaagggggaggaggagaaagagaa |
| | | atttgaatcaaatttgtgtcttattaaataggacacgttttccataagac |
| | | caaaagggcatttcttttgcttatgtactagaaatagaaaatggaaatta |
| | | gatattagattgtataatcgaaagtcaattagaattaatgaacaattcta |
| | | taactagttatactgtaagctaaaatgtgaatgttaaattagggaagtat |
| | | tgatttttatgaaaatttaccagaatttcaggaaaggactttgcttactt |
| | | tcccaagaaacgtctggtaggagtcactgtagaaatttggtgtgttctgt |
| | | acaacactgcccaggaggaactggccacatttcttcctacttggttcttt |
| | | tgtttgtttgttttttgagacagggtcttgctctgttgcctagaccggag |
| | | tgcagtggcacgatctcagctcactgcaacctccgtttcctggattcagg |
| | | caattctcgtgcctcagcctcccaagtaccggtgattacaggcacccgcc |
| | | accacacctgtctaattttttgtatttttagtagagacagggtttcacca |
| | | tgttggccaggctggccttgtactcctggcttcagatgatctgcccgcct |
| | | cggcctcccggagtgctgggattacaggcgtgagccacagtacccagcct |
| | | gctttatttttaattgaagctaaattcacataacttaatgatttttaagg |
| | | tgaacagttcagtggcatatcgcacattcacagtgttgtgcaaccatccc |
| | | ctgggtctaattccgaagcgttttcatcacccctagaaggaaaccctaca |
| | | cccactaagcagccacttcccagctcccaagccctagcacctggcagccg |
| | | ctgtgctgcactttgcatctgcaggatttaccgagtctgggcgtttctta |
| | | tacttggaatcgtaggatacgtggctttgtgtgtctggattctctcgctc |
| | | agcatgtttccaaggttcattcatgttgtggcatggagtagtacctcctt |
| | | tttgttatgggtgcataaaattccattgtatggataaaccacattttgtt |
| | | tttccattcttccattgatggacattagggttgtttctgcctcttggcta |
| | | ttgtggatggtgctgctgggaacactcgtgtacacgtttttgtttgagta |
| | | tctgttttctcagttctttggggcacatacctgggcatggagttgctggg |
| | | tcacgcggtaactctgtttaactttttgacaaacctctgccacttttaaa |
| | | atgaggaacttgggctgggtgcagtggctcacgcctgtaatcccaacact |
| | | ttgggaggctgaggcaggaggatcacttgaggtcaggagttcgagaccag |
| | | cctggccaacagggtgaaaccccgtgtctactaaaaatagaaaaatcagc |
| | | tgcgcatcatggcgcgtgcttataatcccagctactcgggaggctgaggc |
| | | aggagaatcgcttgaacctggaaggcagaggttgcagtgagccgaggtca |
| | | tgccactgcgctccagcctgggtgacagagtgagactctgtctcaaaaaa |
| | | taataaaataaaataaaataataaaataggaacttatatgaagaccctgt |
| | | aaaaagttacctgtggcagtccgatgttttggaaaacatctgaaagctta |
| | | ggtcaatttggtcatagatgtggctggccattatgtttcttgattgaaat |
| | | aattactttgttcactaccattcggaatagttttgtgattaggataaaaa |
| | | ttataatgtagatataagagcctagagactctttttatatgaatgcataa |
| | | gttttggctgttcccttcaaattggcatgaggcttggcaacactggaatt |
| | | gtgtgttagataaacagttggtatctagggcaagcttgaaaaaaaagtag |
| | | tatttaaacattgaccaaaaaaaaaaaatctcaacaactctaggaacttc |
| | | cttgttcaaatgaatccacaccgtctaaatacagcgtgataaaattaggc |
| | | ttccctctggtttccgtggggtcgagtgagccctgagcgtctcgtgccca |
| | | gccaggactctgcctgctggctggcttcggagcacagggaggtgggacgg |
| | | tttccacggccagcagtgttcccacagcacttccttaatatagaacgttt |
| | | ttaaaagcagcgttggccacagaaaaagctcaactcccaaacactgtcat |
| | | cagttcatagttcatttttattttgttttcatgttgttaaaacaagaaat |
| | | agcatgtaaacatgtctgcattaccgaacatcagtagtgtttaggataga |
| | | agaggaacacattgagtttctaagtagaaaaaaaatctgttcatccttaa |
| | | aggcaaaggaaggggcaaggtccctccttgaacaactgggtgaggttgga |
| | | cgcctccagctgtatttgctaagttgggcttggaagtatccatagggcgt |
| | | ggggggcctgctccagggctgacctcagctggagtcccctccaagtcagg |
| | | ggtatcgctcggcacacatttgtatgagttcacctgaaaagtgcccttgt |
| | | tgggggagcttggaatggcctgtagggcagctgggtcacagtggctgccg |
| | | gggatgcctgccaagctgttaagcagccctcgggcccacggagtcccagc |
| | | aagtgtggtttactgtttgttaggatgtaattcaagcctgtgggtaggta |
| | | aaactacccaggattaaggggggttggcttttccagggaacaaaaggatc |
| | | ccaccagggagaggactgttctgggaagtgccctggtcccctgtggccag |
| | | gcttgatttgctctctctttggaagccaccttggcacgcaggcactggtg |
| | | gaaaattttggagtacctgctggaggttgaagatgtgagcacgaagaaac |
| | | catggtcttgtctcgggagcctcccagactttcccctttacggctggtga |
| | | gaacgtgaaatggttcacttgctgcagaagacagtctggcaggtccccaa |
| | | aaggttaagcacggagctaccgtagggcccagccatgccactcctgggtt |
| | | tgtgcctaagaggtgaaaaacacacatccacacaaaaacttataggaatg |
| | | ttcatagcaacattatccacaattgccaaaaagtggaaacagcccaaatg |
| | | cctgtctgctgatgaacacataaacaaaacaagtaacacatcaacaaaac |
| | | gtagcacctctgtaggatagcatacgattcagccttaaaaaggaatggag |
| | | ttctgatacatgtgacaacgtggacgaatactgaaaacatgcccagtgaa |
| | | aaaagccagaccacaaaggccacgtttcgatcgcttgagcccaggagttc |
| | | gagaccagcctgggcaacatgtcaaaatcctgtcaactcaaaaattagcc |
| | | aggcatggtggcatgcacctgtggtcccagcccctcgggaggctgaggcg |
| | | ggaggatcacctgagcccaggaggtggaggttgcagtgagctgagattgc |
| | | ctgggtgacagagcaatctctgtctcaaaacaaaacaaaacaaaaaccca |
| | | aatgccacgttttatatgattccatttctaggaactctagaactacaggc |
| | | aaatccatacacacaaagtagattagtgatggccagggctggggaaactg |
| | | aagggaaatgggaggtgaccgccgatgggtatggatggggtttcctttcg |
| | | gggtgatgaaaatgttctcgaatgggctatggtgatggttgcacaactct |
| | | gtgtgcgaaacctccctgaattataaactataaacgagagaattacacag |
| | | tatatgaattctctctatataaagctgttatgaaaagcgaacctaccaga |
| | | ccaacattaacaattagagtatattgtagtacaagacagttgtagattgg |
| | | caagataagattggacatagagattttgtagattgtggagcggagaggga |
| | | tgaggtggacggtgctgttacagcctcttaggagggacagagatcctgag |
| | | gtgatcaccgagcttcaggatcttgttcataattttgagattaagaatct |
| | | tggtttggaatcccatgcctgtcatcccagcacttcgggaggccaaggtg |
| | | ggcagatcgcttgagctcaggagttcaagaccagcctggttcacatggtg |
| | | aaatcctgtcgctgcaaaatgcaaaaactagatgggaatggtggcacgtg |
| | | cctgtagtcccaactactctggaggctgaggtgggagggtcgcttgagcc |
| | | tgggaagttgaggctacagtgagctgagatcatgctgctgcactccagcc |
| | | tgggcagcacagcaagaccctatctaaaacaaagacaaaaacaaaccaga |
| | | atcttcatttccatcagtcttcagaactgtgtgagtttaggagttgtgat |
| | | tcatgctgaccatgagaccgatgtataaagcaggtgccagtgtattaagt |
| | | tactttggggaatatgatgtacttttccagtggctggaatattaatatct |
| | | acttcccaaggcagttcctccatgagatcaaggtatctgtgaggcctcat |
| | | taatagtaacaagttagccgggcatggtggcacatacctgtagtctcagc |
| | | tactggggaggttgaggcaggagcattgcttgagctcacactgctgcaca |
| | | ccagcctgggtgacagagtgagactctgtcgcaaaaaaacaaaaaaaaag |
| | | aagaagaaaggaagaaagaaacgaaagaaagggagaaagagagggagaga |
| | | gcgagagagacagaaacaaagagaaacaaagaaaagagaaagaaggaagg |
| | | aaggaagagagagagagaaagaaagaaaataaatagtaacaggattgctt |
| | | caggactgtagtatccgagcaaatagtaacaggattgcttcaggactgta |
| | | gtatccgagcaaatagtaacaggattgcttcaggactgtagtatctgagc |
| | | aaaggatctgatttgatccagcacatcccacgagggactgagtgtttatg |
| | | cccacagactcagtgccctccctgaactgtgcagagtaaactctgccaaa |
| | | ctcttagctgttgttgttcttcttttttttttttttttttttatcttttt |
| | | gagatggaatctcgctctgttgcccaggctggagtgcagcaatgtgatca |
| | | tagctcactacagcatccacctcttgggttcaaacaattctcatgtctcg |
| | | gcctcccaagaagctgggattacaggcatgtgccaccacatgtggctaat |
| | | tttttgtatttttaatagagacagggttttgctgtgttggccaggctggt |
| | | ctcgaactcctggtctcaaccagtctgcctgcctcagcctcccaaagtgc |
| | | tgggatgacagacgtgagccaccgtgcccggccttccttgttcttaaaga |
| | | aggagatgttacagaacaaacgaatctccatacaaaacaaaacctcacca |
| | | taaaagaggaaactccctgcttcatgaggggtgcaggtaaatgtagaaac |
| | | gctcaccaagaaagccagagtggatttcacattaatagaatgcaggtaaa |
| | | agacataacttgttgggatgtagggcgcagtatcagaatggaatagggtt |
| | | caggtgagctcctaggatggcaggatttgaagaaggagcagcaccttgat |
| | | taggggagaatgaagaagctttggagggctgggcgtggtggctcacgcct |
| | | gtaattccagcactctgggaggcctaggtgggcggatcacctgaggtcag |
| | | gggtttgagatcagcttgggaaacatggtgaaaccccatctctactaaaa |
| | | atacaaaaattagccgggcgtggtggcgcatgcctgtaattccagctact |
| | | agagaggctgaggcaggaggatcgtttgaacccaggaggcagaggttgca |
| | | gtgagccaagatcatgccattgcacttcagcctgggtgacagagtaagac |
| | | cctgtcttaaaaaaattaataaaaataaaaaacacagtccaatttttttg |
| | | agctaattatggaagtattaatggataaaatctacattttgtactggtgc |
| | | tgagagtgagctgtcccctaagtagattatgtttcatgattctgtgtcaa |
| | | ctttctctgggctgatgggccctctttggacactgacgtctcctcacgtc |
| | | catgatccctcctgggtccgtgccagctgcgcgtgtcatcagagcggagc |
| | | gtgcgcttgtccttcatcacaggcctgggtgatgtgtggctgacatgcgt |
| | | cacatgggataggctctccgatctcagggctcgggctgccacccctgcca |
| | | cgtagtacatggcatgtggctgattctccacttgcttgcaatggaccctc |
| | | tgtggaaggcaccggagaggcggtttccctgtctcttctcctcctgtgtt |
| | | gccctcctccccctcgatagatgtgacctcccttccagctttgtgccagt |
| | | gtctggcagcacttgattttttttctccttgtccttcatctgttttcatt |
| | | gcgtcgccaccttccccacccccaagcagcatccttctgccccttggtgc |
| | | tctccgcgcggccgtctgcctacctgtgagtacgctgaccagggctggtt |
| | | gactgcctacacattagcggagtctttctcattttttagggcacagtaga |
| | | tactcagcgagtgttgttttttttttaataattatgcaataaatgaatgg |
| | | atgtccgcacaccaccctgaacaccagcacccactggtcatccctggaca |
| | | cgcatgggtggaaggaccacactggttgtccctggaagagtagctcactc |
| | | tcgagcctgcaccgatgtcatctgagtcaaaacacagacgtctgggcccc |
| | | accctagagagtctggctcagcgggtccagggtggggcaagaatgtgcat |
| | | tcctgcaagttcccaggtgatactgaggcggctgctggggggataccatt |
| | | tcaagaagcactgcttggaggcaacgcagacctctccctcttcgacgagg |
| | | tcctgttcacttccatgtaaccagtcagggttggaatcccacatggagtt |
| | | cccctcctttggtgaccctctgctgcagctcttgggagcctcccctttag |
| | | atgagttcctccctgctctgaccaggagttgctttttctataccacctca |
| | | gctgatggtgttcacacacacattcatttaacacacatttacggagggct |
| | | cctgggggctgggtgcacacacttggtgcggctctcttttggaacctggt |
| | | gccttctctctctccttcatttttgaggcagagtcttgctctgtcgccca |
| | | ggctggagtgcagtggtgcgatctcggctcatggcaatctctgcgtccca |
| | | ggcttaagtgattcttccacctcagcttcccaagtagctgggattatagg |
| | | tatgcgccaccatgccccgctaatttttgtatttttagtagagacagggt |
| | | ttggccatgttggccaggctggtctcgaactcctgacctcaagcgatctg |
| | | cccaccttgacctcccaaagtgctgggattacaggcgtgagccactgcgt |
| | | ccagcttccagtgtcctccctttcagcctctgggtgttgccagccagtgc |
| | | caccactcagcactgtttatacctaaggcaccaatgggtaaatggagaga |
| | | caggcgtctgaagacttttaaaaatgtgtactttatttggctgtagctaa |
| | | accagttagaaggccgggagttgaagttgatatgagtatcataaatgcgc |
| | | aagttaagtagttacttaagaagtcacaggaatatgggtcgttccttgtt |
| | | gggtttctgataactagcaggcggctcagtttggctatgttaataacaga |
| | | aacacagagattgccactgattctatcacatttcctgtttgtttgttttt |
| | | gtttttgtttttgagacagtctcactctgtcatccaggctggagtgcagt |
| | | ggtgcaatcatggctcactgaagcctcgaccacccaggctcaagtgatcc |
| | | ttccacctcagcctcctgagtagctggagctagaggtgtgcaccacctaa |
| | | tttttaaactttctgaattttttgtagagatggggtctcactgtgttgtc |
| | | cagtctggtttcaatctcctggcctcaagcaatacccccaccttggcctc |
| | | ccagagtgttgagattacaggtgtgagccaccgtgcccagccaacatttc |
| | | attttttaaaaaatgctttattgaggctgggcccggtggctcaggcctgt |
| | | aaacccagcactttgggaggctgaggcaggcagatcacttgaggtcagga |
| | | gttcgagaccagcctggccaacatggtaaaaccctgtctctactaaaaat |
| | | ataaaaattagctgggcgtggtggtgggtgcctgtaatcccagctacttg |
| | | ggaggctgaggcaggagaatcacttgaacccaggaggcggaggttgctgt |
| | | gagccgagattgtgccactgcattccagcctgggtgacagggtgagaccc |
| | | tgtgtcaaaaacaaaaacaaaacaaaacaaacaaaagaaagctttattga |
| | | gatgtaattcacctgtcacccagcctgggtgacagggtgagaccctgtgt |
| | | caaaaagaaaaacacaaaacaaacaaaagaaagctttattgagatgtaat |
| | | tcacatgtcacacaattcagcagtttaagtgtacagttcctatactttga |
| | | catccatcactacagacaatttggggacgttttcttcactcgacagagaa |
| | | gcccggcacacccgagctgccacccccagtgctcccagcccctggcagcc |
| | | cctaagctgctttctgtctctttgcatttgcctgttctggacatttcata |
| | | aaaatggaatcacatcatatgtggcccttcatgactggcttctctcactg |
| | | agcctaatgttttctgggttcacccatggtgtagcatgtattagtgcttt |
| | | cttccttttcatggctgaataatattccattgcgttttgtttatctttgt |
| | | tgattatccattcctcagttgatggatatttgggttgtttcactttttga |
| | | ctatgatgaatagtgtttctatgaatgttggcagacacgttttcctgtgg |
| | | acatatgttttcatttctcttgggtatgtacctaggagtggaattgctgg |
| | | gtcatatggtaactctgtttaactttttgaggaatgtctggagtttgttt |
| | | taaagtggctatgccattttctcttcccaccagagtgtgtgcgggccctg |
| | | atttccccacgtctccataaacacttgctgctgttcgtcttttttattat |
| | | agccacactagtggaaagacacttttgtatcccacacaaacagaaaactt |
| | | atgtataggtgatttttctaaaaaaaagactttatgttttgataaaataa |
| | | gcatgttatattggttaagatggtttttgttgtgagttaacgaaaaccta |
| | | actaaaattacctaaaacaaacaaacaaaaaaacgcatttactatctcat |
| | | gtaacaagaagtatggcagttccagggttgattaattcagaggtgtgatg |
| | | acatttccaggaccccaggtgcttttcatctttgctctctgctgtctttg |
| | | ccacgtgggagatgtcttcccttgtggcacaaagtggctgcagccattcc |
| | | gaatgacacagtgatatctagtaaagaagaggggcttttccaatcctgca |
| | | tgtcttattttactggcatggaaactaagcagacatctctcagctccctt |
| | | tggccaaggcggggccatgtgccacatcctccttagttactggaaagggg |
| | | agggaagccccattggggtgaacttaccctgctccagctacttactgcgg |
| | | gtgggctgtgtgtgcgcatgcatggtgtgtgtgcgtgtgcgtccatgcgt |
| | | gggtgtgcatgtgtgtgcgtgcatgcgtgggtgtgcatatatgtgtgcat |
| | | gcatgcatgggtgtgcatgggtgtgcatatatgtgtgcgtgcatgcgtgg |
| | | gtgtgcctgcatgtgtgtgtgcgtgcttgcatgggtgtgcctgtgtgcat |
| | | gcatgcatgtgtgtgtgtgcgtgtgtgcatctccttcctaagcaggtaga |
| | | agggagataccccaagcagaatcaggcttctgctggtaaggaaggtaggt |
| | | gtgtgggaacggggaggacatgaagaaagaaaatgattcttagggatgca |
| | | atgcattagtggctgtgactttagttaaaacatagaattttgtttgtttt |
| | | ttttcttttgagacagggtcctgttctgttgccctggctggtgtgcagtg |
| | | gcaatagtggctcactgtagccttgatctcctgggctctagccatcctcc |
| | | tgcctcagcctcttgagtatctgggaatacagtcgtgtaccaccatgcct |
| | | ggctaattttcaattttttttttgtagagacagggtctcgctatgttgcc |
| | | gaggctggtctttaactcctggtgtcaagtgacactcttgcctcagtctc |
| | | ccaaagtgctgggattacaggtgtgagccactgcccaaccttcatgcagc |
| | | attttaaaaaatgaaaatgtagctgcttgttctaggagaaacttgtgaaa |
| | | ttggatcatttaaccaactctgcctgtgcgtgacttgccttcagttctcc |
| | | agcttggcctcaatccctttttcttgctgcttggaacccacatcaccacc |
| | | agtgacctcacaaggcagtgcccctccgcacccctcccttctccaagact |
| | | cccccgccgccccctttcttgctcttacccacacgccactgcagacacag |
| | | cgccctcctcccctgctgcccccctcttgctcttacccacacgcccctgc |
| | | agacacagcaccctccccccacccaccgttacaccctctcttgcttttac |
| | | ccacacgcccctgcagacacagcgccctcctcccctcctgcaccctgggg |
| | | gctccccgcatgtgctgtgatgttgaacatgtgggtgtcaggatgaaagg |
| | | aagaaatctagatttgatgtggtgggaggtaaaagcactctcgaaggctt |
| | | tcgagctgggctatgactgtcaggatgaatgtggttttaatatgtggaag |
| | | agatttatttctttaatagtcacctgttaagagtcttagatatcactgat |
| | | gtgtcttgttaagaaaccttataacaaacgtatttcttaaacttacatct |
| | | ggagctctgcaaagcattagctgttctgcaggaatgacagagggtctcct |
| | | gtgcggccagctcctctggagtcagggagccatggagccactgctggttc |
| | | tgaggatggtgacagcaggaggtggagatggcttcatgatctgctgtgag |
| | | caacgtgcaaccttctccctgctgttaagtgactagaattcccaggtgcc |
| | | gcctgctgattagttccagtttgtgttccacgagagcagatgtgtgtgag |
| | | gtgcagggaatgccccgctgcgggagatggttttctcctgccaccgtccc |
| | | tccccaggccacaccatccacacacatgagcacgctcgtcttcctttctc |
| | | ataaagcacagacctagaaatatttctcggtttaaaaatagtgttttccc |
| | | tacctcgagtcaaggttcagttctcatctctctaattttagttccttgtt |
| | | tttatgaactttgaaatgttttccttaaccagaaaacagaaaaaagcaat |
| | | cccagagacaagggaaatttcttgctccatttccctgtagtaaaggcatt |
| | | tctatgtgagattttctttttatcctgagcaggttatcagaaaccaaaat |
| | | tttgaattaggagtttaaagaagaggcaagggggaaaaagtgctctctac |
| | | gggaccttggttgccatgtggaatgtttttggagcataaaattgatattt |
| | | ttacaaaaaaaaaaaaacgaaaaaaatgtttctggtagaggcctggagaa |
| | | cgccgtcatgattctgaaggtgtatatattttttattgtttctgtcaaac |
| | | taattttatataagacattaatggaaaggtcaaaacacataattctctga |
| | | agcctattagaaaaatttcttccgttgggctgggaacggtgggtcatgcc |
| | | tgaaatcccagcactttgggaggttgaggcgggaggatcactcttgaggc |
| | | caagagtttgagactagcctggccaacgtggcaaaaccccatttctatca |
| | | aaaaatgtatatatatatacaaaaattaatcgggtgtggtggcgcacgcc |
| | | tgtagtctcagctactcgggaggctaaggcaggagaatcacttgagcctg |
| | | ggagatggaggttgcagtgaaccaagagcatgccactgcactagagcctg |
| | | ggcaacagagcaagactgtctcaaaaaaaaagtaaagaaaagaaaaatta |
| | | cctctgttaacttctttaaattcttaggtacaggtggtaatttttttctt |
| | | tgttaaaattgatgttggtcattttgtgtttgagttttctgttgctattt |
| | | catggggattttcctgtgcatctacataatatagtatttctttgaattaa |
| | | tatgccatagtttaatcatttcctgtttgagctttttagattgcttgaag |
| | | gtttttctttcctttgtaaatactgcagctttaaggaatgtgtgactttt |
| | | gaagctctctgttttggcagatggcaaagggggtgaccatcttattttta |
| | | ttaattttttattataaaagtaaaacatgtttttggtgttttagaaaaga |
| | | cagataagcaaaaagaagattaaaaaaacatttaccaactagagatagcc |
| | | attttgaatgtttcaacatgaggtttttagactttttcagtgcatatacg |
| | | atatatatcaaatatttattcaaaatcaaatatatatcaatgcatataaa |
| | | tatatatagatataatggtgtcatatgaaacaccattaacaccattaaac |
| | | accattaacaccattaaacaccattaacaccattaaacaccattcatgga |
| | | tcatgaatacctttccacttaaatctgtatgtacatctttgtttatttta |
| | | ttttattttatttttttgagacagagtcttgctctgtcatccaggctgaa |
| | | gtgcaggggcatgatctcggctcactgcaacctctgcctcccgggttcaa |
| | | gagattctcctgcctcagactccccagtagctgggactacaggcgtccgc |
| | | caccacacccagctaatttttgtatttttagtagagacggggtttcaaca |
| | | tgttggccaggatggtctccaactcctgacctcaggtgatccacccacct |
| | | tggcctcccaaagtgctgggattacatgcgtgagccaccatgcctggcca |
| | | agtctgtacatacatctgtatcatcattttaaatgacttagattttattc |
| | | tggggatgtactataatttattcaggaaatatctcttgttggaaatgtag |
| | | attgtttccagctttgaatcattgtatatcacaatatgaaactgttaata |
| | | atccagccatccctcacatcaggatgcattgtataatttaatggtatgtc |
| | | atagtttaattggcagcactttgcattatattttaatagaacaaaaagta |
| | | atgaaacatcatcattggcatcttagactcgatgaaatataatattgtca |
| | | attataacagtgtgatgaatatccttatttgccgttgtctttgcccacga |
| | | taattagtagtggggccaaatctctttttcatatgtgtgttgcccttttg |
| | | tatttcttcatgagttgcttgtgaattggttgtcttttccttactgattt |
| | | gtgaaagttctttatttatgtaagtatcttaaatctttgtcttccatata |
| | | tgttgtaaaatgcctcccattttctaatttgtctttcaatcgtgatattt |
| | | cttgatatgtggaaatttaaaatctttatgttggtcttatctatcaatca |
| | | ttgtttatggtttctacctttagtatcatggttagaaaagccttccccac |
| | | ctgaggactatgtaaaatattaacctatattttcttctaatacttttatg |
| | | tttttgttttcttttttttttttctttttttgagacagggtctcactcta |
| | | ggcaggctggagtgcagtggcgtgatctcactgcagctttgacctcctag |
| | | gcttaggtgatcttcccacctcagcctcccaagtagctgggactacaggt |
| | | gtacaccaccacacctggctaatttttaagtattttgtagagatggaggt |
| | | ctcattatattgcccagtctggtctcaaacttctgagttcaagcaatcct |
| | | cccaccttggcctcccaaagtgttgggataacaagcatgaaccattgtgc |
| | | ctggactggatataccaccattttaaaaaatcagtttatcagttgatgaa |
| | | tatttgagttgcttgcagttcttggctattatgaatagagttgctgtgaa |
| | | cattcacaagtcttccgtggatgtatatttttatttgtttcggttaaatg |
| | | cataggagtggagtgactgggttgtatgatgagtgtgtttaactttataa |
| | | gaaactatgaagctgaacatgttatttttctttgtaactggacacttaat |
| | | tgaatgcttattgtgtctgaattttttttttttttctgaagcagagtctc |
| | | actttattgcccaggctggagtgcagtggcaccatcttggctcactacca |
| | | cctctgcctcctgggttcaagcaattctgccgaagactcccaagtactag |
| | | gattacaggtgtgcaccaccacgcctggctaatttcttttgtatttttag |
| | | tagagatggcgttttgccatgctggccaggctggtctcaaactcctgacc |
| | | tcaagtaatccacccacctcagccttccaaagtgttgggattacaggtgt |
| | | gagtcaccgcgcctggcctgtgtctgatttttaaaaatcagatagcctta |
| | | gatttttcttggcagaaaatcatataaatttaaaatcccctccttttcag |
| | | tttttatatctcattttaaaaaattggactgaaaggtaaaggccaaaaat |
| | | atgcctattctctgcttaggagttacagtaacttttgatgtgtgtgtgtg |
| | | catatacctgtgtgcgtgtgtgtgggtgttttcccgctgtaagaaaagaa |
| | | ggcatgatgtaatgtgcaggaaataatcataataaagagaatgctggcgt |
| | | acttcttgagtgctcattgttgctaaattggtcatttactcctaaaggac |
| | | gttggggaagggtgtgtttgatctctctacagccagaaagaccacctgaa |
| | | aaccctcctgggcctcgtgaatctgctcatctctccagatgtcacaggaa |
| | | gtgtcctcggggggggacaaggctgtgctgttccatgcaggcatggtctc |
| | | aggctgttcccaattgtgagtgagttttgaacaaaactgtaacttccctg |
| | | tatagggcttacagttggcctcatcttacaccctgtagtccagtgagtcc |
| | | gcatgctagctgtggaaagcgtgtacagaaaggaggctgattcttttttt |
| | | ttttttttgagacgaagtctcaaccttgtcccccaggctggaatgcaatg |
| | | gcgtgatctcggctcactgcaacgtccacctctgaggttcaagcgattct |
| | | ccttcttcagccccctgagtagctgggattacaggtgcctgccaccatgc |
| | | ccggctaatttttgtatttttagtagagacggggtttcaccatgttggcc |
| | | aagctggtctcggactcctcacctcaggtgatcctcctcccttggcctcc |
| | | caaagtgctgggattacaggcgtgagccaccgcccccagccaggaggctg |
| | | attcttaattattgatgcactgcagcctgggcaacagagcaagaccttgt |
| | | ctaaaaaaaaaattattgatgccatttggttctctttgaaactaattaat |
| | | ggctccttaatttgtcagtgcgtctatcttctagcgatcacagatgctgt |
| | | ttgggtgctcttgtcagtttttggtaaccatttctggtattttcttttca |
| | | aatccccagcctcactttgtgctggttatcacaagatccacttagacctt |
| | | agaggtgagagcttgggaatatttcatctgcaagaaaaatcattcgtctg |
| | | tctttgtgtgggacgagttaggttatgtaagctgatggaacacgaaacag |
| | | aaaactggagcctttttctttgggcacacaaaaatgcaaaaatctaaatc |
| | | tcttattttgttgaatttttaagtggaaggagttgtagatttgccaccat |
| | | cagtcctggagctgacttgaatttttcttttaggctcttgttagcttttc |
| | | taacagagatgtcacagattagcaactaggcataaacctgagaaattggg |
| | | cactaattttgttttgcatttttgttttgtaagacgggggtcttgctgtg |
| | | ttgcacaggctgcagtacagtggctgttcacaggtgtgatggtagcgcac |
| | | tgcagcctcgaactcgtgggctcaagtgatcctcccacctcagcctccca |
| | | agtagctgggaccacaggcatgtgctacgccacccagctgttattttgca |
| | | tttgctctgtcatcacgttgtgcttttgtgcctgccttttctcctcatta |
| | | actagatatctgtcacttaaagaaataacagcgatgcatttcaggataat |
| | | agaacccctatgaactcttccagacttaaaatttatctgtttctccaaaa |
| | | aacactgcctgcatttttcccaaataagccagatactctcagccaggtat |
| | | ctgtgcaggttggacccctgcccaccttacaagatccagtagcatctccg |
| | | ttggaaagcttttcctgagccctcccgcctggcccccccgctgccccagc |
| | | cgccattccttgccaggtctctttttacctccttttgcgttaatgggtct |
| | | ttggagttgtgtctccccttcactgagctctccaggacacattttgcaac |
| | | atttttgtgtccctggtgtatggtgtttagcagacgctcagtatttacat |
| | | gttgacagattgcacacatgggaccgggtacatttcttgccagtacatcc |
| | | agttttcccttacctcttctgctgtaaattaatgttcctctggggtttaa |
| | | aaaattaatttagtggttaatttagtaagagattaaaattaggcactggc |
| | | caggcacggtggctcatgcctgtaatcccagcactttgggaggctgatgt |
| | | ggacagatcaccagaggtcaggagttcgagactagcctggccaacatggc |
| | | aaaaccccatctctactaaaaatacaaaaattatctgggcgtggtggcac |
| | | atgcctgtaatcccagctacttgggaggctgggacaggagaattgcttga |
| | | acccgggaggcggaggttgcagtgagccaagatggtgccactgcactcca |
| | | tatatatatagtaacaggtctcaccttgtcacccaggctggagtggagtg |
| | | gcatgatcacagctcactgtagcctcagcttcctgggctgaagtgatcct |
| | | cctacctcagcttcctgagtagctgggactacaggcatgcaccaccatag |
| | | ctggctaatttttctatttttttgtggagacggggtctcaccatgttgcc |
| | | caggctggccttgaactcctgggctcaagcgatcctcctgcctcagcctc |
| | | tcaaaagtattgggattacaggtgtgagccactatacccggccataaagt |
| | | agtacttaaaagtgtaacaactttcaataaatctttgtgagattcagaac |
| | | aaccttttgaattgtctatgtaaagaaataccgaactgcttttttttccc |
| | | ctcgatcactttttaaccttccactttgtagacaatttgctagtgtgttt |
| | | tctatttttttttttttttgccactgggtattgccctgtcatccagactg |
| | | gggtacaatggcatgatcgtagctcactgaagcctcgacctcctgggctc |
| | | aagcgacccacccgcctctgtcgcccaagtagctgggaatataggcgtac |
| | | accaccatgcccggctaattgtttttttaatttaaattttgttttttttt |
| | | ttttgcggaggcagatcctcactatgttgcccagcgtggtcttgaactcc |
| | | tggcctcaagtgctcctcccacctctacctcccaaagtgctgggattaca |
| | | ggcatgagccactgtgcccagccagatttttgtcatgattctatttttat |
| | | ctcttctatcgatttatttcttatatattttttctcacttattttaagtc |
| | | attgcccgaaatttacaaagtacattttttatcagtctgcctccaaatat |
| | | tataccatttattatataagaacctcgtaagaattcttcataactgtaag |
| | | ttactacaaggttctatgagaaccttgtaaatgtttatgatgctaatagt |
| | | gtcataataatgtattttatatatattataaacctcataacatgttattt |
| | | ttacagaaaattaccttttagagcaatgctgtttgatgaaacttcctgca |
| | | atgatggaaatgttttatatctagactatacaatatggtagctactatcc |
| | | atgtgtgaccattgagtacctgaaaacaactgaattttaaataaactagt |
| | | ttaatttaagtaatttaaatttaagcggctgccatatagacagtgtagtt |
| | | ttagaacaatggaagattttcaaaatacttttgaaaattattttgaaaat |
| | | ctttcttctttttaggccacttcaggaactcttcgtttctttgtgtagat |
| | | ctataggtttctgtctcatctcccgactgaaaaacttcctttaacatttt |
| | | ttatgctacaggtcttctgggaataaatgatctcagcctttgtctgtctg |
| | | aaaaagtttttatttccctcccattagaaaatgtttttaggccgggcaca |
| | | atggctcatgcctgttattcccagcactttgggaagccgaggtaggtgga |
| | | tcacctgcagtcaggagttcaagaccagcctggccaacatggcaaaaccc |
| | | tgtctctactaaaaatacaaaaattagctggatatggtggtgagagcctg |
| | | tcatcccagctacttgggaggctgaggcaggagagtcatttgaacccagg |
| | | aggtggaggttgcagtgagctgagatcgcgccattgcattccagtctggg |
| | | caactgagtgagacttttgtctcaaaaaaaaaaaaaagtttttaaagagg |
| | | gctgggtgcggtgactcatgcttgtaattccagcactttgggaggctgag |
| | | gagagttgatcacttgaggccaggagttggagacctgcctggctgacaag |
| | | gcgaaaccccgtctctaccaaaaattagctgggcatggtggcgcatgcct |
| | | gtagttccagctacttgggaggccgaggcaggagaattacttgaacccag |
| | | gaggcggaggttgcagagagctgagatcacgtcactgcattccagcctgg |
| | | gcaatagagtgagactctttctgaaaaaaaagaaaatgtttaaagaaaaa |
| | | tttcataccaaggagatccaagagacactcatttttaagagtcggttttt |
| | | tcttttttttggttttgtttgtttcttttttttttttttttcttttagga |
| | | tatgaaggtttgtgttgaaaaattttttttgcaacttattcttttagtac |
| | | tttaaagatatcacaccactgccttctggcttttgttgttttggtgagaa |
| | | gtctgctataattcttttctttgttcctctgaatgtaatatgtttcccaa |
| | | ttcccactgtcaccccggcagcttttaagattttctctttatttttagtt |
| | | ttcgacaatttggacatgatgttgttttggtatttctcttttttggagtc |
| | | ctctaaacttcttggatctgtgctttgatgtcttttattatgtttaaaaa |
| | | attattggccattatctcttcaaatatttccttctatcctttctttcttc |
| | | ttcaagcattttcttttttcttagaaacatttaataggggcttatgcaca |
| | | gaagctatgtctgttctcaagcttctgttcattagtctgtgaactggggc |
| | | tgcagcgagaagagacagtggatccctgcaccattatcccctggtcccag |
| | | ggcttgtataccgtagggaaggggtgattcagagggatgtgtaggtcagt |
| | | gtgagtacaataacatcaaagttgtttgacctaaggtcaggctttatggt |
| | | aagttcttggtcttacacgaggaacagcgcataagctggaaatctcagag |
| | | ggtttcctggaactggggttaatcagaagccacatggcggagtagcatcc |
| | | acgatggagctacttcagcctccacagtccactcttgtaatccagctctt |
| | | gtaatctcacatgccctcttcttccttcatggtccctgagcctttaagga |
| | | gggtgcttgatactgtatagctttagcagcagtgccttggtcatggaaaa |
| | | cagatcgggcccagtgggattccaaacaagggagattcacaggcttttaa |
| | | atcgtcttcagtcttcagaatgcagtggctttggttctgtcagaataggc |
| | | aaaacaatgtgagatatataacatcgatcactcggatagtagaattctgt |
| | | gcacacagagattgcaatagaaagagaatcgcctgtaatcccagctactc |
| | | aggaggctgaggcgcgagaatcgcttgaacccgggaggtggaggttgcag |
| | | ggagccgagaccgcaccaccgcactcccgcctggatgacagtgagactct |
| | | gtctcaaaaaccaaacaaaaaaacccagaattcgtgggccagaacaacaa |
| | | tgaaaaaagaacgtattctgttaagaagccaactaaaatgcatcatgatg |
| | | aaaattaaaactcggttcttctttagagacttgttgcagccaggaaatgg |
| | | ttcaggattagcccaaattgtaggcagataataaaaactcacaaacaatg |
| | | gttaaggctagaatctaaatttttctttctccagtttccccatttctacc |
| | | agggataaattatatatataatagtaagaccaatttatttgcaaaacaaa |
| | | ttttagttgctttatacttggcctgaagatttgcatcaaatgtagcaaga |
| | | attgtgatccaccatacggaccctttttaagttggctttgcaggaacctt |
| | | tataaggaattttggatttgactttttaaaagcctcaatgccagatattg |
| | | atctgttttcaacttttttgattttttttttttttttttttttttttttt |
| | | tttggagactgagtttcgcttttgttgcccaggctggagtgcaagtagca |
| | | ccatctctgctcactgcaacctccgcttcccgggttcaattgattctccc |
| | | gcctcagcctcccgagtagctaggattacagacatggatcaccatggctg |
| | | gctgatttttgtatttttaggagagatgggatattaccatattggccagg |
| | | ctggcctcgaactcctggcctcaagtgatctgcccacctgggcctcccaa |
| | | agtgccgggattacaggtgtgagccaccacgcctggctactacttggtta |
| | | ctttgtaaccccgcctctttgatgggtcaagaaaagttgtggttatatta |
| | | tttatcttggtttttattattattattattttattttttgagacggagac |
| | | tctctctgttgccaagactggagtgcagtggtctgatcatggctcactga |
| | | agccttgaccttctgggctcaaagtaatcctcccatctcagactcctgag |
| | | tagctaggactacatgcatgcatcatcacacccagctcatttttatgtat |
| | | ttttgtgtgtagatggggtcttatgttactcaggctggtctggaactcct |
| | | ggggtcaagtagtcctcctgcctcggcctcccaaagtgataggattacag |
| | | gtgtgagccactgcacttcgcttttttctttttaaagagaaaggatctct |
| | | ctatgttgcccaggctggacttgaactcctggactcaagcgatcctcctg |
| | | ccttggtcttcgaaagcactgggattacaggtgtgagccaccaggcccag |
| | | ccagttttaatttctgttaggtggaatgacattccaggtttctaaaaata |
| | | acttctacttggttatttttcaaaagttttgttgagttcagtaattatat |
| | | aaggtttataacataatattatttgaaactttcatccacatcctattctt |
| | | tactggtagagaaaagccacatataagaaatacatgtgtgtttgtctttt |
| | | ataaatatatgttacccagttttccagttactagctgcaagttttttttc |
| | | caggtcataatttggagaaaaatatatgcgtgaaaattataacgataaag |
| | | cttattttctatacaacaaacagtgagtgatatggaaatgtatgtcctta |
| | | ctagtaattttaaaagtacaaaagagaatgccttaaaagtacacacacac |
| | | acacacacacacacatacacacacatacatatttttttgagacagagtct |
| | | cactctgttgccgaggctggagtgcagtagcgcaatcttaactcactgca |
| | | acctttgcctcccgggttcaagcaattcttgtgcctcaccctcctgagtg |
| | | gctgggattacaggcatgtgccaccacgcctggctaatttttttgtactt |
| | | ttagtagagacagggtttcaccatcttgggcagactggtctcaaactcct |
| | | gccctcaagtgatccacctgcctcagcctcccaaagtgctgggattacaa |
| | | gtgtgagccaccatgcctggccgcattatatactttttaaggcagagaaa |
| | | attttctaaaacttgatgggtggcctgttgttgctggttcacttcttcta |
| | | aagaacagaagtaagaactgtaacactgtccgaatcctgggagagtcgcc |
| | | ttagaagatggtcctgtagatacaagagaataatttactcaatgatacga |
| | | atataattgagaatagcaaaaattgaacagcagccaaattggtgatgagt |
| | | gaggtaactctggtaagtcaccctgatagaatatattccatttattcatt |
| | | gttcatagaatatattctatttattcgttgttcttccaacctttgttgaa |
| | | gccccactctgtcagactcggcaccaagtgtctgagatgccttcgctcat |
| | | tacatcttcacggctcccccgagaagtagatgttactttcattatctgca |
| | | ttttacaaccaaggaaccgggatattaaattacttctctaggtcatgcag |
| | | ctagtgtgcggtgggctaaatgtacaatccccctaataataataataata |
| | | ataataataataacttacaacaacagcatattgaaataagaaaaaaaatt |
| | | tgaaaaatctctttgattcgtaaagatgaaaaaattgattctagttgttg |
| | | gtggtgagaggaaagggaacgcaggccctctgaaacactgttggtgggac |
| | | tacgaattgcagaaacctttttgaggacattttgcatatcttcagaattt |
| | | cacatgtcatgccatttgaccaagtcctttcacttttagggatttaccct |
| | | atggcaatatttgcacaagtacaaaaatgtatgtatgagaatcttggtta |
| | | ctgtctttcttgtaatagcagaaagtgggaaatcacttgaacatctatta |
| | | ggtggaaattatggtctgtccttatggtggaatagttgtaaccatttgta |
| | | agaatgagatagatctgcgtgtacccatatgtgcaaaagagccaccgtga |
| | | attgtttttttttttttttgagatggagtctcgctttgtcactcatgctg |
| | | gagtgcagtggctcgatcttggctcactgcaagctccgcctcctgggttc |
| | | acaccattctcctgcctcagcctcctgagtagctgggactacaggcgccc |
| | | gccaccatgcccagctaatttttttttgtatttttagtagagacggggtt |
| | | tcaccgtgttagccaggatggtctcgatctcctgacctcatgatccgctc |
| | | atctaggcctcccaaagtgccgggattacaggtgtgagccaccgtgcccg |
| | | gccatgaactgtgaattgaaaaaagcaagcaactgtaccatatgtaaaac |
| | | atgatctcattttccataaaatagcgtgtttaataatgtgggtctagaga |
| | | gataataatacatgcattgaaggatatatacaaatttatatatagcgcgt |
| | | gttctttacagtgggattagaggaacttgtactaatattttttttgcttt |
| | | gttttttttttgcaacagatatgattttattattagaaaaaccagattta |
| | | aaaaaagccattatatgagttttcctagttggcaaaaattcattatatga |
| | | gttatcctagttggcaaaaaaggcacttaagtgaggaaaggagatgcaaa |
| | | atcatccttgactgtaaaggggtgcaggtgggatatgagcttgtctgggt |
| | | atgagaatatcatgttctatgcattttttatttcctaaactgcttaaatt |
| | | ataaatcattaatggtaattattaaaagactttgttttccctacaattag |
| | | aaaaacttatttgaagacctcaaatcccaaaagtgaaagaggaaaaaaaa |
| | | aattattgccacggtttttcccttaacccatttaattaaagaattaatta |
| | | aattctatgttgcagtttttcaccaaaagtgcaacatagagttagttcag |
| | | tcaccacaaaaagattcagagattggattttgctgacttagtgaagctac |
| | | taagatacaaattcacaaagtaaatgtttgcacacattagctacagtggc |
| | | aaagtgtttgggaggttgcagtccacctcccggtgagggagatccatcct |
| | | tttggtcagcaaatgccctgaattgaccctctcccgagggtcagctgcag |
| | | gctgtgctgatctcggaacttctctgggggtccagttctgtttaggacag |
| | | accataaaatacatctctctggggtgggtagagctaccccagtacttacc |
| | | ctggaaatggactcagctgtccctggagggtgactcagttgaggcagaat |
| | | tgtcttaatttggtaactctcctaatttgaagcagaagtcctgggtttga |
| | | cccagacccatcttggtctggagtggagttgtggtgtgtctaatgctgcc |
| | | agatcatgttcactgttagaagcctggggaagggaaggaacttgctcagc |
| | | caactcagaagcaggcatagcgcaagagggaggattttaagaattagtat |
| | | tttactatccttgtgagctggtgttattgacaaacattatttcaatctgt |
| | | ttaacgattttaagaattagtattttactacccttgtgagctggtgttgt |
| | | ttctaaatattatttcaatctgtttaacaattccattttttaaaaaccct |
| | | gcactttgggcaggagttcgtatttctaatttaggatgaattttttctca |
| | | tctccccccattctgtgattctaattttttttaagttatttcagttctta |
| | | aaactctttcatagagctgggcatcagccgcaattttctctaaatctgtc |
| | | tcagatgagcacttgcagtctaattgtataaaagatcaggaaatttagta |
| | | ttgaagttgcacttgctttgatgtaatgatgaatgtttccttcatctgaa |
| | | gagcatttgaaatttatggaaaagtaaatctttttgaattatatgtaaat |
| | | gaaaacgttgggatgaaccaggttatgtttaactgggcagttttgaaatt |
| | | ctaatagctcctacaagcgggaatcttttaaagctaattttgtgatatga |
| | | attgtttattattctaaagaataatattcacttttttttcatggatctca |
| | | tgtaaatgttactggacaatatgacccatctctttaaaaagtgaaattgt |
| | | tagtgggaagctgtggctcacacttataattccagcactttgggaggctg |
| | | aggcgggaggatcacctgagcccaggcatttgagaccagcctgggcaaca |
| | | gcgagacctcatttctacaaataataaaaacattagctgggtgtggtgac |
| | | atgcatctttggtccaagctactctggaggctgaggtaggaggattgttt |
| | | gaacctgagaggtggagcctgcagtgagccctgatcgtgcccctgcactc |
| | | cagcctgggcaacagagtgagacctccatctctaaataaataggtaaagg |
| | | taaaatcactgctcagtcactagagagaggagagagaaaagtaaatatac |
| | | tcaaggagtgggaaaagctttgaagtggcatatcaaaatgagaaaaataa |
| | | gtgttttttagagagaaaagatttgtggagaattatatattagaactatt |
| | | taagacttcttaaaacttcacgattatatggttttgtaaggtttttgggg |
| | | attttcataatgagatgtagatggaacgtccccattgcccatcatagact |
| | | attcaggcaagttcatacccattcctagaatggagaaccgtctcctctgc |
| | | ggtgaagaagcgcccctgttccttaagaggcagcagcaggaggtggaaaa |
| | | caacatcccgcttgctaaagtcatgggccttagtccaggcgcggaggctc |
| | | acgcctgtaatcccagcactttgggaggccaaggcgggcagatcacctga |
| | | ggtcaggagttcgagaccagcctggccaacatggtgagacccagtctcta |
| | | ctaaaaatacaaaactgagccgggtgtggcagtgggcgcctgtaatccca |
| | | ggactttgggaggccgaggcgggcagatcacctgaggtcaggagttcgag |
| | | atcagcctggccaacatggcgaaaccccgcctctactaaaaatacaaaaa |
| | | ttagccaggtgtggtggctcatgcctgtaatcccagctgttcgggaggct |
| | | gaggcagaattgcttaaatccgggaggtggaggttgcagtgagccaaaat |
| | | agcgccattgccctgcagcctgggtgacagagtgagactccgtctcaata |
| | | aataaataaatacataaaaataaagccatgggcctcgatgggaggctgta |
| | | cattgacgtgcgacattcgacttctgccattatttcagatttcgcacctg |
| | | cccttctggcaacatggtgttgctgtgaggatcttgagaggagatgaatg |
| | | atgctttccaagtagtaagtattattccattagggaaaataggaataaaa |
| | | ttggaaagcatcaagtatttgaaaggaaacgaaaacagggcttactttcc |
| | | ctttcggccagcgttagaagggtgcagtttggaattgaaagtgaaaggag |
| | | catgcacgtgttcggggcaggttgcggggagagtgaagatcattttaaag |
| | | accctggtgctttgatcttagagcaccactcatttgtactcagttcagat |
| | | ctaagtgaattactgtgcaaacgacatgacgagagaggggcattttccaa |
| | | tagtagcttctggtctggcacatgagccagttagagattgacacacgcag |
| | | tcatgtagctgctataaattgaagcctggtgatggacgtactacaggcat |
| | | ggctgcagcgaatgcctatgacctttcacttctcctccctctgtcactct |
| | | gtgtattcgctctctccattttcctccctcacctattcaccagccatgaa |
| | | tcaggagtggactgggcaaggatcagcctgtagggaaatgatgtgggccg |
| | | agggtaaagagtgaggaataattaaggcagcccttgaattagttgtacat |
| | | ttttttcttttgtgtagtcttcttttttttttgtaagcaaacaaaaacca |
| | | cgttgctggccaaatgtagtggctcatgcctgtgatcccagcactttggg |
| | | aggtccaggtgggaggattgcttgagtccaggagtttgagaccagcctgg |
| | | gcaacatggtgagaccctgtctctataaacaaataaattaaaaaaattag |
| | | ctgggcatagtggtacccacctggagtcccagctaatggggagactgagg |
| | | tgggaggattgcttgagcccaggaaactgaggctgcagtgagctctgatt |
| | | gtgccattgtactccagcccgagtgacagagtgagaccctgtctctcaaa |
| | | aacaaaaaacaacactttattatggatgatttcaaacataaacaaaagtg |
| | | aagagagtagtataatcaactctcaggtacacgttacccagcttcagtta |
| | | cccactcataacctttcttgttaatttctacaaaaagcttgctgggtctt |
| | | gttgaatctgttgatcaatttgggaagaaatgccatcttaacaatactga |
| | | gtccttcaatccaagaacctggtatctccttacatttatttagatattat |
| | | ttaatgtatcaacaaagttttgtaattttaacttactaattttgatgcta |
| | | ttacaaatggaattatttcttaattttgtttttggattgttcattgctgg |
| | | tatacacaaaacagttgatttcctgatattgattttgtatcttatgaaca |
| | | tgttaaacttgcttattagttctagtagttttttgtagactcctgaagat |
| | | tttctatatatagagctagtgaatatgaagagttttacttgttttccaat |
| | | ctgtgtcttttaaatttctttttcctgccttattgttctggatagcacct |
| | | ccagtacaatgttgagtagaaatggtgagagtagacatccttgttttgtt |
| | | tctgaacaaagaaccaacttttggtttcatcagttttctctattactttc |
| | | cctaattcattgatttctggtctagtatttattattgccttccttctgct |
| | | ttttttgggatttagtttactctcctttttatagtttcttaaggtggaaa |
| | | ctcaggtttgtgttttttttttttgagacatgatctcgccccgtggccca |
| | | ggctggagtgcagtggtgcgatactggctcactgcagcttccaccttcca |
| | | ggctcaagagattctctcacctcagccccccaaggagctgaggctacagg |
| | | tgtgtaccattgggccagctaatttttatttttattttttgtagacacag |
| | | ggtctcactatattaccaaggctcttcttcaactcctgggcttgagcgat |
| | | cgtcccagcttggccttccaaagtgttgagattacaggtgtgagccactg |
| | | ttccctgctgatgctcagtttattaacttgagacttttcttttttttttc |
| | | ctgatacaggcttttaacattataaatttctccctaagtacttctcatag |
| | | ctgcatcctaaattttgatctgctgtgtttttcttttcatccagtttgaa |
| | | gtatttttaaacttcccttgtgatttcttctttgacctttgaatccttta |
| | | gatatgttttctaatttctaagtagttggggagtccacaaatttatttct |
| | | gttcttgatttttaaaaatttcattttggtagagaacatgctttatatga |
| | | ttttaattcttttaaatttattgatacttgttttatgacctagcccatga |
| | | ttcattctagacaatgcgatatgtacacttgaaaagaatatatattctat |
| | | ttgttgttgcttgacatggtctacagatgttagttacgtcaggttggttg |
| | | acaatgctgttcaaagtctgtatatccttgctgattttctgtttaactat |
| | | tatgtcaattattgagagtgggatattgaagtctccaaacattattgtga |
| | | attgcctatttctccctttggttcagtcagtgtttgctttattgtatttt |
| | | gtgctctgctattaagtgcttatgtatttatgtttttatatattcctcat |
| | | gagttgacccttttattgttacaaaatttctttttctcttataacttttt |
| | | tgtcttaaagtttattcagtctgtgggatttttttgtgtttttgtttgtt |
| | | tgttttgagacagggtatcactctgtcacccaggctggagtgcagtggca |
| | | caatcatagctcactataaccttgaattcctgtactcaagcaatccacct |
| | | gccacagcctcccatacttactttgatattattatagccactgcaacttt |
| | | cttatggttttgtttgcatagtacatttttctccatctgcatctcttgca |
| | | gacagcacatggttagaacttctaaaaaatttaatctgacagtcttcacc |
| | | ttttattggagtgtttaatccatttacatttaatgcaattattgatatgc |
| | | ttggattacatctaacattttgctatgttttctctgtctcacatcttttt |
| | | gttctgtttctccttactgttttcttttgtgttaaatagatattttctag |
| | | tgttctgttttcatctgtttggtgaggtttttaatattttttggttatat |
| | | ttttagtggttactctaaggattgcaatatgcatctcatcacagtctact |
| | | ttttaaagatctatatatagcttttaaaaagtagagatggggttttgcca |
| | | tgttgcccaggctggtttcaaactcctggattcaagtgatccttccatct |
| | | cagcctcccaaagtgctgggattacaggcatgagccactgtgcctagagc |
| | | tcacagtctactgtagattaatgctaattcattttcaataaaatatagaa |
| | | actttgttccattaaatgtctatgtctatctacctcctctgtggtattat |
| | | gtcatgtatatgatatctttgtatgtcataagcccagtagtaaaagtctg |
| | | tagttattgttatatgtacttgtcttttaaatcagttaagtgtaaaaaag |
| | | agactatttataaatctgctgtatatttgcctatgtaattagtattacta |
| | | gcattctttatttcttcatgtggattcaggatataatctggtgttttttt |
| | | ctttcatcttgaagggcttcctttagtatttcttgtaagtagggtctgcc |
| | | agtaacaaattttctgtctttgtttatctaggaatatctttatttctact |
| | | tgttagttttggggtatggttttgatggatatggaattcttgattggaag |
| | | tttttctttcagtgctttgaatatgtcattttactgttttctggtctctg |
| | | ttaatttctaacgtgaagtcagctgttaatcttatggtagtgctcctttt |
| | | catgaagaaccattttgtcttcatgcagtcaagattttctctttgttttt |
| | | ggcttccagcagtttggctatgatgtgtcatggtggctcactttattgag |
| | | ttggagttttttgagcttactttgagtttgttggatgtgaacattaattt |
| | | tttaaaatcaaatgtgagaagtttatgccattagtttttcaaaaaatttt |
| | | ttctgcttccttttgcttcgttttcttttgaaacttcctttatatatatg |
| | | ttggcatgcttgatggtatccctgtgtttctgaggttctttttatttttc |
| | | tttatgcttttttatttcttcttcagattggataattttaagttcactga |
| | | ttctttcttctcccatgtcaactctgttgttgagcccctctagtgaactt |
| | | ttcattacagttattatacttttgaactctagaatttctgtttggtttct |
| | | ttttataatttctgtctctttattagtattctctatttgatgagtcattg |
| | | ttacatactttcctttagttctttaaacatgaattccttcagttccattc |
| | | ttacagtagtttcttataactttgaaatctttgttaagtccaatatctgg |
| | | gcccccttagagtttccagttatgactcttgcctgagtataattcacaat |
| | | actttatgtttctttgcatgtcttatattttatttttttaagctctgtat |
| | | tttagttaatacgttgtagcaactctggattatgatcacctttggcaaaa |
| | | cgcaggtttgttgtttatttctttttgtttgtgacttgcctggattaatt |
| | | ctgtgaagtctgtctctgtagcagctgctgatgatcctgctcattttttt |
| | | tttcttatttttaaaatgtgtttaagcctggctttgcaggggttaaccca |
| | | ggtcagcatagcttagtggtcaggcaatgattattcagaggtgtgctgaa |
| | | acaccttgagctactaatgcttttcacagtttgcggtggatatgtgtgta |
| | | cgtttagggacatgttcagaattcaggcatatattatacgagcctgtctt |
| | | ggtttttcctttttgccaggccttttctggcctcctttgtgcgtggggta |
| | | ggcctcactttagccaaggatgagtggataaccagggccctcttcagtct |
| | | ttcctgcctgtgtgcagccttccagagcacgaagaatgtgtgggacttca |
| | | gggactttaccactgcccgtgatagctcccttattctctgtatcttccgg |
| | | ctaggtttttggctggtctcctgatctgttggttgccacaaccggcattg |
| | | caggctcagggtggctgagatgttgaccttctgtacatttcctgttgttt |
| | | acaggaaatcaccgtggtttatgtcagtgcctttgggcatgtgctctcct |
| | | ccaaatcaagtcaacccctccagcagtggagccaccaattttcacagctc |
| | | gtcccaccctggtagatgtagcagcatgggtgagctgaggggaaggaggg |
| | | gagcccctcaagccaaagcatcacagaacaccaccgctctaactcaagat |
| | | ttggtcatttttcttcttcattttattttttaattcttgagatgaggcct |
| | | tcctctgtcactcaagctggcgtgcagtggcacaatcatagttcactgta |
| | | acctcaagctcccaggctcaagagattttcccacctcagcctcctgagtg |
| | | gctgagactacaggtgtgcatcaccacgccaggctgacttttttgttttt |
| | | cgttgagacggggtcttgatatgttgcccaggctggtctctgactcccag |
| | | cctcaagcaatcctcctgcctcggcctcccaaagtgctacaattgcaggt |
| | | gggagccaccatgcccagctgcgtagtttttcttgaataaacatttttca |
| | | attttttgtatgtctttggttgatttccaaccctgtggtggttgtttttg |
| | | accgacttgtctagttttgttgttactttttgaggagaggttttgccaag |
| | | cttctcacttcattattccagaagttcttcccctctccccaggtcatctt |
| | | tttggttttttcttctaactttctttctttctttttttttcttttttttt |
| | | ttttttcgatggattctcgctgtgtcgcccaggctggagtgcagtggcat |
| | | gatctcggctcactgcagcctctgccttccaggttctagcacttctcctg |
| | | cctcagcctcccaggttgctgggattacaggcacccgccaccagctaatt |
| | | tttgtattttttagtagagatggagtttcatcatgttggccaggctggtc |
| | | tcgaactcctgacctcaggtgatctgcccacctcggcctcccagagtgct |
| | | gggattaaaggcgtgagccatcgtgcacggccggtttttcttccaacttt |
| | | ctaagtactataagcttgttgtaaaatttggcaatatagaaatgtataaa |
| | | atggaaatgaaaatctttactaatcccatcccttaaagatagccactgtt |
| | | aataagttagtgacttgctttccaagctttaatcatctttctttaatcat |
| | | catatttttgattatatcctcttaccaaagttcctttacccctcaccaca |
| | | aaggtttctaattcagagacttcctttagctcccttgattcaaggagata |
| | | aagttaattagcaatcaggagatgaggagccctttcctggcaaggacaga |
| | | attctaagacggagggatttttagctcctcggtggactcagttccgctgc |
| | | accttttgcagcatcttgcctgctgggtctcctctctgcagttttcttgt |
| | | tccctggccttttatcctcctgatcacacataccttcccaggttctccag |
| | | gaaagtagtggatcagtcacttccccctgcaacgtcattgtaggcatgat |
| | | tacatcttggagtgttctctcaacaagagtcagtgtagcatggggttccg |
| | | ggcctgagctctggtgtcacattgcttggagttcaaatttcaattcttct |
| | | tcttttttttttttttttcttggagacaagagtttctctgtttcccaggc |
| | | tggagtgcagtggggcgatcttggctcactgcaacctctgcctcccaggt |
| | | tcaagtgattctcctgcctcagcctcctgagtagctggtactacaggtgc |
| | | acgccaccatgcctggctaatttttgtatttttagtagagacgggggttt |
| | | taccgtgttggccagggtggttttgaacccctgacctcaagtgatcttgc |
| | | caccttggcctcccaaagtgttgggattacaggcatgagccactgcgccc |
| | | agcctcaactctctttttttttttttttagacagagtctcactctgtcac |
| | | tcaggctggagtgcagtggcatgatctcggctcactgcaacctccacctc |
| | | ctgggttcaagcgattcttctgcctcgacctcccaagtagctgggattac |
| | | aggtgcccaccaccacgcctggctaatttttgtatttttagtagagatgg |
| | | ggtttcactatgttggccaggctggtctcgaactcttgacctgaagcgat |
| | | ctgcccaccttggcctcccaaagtgctgggattactcaactctccttctt |
| | | agagtcctttgtgaccttgggttccctgggccttggttttctcatctctg |
| | | aagtggggctgggagcaactcttgcagcactgggttggaacgtgctggcc |
| | | acagtgctcacacatcgagaacacgccgtaaatgctccccgggcctccgc |
| | | ttcctggtgtcgtcatccttattcagaatgaacaagaaatcactccttca |
| | | gagtaatgtttcagacagaggagagagctttgtgggggccctctggcgtg |
| | | ctcacagaagctttggtattcgtcaaccactgtaggagccaagcatttcc |
| | | tacagaatgcagggagcagggctggcggggagatagtgcgtgaaaagaga |
| | | ggctggtgttgggaaagaaacagagacagaacggtggccggcggacaaac |
| | | gacaataagccaggatgatggtgtcactgcagccgggtgcggagtttctt |
| | | tctggggtgatgaaagtgttctaggttgatggtggtgatggttgcacagc |
| | | cctgtgtgaatatgctagacaccactgaattgtaccctggaaacagatga |
| | | attgtacggtatgtgaattatatctcaataaagctgttattaaaaaaaag |
| | | atggacagtcccagctactcaggaagctgaggcaggagtattgcttgagg |
| | | ccaggggttggaggctgcagtgagccgagattgcaccacggcactccagc |
| | | ctaggcaacagaaccagacgcttctctttaaacaaaaacctaaacaattg |
| | | tagatttttttaaaatgaggctttttgttttttttatgccaaaacccttc |
| | | atagacatgaaataacagaagagtaagattggcggggggggggggggcgg |
| | | tttcatcagtacaggttacctgaaagaactgagttgttggtctgacctct |
| | | gctaggactcgccatgtgtcatggcaggcctggcacctgaaggagagctt |
| | | tagcaatttgcactcacatcatcctggacctcagcctgggtgatggtcac |
| | | tgtcttgagacagcttcagaacaggcaacctgggtgtggccgccttgtca |
| | | accagagataataaaaacacaggtaaacctctgcacctccatcgtcatgc |
| | | cgaattataacaacagcacatatttgttgaagtcttactatgcactaggc |
| | | acagtgctgagtgcccatgatgcagaggggaatttgagagtgtcagtcat |
| | | ttctgccccttcggatcacgcactgctttccagtgttcccaggagaccag |
| | | ttttgaggttagaattctgcttcttacagtgaggactgattctgactcct |
| | | aggaggacgaaagccccttttaaatttactttggctgggtgtggtggctc |
| | | acggctgtaatcccagcactctgggaggccgaggtgggtggatcacttga |
| | | ggtcagcattttcagaccagcctggccaacatggtgaaacaccatctcta |
| | | ctaaaaatacaaaaattacctaagtgtggtggtgcacgcctatcgtccca |
| | | gctacttgggaggctgaggcaggagaatccctggagcccgggagcggagg |
| | | ttacagtgagctgagattgcatcactgcactccagcctggatgacagagt |
| | | gagactcgtctcaaaaacaaaacaaaacacaacaaaacattactttgaag |
| | | ccacagcattttgcacaggtaggaaaactgctgggcctggagatcttcta |
| | | taaatatgttgagagctgatgttgaagagattattgaagaatcctgatgt |
| | | cacagaattttctatagacaacctccttgcggggagggcgcagctttcgg |
| | | gttctgtatcgcacaacaccggctgggagctccctgcatgtgtgtttcca |
| | | gcactgcctgtctatgggagctaaggaagtggcttcaaaaccgcttgcca |
| | | gagacgcgcagtcacccagtcgttgcagcgcttgtacttgtgggagctgt |
| | | cttgcaggctcaggcatctttgataaggaattcctcatacttagaagtgg |
| | | cacctttgttctgctggggaattgttcccaaaaggtatcttttttttttt |
| | | ttgaaacggatcctcgctctgtcgcccacgctggagtgcagtggcaccat |
| | | ctcggctcactgcaagctctgccttctgggttcatgccgttctcctgcct |
| | | cagcctcccaagtagctgggactacaggcgcccggcgccacgcctggcta |
| | | atttttttgtatttttagtagagacggggtttcaccgtgttagccgggat |
| | | ggtctcgatctcctgacctcgtgatccccccaccccggcctcccaaagtg |
| | | ctgggattgtaggtgtgagccaccgcgtctggcctttttttttttccttt |
| | | ttgagacagagtcttgctctgctgcccaggctggagtacaatggcctgat |
| | | cttgactcactgcaacctctacctcctgggttcaagcaattctcctgtct |
| | | cagcctcccgagtagctgggattataggtgtgtgccactatgcccagcta |
| | | attttttgtatgtttagtagagacagggtttcaccatgttggccaggttg |
| | | gtcttgaactcctgacctcaagtgatctgctcgcctcagcctcccaaagt |
| | | actgggattacaggtgtaagccaccacgcctgaccccaaaagacatctaa |
| | | aaaggctttttcacttgtgaaatctgctgaatagagctggcttcctgagg |
| | | cctgggggcccttcccggtgtggaagaatggctctgctgttggctaccac |
| | | agtgctggggcctgtactcccacctcctctgcctctgtctgccttgtgtg |
| | | cctccccttccgcgggggccctgctctctgcctctttctgaccttccaga |
| | | gtttttgaggtgtgagtgtgttgttgaatttcatttacataataattaaa |
| | | gagtcagggtttcatgatctactgatgtaaaacccgcaaaaaccctaaaa |
| | | tctcgagtggtgttgtacagcatgtgggattcttttgtataacttgatga |
| | | ttttcaaaatatccatttcggctgggcacagtggctcacacctgtaatcc |
| | | cagcactttgggaggtcgaggcaggtggatcacgaggtcaggagatttag |
| | | accatcctggctaacacagtgaaacccgtctctactaacaatacaaaatt |
| | | agccgggcatggtggcgggcacctgtagtcccagctactcgggaggctga |
| | | agcaggagaattgcttgaacccgggaggcgtagcttgcagtgagccgaga |
| | | ttgtgccactgcactccagcctgggtgacagagcgagagtccgtctcaaa |
| | | aaaaaaaaaagaaaaaaatccatttaatcagagccatacttgttgattac |
| | | tacaggtgcttcccgtatctaatacctccagcaagaaggtttgcgaaatg |
| | | ggcagagagtaataggaagagtttcaggaagatggtttgagtgaaaatca |
| | | tttcagaaccgagccctgcgaagtcttggtttctgcaatccaccacagtt |
| | | tgggaaaatcgggaattttgtttagtccatttctatttttgtgcaccaca |
| | | aatatgtaaacaaagctgggatcttcgtcgggggcggagggaagggcgac |
| | | agaaccctgacatctaatactggtcatcggtatttccatgtgtttcctga |
| | | tcccattgtctctgctatttaaattaattaattccacaagtatgcactca |
| | | gtaaatattgtgagccaggcactattctcggctctgtggttgcatcagtg |
| | | aacaaaacagacgctaatcctgctttcgtgagccatctataatagaatgg |
| | | gcgagtcaagaaattgacacatgcattagaaaaatatgtagcgtgtcgct |
| | | ggtaagtgcttgagagctcatcagggaagggcaggagtttgtggagtggg |
| | | ggtgagggatgtggggtggggcggggcaagggtggcaagaggcctggggc |
| | | tgctggttgcaactgtaaccagagtggccagggaaatccttgctcctctg |
| | | ggtaatgccctatccagaagacccttgcaaatttctaaaatgctgtgttt |
| | | tattaccgagaaagagtcgtctgggagctctccgaggcaaataacttttg |
| | | aagttggcatgagcaggatattgtgcaatggggctcactggcttcttccc |
| | | aggcctccgcatctgatgtgaaccacagctttgcaatcgggtccccgtgc |
| | | catccaaaaatattttgtgaccttgccggttggcagttttttaaaaatgt |
| | | tattttcagtctccaaaaaagggaaacatcaaagtgcaggctgtccactc |
| | | ctgcccttcttgatagacacagacagctgggccacacaatcagccattgt |
| | | ggcgacaagtgcccaactgttataaatagacggcgggctcgcacgtgaag |
| | | cgctccagaggaagggcaagtgctctggggacactgccaactgctgtttc |
| | | cgtcgagagctcttctcaaagatcaatattattttatgtctcagtgactg |
| | | aatttgaaaaaacgactgtgaaagcacctggtaacttaactgtctccagg |
| | | attccattccaaacgcatcttctctcttcactgttaatcttagatgtttc |
| | | ccatgcttatttagtaatcattgcctgacggcccacttcactgcgattta |
| | | cagtctcttgcggctcacttgaaatggtagagttatggataaccgcgctg |
| | | gtgtatgtgcgtgtgatgcgtggccggaagcgcatgcccattgaataatc |
| | | acgttcgtcccacatcttagcccctcttccaagacatgtttactatcgtt |
| | | tacgtgtgacatgtcaaaacatcttaaaactatgccacgcatgtaatagg |
| | | ctctcagtgattaaaaaatcagtttgtctcttaaggagaaaagaataaca |
| | | gcatgtgactcttgtgagtccctgacaaagaatctgttgttccccgtgca |
| | | cttgaagagccgctcatgtcaagaagcaacttgtgattatgcaatgtgtg |
| | | acttaaggcattgtcagatgcgttcggctgaaacgggaccccgccccagc |
| | | agtttgccttactaagttaacatggtaggtcagattttagtattgtagaa |
| | | aaagccacattcaatattctttgagtagcatatttatttcctagggctac |
| | | tgtaccaaagtacctcaaaccaggtggcttcaaacaacagagttacacgc |
| | | tagttgatatctagtgattcaaagaacagaattccacactagttgttatc |
| | | taggaatgcggagtgagtgatagagggggttttggtggcgttatattcta |
| | | attttgaagtaagtttaaatttacagagatgctcccaaacagtataaaga |
| | | attcccaaatactcttcacccagattccctgaattaacattccgacatgt |
| | | ttgtctgtctgtctctccacatacacacatcccttttttccggaataatt |
| | | tgagaatagactgcagagagtgcctctagattctcctgtgtgggtttcct |
| | | aaaaacaaggacgctgtcccggatagcctgcaccgacctgcccaaatcag |
| | | gacgtgggcactgtcacaccatgccaccaaatgcagaggccccggagccc |
| | | gcccaggaccacaggcgccgtctgtgtcaatggctctcgagtctccttca |
| | | atctggggctgttccttcgtctctgtctttcacgaccttgacattttttt |
| | | aagagaacaggccagttactttgtagaatgtccctcggtttgggctggtc |
| | | tcatattccttcatgatgagaatcaggtggtaggttcctggcaggagagt |
| | | ccctgcagggttgtcacgacaggaacgcacacaggtctgatcagtcccat |
| | | ttctggagaagttaacttatcatttggttgaggtggtgtctaccaggttt |
| | | ctccactgtaaaattaatttgttggtattttgtaattatcagttaaaaag |
| | | tactgtttctggtggggagattttttggggactcggaaaatatcctgttt |
| | | ctcattaagctttttgcccatttgttttagcatcctgggatgattcttgc |
| | | ctgagggaattagtatgagcattgccaaatggtgatgtttctagttgcat |
| | | tgtttcttttgcattgattagttggcattccactgcaagctggagcttgc |
| | | ctgctgtctttctctccttcctatcaatacactcatagactcctatttta |
| | | cccattgggttataattcattaccattatcatttgcttcgatacccagat |
| | | tgtcccagatttggccaaaagcagcgcctccaagctggcccctgtgttct |
| | | tttgatgtgttcatgtcattctttgagcacatctttacttcttcttcttt |
| | | tttttttttttttagatggagtcttgctctgttgcccaggtgggagttca |
| | | gtggcgtgatctctgctcattgcaagctccgcctcccgggttcacgccat |
| | | tctcccgcctcagtctcccgagtagctgggactacaggcgcccgccacca |
| | | cgcccggctaatttttgtatttttagtagagacggggtttcactgtgtta |
| | | accaggatggtctcaatcccctgacctcgtgatctgcctgcctcagcctc |
| | | ccaaagtgttgggattacaggtgagagccaccatgcctggccaggttttt |
| | | tcgtttttgtttttgttttttttgagatggaatctcactctgttgccccg |
| | | gctggagtgcaatggcgtgatcatggctcactgaaacctctgcctcctgg |
| | | gtccatgcaattctcctgcctcagcctcccatgtagctgggattacaggc |
| | | gtgcgccaccacacccagctaatttttgtatatttagtagagatggggtt |
| | | tcaccatgttggccaggctgatctccaacttgtaacctcaggtgatctgc |
| | | ctgcctcgtcctcccaaagtgctgggatgacaggtgtgagccactgctcc |
| | | cggccacatgtttagttcttggttctaggctcacccggaactttccttgc |
| | | tccagccctggaatcagccatttctccaataagccctggttccttttagt |
| | | agaaagtggtttttagaaaccaggatctaagtattaggtgtgttcacgct |
| | | actgggctattacggctacaaggacttctcaagcagcgtagttaggaatt |
| | | ttttttttttttttttttttttgagacggagtttcactcttgttgccaag |
| | | actggcatgcagtggcaggaacttggctcaccgcaacctctgcctcccgg |
| | | ttcaagcagttctcctgcctcagcctccctagtagctgggattacaggca |
| | | tgcgccaccacgtccggctaattttgtatttttagtagagacggggtttc |
| | | accatgttggtcaggctgggtctcaaactcctgacctcaggtgacccatc |
| | | catcttggcttcccagagtgctgggattacaggcatgagccaccgcgcct |
| | | ggcatagctaggaaattaaacatatgcatgtacttccctacacgtacaca |
| | | tatgtttgtacatacacacacgggcacacatgcgtttgtagttctttgta |
| | | tccatctgtcccgctcgtatctatgtatccattcatctcatataaaaatc |
| | | atgataccatccaagtcaaatggtttgtttcagctttctcccctttcctt |
| | | ggctgtgcccctcttttctaacaataacagtaacagctcttatcatatta |
| | | actgtttttatttattgacccttgtatgtcaccagtcttctgacatgcag |
| | | gccatccagtccatgttcctggcccaggaacatggcagccaagggtcccc |
| | | acccccacttcggcccccaacagtggactctcgggatagttctttttttt |
| | | tttttgagacacagtctcactctgtcacccaggctggagtatagtggtgt |
| | | gatctcggctcactgcagccactaactcctgggttcaagcagtcctccca |
| | | ccctagcttcctgagtagcagggactacaggtgtacaccatcatgcccgt |
| | | ctacttttgtattttttgtagaaacagggtcggtcttaccttgttgccca |
| | | ggctggtggatagttctttatagcagtagcctaaacagaaaaacatatac |
| | | aacataagctaagaaaaaggcctcccttatcgatatttctgggttaatct |
| | | gaaagattttgaagtctggaagaatcagctggttgttggtaaagggacgg |
| | | tggacatgaaggatgcggaagggatgggttggaggcgggaggtgagtttc |
| | | atgtggtgtttttgactcagagggtcacttttattgttctgccttgtagt |
| | | cagaagaaaaatcttttcacgtggtttcggctcagaagtatgttagaaag |
| | | tagactacagagactgaaggtgacattccagagactttttaaggataatc |
| | | tggccaaaggtgtttactatttgattagccagcatccttgggctcggcta |
| | | gggtcttggtctggtctgagaacatgttgggggattgtgccagtgtcggt |
| | | ggtgggtgcctgtgcctggtcatctatgggcagctaggggtgggtgctgc |
| | | cccttccgactttctctccactgcagagacgaagccttcagcagcatttc |
| | | cctggaccctcttctctctggtcccaggtcaggttctgtacagcaatgga |
| | | aagcaggggagaggatgcccacgctgtgcgtggccgtttggagcagactc |
| | | agggaccagtgggacctcctcttctgggtgagcttggcaaccactctttt |
| | | aggaattccagtttgtaatgccttccgggtgagccctgtgagtcgcttgc |
| | | tcttagtagtgtcctttgctgccccagccttctgcttccctgtatttaaa |
| | | acctttctgagccaggattacgatttcctgataccatgactgacccttgg |
| | | tcgttatgagtcctgatggtcccaggagcttttccttcatttgcttgggt |
| | | tttgagttgtgagctggagaccaggggttttgcttgatggggacattcct |
| | | ggctcttctcctggggagtgaccatgggccctgcctaggtgagatcctct |
| | | ggattgtgaggagggacttgagcagccctactagagtgccgtctatggga |
| | | accctcagcatcgcctgtccaagccagcatctagtggacgctcacgctgt |
| | | gtgagacactgagcttatccagcaagggcagaaatggcagctcttgggaa |
| | | agaagggagtggggcatcgctgtgtctcctattatttgccattgtggcct |
| | | ccctctccaggtgcaccgaggggcctatctgtgtgattcttgcaggggta |
| | | ctgacgtccgtttcatctcacccgtgtccctctacaagtgcaggagttgt |
| | | agggctatgttattcttaggtagcttaatggatccattttcataataaac |
| | | tccattttattttgtttgccattgtactgccatttgcggaacaaagagaa |
| | | gttatataaaccacagaagggggagaatgtgacccaggaataaactgttg |
| | | tgtttcacagtaagtgtatggaactaatttagaatgatttttcattgtca |
| | | aacaaagaacaaatggaaatttgaacaatgcatggttacttttaacaaag |
| | | acttcattttaatgttattagtgttttctcattttaaaatggcagtgcaa |
| | | atataaccttaaaatagggtttatgactaattacatttcttttttgtatg |
| | | tctgacaatttccaattattgggtgtttgtgttccccagcccaccctccc |
| | | ccaaaaatgacagggaacagaaaccagtgaaaagatctctctttggcgcc |
| | | cttcccagactaggacctgttttgttcagggtgagcacagaggagaactg |
| | | ggtcctcttgtcaggctgaaggggacgtcacagaagccccaagtgggtca |
| | | cggcagagcgtcagccagaacctccagggctcactcccaggcctcagtca |
| | | ccagatgcttcgtggctacctgggtaagggagggttcagggcaagttagg |
| | | gactgcaatgagaaaacaacacactctggccggatgcggtggctcacgcc |
| | | tgtaatcccagcactttgggaggccgaggcgggcggatcacctgaggtca |
| | | ggagttcgagatcagcctggccaacgtggatttcaaccaaggacctgctt |
| | | ggtcatagatttcaaccgaaggacctgattggtcatggatttcaaccaaa |
| | | ggacctgattggtcatggatttcaaccaaaggacctgattggtcatgcag |
| | | acagaactttgaaggctgtcatgggttgtgtccatgttgtttggttttcc |
| | | atcttgaacgaataccagtgtgcgtctaacagctcacacagaagcccatt |
| | | ggttacaaaggaattgcaattgtcaatccaattgccccccagggagaact |
| | | gaagatttttttaaataatgtcttcgtaacagctgtgtaagtcccccagg |
| | | cccttaactagagaaaccacatcacagaattttatttactttataaattc |
| | | taatattttagaattaagataatttgtagtttagatgatttttgctaagc |
| | | tagctctctctttctttttatctcttcctttctctcttttctttctttct |
| | | ttgatggagtctcaaaaaaaggagaagggtgtccaaacatcctggatgaa |
| | | aatggctccatgctgtcatagaattttatttactttataaatcctaatat |
| | | tttagaacttaaaaaagatactttgtagtttagatggttatagttaagct |
| | | atttcattttttttaaaaaacgaagaaattagcattcaatgtctgcctag |
| | | aaacaatcttatatacagtccttatgtacaaatcttttttgtggaatatg |
| | | ttttcgtagttttttgttttttgttttagattgggtcttgctctgttgac |
| | | caggctggagtccagtggcgtacttatagcttactgcagcctcgacctcc |
| | | agagctcagtcagtcctcctacctcagcctcccaagtagctgggactgca |
| | | ggtgcatgccagcacacttggataatttaaaaaaaaaactttttttttgt |
| | | tgttttgttgagacggggtctcacttatgttgcccaggctggtctcgaac |
| | | ttctgggctctagcgaagctctttcattttagaaaaggatcatgtcacct |
| | | cgatatgtgaggtctgtggaaaatcttgctttaagatgatctgcgtgaac |
| | | tagtagcttagattattgagtcttattttcttcagaactctgataggcac |
| | | aattactagatttaagttaaagggctgtaaaacagcctcagtactttttc |
| | | atgaagtagacaagtttcacagtgctttggaaatgactactaagctactc |
| | | ccagcctgctcattgagaactcacgatttcctctggatgagtgggaatgt |
| | | tgttggttctgcatttgtcatcagtgcttaggacacacacacacaagcaa |
| | | cgtgtgctggcccatgcagtttttccttatttacaaagctcatgtgagaa |
| | | ttgtgactctgcccattcgtggatctgcacaacatatggtgacgaatata |
| | | agttcgggggcgtggggggaatgcgcatgtgctatggttcagtgctgggc |
| | | actgggtgttgtggttcagtgctgggcactgtgtgccgtggttcagtgct |
| | | gggcactgtgctgattgcttctgcgactccggacagatacttaaggctca |
| | | gctcaggttcgtcattaacacgaatcacaaattcagacctggtgccgtgg |
| | | ctcacacctgtgatcccagcactttgggaggccgaggcaggagtatcact |
| | | tgagcccaggatttggaggccagcccgggaaacataggaagacccccaac |
| | | tctacaaaaaatatgaaaaaaagtttagccaggtgtggtggtatgcaact |
| | | gtagtcccagctactggggaggctgaaatgggaggatcgcttgagcctag |
| | | gagttcaagaccagcctgggcaacatgatgaaacctcatctctacaaaaa |
| | | aaaaaaaaaaaaaaaagaaagaaaaaaagcatggtggtgcatgtctgtag |
| | | tcccagctactcgggcggctgaggtgaaaggatcacttgagccaaggagg |
| | | tcgaggctgcagtgagccaagatcgcgccactgtactgcagcctgggcaa |
| | | cagagtgagaccctatctcaaaaaattcacaagggttttcaaagctagtt |
| | | actgcctgagaccatcttcccctcttggatggatgcagcaaattgggagg |
| | | caaaattatttggctctttttttttttttttttttttttttaaggcagag |
| | | tcttgctctgttgcccaggctggagtgcagtggtccgatctcagctcact |
| | | gcaacctcagcctcctggattcaagcaattctcctgcctcagcctcctga |
| | | gtagctgggattacaggtacacgccaccacacctggctaatttttgtatt |
| | | tttagtagagacagggtttcaccatgttagtcaggctggtcgcgaactcc |
| | | tgaccttgtgatctgcccgtcttggcctctgaaagtgctgggattacagg |
| | | cgtgaaccaccgtgcccagccgcgtttggctccttcctaactaaacagag |
| | | attaaactgtttattgttacctgggctttcattttttcatagcagtcact |
| | | ttattttcatctctttgtcctcagcttcccctcaaatatttgtcaagtgt |
| | | ctaccaaggctgggaatgatagtaaaaggagtttaaaaaacaaaagaggc |
| | | tctaaaaatggtactagactcttccattcaggactttgtccaaagcctca |
| | | ctttataatgcttttgtgatttatcctcattcccataaactcagatgata |
| | | cgaatttatacgagtttgcattaataaatatttcttgcatgcttgtctta |
| | | tcaaccataccgagccagcacagcctcatctcgtaaagagttggcagtca |
| | | cgtgcgggagccgggagcccgttgctgtgaggccgggaagtggattgttg |
| | | gtctggggaaagatttattttctgggggcaaaagaggcggggtggagagt |
| | | ccctccgcctgcctggagtgctgatgtggcctcagagctaggtcctgaaa |
| | | aattgctgggatttagatgcaggctctacatgtagagagattgtttaaat |
| | | gtataatggttcagaagtgacactctcttgctatgtttctctgaaaatag |
| | | atcagccttctctgttgggatctatgaagctccagagataaaactgatat |
| | | acattaaagtggctcgtggttaaaggaagtatgtttgttaatattaaacc |
| | | gaggtttctctggtaactagattttaaatgatttgattccataggtttat |
| | | aatggagaaatccagatctccaaaatgtaagttttactatatgggaagcc |
| | | tttttttgatttcctttgctgtgtttggtaaatagcctggaagaaaatgg |
| | | ctccgtcctatcacagaattttattatttacttcataaacctagtgtttt |
| | | agaattttttttttgtttttttgtttttttgttttttttgagatggagtc |
| | | tcactctgttgctcaggatggagtgcagtggtgcgatctcagcccactgc |
| | | aacctccgcctcctgggttcaagcgattctcctgcttcagcctccggagt |
| | | agctgggattacaggcacacaccaccacacctggctaatttttgtatttt |
| | | tagtagagatggggttgcaccatgttggctagactggtgtcgaactcctg |
| | | atctcaggtgatccgcccacctgggtctcccaaagtgctgggattacagg |
| | | catgagccaccgtgcctggcctagaattttaaaaagattctttgtagttt |
| | | agatggttctagttaagctatttcatttttttaaaaaggaagaaactagc |
| | | attcaatgtctgcctagaaaacaaaaataaaaaaacaacaaaaaaggaag |
| | | aaactgaggtctgtggcacttaagtgccccactcaaggttgcagagttcc |
| | | tagtgatgttattttttaatgaacttttcttctccttctcttttttgtta |
| | | aattttggcagactgttttaccaaagcctaactgtacttttcttaattaa |
| | | ctttggaatgaattctggatatactcattccagcaatgcacagaaaattc |
| | | taggcaaagaataaaagactccacattgtttattattattatttttgaga |
| | | tgtagtcttgctctgttgctcaggttggaatgcagtggcggaatcttggt |
| | | tcactgcaacctctgcctctcatgttcaagtgagtctcctgcctcggcct |
| | | cccaagtagatgagattacgggcagctgagattacaggcacgtgccacca |
| | | tgcctggctaatttttgtatttttagtagagatggggtttcaccatgttg |
| | | gccaggctggtctcgaactcctgacctcaagtgatcctcctgcctcggct |
| | | tcccaaagtgctgggattataggtgtgagccaccgcgccgggccagactc |
| | | cacattgtgaaaaaacgagggaaccgcttctgattttgctccagtggtgt |
| | | attcctctgtcttgttataatgctttatgtttacatataacacattatac |
| | | gatttttaaagcaaccccacagggaagagttttaattttttgtttgtttg |
| | | tttttgagacagagtcttgctctgtcgcccaggctggagtgcagtggcgc |
| | | catctgggctcactgcaagctccgcctcccgggttcacgccattctcctg |
| | | cctcagcctcccaagtagctgggacttcagtcgcccgccaccacgcccgg |
| | | ctaattttttgtatttttagtagagatgaggtttcagcatgttagtcagg |
| | | atggtctcgatctcctgacctcgtgatctgcccgcctcggcctcccaaag |
| | | tgctgggattacaggcgtgagccactgcgcccggccgagttttaatattt |
| | | ttagtcctgttttacaaaggggagaaacgaagcacagagaagtttaggaa |
| | | cttgcccaaatcatacagttgataagttagcagaccaaaaccggaatcgt |
| | | ccaaacggagtggcagccagaaagcattttataggtaaagtcacatccta |
| | | cactgtaaataaaaacctcttggaacacagagttcgccaggtggctgttg |
| | | gattttcatgtcagcgctggtctagctccgttttgcaggattttttgttg |
| | | ttgttatcacagtatattctaaaaataactatatgttcatttccactctt |
| | | tcagataattaatatttataaatcctagtcactaggaaatatagttcaac |
| | | caacaattgaatggaatgcaggtcctctgggaacttatctaaaattaaat |
| | | cctattttaacagaattagctagattaaggtcatggtgtaaaaactgatt |
| | | tttcagaaggaaataaaaatgcaaaatatcctaagaattcattttgtaaa |
| | | taattgaatcaaataacaatggggcggggaagaagccatatatttttcct |
| | | ctctttttagaaatgaattgaaggccagacatgctggttcacatgtaatc |
| | | ccagcactttgagaggccaagcgggagaattgcttaagcccaagagtttg |
| | | agaccagcctgtgcaacataggaaaaccctgtctctacaaaagaatttaa |
| | | aaacagctgagtcagctgggcgccgtggctcacgcttgtaatcccagcac |
| | | tttcggaggctgaggtggatggatcataaggtcaggagtttgagaccagc |
| | | ctggccaacatggtgaaacccccgtctctgctaaaaaaacaaaaattagc |
| | | caggagtggtggcggacgcctgtagtcccagctactcaggaggatgaggc |
| | | aggagatctgcttgaacccgggaggcagaggttgcagtgagtcgaaatca |
| | | caccagtacacttcagcctgggcgacagagcaagactccgtctcaaaaaa |
| | | aaaaaaaaaaaaaaaacttagctgggggcagtggcacatccctgaggtcc |
| | | cagctacttgggaggccgaggtgggaagatggcttgggcccaggtgatcg |
| | | aggctttagtgagctgtgattactccaccgtactccagcatgggcaacac |
| | | ggtgaaaccttgtctcaaaaaaaaaaaaaacaaaaaaaatcagataaata |
| | | aaaacaaaaaattgtttagactcatgaatcatcaactttttctttttctt |
| | | tgcccattttcttttttaagtattagcttggcattaaagcagtagtatat |
| | | gaagatggaatttgctagtactttatttgtgattccagagttaaatgcta |
| | | ctgcaatcaagtcttccttgaatttagcaatagatttaaaagtttagaaa |
| | | gagcctcttttttttttttttttttttttggttttaatacggggccttgc |
| | | tttgtcgcccttgctcgatcgcccagtgatctcagctcactgcaacttct |
| | | gcctcctgggtttaagtgatcctcctgcttcaggctcccaagtagctggg |
| | | atgacaggcgtaaaccactacgcccagctacattttgtgtttttagtaga |
| | | gacggaggtgtcaccatattggccaggctggtctcaaactcctgacctca |
| | | ggtgatccacctgccttggcctcccacagtgctgggattacaggcgtgag |
| | | ccaccatgcccagcccaagtctagctctttagaaggaaaaaataagggta |
| | | gaaattcagagaaggatgagtgtggagtggcttccttgctctatttctag |
| | | aatatgccatttcttgggaagtcttatgaccctgctggtctttaatgtcc |
| | | cctatatgattttcctccctgggctttgattctctgctttatctcctgtg |
| | | gtcttttccatccagaaattttgaaggggtatttaccttcaacttaaaac |
| | | tgactactacaggtaagtagtggccctgctgtttccaccctcccatgtct |
| | | actggaagcagaattgtttcacctgtctcgttggaaaatcaaatgaggta |
| | | atggaaatgaaaagtactacacaagtgtgattattaaagacagttcagtc |
| | | aataggtgaaaagactcgtgtagatactttatttaacctggaatcaatgt |
| | | cttatactcagtagacactccttagatgtgagtttaaagatgtcattact |
| | | cctataataccagcactgtgggaggccaaggcgggtggatcacctgaggt |
| | | caggagttcgagaccagtctggccaacatggtgaaatctcgtctctacta |
| | | aaaatacaaaaattaaccaggtgtggtggcggctgcctgtaatcctagct |
| | | actcgggaggccgaagtgggaggatcgcatgaacccaggaggtggagatt |
| | | gcagtgagccgagattgcgccactgcactctagcttgggtgacagagcga |
| | | gactctatctcaaaaaaaaaaaaaaaattaagaagatgtcattttaatga |
| | | gcagttctttgttaagtaaagaagaagaggaaaccaggaaatatgatagt |
| | | gcttggccacctcaccaagtgtttgtcttaaaagaagctttcttgattaa |
| | | gtaaaaatttaaaaggaaggaaagaaaaacccaacctcaccgcaacaaac |
| | | taccctagtctgaggtaattgtgtagtgtaatgtccctacacctgaattt |
| | | ttcatgcacctgcagatttaggggctctgcctatctttgagtataaatgt |
| | | ccactgaattattagcaaaatgatttgttctcagaaaccaaaattttagc |
| | | ctagatgaatgccgccattggagttatttatatgaaaaggcgtgttaagc |
| | | tcaatggtcttctcttctcttctcttgttttcttttcttttggatttgag |
| | | acagggttttgctctgccacccagcctggagtgcagtggtgcaatcatgg |
| | | gtcaccgcagcccctacctcctgggctcaggtgatcctcccacttcagcc |
| | | tcccaagtagctgggactacaggtgtgcaccatcatgcccagctaatttt |
| | | tgcatttttcgtagagatgggatctcactatgttacccaggctggtcttg |
| | | aacacccgagctcaaacgatccgcctgcctcgacctcccaaagggctggg |
| | | attacaggcgtgagtccctgcacctgacctcaattgttcctttcagcaaa |
| | | agtaaaattttaactttcaagggtgcaatcctgctttttttttttttttt |
| | | ttttttgagacagagtttcactcttgttgcccaggctggaatgaaatggt |
| | | gcgatctcggctcactgcaacctctgcctcccaggttcaagtgattctcc |
| | | tatctcagcgtcccgagtagttgagattacagatacatgccaccgtgcct |
| | | ggctaatttttgtatttttagtagagatggagtttcatcttattggtcag |
| | | gctggcctcgaactcctgacctcagaacatctgcctgcctcagcctccca |
| | | aagtgctgggattacaggcgtgagccaccgcacctggcctgcaatcctgc |
| | | atttaaagcatttttaaagtgtcgttggaacacttaaaaagacaagaatt |
| | | aaaacagtagcagaacacctaaaatggcaaggaaacggcatgagggcgtg |
| | | taacacagaaaggggtgggtgtttgagaaacagcaccgcaagtacgtggg |
| | | gaagcggcaaagatggaggacatgggagatgggggtggaaaggggcttgg |
| | | ggcctggtagtgaaagactttgcatgccacgttagagtctctgctctgtt |
| | | ctgttagcaggagtttgtagtagtacctattcacagactggtgcgggtta |
| | | actgtgagggtaatgcgtgtaaggtgcttagaacagtgtttgacacataa |
| | | tccgtattctctaaatcttagttgtaggagatgttgttgctattattttt |
| | | ataaaagggcaagcattacgagtttcagagcaaggcttatggggtgataa |
| | | tataatggaggatgaattggaagaggagaaactggaagcctggagaatgg |
| | | tttggaagctctttgctggggcccgagagacattcagggttcaggcagtg |
| | | ggagtggaaggaaagtagtggattccacagcgagacttggcagggcttga |
| | | tggtgagggaaaggaaggagaagaaagtgacctggaggcttctggtttgg |
| | | atggttggggagtgatgaccccgagagaagagaggagagaaacagggttg |
| | | tgaggatgagacccaactactctgttcctcttttctcaactgaaaccatg |
| | | ctgacttcatactgcaaagagtgtacagcgacacggtatctcagtccgtt |
| | | ttactgctgcaacaaaatacctgcaactgggtgatttccaaacaatagaa |
| | | atttatttcttacagttctggaggcagttctggaggccgagaagtccagg |
| | | atcgaggccttggcattgggtgtctggtgccatgtcctcacatgacagaa |
| | | gagcaaaggctacggactcgttccctcaggcctttcatgcggttgctcac |
| | | ccctaagccctctcgtattagggtcttcatcccatttgagaaggtggagc |
| | | actcatgacttcttccttacctcctaaagaccccactgcctttttttttt |
| | | tttttgagacagaatcgtactctgtcgcccaagctggagtgcaggggtgc |
| | | aatcacggctcactgcagcctggacctcctgggctcaggcgatcccccca |
| | | cctcagcctcccttgtagctgagaccacaggcgtgtgccaccacgtctgg |
| | | ctcattaaaaacaattttgtttttgtagagacggagtcttcgctgtgttg |
| | | cctaggctggtcttggactcctggactcaagtgattcttccacctcagcc |
| | | tcccaaactgttgggattacaggtgttgagccactgtgccagtaagaccc |
| | | cacttaataccatcccttcgtgatgaagtttaaacacgtcaattttgggg |
| | | gacatttagaccatagcacataggtttcaaaaatgagtcaaagccttaaa |
| | | tggattgtctcttttctagtaccagctgataggcctcacgtctgttcact |
| | | gccttctgatgctcctccttaaccctgtgtaggactctgtagtacaggtc |
| | | tccgctcaaagacagttgtaaacgtggcacagttagtcaagagcattaca |
| | | ataaaaaatgctcagcatcactcatcacagagaaatgcaaatcaaaacca |
| | | gaaggaaatatcaccttatgccagtcagaatggctattagaattatatga |
| | | gaataatattaagaagtggggtcttactgggcacaatggctattattaat |
| | | cccactactagatatctacccagaggaaaagaaatcgatctatcaaaggg |
| | | atacctgcacttgtatgttcatcacagcaatgttggcaatagcaaaggta |
| | | tggaatcaatgtggccatcatcgaatggatgggtaaagaaactgtggtat |
| | | atgtatatgcagtgggatactgtccagccatcaaggggaacaaaaccctg |
| | | ttatttacaataacatggatggaactgaaggtcattattattatgattat |
| | | tttttatttttattttttagagaagagtcttgctctgttgcccaggttag |
| | | agtgcagtggcacaatcatagctcactgcagccttgacctcctgggctca |
| | | aatgatcctcccacctcagccacacgagtagttgggaccacaggcatgta |
| | | ccaccatgcctgtctaattttgtttattttttgtagacatggggtctcac |
| | | tatgttgcccaggctggtcttgaactcctggggctcaagcgattgtccca |
| | | ccttggcctctcgaagttctggagttacaggcatgagccaccatgcccag |
| | | cttggaagtcattaagtgaaggaagccagacacaaaagacatattgcatg |
| | | ttcttatatgtgggagctaaaaggtttgatcacatggaggtagagagtgg |
| | | caagaagggtaacagaggctgggaagggtgagtggcggggaggagaaggg |
| | | tcaagagaagtcggttaaagggtaccatcatgcagtaacgtagaaggaat |
| | | aagttcaaggcttgagagcagagtaatattattctaatattatatatatt |
| | | ctcacctaacaaaaaatgtattgtggctgggcatggtggctcccgcctat |
| | | aatcccagcactttgggaggccaagacaggcagatcacctgaggtcagga |
| | | gttcgagaccagcctggccaacatggcgaaaccctttctctactaaaaat |
| | | acaaaaattagccgggtggttggtgggtgcctgtaatcccagctactctg |
| | | gaggctgaggcaggagaattgcttgaacctgggaggcggaggttgcagtg |
| | | agccgagattgcaccactgcactccagcctgggtgacagagcgagattgt |
| | | ctcaaaaaaaaaaaaaaaaaaaacaaacaaaccccaaaaccaaaatgtat |
| | | tgtatttgagtgatgggcaccctgaatgccctgacttcatcactgcgcat |
| | | tatatacatgccataaaatttctcacataccctccaagtttgtataaata |
| | | aaaagggggtgttgaatacagactttcaaagaaaaagcattctgctcaaa |
| | | actcataaggggacgtggtgtgtgtgtgagttctgaaagaagtttgaagg |
| | | accagggttgaccacatgatcacgggaaacagcagctcctgggttgcttc |
| | | aaggtgctgtggctgttggtggatgaaatcagcctactaggagataggaa |
| | | gatacaaggagcttctccagtgtactggaagatcacagctctggcatcat |
| | | gggatctaaggagctggcttgtctttagtgaagggaggggggctggggcc |
| | | ccttggaatctttttttttttttttttttttttttttgagatggagtttc |
| | | actcatttgcccaggctggagtgcaatggcacgatttgggctcaccacaa |
| | | cctccgccttccgggttcaagtgattctccaccctcagcctcccaagtag |
| | | ctgggattacaatcatgtgccaccacgcctgtctaattttgtatttttag |
| | | tagagacggggtttttccatgttggtcaggctggtctcaaaactcccgac |
| | | ctcaggtgatccgcctgcctcggcatcccaaagtgctgggattccaggtg |
| | | tgagccaccgtgcccggcccccttggaatctttaaggccaagacctggga |
| | | ggctggaggtcgagcatgaaacaaagcgaaatttccctgagaaggaagaa |
| | | agaagtgaggattccctgaaaccaagaagcagctctgtgttcccatcttt |
| | | cctcgtgtgtgtcattggcttcccatgcatataggctttgcgaggcaatc |
| | | taagcatgtgtgcacgtgtgttttgtgtttttgtgtataattgagtatgg |
| | | ctgacttttgagtgggcacccataatcactctgtatggcaattacactta |
| | | tattaactttaatccatttcagagtaatgactcataatatttaataccca |
| | | catatcattgtgtattatatattaagtaattattaagatataatagctgt |
| | | cctgtggttctttctgcagttctgcaatgcataagtaaatatatatctac |
| | | ttagcacctggcagaattctgacactggttccctggagtgagggctgttg |
| | | tggtagcaaaggccaagtggaagctgccagaactgcctctgtctaccaaa |
| | | atagtcaacccaaagcaattatcacattcctgggaggatggcagagatta |
| | | gtgccaccatcagggacttgaaagatgcaagggtggtgatcgtgtcacat |
| | | ctccgtttaatccatatttggcttgtctagaagacggatggatcttagag |
| | | aatgacagcaggttgtcataaacttaaactaggcggtgactgcagttaca |
| | | gctgctgtgccagatgtgtttttgttgctggaacaaatcaacacatttcc |
| | | tggtacctggtatgcggtgagtgtcttctccttctctacctggtagtaaa |
| | | gatgaccagaagggtcttgcttccagccggcaaggtccacagcccgcctt |
| | | catagttacacttcaggggtctgtcagctaccagccttatgttatcactt |
| | | cagtctccttcttcattccatggggtgccaacggataaccactcagatag |
| | | aatgtaaaaggactttgggccaggtgcagtggctcatgcctgtaatccca |
| | | gcgctttgggaggctgaggtaggcagatcacttgaggtcaggagtttgac |
| | | accagcctggccaatatggtgaaaccccatctaataaacccctactaata |
| | | atacaaaaattagctgggcctggtggcaggtgcctgtaatcccagctact |
| | | caggaggctgaggcaggagaaactcttgaacctgggaggcggaggttgca |
| | | gtgagccgaaatcgtaccagtgcactccagcctgggtgacagagtgagac |
| | | cttgtctcaaaaaaagttcataaaaggactttggctcctcttctggggag |
| | | agggttagcatggttttggttgtaagacggatagtagcattatatggcat |
| | | ggaagcctagttgctatttacttgatttggaagttaagtacggttgaaaa |
| | | gaggagtagatatggacaccaagttgacaaggggtacgtgtagtgagttc |
| | | gttttgcgtcaccttggctaagttggagctctgtatgtaacttgagccac |
| | | aagagaccatttgtgtaagaccgagaaggcagaggggacacagcagccac |
| | | atgcactgtggctcatgcacgtggtctctgatctgcaggctcaccttgtt |
| | | ggcgcacggcagccatcaggcctgtacctcttctgaaacccaccaaatct |
| | | ttcgttgttcccctcatctgcgtatgtggcagacagtttttctatgtttt |
| | | caggcaattgttgagcaaataataatagaatttttgaacaggccagcatc |
| | | ggtgctaatatgtgccaggcacagatctgagccctttagagtatttactc |
| | | atttaatcctcaaaaccctgtaaagtcactgatgttgccataatctgtca |
| | | gactcagaaactgaggcacaaagaggttaagtaattgcttaagccttcac |
| | | aggaattgggatctgaacccaggcattttggtgtcgagtccatgaactga |
| | | cgaagaacatagataactgttgtcagcttgtcaaggctcatgcctgtaat |
| | | cccagcactttgggaggccgaggcgggtggatcacaaggtcaggagattg |
| | | agaccatcctggccaccatggtgaaaccctatctctactaaatcataaaa |
| | | aattagcgtctgtagtcccagctactcaggaggctgaggcaggggagtcg |
| | | cttgaacctcggtggtggaagttgcagtgagccaagattacaccactgca |
| | | ctctggcctggcgacagagcaagactccgtctcaaaaaaaaaaaaaaaaa |
| | | aaattacttcaaggagagactgctgagggttcctctgtctcctgtgtgga |
| | | tggaatttggcctccatttggaatcgggaatcttgcatatttagttctgc |
| | | agtattacgtgaatctacggaagcaactcatttggaattagattccaagc |
| | | cgttataaataataaattttttgtctctgtcatttttgtagtaacagcaa |
| | | gagaaaagaatttaggaccaaaacttatgagagttttaaatattttttgc |
| | | taagtgcttatatgtgatttatgggaaaatcataatttttaccctggaag |
| | | gcagccttactcataaagaatggccatcttgtagcacctccaggacatca |
| | | gtctgaagtctaagttaggtgtgtttgcacagcaaccaaatggtgagagc |
| | | atttagcagctgggggacggcggcattgttgtgattgggttgccacgtgg |
| | | tggggcgggtgagggacggcagcagagccttggcagagcggagctgtgtg |
| | | gccgacggcttcctctttcttcctccagcgtgcgattataacttgttgct |
| | | catgcaccggcatgcagctctgtttgctcagtctgtttttctgtaaagtc |
| | | tctttttaaaaaagtatatgatacaggctgggcacggtggctcatgcctg |
| | | taatccgagcactttgggaagccaaggcaggtggatcacttgaggtcagg |
| | | agttcaagaccagcctggccaacatggtgaaacctcacctgtactaaaaa |
| | | tacaaaaaattagccgggcatggtggtgggcacctgtaatcccagctaca |
| | | tgggaggctgaggcacgagaatctcttgaaccagggaggcagaggttgca |
| | | gtgagccgagatctcaccattgcactccagcctgggcaacaagagcaaaa |
| | | ctccatctcaaaaaaaaaaaaaaattgtgtgatatagatggaagaccacg |
| | | taaaataaatgtacaatctgattgttataaggtgaccgcctttgcatatc |
| | | taccaggcaggttgcaggagagcacagccagccacccagcagccgtcctg |
| | | ccctccacctgccgcagggggccagatcttgccattcctggtggtcccgt |
| | | ctctgctcttcttggttttctttctttcgttctttctttttttttttgag |
| | | atggagtcttgctctgttgcccaggctggagtgcagtggtgccatctctg |
| | | ctcaccacaacctccacctgctgggttcaagcaattctattgcctcagcc |
| | | tcccgagtagctgggattacaggcacgggccaccatgcctggctaatttt |
| | | tgtatttttagtagagatggggtttcaccatgttggccaggctggtctcg |
| | | aactcctgaccctgtgatctgcccggctcggcctcccaaagtgctgggga |
| | | ggtgtgagccactgcgcccggactttttggttttcttatccaactgtgag |
| | | tccttcaacacttggcgtcgggtgtttggcttgtctgttttttggcttgc |
| | | ttttgcttttttctccaacttttaatttaattttaattaattaattttat |
| | | ttatttattgagacagggtctcagtctgtcactcaggctggagtgtagtg |
| | | gcacgatgatggctcacctctgcctcgacctcccaggctcagatgatcct |
| | | ctcacctctgccccctgaggaactggaactacaggtgtgcaccaccatgc |
| | | ctgattaatttttgtattagccagtggtttcgccatgttacccaggctgg |
| | | tctcaaactcctgggctcaaacaatccacccgcctcggcctcccaaagtg |
| | | ctgcgattccaggtgtgagctactgtgcctggccttcgcaacttttcatt |
| | | ttgataaatttcaaacctacagaaaagttgaaataatagtataatgaaca |
| | | ctaatatcccacttcacctgattgacccgtggttaacattttgccaaatt |
| | | tctttggctgtctcagtgtaggtgtgtatatatgtatgtgtgaatataat |
| | | tatacacacatttgtgcaagaaccctgaatgggatataatacagtacaca |
| | | catatgtatgtgcatatgtgtatgtgtgtgtatctatatttgtcctcttt |
| | | tttctttgctgagccattgaaccgttaactgactaaattgcagacattgt |
| | | ggccctgcacccttaaattcttcagcatgtatctcctatgaacaaggaca |
| | | ttctcctgtacagacaaaacaaattatcctactcaagaaatataacattg |
| | | acagaatactcttataaaatatatagttcatgttcaaatttccccagcat |
| | | ctaacagtatcctttagagcttttatggtgttcatccaggatccaatcag |
| | | ggatgatcttttcctaaaagaggacactcaagatctagatctagaacagt |
| | | tccgcacatttttttttctttactgctattggcatttttgaagagtttgg |
| | | tttcctgtttcttaaaataaattttgcattgaagtgtaatatgcatacat |
| | | aaaatgcataagtgtacagtttgaagaattatcacaaaaggaacacatcc |
| | | actaaaccagcacccagatgaaacagaatatcccagaagcctccctcgtg |
| | | tccctcccaacgctacccacttcctccttctaaaggtaaccatgtcctga |
| | | cttctggcaccataaattgcgtttgcctgtgtttgaatttcgtacaaatg |
| | | gaacctgcagtgtgtattctttttttttttttgagaccgagttttgctgt |
| | | tgttgcccaggctggagcgcgatgccatgacctcgactcactgcaacctc |
| | | cacctcccaggttcaagtgattctcctgcctcagcttcctgaggagctgg |
| | | gactacaggcacccaccaccatgcctggctgattttttgtatttttagta |
| | | gagacggggtttcaccatgttggccaggttggtctcaaactcctgacctc |
| | | aggtaatccacccgcctaggcctcccaaagtgctgggattacggacttga |
| | | gccactgcccctggtcagtatatattcttttgtggctgcctctgtcactc |
| | | agtgtaaagtttgtgagcttcattcctgttgcctgttgcttacgctgggg |
| | | ttccttcatcgtcactgctgttgtgccttctcggtcatatcacaacgtat |
| | | ccatcgatcctcctcttgatggatacatgggttgtttctgggctttgact |
| | | tttacaataatccctgtgaatacttacgtactgtatcctggtgcacactt |
| | | gcatgcatttctttggccgaaatcaatgtctagaaggggaattattgagc |
| | | catagagtgtgagtgtcttcaacttccatcggtaataccgacttgtttta |
| | | caaatggtgataacaaatggtgataacattgttatcacctcatacccgtc |
| | | agcagagtatgagggttcctgtgggtggctctgcatcctcaccagcgctt |
| | | gtcactctgttgaattgcagccactctggtgggtgggtgcagtttctttt |
| | | ggtaattttcatttgtgattctgtatttgatttgcagttccctggctgag |
| | | tgtcttttcatatgtctgctggccacctgggttggctcttcagtgaagtg |
| | | cctgttcagctttcttacacatttttctactgagtggtctggttttgttt |
| | | tcttattgttgggcttgttttgttttgttttgtattgtcttgagacagag |
| | | tctcactctgtcgcccaggctggagtgcagtggcacaatctcggctcatt |
| | | gcaacctccacctcccgggttcaacattcttttgcctcagtctccccagt |
| | | agctgggattacaggcacgtgccacctcacccagctaatttttgtatttt |
| | | tagtagagatggggtttcaccatgttggccaggctgctcttcaactcccg |
| | | acctcgggtggtccaccaaccttggcctcctaaagtgctgagattacatg |
| | | tgtgagcccccgggcctggcctcttactgattttgaggagttctttatat |
| | | tcaggctaccagccctttgtcagttatatgtgaggcaaataccttctccc |
| | | actctagttttgcctttttactacgaaattgccatttttgtagatcgaaa |
| | | tggtcaaatattggcaattggcatatggttcagcctaccataaaattgat |
| | | ccactttttttctttgttaggtgaggactttttttgtcctatttctctac |
| | | ttctgaggtcatgaaactataatattatttcctagatactctattgtttc |
| | | acttttcagatttatgtctataatccacgtgaaatgtgaactgatatttt |
| | | ttgtgtacactatgaggtagggatcaagttttactttaaaaaatatatag |
| | | ctaggccgggcgcagtggctcacgtctgtaatcccagcactttgggaggc |
| | | cgaggagggtggatcacgaggtgaggagttcgagaccagcctgaccaaga |
| | | tggtgaaaccccgtctctactaaaaatacgaaaattagccgcgtgcagtg |
| | | gcaggtgcctgtagtcctagctactcaagagactgaggcaggagaattgc |
| | | ttgaacccgggaggcagaggttgcagtgagccgagatcgcgccactgcac |
| | | tctagcttgggtgacagagcaagactccatctcaaagaaaaaaaaaagaa |
| | | aaaaagaaaatagataacgaattgagcactttttattaaagacagtttta |
| | | ctaaggtatcatttgcacactatgaagctcagctgtgaaaagtatacaat |
| | | tttatttttagaaaatttagggtatcatctggttttagaacatttccatc |
| | | atcccatgaggtccatcatatctggtttcaggcaatacctgcttccagtc |
| | | ccaaaccacaaatctactttctgtgtctatagatttgctttttctagaca |
| | | tttcatataattggaatcatgcactattgtagtcttttgtagctgacttc |
| | | tcaattagcttattgtttttgaggttcttctgtattgtagcacagatcaa |
| | | gtatttaggtcctttttcgtgagttaaattcggtggtgtggatatatcac |
| | | agtgtgtccatccattcaccagctgctggacacgtggattattcccacgt |
| | | cttggctgtaataaacagagctgctctgaacatcgcttgcaagtcttagt |
| | | tgtttccatttctcgtgagtagaggcttggattggaattgcaagattgta |
| | | tggtaacgtataattttattttgctaattatcatttgtgaactgactttt |
| | | ttctcttgctatacggttctaaggatcataacacatgtataggttttgta |
| | | gccaccagtacaatcaggatacagaacagtttaatcaccccccaaaaatc |
| | | tttcccaagggttgaccttttaatatgatttggctgtgtccccacccaaa |
| | | tctcatcttgaattgtgactcccacaattcccacatgttgtgggagggac |
| | | ccagtgggaggtaattgaatcgtgggggcaggtcattcccatgctgttct |
| | | cgtgatagtgaataagtctcacgagatctgatgggttttttttctttttt |
| | | ttttttttgaaacggaattttgctcttgtcacccaggctggagtgcaatg |
| | | gcatgatcttggctcaccacaacctctgcctcctgggttcaagcgattct |
| | | cctgcctcagcctcccaagtagctgggattacaggcatggaccaccacac |
| | | ccagctaatttttgttttactagtagagatggggtttcaccatgttggcc |
| | | aggctggtctcgaactcttgagctcaggtgatatgcccacctcggcctcc |
| | | cacagtgctgggattacaggcgtgagccaccacgtccagttgatctgatg |
| | | gttttataaagaggagttcccctgcacacactctctctcttccctgccgt |
| | | catggaagacgtgacttgctcctccttgccctctgccatgattgtgaggc |
| | | ctccctagccatggggaactgcaagccagttaaacctttccttcgtaatt |
| | | acccagtctcggataattataaagtctttatgaacagtgtgaaaacagac |
| | | taatacaccctttatgggtgttaccagtggagggtcttgactacaagttg |
| | | tctgggttcttaaccagattcttggcattttgaacaaagaattggacaaa |
| | | atgcacaaagaaagcaacgagaaaacgaagcaagggaagcacagatttac |
| | | tgaaatgaaagtacactccacggagtgggagcaggctcgagcaggagcaa |
| | | gtggctgattagagaattttctggagccttaataccctgtagaggtttac |
| | | cattggttacttggttacaccctatgtaagtgaaggagtggcctgtgacc |
| | | agtctgattggttgcaggaggcgaccaatcagaggttgaagtgaagttgc |
| | | aacgttacacctgaagacctggcctgggaccagtctggttggttgcggga |
| | | gggaaccaatcagaggtactttccatttttcatctgccacgaggtgcaaa |
| | | gggagtagtagcctctgatccttttgttacctgggtatggttttcctttt |
| | | gattcagttctaaaaagtcagggtgaatcggccttaagttccctgcctcc |
| | | aggcccctgccgacccagcaacaactatccattccctgtagttttgcctt |
| | | tgggagcctgtcattaaaatggaaccacacagcatgtaccctttgaagac |
| | | tggtttcttccacttagtctagagctattgagattcgtcctggatgctgc |
| | | atgggtctctagttcattccttgtgatgctgagtagtgccccattgtggg |
| | | gagggaccacagtgtgtttatccattcatcctgggaaggacattaggttg |
| | | gtttccaggttttggcaattatgaatagtgccgctacaaacatttgcgta |
| | | cagcgttttttgtaaacataaggtttgatttctctagggtaaatatccat |
| | | gattggggtttctgggtgtatggtcattgcaggattaacttttttttttt |
| | | ttttttttttgagacggagtcttgctgttgttggctcaggctggagtgca |
| | | atggcgtgatctcagctcactgcaatgtccgcctcccgggttcaagtagt |
| | | tctcctgtctcagcctcccgagtagctgggactacaggtgacgccaccat |
| | | gcccagctaatttttgtatttttagtagagacagggtttcaccatgttgc |
| | | ctacgctggtctcagactcctgacctcaggtgatccacctgccttgacct |
| | | cccaaagtgctgggattataggcgtgatccaccgcgcccagcctctactt |
| | | tagtttcataatgggctttaatttgtgaattaacttcttgatgtatctgc |
| | | tcatccatccattcattcattcatttctatattcacccaacaaatagtta |
| | | agtgccttacttctgtctttgctatttctttgcctttggagcactgtttt |
| | | gaaagagccagcgcccactgtcagctgtccagctggtagcactgtcatct |
| | | ggaagattttagatactttctcatgagtgattctacaccagcctgaatct |
| | | cacactacctgacatcctctcaatcgtagccacacccaagggtcctactt |
| | | tgctgtccccactgccaccagaccaaacacacccccttccctgaaagtct |
| | | gggagtcggtttcttcttcacaatagaagttggcctctgctggtcatgcc |
| | | ccaaaccttctggagctgcgggttctgccttctctgcagctgccttcccg |
| | | tgggctgccctggtcagtgcaggacatggcacaggacctccgccctgcca |
| | | cacccccgggccatttcctcggcaggcctcgtgccagacttctccaaagg |
| | | actgaccgcgtgtgccttctccaccttgcacacactcgcacaccaacctg |
| | | ccagcctggcctgtgctcttcgccgcccaaacgcacctctccctggtgtg |
| | | gtcactgacagtctttgcgtggccagtggccaaacccaggctggctttgc |
| | | tagtggcctcatcttctctctgcagcgttcacagtgatccacttcttttt |
| | | ttttttttttggagacagagtcttgctctgttgccctggctggagtgcag |
| | | tggcaccaatgtccgctcactgcaacctccacctcccgggttcaaggcaa |
| | | ttctcctgcctcagcctcctgagtagctgggattacagtcatacaccacc |
| | | atgcctggctaatttttgtatatatatattttttaatagagacgggggtt |
| | | tcaccatgttagccaggctggtctcaaactcctgagctcaggtgatccac |
| | | ccgcctcggcctctcaaagtgctgggattacaggctttagccatcacacc |
| | | cagcccccctccctacccctttgtccctcctcagagccctgctggcccac |
| | | ttccctggccgttcaaaacattgcagctccaaggctgcttcccaggcctc |
| | | cccacctgctgtcttctccctggtgatattatggacagtggatctctggc |
| | | cctgagctcccttctgatttcatggtatggccacctccttgtccctatcg |
| | | cttgagcgtgtcaccagcatcttcagcttgggaggtccagaactgagctc |
| | | ctgattttgccccctaaacttttacttcttctggtctcactcatccgtcc |
| | | acgtactgaaggcagaacccaggaacccaggaagtgtccttgttcccttt |
| | | gttcacatcctgtcggttctgcctagaccttaaatcatgaacctgaccta |
| | | aggccactccagcttttgcctggaccacagcaaaaggccaggagtgagcc |
| | | cccgcttctactcctgacacctccggagccattcgccacccagagctgtg |
| | | taggctttaaaaagagccagccagccgcgtgggatcctggatgggatcct |
| | | ggaacataaatcaggtgaaaaattggtgaaactcaaatcaagttcatcgt |
| | | ctagttaatttcctgttcttgattatcaagctgtgcttgacgtcgtacaa |
| | | taagacattcacgttaggagaagtggagcgagggatatatgagaactccc |
| | | tgtaccgtatatgtaactttcctatatgattaacattagtttgaattaaa |
| | | aagttttaaaaaattggatgggtgcagtggcttatgcctataatcccggc |
| | | actttgggaggctgaggtggccggatcatgaggtcaggactttgagacga |
| | | gcctgaccaacatggtgaaaccccgtctctactaaaaatacaaaaattag |
| | | ccaggcatggtggttggtgcctgtaatcccagctactcaggaggctgagg |
| | | caggagaatcactcgaacccaggaggcggaggttgcagtgagccgagatt |
| | | gtgtcattgcactccagcctgggcaacagagcaagactcagtctcaagag |
| | | aaaaaaaaaaagttaaaaaattaaaatgcaaaataagaaagaatcgaact |
| | | gtgtgctgtcttacttaaaacactgcagagctcttgtgtgtgcctagcat |
| | | gtaagtcagatgcctcttgtcgttttgagacagagtctcctaccgtcccc |
| | | caggctggagtgcagtggcgtaatctcggcccactgcagcctccacctcc |
| | | tgggttcaagcgatgctcgtgcctcagcctcccaagtagctgggatcaca |
| | | ggcatgtaccccgcctggctaatttttgtatttttagtagacacggggtt |
| | | tcatcatgctagccaggatggtctcgaactccttacctcaagtgatccgc |
| | | ctacctcggcctcccaaagtgctgggattacaggtgtgaatcactgcacc |
| | | tggccaggcagaaacctcttaatggctcccagggcctggcccacctcccc |
| | | agcctcctctcatgccacactttcttccctcagtctgcatcgggaacagt |
| | | tgccacctttcagccacgtgagcatgctgggaccgtcctttccatgctga |
| | | tccctgcatccctcgtgccttccccagacctacaccaccgcaattgcctc |
| | | catctcactgtgatatctccacttcagtctcatttcttcagaaaggacgt |
| | | ccctgactctgtccaaaccgcatgttgggtattctctattgcaggaagct |
| | | tttgctttcttcctggctcttaatcacagctggtgactcgggtccttgct |
| | | tgtggattctccctgccactggttcctaccactgagtcaggtgatggacg |
| | | ccatgttcacagttttatcaccagcgtctagcgtggcctgcagcgggtgc |
| | | tcccatagaacccataaacaaacaacttcttgtttcttattatgtgaaga |
| | | aagttggtctgtgtaattcaccctccagagaaatgacagcactttcagta |
| | | ctttgttctgcccttccttgatctccacgctgtttgcagatggaccaccg |
| | | ttgtagagatgtgatttgattcttgacgtttctgagtgtccacggtgctg |
| | | cgccttcccccaggtactacagtactgagattaataagacagagtcaatg |
| | | taaggtgttagtaggatttagaataggtctaatctcattcttaacggatt |
| | | tcaggagcttattatgtgttgaaagaatgaataggataaaagttgttaag |
| | | aaattttctccgtttaatttttatttattaatttatattttagagacagg |
| | | gtcttactctgatacccaggctggagtgcaatggtgcagtcacagctcac |
| | | tgaggctttgaattcctgggttcaagcaatccttctgcttcagcctccca |
| | | aattgcggggattacaagcatgagccactggctctggcctattttctcct |
| | | tttaaaaaaaattgctgcaggtatacacatatgcacggatatacacatac |
| | | aaatatgtaccttatacatatatgtatataataaatacatgcatatgcgg |
| | | aattgtggcatagtcttagtcttttttttttttttttttttttttgagac |
| | | ggagtctcgctctggtgcccaggctggagtgcagtggtgcaatctcggct |
| | | cactgcaagctccgcttcccgggttcacgccattctcatgcctcagcctc |
| | | ctgaggaactgtgattacagcatgggccaccaggcctggctaatttttgt |
| | | gtttttagtagagatggggtttcaccatgttggccaggctggtctcaaac |
| | | tcatgacctcaagtgatcctcccacctcggcctcccaaattgctgggatt |
| | | acaagcatgagccaccatgcctggttgtatttagtctttaacctgtcctc |
| | | aggctgtttctctctgggcagggagaagcatggaaagggggatgggccct |
| | | ggtggccacctttgaagcatggaggctccggtgccaccctgcccttaggc |
| | | tgggcacaaaggtggctgagggaaggctagtcagctgctggaggtgtcct |
| | | ttatggtctcagtgattaaaggttatgttgtccatgaatttaattttatt |
| | | tcatgatggaaaagaggacattgcaaattaagtatgtcacagaaatgtaa |
| | | ggagccatggggtctgatagggctgagggtgagtggcttgccgtcgagtt |
| | | tccagaaggcgttattatggaccctaaaggggatttcatgcgtttcaaag |
| | | actctaacagaagtgttctgatgaagtggagtctggtgctgggacctgcc |
| | | acttcagcctggaattgtcctcgtgtgaagcgcagtaaaaacccagaatg |
| | | gatgtcggcaataagggaatttctgggatatgatgggggaacgaatattg |
| | | tcacctccatttgtgtcaggaagtctcagtatgcagatgagtcagctgca |
| | | gggggtggcctcccaggctgctggtggagcccagaagcagggggcaggcg |
| | | tttggcctctgatactctcctagctctgagttgggacggagctcgtgtat |
| | | ctacagtgtgctcacacagacttggtgccggtagatggtgaggggatagg |
| | | ttggcaggtgggtagggggcctcccgaggcagtagggacctggaggaggc |
| | | tggtagaaagtggagattggccaaagtgacctgaattagattctacctgg |
| | | ttcatgaatttgcctgaggctaaggcttcagcgcaagggatggtggagta |
| | | atacctttcctgaaacacctgctgccggcgtggccttgtctgtctctgcc |
| | | agatcttgttagcactgtggcttcaggcctagttaaacattcaagcccaa |
| | | tgcggttggagagcacccatggcacgcccattgtgcagcagtgtgtttaa |
| | | tacatgtgctgcttttaatgggtgtgaaagatcagagagcttgggtgtgt |
| | | gttttaccaaccactttttaaactcacatagtaaaatttatagtatataa |
| | | aaagcagaaaatatggtgtatcttctaaatattcacatttaaaacatttt |
| | | ttagaaataatgtatgtcaagcaaggtcaagctaatgtgtttatataggc |
| | | tctaaattagagttgtgaaccagtatctcatcccacttatagggtttttt |
| | | ttgaaaacgttcaaatgtttccagctcttttgtttctttattctaaaaca |
| | | ttatctgctattttgttcgatagagtaactgtttctattccactttcttt |
| | | ttttttttttgagacggagtctcgctctgtcgcccaggctggagtgcagt |
| | | ggcgcgatcttagctcactgcaagctccgcctcctgggttcacgccattc |
| | | tcctgcctcagcctcccaagcagctgggactacagacgtgtgccaccaag |
| | | cccagctaattttttgtatattttttagtagagaggggttttcaccttgt |
| | | tggccaggctggtctcgaactcctggcctcaagtgatccacccgcctcag |
| | | cctctcaaagtgctgggattacagacgtgagccaccacgcctggcttatt |
| | | ccactttgtaattggctatccctgctattaatatacagggtactctccct |
| | | ctcgtatttgcgtctaaatgtcatctataataaataacagtgaatactta |
| | | ctggtttcatatttagttttacttttatcccacccattttcttttagtag |
| | | ttcttatgacttcacgtttatttaataaaggtaaaaaagaactttctgtt |
| | | tccaattcattcctttcaccaataaggaaggatgtggatcttcactgctt |
| | | cctacatagctgccagcctcatgtagctgtttaaaatttaaattataatt |
| | | gaaatcattaaaaaattaattccttgttcatactagctacatttcgagtg |
| | | gtcagtagccacatgtgcctggaggccacagaactggacggctacaggat |
| | | gtacccatcatcacgggaagttcatagaaagttctttttctttttctttt |
| | | tctttttttgagatggagtctgctgtgttgcccaggctggagtgcagtgg |
| | | cgcgatctcagctcactgcagccttcacccactgggttcaagtgattctt |
| | | atgcctcagcctcccgagtagctgggattacaggctcccgccaccacgcc |
| | | cggttaatttttgtatttttagtggagacagggtttcaccatgttggcca |
| | | ggctggtctcaaactcttgacctcacgtgatccttccaccttggcctccc |
| | | aaagtgctgggattataggcatgagctaccatgcctggcatgtatctctt |
| | | cttctctaagtactcagctgtcttgtttgaggctagtacctccactagtc |
| | | tggaaccctccttcctgggctttctgcccacttccatctctcctttgaca |
| | | acagtggtttttccagggtctgtgcagagctcagctttctgccataagac |
| | | aggggagtttctgaagactcctcaccccttttgggagtgagggctgctga |
| | | ctgaagctccaggtcactgttttatgagctagctctccgtttgttgatgg |
| | | ccggggacccagcctttgtcccctcctctcgtgcttgggcactcactact |
| | | cactgcctcggttttgctttcagttttgattgtgcattgagtccacaact |
| | | tcaatgggattttcttaaggcataaatgacagttgcatggtgtgcagtgg |
| | | gagaacaaatcgcccagcctgggacatccctgggtggcagagtggacctg |
| | | ctctcaacttatggaatgtgcagcaacaccttgaggttattaataaatac |
| | | ctgagtctgggcatggtggttcatgcctgtaattccagcactttggcagg |
| | | ctgaggccggaggatcacttcaggccaggagttcaagcccagcctgggca |
| | | acatagtgagattccatctttaaaaaaaaatttttttttaaaagaaggtg |
| | | ggcttggtggtgcacacctgtcatcccagctacttgggaagatgaggtgg |
| | | gaggatcacttgagcccaggacgtcgaggctgcagtgagccgtgatcatg |
| | | ccactgcactgcagcctgggtgacagagaaagaccttttctcttaaaaaa |
| | | acaaaacaaaacaaaacacacacacacacacacaactgcgattcttccag |
| | | aaaagttcagaaaaaggaagtccgaaatacatagtccatagtatttcagc |
| | | caccagtgctgctgaagtgaaattagagatagcagagatatctcaggaga |
| | | aaatggaggtttttgggtttctccgttagatgactcgaacaagtctgcat |
| | | gtgctctgtaaacaagaaaacacagtctgggaaactgtcagcctctgccc |
| | | cagccagtgtgcaaaatactcttcaaaagagagaaaactagaaaacaagt |
| | | tgaaaaccgggtgatctgacaagtgtggaaattcacagcaagcatgtgcc |
| | | tgtgctgcagatacgatctgggagagagccagcagcctggatccacattt |
| | | cccagaagcaacaggtggtgaaggcagcggcatgactgctcccgcggaag |
| | | gttagtcacgcatgttcagcatcagccgggcgcggtggctcacgcctgta |
| | | attccagcactttgggaggccgaggcgggtggatcacttgaggtcaggag |
| | | ttcaagaccagcctggccaacatggtgaaaccccaactgtactaaaaaca |
| | | caaaaattagccgggcatggtggcaggtgcctgtaatcccagctactcag |
| | | gaggctgtggaagagaattgcttgaacccgggaggcagaggttgcagtga |
| | | gccgagatcatgccactgcactcctgcctgggcaacagagcgagactcca |
| | | tctcaaaaaaaaaaaaaaaaagagaagaatattccgcaacttagagcttt |
| | | tcctttgcatgatatgatgtttacaggcgaggtggctccgcagcctcggt |
| | | ggcctttcttccagctgaagatgcccacaggaggtgtcagtgcaggtata |
| | | accacctgtgggtcagaagaaaatgcctttgcaagtaaatctcctcctgg |
| | | atgaaaaggcccatctgtgttccaggcaaggacagtggccgcttctgcgt |
| | | ttctgtggccaagcgtctggcgtgcgggtgtggctgggctcaccatgctg |
| | | tgcgggctacacctgctgcaggagaagtgtgtttgtgctcgaagaagaca |
| | | atgtgggtgtcggcccggcgtttttaggttggactacaatagtgagagct |
| | | gcaaattggatgattcttggagctatttcatgaaggtcaaattttctttt |
| | | gttttgttttatttaatctctcccagacttctgaacttttgctatgttga |
| | | atttttagttcttcaaggttatttggccttattcttgacttgcatgtttt |
| | | tctgccctagctctaccacattatctttttttttttttttttgagaagga |
| | | gtttcactctgctgcccaggctggagtgcaatggtgcgatcttggctcac |
| | | tgcaacctctgcctcctgggttcaagtgatcctcctgcctcagcctccca |
| | | agtagctgggattacaggcgtgcatcaccacgcctggctaattttcgtat |
| | | tttcagtagagacagggttttaccatgttggtcaggctggtcttgaactc |
| | | ctgacctcaagcaaccccccgcctcggcctcccaaagtgctgggattaca |
| | | ggtgtgagccaccatgcctggcccacattatctatttctaagcctttaat |
| | | atttttcatttacctgctactgctcactctaccttgtattctcagggagg |
| | | gtgtatttcttcccagctttattttatttcttctctaaatctcagaatta |
| | | ttgcaccttgttgatgttcacattactgtatttagtttattttattgtat |
| | | tattaatattattattatatttgagacagggtctcaccctatcacctagg |
| | | ctggagcatagtgacacagtcatagctcactgcagccttcatttcctggg |
| | | ctcaagcagtcctcctgcctcagcctcccaagtagctgagatcacagaaa |
| | | tgcaccaccacacccagcttccttttctttttcaaaaaaacagctttatt |
| | | gacgtataattcacatatgatgcaattcaccctctcaagtgtatgatttg |
| | | gtggtttttagtatattcatggtgttgtgcaaccatcaccacaacctaat |
| | | tttacattttcgtcaccccaaaaagtaaccctgcatccgttagcagttac |
| | | accctctctctcctaaacccatggcagccactcatctgctttctgtgtct |
| | | acagatctccctatgccagacatttcatataaaggggatcacctagttcg |
| | | tggccttttgggactggcttctttaactaaacatcatacagctttatttc |
| | | cattttcagtgtattccctttttttgttttcaagtatttagtggctacct |
| | | agtctcagattttaaatttagtttttcctttctcttctttctataatcta |
| | | tttttcatcatctacactgtgtgcttgaatcagcacctcttagaaaccta |
| | | cggatgagctgttagctctctagggacaagaacacggtctgggacccccg |
| | | tccctgctgcaggtgtgacctgaccccctccctctgccgacgtgacctat |
| | | ccccctccctcccgtctgtggcatctggtgcatttggtgctgtcatttca |
| | | caaataaaaggtgatcagctggtggtctgtttcagttctggtatctttcc |
| | | ccagctctcttgtttcatagcctcttcccattatttaaactctgctctgt |
| | | tacaggattcttaatagttattgcagaccttcatttccattttgttgtct |
| | | gcaactcattgttgaagtgccatttgttgcttctgaacagcccatagaca |
| | | cttggacttgaatgcatatgactctcttcttgtgggtccagatcgtcact |
| | | cactgactgtcctcacattcacagctgttctcaggacaagcaccgtcttt |
| | | ccctttttcggatgtccctgtttacctgaatcaactcatttaatcctcac |
| | | aacagccctgtgctatgatatgaccatcatatcacagcattttcgttttc |
| | | tttttcttttatttttttgagacagggtctcattctgtcacccaggctag |
| | | agtgcagtggcgtgatctcggctatctgcaacctctgcctcccgggttcg |
| | | agagattttcctgccttagcctcccgagtagctgggacttacaggcacct |
| | | gccaccacagctggctaatttttgtagttttagtagagacagggttttgc |
| | | catgttggccaagctggtcttgaactcccgacctcaagtgatctgcccac |
| | | ctcggcctctcaaagtcctgggattataggcgtgagtcaccgcgccggcc |
| | | agcatttccattatctagacaagggaactgaggtcaagcattggagtgaa |
| | | agtcacctaggtggctttttttcttttttaacctatttatttctttcaac |
| | | ccttagcccagatcctaaccttcggtaagtactcagtaaacatttagtct |
| | | gctgggtgcatgaacgagcgaatgagcaaatgaatgacagcgatagcacc |
| | | tggtgggctgttcgctggtgtgctgggtgtgctgggtgtgctgggtgtcg |
| | | tctctcagagactcatttctacagtctttgtgaaggctcgtgctagtaaa |
| | | ccctaattcaagaggtttcttagaggtgggaggcgaaggtctctgaagtg |
| | | tgtggactcctggactctccctatgttagcattctggaatgtagcattta |
| | | gcagtggggcatttattaactcggaatacagaagataagaagccaagcca |
| | | ggcacggtcgctcacgcctgtaatcccagcactttgggaggctgaggtgg |
| | | gcggatcatgaggtcaggagatcgagaccatcctggctaacacggtgaaa |
| | | ccccatctctactaaaaatacaaaaaaaattagccaggcatagtggtggg |
| | | tgcctgtagtcccagctgctcgggaggctgaggcaggagaatggcgtgaa |
| | | cccgggaggcagagcttgcagtgagctgagggagccactgtactccagcc |
| | | tgggccacacagcgagactctgtctcaaaaaaaaaaaaaaaaaaaaggcc |
| | | aaaacaggggaatgtggtcactcaggggatgcctgtcattcccatttggc |
| | | tgtgatggcagctgtttagccattactgggagagagagggaagctgggta |
| | | tagaaggtacctgtttagaagcaggggtggggaagcgggagggagagaga |
| | | gagagaatgcgtgcacatgctctaaaattttattttctggctgggtgtgg |
| | | tggctcacgcctttaatcccagcactttgggagacagaggcaggcggatc |
| | | atttgaggtcaggagttcgagaccagcctgtccagcatggcaaaaccccc |
| | | tctctgctaaaaacacaaaaattagccaggcatggtggcacacacctgta |
| | | attccagctactcaggaggctgaggcaggagtattccttgaacccgggag |
| | | gcggaggttgcagtgagctgagatcgcgccactacgctccagcctgggca |
| | | acagagcaagactctgtctcaaaaaaaaacaaaaaaatttttttttggtt |
| | | aagcagaagatcaaattaaacaggcaaggtgcctaccctttgtttctttc |
| | | actttcttccataaatccatctcatattttattatttgggacttcttttt |
| | | ttttttttttcttttttgagacaaagtctcactctgtcacccaggctgga |
| | | gtgcaatggtgcgatctcggctgactgcaacctccgcctcctgggttcaa |
| | | gcaattctcctgcctcaacctcctgagtagctgggattacaggcatgtgc |
| | | caccacgcccagttaatttttgtatttttagtagagacgggatttcgcca |
| | | tgttggccaggctggtctcaaactcctgagcacaactgatgctcccgtct |
| | | cggcctccctaagtgctgggattacaggagtgagctaccgcgcctgacct |
| | | gggacttcatttgttaagaagaggttaagtttcaccagcagccatttgta |
| | | gtaggaacttgagatttatcatcaaatattgggacaacaagaacaaatca |
| | | gttcttatgattatttatggtgcatatgagtaggaaggacttacaaccct |
| | | ccggagagcattttaatgttaacatatgaaagatattctctcttttgtac |
| | | caaaacttttgttcttcttacgcagaaagaatagataattataaaatata |
| | | tctatgactccataggctttggctatttggtagattaagagtatttttat |
| | | ctttatttttcttattggctgattacagtttagtttaaataactctcatg |
| | | ccgcttggtcgtttccgacctagtcgtgaggaagaatgcaatgcaaagtg |
| | | aaagacgtttttggaatgtttctgaaccagcccaagctgattgcctcttt |
| | | tctgtttattgttttaagtcataagtgacgtaagcttctaggcatttgtt |
| | | atgctatgtggctggctcccaaagtcatgtcttaaaacattctttccttc |
| | | ctgattattcttctctttcttgtttttcttttcttctttttcttttaatt |
| | | tttttttcttttttgagacggagtttcactcttgttgcccaggctgaagt |
| | | gcaatggcacgatctcagctcactgcaacctccgcctctcaagtttaagc |
| | | gattctcctgcctcagcctcccaagtagctgggatttacaggcgtgtgcc |
| | | gccacgcctggcttgtttttctttttctttttttttttgagacagggtct |
| | | cactctcttgcccaggctggagtgcaacggcgcactcacagctcactgca |
| | | gcctcaacctcccgggctcaagtgatcctcccatcttagcctcctgggta |
| | | gctgggaccacaggtgccaccacacccagctgatttttaaattttttata |
| | | gagatgggatctcgctatgttgtccaggctggtctcaaactcctgggctc |
| | | aagtgatcctcctgccgcggcctccgagagtgccggggtcacagctgtga |
| | | tccccgtgcctcgctgttgttctctttctgttgagttggcagtcaggtcc |
| | | ttgattcttggtggctgagggtatctcagtccaaggcaagaatcccacag |
| | | aaagagactgggacaaatgaggccagaaatgggcctcgcccatcactctt |
| | | ttccaaaatgggctttgtcagtcactcttcctatacttgggtcaccaaac |
| | | ctgatcaccactatgtgcaaagtgcagttttagttttcaaagaagtgaca |
| | | ctatacttaagaatacttaagaacgttaactgcgttaacctccacgcaca |
| | | cagccatagacaaattcccccgtcagacaggagcccattaggaaatgccc |
| | | acccctctctgtagcctttgtaaaattaagctgataaaagacatcgctgt |
| | | tgttgaggtttaaaatcaatggataaccaggaaagaatataagaaatcat |
| | | attaattattctcctgtttacactacacttaaatcatttagctgtctttt |
| | | ttttttttttggaccttctgctgcaaaagagccattctaatgaacttgga |
| | | ccccagggtccatgtgtgtggttattcattaaatcatgtcgcctgcagag |
| | | tctcctgacttaagactttcgctgggcttgaactcagtaacctgtcctga |
| | | tgtatttctcaggcgtttgtgattaggcagctgggctctgccttacctgt |
| | | cagggtgataacccttctggttctgacaaaaggtttcactttaaatttag |
| | | ctttccatattgaagcataaataaaaagataatttacttcccgattttgc |
| | | tcgttgtggacacggaggtgtgtttgaccttcgtaaagctagtaggatca |
| | | tgttatcactatgggttggtcattcagcgcttcctaaagaaaaactcaag |
| | | tcagaaacaaatcaggagcaattattgatttaacagttttgcaaatgctt |
| | | ttgtatgtagaaggtgcatgaaaaagattccagtgatacgcagatcaaac |
| | | actgcgtcgataatcacagctaatgtcgagtgcttgctgcgtgcgtgttg |
| | | atcatttaagcctcacatgcgagctgtgtggtaagccctcctcctgccct |
| | | cgccttagagatgaggaaactgagaggcagcgatgaagcagctcactagc |
| | | cctgctcctgtcctccccttagagatgaggaaactgagaggcagcgatga |
| | | aacagctcactagccctgctcctgtcctccccttagagatgaggaaactg |
| | | agaggcagcgatgaagcagctcactagctctgctcctgccctccccttag |
| | | agatgaggaaactgagaggcagagatgaagcagctcacttagatcacggg |
| | | attagggggcggacctggacccgactccgaggccccctccagcagccagg |
| | | ccctcgcccatcacgcttggttgcttgtaatgggagccttgcctctcgcc |
| | | agcagtttgtggggagtaggtttcccgtttccactgttatttcctgactc |
| | | ccgtgcctcccaacttcccacccgctgtggctggcatttccaccggctcc |
| | | ctggcttgtcctacgcctcatgcctcctatgtggctcttctttctcctcc |
| | | cgcccccctgctgcccgtggccccagaatggaggcttcccaggatggccc |
| | | acacagtgccctgcacaggtgctgtggctcagccctggccattccctcca |
| | | cacgttctctgctccagccacactgagctccaagccatcctttagctatg |
| | | cctgctgcaggccacctctgtagagggcctttcatgacctgttctagtaa |
| | | cccccctgcctggtgtcagccacccccgcccctggcctggtttgctattg |
| | | ccgggctgacctcgccccgtctttcagttctccgtgtggagggtgctgat |
| | | ccgtccgtctccatctgcttgtgaagcaataacagaacagcataggctgg |
| | | gtaatctatacagcacggaaatttctgtcctcatgcttcaggaggctggg |
| | | aagtccaagatcaaggcaccagcaggttcaggtgtctggggagggctgca |
| | | tcttctggaagggagaaggaaactggagggcaagcgagagagccccttac |
| | | agcaagcccctttatgcaggcgcctgatcccatcgcaggggaggagccct |
| | | cctggcctcgtcacctcttaaagccctgcttccttaagactctcactcca |
| | | caacgcctgaactttggaggggacacgttcgagccacagccccttccttc |
| | | ctgaggcttctcaagatgccccctcctgtgcttcctggagcacccgtcct |
| | | tccttgtcctgggacttggcacgcctgaccatagtcatacctgcttgtcc |
| | | tcttggcccctcttcaggtaatgggctccacacaggtcacgatctcggcc |
| | | aaaatagtcacacatcgtccctgcacccatcagtgtgtagtaaacaggaa |
| | | gtgcttaataaatgtttgatgaattaagagtgactcagtaatcccagtac |
| | | aaaaaagcaacagcagacgttggagatgtgcttgaggcacgtgcccagct |
| | | tgtgagcagaggccgccgctgccactccgtgctccccgtggactgccgtg |
| | | ctgagcgctgtgaatgtgctggctgtcccgtcgtgctccagctcttcagg |
| | | cctccctgctcagcctctctcagcttcagttgccacgcacttctaggaga |
| | | ggccatcttgactttctcttgcagtcccggagccacacagggagcaggtg |
| | | cgcagtcacctgggtgagggccccttcctgggtttcagaggcagcccagt |
| | | tcatgctcccagggcagcccttgtgcatggggagccgtagcctcaggcag |
| | | gggcaccctgagccaacagcaccttgcagggccagccacagacataatgg |
| | | aaaacctcatccttcagccgcaacttttcctacctcccatcaaaaaaatg |
| | | tccctcctctctcagtatgtggggactgcggtctccagagtggccatcca |
| | | cagtcacactggctgtcagtggctcctgtcaccaccctgccacctggtga |
| | | cttgtgggccacactcaggagtcatgggaaggagacccttagagcagccc |
| | | caggacaggcctccttgcgtgtacttctttgatgaaggcgggtataagcc |
| | | ctggcagctcattgtcctctcttgagtccacataggttgcattttttaac |
| | | tctggcaaagtgaagttacaaagtcagtctggtgtcgcctgggacaggtc |
| | | cgagaaattagtataaagatggcttcactcctgcgtcgtattccagttca |
| | | cgtttccatttcagtgtatacatgttacagtcgtgattgatcctgtgcat |
| | | aaattgggatgtgggtttttgttggagaaaaaaatgcacaaaagttcagg |
| | | agctaagtaagagtgaaagattttcattggaccaggtgtggtggctcaca |
| | | cctgcaatcccagcactttgggaggccaagatgggcggatcacttgagtc |
| | | taggagttcgagaccagcctggccaacacagtgagacccccatctcaaaa |
| | | aagaaagaaagaaatagttggacatagtggcggacgtttgtagtcccagc |
| | | tactcaggaggctgaggctataggaggtgggggctgtagggagccatggt |
| | | tgcaccactgcgctccagcctgggcaacagagcaagaccctatctctaaa |
| | | ggaaaaacaaaacaaaacaaaacaaaacaaaacatgaagagtttcttggg |
| | | gccagttggcatctaaaaatatctaagtcttttgattctcagctatgacc |
| | | tggctacgttctaggctgtctttacgtgtttctaggactcttggcaaatt |
| | | ctcagaacagttgacttaaaaagattagccacgtagtatttttacaagga |
| | | gagagggaggtgaagtgatgagatggggccatacttataaaatgattcgg |
| | | gattattgatgaagtagtccctaggtttctgattaattaattttaaagcg |
| | | ctgaccaaatatgtacccagcgtaagaagccagagtcgatcagtgttgta |
| | | gacacgtcgcctctcgatggcatctttccatgccacacccttcacgacct |
| | | gaagctggagcttaaacccttcaagaagggtccctgcgaccgggtgcagt |
| | | ggctcacacctgtcatctttgggaggccaaggtgggcagatcactaggag |
| | | ttctaggccagtctggccgacatggagaaaccctgtctctactaaaaata |
| | | caaaaattagccaggcgtggtggtggacacctgcctgctcacaggctgag |
| | | tcaggagaattgcttgaacccaggaggtggaggctgcagtgagctgagat |
| | | cgcaccattgcactccatcctgggtgagagagtgagactccatctcaatt |
| | | aaaaaaaaaaaaaagaagggaccctgctttgttcatgattttattctcgg |
| | | tgtgtggcatacactaggcctgaaataaatgttatttaaatgttaaatta |
| | | cattgtccctccttccgatacatttgccatctggttaaattccagtgaag |
| | | tcaaaggatggatttctttaaataaagtttgcttctttttcaaaaattcc |
| | | ttgggctctttctagaaaagctgctcctttgccatcttcccagcagattc |
| | | ccggggcaggatccacccacctgttcacagctccttccagcgcagtgtgg |
| | | gtccggctgttagctcagcctcaaaggagaattgtaaagatacaggtcaa |
| | | gtgtccttttgaccaccttttttttttttttctatatgcgggattttgtg |
| | | agacaataaaatgcatgtggacctcagcttaacaaaagctgaatgttcct |
| | | ggggtgagtgtttgaatccaacaggaaatagatgctcaacagctctgagt |
| | | ctggggaggttttaatggcctcttggtccctggtagggtcttgtttttcg |
| | | gggtagttttgcagcctctgacacaggtaagatggtcagtaccggaagct |
| | | gccatgacgtctagactgaccgttaggaaggtactcactggactggtatt |
| | | cacagatggaaataaaccaaagcagtgtctagggtcaaccaatctgcttt |
| | | gctgccttcacggggaggtcctgggaccctttcatggaggaactttttaa |
| | | tgcttattttaaccctgaaggtaaaacatgaaacatgcagccattactcc |
| | | aagaaaacgtaacgtgggtgagtggtgtgtctcgggggcccacggtctcc |
| | | ctgaggccgcatctctctggagagatcgtggacgtccgtcgtaaactatg |
| | | acctattcacattttcactttactctcaactttccaggagtgagtcctct |
| | | ccagagagcaagacggagctgcgtttcaacagacggtgctttcaatctgt |
| | | gctgactgtaacttttatccatcatagGTTTGAAGACATACCTGATATCG |
| | | GGTCAGAGAGCCAAGGAGTCTCGCTGCAGCTGTGCAGAGGCCTTGCTTTC |
| | | TGGCTTTGAGGTCATTGACGGCTCACAGGTGTCCTCAGGCCCTAGGGGAC |
| | | AGGGGACAGCGTCATCAGGGAATGTCAGTGACTTGGCGCAGACTGTCAAA |
| | | ACCTTTGATAACCTTAAGgttagtttttttgttttttttttcctacttct |
| | | aaaactttctattaattctgcagtagagcacgtgaaataataagcagcct |
| | | ccaggtctgaggcaaacaacttggaatgaccttcgatttggttttcacga |
| | | ttttaatatttagtctaaagttagtagcattctgcatttttaacatcctt |
| | | gctttttgacatatgacgcacctccattgcctttccattgcacaaggatg |
| | | cttctcatttgaattattagttattttctcttcctgggacctgtggtgcc |
| | | ttttgggatttctagaagctggtgataaatgacagtaaaggtggctttcc |
| | | ttggcaccttgggtggagagctagttgataatctgatttcagagaccact |
| | | ttctcaaatacttctacgtctcttgaaatccactattttatgattacttg |
| | | aaatgatgttggtgtgggtgaatcctaggacacagctcactgagaggaca |
| | | ggctgccgagaacattgtagtatttcattattggaaccgggatctcctgg |
| | | tagctgcctagaagtgcccgtttctggactgacctgtcatcataaactac |
| | | cagggaattggcgcggtcgaccttgggtgtgtgaaatcatttcagtttat |
| | | gagctgttcctttgctttcacatttgatttagtttccccactggtacctt |
| | | ctgtttatttttatagtttaaacatctactgtctccattcttaaaatgag |
| | | acagagaatcaaaacagctctcctatgctgctttgtgttctcagataaca |
| | | cagtggggtatgtgtgaggatggcggccagtggttacagcttcacccaat |
| | | acgtccctgcctctccacattcattcacaggtttgaaagccgctattggt |
| | | atttacgaggaacagtcttcattatgtggcagtcagctggggtatgcaga |
| | | ttacttactgggtcagaatacatagtgcctgtcgcccagttacacgtaat |
| | | gattgcactcgggttctgtcaactccgctcgtggatcgcggtcgttgggt |
| | | gctttgttagtgtaatgcagggcttctcaccttggcaccgctggcatttt |
| | | ggaccaggtagtcatgtgttgtggggctgtcctgtgcactgtcagatggc |
| | | tggcagcatccctagccgccacctgccagatggcagcatacccacccgta |
| | | ccctactccccagctgtgccaaccaaaaatgtctccagacatggcccagt |
| | | gtcccctgggggtgcagagtcacccctgggtgaaaaccaccgggacagtg |
| | | gtgttggaccctctcttccctgtactcttggcacatgtgtaaagctcatg |
| | | tatctgagattccctggatgcctgcaaactttagagacagccgccttgcc |
| | | tgggctgacgttgtgcaggagtcagaaaatggagctctgggctcctagac |
| | | agcggacccgggcagggaggggctggcgtggggtggaatcgggaatggtt |
| | | ttgctatttggggaagaattgtttcctccagcctgcaaactgagctgtat |
| | | ttaagtcgtgaaagacagccccgagccaaaaatatataattcgttgtgtt |
| | | tgccattttagcttccacatatgtgtgtttctgtccgttaggggatgcgg |
| | | tcgcagagttcaccatatcacatgctcacgggtttagtgaccacggtttt |
| | | agcaatggcgattctacactacattgcactctagtcatatatatttgttt |
| | | tggtcggaatgagcaaaaaccatttagcgtatcttcttggcatgtggaga |
| | | ttttccaaatattgttgaaagtttctgagaatcttgtgatctatcaagtt |
| | | gacagaaggccgggcatggtggctcacgcctgtaatcctggtgctgtggg |
| | | cggccgaggtgggagggatgcctgaggctaggatttccagaccagcctgg |
| | | tcatcgtagcaagacatcatctctacaaaaaaattttaaaaattaactgg |
| | | gcttggtggcctgtgcctgcggtcccagctacttgggaggctgagccaag |
| | | aggatcacttgagcccaggaatttaggctgcatgagctatgattttgcca |
| | | ctgcactccagcttaggccacagagtgagaccttgtcctgtctccaaaaa |
| | | aagggaggagggtgctcagtggtaaagtaaattcgggaagcttgggatat |
| | | tcaacacagaaagggtttctttactgcaggacttctcggagccttcacta |
| | | ggctagtagacagtagctaggccttcagaggaggacgcggtatatacagc |
| | | attctcaggcctctttgagcacaattgttttttttcaagctgcaccttgc |
| | | agggctaatgtggtcacaccttttgggggaatgctcatgtagtaaaagga |
| | | gtttcatgccaaggaccaatacattagagaaaaggtgttcaggaaacgtt |
| | | ccctgaaggagcatgcgtagattttcacgtgtgtcctgttcctgctgctg |
| | | ctcacttgagatactcatggcttctctttcccctgaactcctgtctgcat |
| | | tcaccgttcagctccaccacgccttcccgtgcctccgcagctgataacgc |
| | | tgttccctcgtctccaaattctcagtcccttcagtctgtcccacttggct |
| | | gtaacatacacgatcttaaatctttgtttcttatgcgtcttcctgggagc |
| | | tctttgcattcagagactgagtgttaaataacttaaatatttcccaagtg |
| | | tcgagcatggtgcctggcatgtagtaattaacagtttctttttggctttt |
| | | taattctttaattaaacacatggagcgatgcagctctgcagagccaggcg |
| | | aaggactgaagtcagaagcccgagcagaactgtgtagaaccagggcagcg |
| | | cccacgaagggtttatttcctgcccttagcatgtgtgtcctctgtgacct |
| | | taagtcatctcctaggtcatagctggaaaattcttatctaatgtaacata |
| | | acctgtgtactgtaacgacctaccaggaaatgatagtgatgtcaaaggcc |
| | | agggctttggtcctgaagtcatagctcaaagggaaccctttctacaccca |
| | | agtgttggcttcttttttttttttttttttttttaaagagatgggatctc |
| | | gctctgttgcccaggctagagtaccagtggtatgatcttagctcactgca |
| | | gctcgaaactctcaggctcaagtgatcctcccaagtcaacctgctgagta |
| | | gctaggaccacaggcatgtgccactatgcctagctaatttttaaattttt |
| | | ttttttttgctatattgcccaggatggttgccaactcctggggttatacc |
| | | atcctcctgccttggcctcttaaaatgctgggattacagtcatgagccac |
| | | tgtgcctggccttgggttcttgaaatgaccagcagtgagaggtcctcatc |
| | | ctaagggccctgacattgcataatgtgggggacagatactgcccgagtga |
| | | aggaaggagaggtcaagaaggaaaccaaggagtaagcattagacatgagg |
| | | acgccactgtcagcgtcgcggaggggaggcggaccacggcgggcaggggc |
| | | gcaacttgaagcaagacccttctcaagccttggctttgcgactgtgaaga |
| | | aactgcagccaggtgcagtggctcatgcctgtagtcctagccactcagac |
| | | agctgaggcaggaagatcgcttgagcccaggagttagaggctgcagtgag |
| | | ctgtgatgatgccactgcactccaaccagggtgacagaatgagacactgt |
| | | ctcaaaaaaaaaaaaaaaaaaagaaaagaaagagcaaacacacacttaat |
| | | tcctctaagtgagagttttcctcttcagtgcggtggtgataaggaagaga |
| | | cctctcgggattgctgggagatgagccggaatggtgcctgtggacgccag |
| | | ggctgagaagaagcctcgctcacactcggcacgcacgccttcactccacg |
| | | cagagcagggacagtgaccacagacggagccatggaggactgggaggggc |
| | | cgagagacggtcccagcttgcctgcaggaagtcactgactctctggggaa |
| | | ggtcccatcttctccacggttccgattcgagggagattctcacagtgatt |
| | | gcggctgagcattgctgtgggttggatgctctcggccgtaaccaagtcac |
| | | ttgtatgacacgggagcatgtgtgccttggcctgttgaatgtgacgtcag |
| | | cgctccagcaggcatttgctttccattttctcatgaaccctgtgggtgat |
| | | gagtggatctagatgccatatatcaataaaaacagcttttccaacatctt |
| | | catgaagaaaaagaaaacccaactgggctcagtggctcatgcctgtaatc |
| | | ccaacactttgggaggccgaggcagagagatcacctgcggtcaggagttc |
| | | aaggccagcctggccaacatggtgaaaccccatctctactaaaaatacaa |
| | | aaattagccaggtgtggtggcatatgcctgtagtcctaagccactcagga |
| | | ggctgaggcaggaggatcccttgaacctagtaggtgaaggttgcagtgag |
| | | ctgagaccacgccactgcacttcagactgggcaatagagtgagactccat |
| | | ctcgaaaagaaaagaaaacccaaaattgaattacttgtatcacattactt |
| | | ctgatcagggaaccatttgagctgtgaaagcatcttagtacatctctgaa |
| | | aggcctccctggagcgcacctgcgtggctggtcttcctgagtttaccttc |
| | | atctattattcagaaccccgggctgcttggccactgggtgtttctacatg |
| | | gaccagtcagtaatgaagctagtgatgtagatttacccatacggggatga |
| | | ttcagagaggacctgtcctgctttcaaaaggtttctccttcccctttaag |
| | | gccactcacctcaggaaccatccttttgtgacagtgaattttgaaaatga |
| | | ccctaaatgataacttcttttatctacttgttttctatgtgtcaagtcat |
| | | tatgagtaggctcagttggcccaatttgaggaagtaaggggcttttcatt |
| | | cgttatagaaattgaaaaaatgatttcatattttatagacacatacatat |
| | | ataaatatatatagcttagaaattaaataggtatttattataagtggtag |
| | | tctgaattgatagtggtgatgcttgcacatccctgtgaatatgctataaa |
| | | ccctggagttatatactttaaatgggtgcattatatgctttgcaaatgac |
| | | atcttaagaaagctgttgtaaaactaaagggtagtctccgccaggtgagg |
| | | tggtcacacctgtaattccagcactttgggaggccaaggcagatggatca |
| | | cctgaggtcaagagttcgagaccagcctggccaacatggtgaaaccccat |
| | | gtctactaaaaatttaaaaattagccgggtgtggtggctggtgcctgtag |
| | | tcccagctacttgggagactgaggcaggagaatctcttgaacctggaggc |
| | | ggaggttgcagtgagctgagatcatgtcactgccctctagcctgggcaac |
| | | agagtgagattccatctcaaaaaaaaaaaacaataaaaaaaaaaaaccat |
| | | ctctacaaaaactataaaagttagccaggcatgttggtatccacctgtaa |
| | | tcccagctacctgggaggctgaggtgggaggatcgcttgagtctgggagg |
| | | cagaggctgcagtgagccaagattgcaccattgcactctagcctgggcac |
| | | tagaatgagaccccatctcaaaaaagaaaggtggggggcggggtaggaat |
| | | aggaacttcacagtggacaaaccgggcagatgccaccttcaccaaatgat |
| | | ccaggtgagcctccctgctcactgccatccggtaccctagtgtggtgtgc |
| | | tgagaagggcatgtcacctctgcggcattctttccgcaaaccccaaacct |
| | | cagtctgctctgagaaagcaccaggcaggtccagatctggacatttgaca |
| | | aaatgcctggccgggacttcaaacgcagcagggccagaagagacagagaa |
| | | agacagaggaacccccataggctgggggtgagcagggggcatgagggcta |
| | | aatgcaacacgggacctagaggagacccgaacagcaaagggacggtgcgg |
| | | aaactggggacatcagaataaagccagcagcttagttaacggtatcgcgc |
| | | cagcatgagttgctttggttttgataactgtacggtgagtatgtacggag |
| | | gatatatgcagagtccttgaactgtctttcccactctctgcaagtctaaa |
| | | attatttcaaagtaagaaattatatatgtgtgtgtgtgtgtgtgtgtgtg |
| | | tgtgtgtgtgtgtgtgtagagagagggatggaaatgtatgtgcggaatca |
| | | ttgaactggcattgccagagagtgaaatattgcatttccatgaaatttcc |
| | | ccagatgccaaatattttcttatcttgacttcatgatggactgtttccac |
| | | agctggtaacagtcttcctgcctgacagtctgctgagtatattttcatct |
| | | attcttgactcggatcgccatcaccagcctggttattgagcccagtggtc |
| | | ctttagggctaagggattgagtgagaggtccagggacttttcgactccgc |
| | | cccgtgtggctctgagattgtgtgtgtgagttttacacgtgtgtgttcat |
| | | gtggtgacttgtctagtattctcaggccatctgctgtctgttcttactgc |
| | | tttggggatgtaagaaaatttctttttctgtgataattactgcttatctt |
| | | ttcttctaatattctgcttctatttttctctaatgttgcattagcagaaa |
| | | tactctattttgtacttatgcccgtacctaacctgttagcttaaacagca |
| | | atgccagggacatagagttagccccatggctctgccgcactgacaggctg |
| | | ccccgttcacctggagatcgttggggccaggagctctgctgcctgggctc |
| | | accgcctctttacgcggttccgaccgtaccgtgtggctctgggccagtgc |
| | | ttgctctgtctcagtgtcctcatctgtaaaatggaaccgatcatagtacc |
| | | tgcatcgtggttgcttacgagggttaagcagatacaaggagtttcaaaga |
| | | acacctcaggaaattgtgtttgtggttattgtggttgcaatatcatgact |
| | | attcctgctcaccatgttaacatcattttgagtttgggagcaaaggatgc |
| | | ttctgataacacgacatcttgcaaaacagtcgtgttgagaagacgaagca |
| | | ggcaaagccagggctcttctactggtgtatcctcctcatttctgagaaaa |
| | | gaggtttccccactccctcccttgacagttttgaaaagggacagctgttg |
| | | gcttagaaatgatttatcttgtgatgagaaataaccttgaagaaaaaacg |
| | | attttatagctttgtgccatatattttgaccggaatgtagggtgcacgtc |
| | | ctgaaatgtgttaaaaacaaatgcaaatgtgcccaagtgtgttagagaaa |
| | | tggctacactcttccctccaaagcccacgtccacatgggggaaggagcaa |
| | | agataatccggaaaaatgcagagtttgctcttgggcgttctccagggaat |
| | | ctcaggttacccaggcaacagccagcaaactccctggcccgccaggcgtg |
| | | ctgctgggaggaccttagaagtcttgagacttcccttgatgccggagggc |
| | | ccagggtgctgcttggaagaaccaggaggacaggcatagctgctcaggtc |
| | | actctagggaaacaaggcccaggagagcaaggtgccaggattattcctgg |
| | | gtcctccttgtgccaggaaagggggccagagccatatgacggtggtccgg |
| | | gtcccagggccctggagtctgtggccgtctgcggaatgatcattgctgcc |
| | | tcacagtgtggctgcaaaggacgagatcatacagtcgagactgcagatat |
| | | tgcgtggatgggggcccgacttgttcacgggtaggatcttggcccataga |
| | | cattcctaagtcatagtagctactgtcattatgacactgctttttctcta |
| | | atgtgcctgcccgtgtggctccctgtgagccagctgctagggttgtctcc |
| | | caaggcagtgaggtgcttccatgcctgcagcttcctggagtcagaaatac |
| | | tcttcccagggagggcttctcagcaggggtggagatgcctgcagctgtgc |
| | | agggggagacgctgtttagccggctgggaaacatttcacagaggcatctg |
| | | tacttggcaaaaaacatgttgtgaaatgtcttaatccagattaaaattaa |
| | | gctaatgaatcgctcaaggaaaattgccatgggggttggggggtaggagg |
| | | tcaggggagggaaacaaacccacccaccattgcatggtgggaccctcctg |
| | | gtacagaaggggcctctaactacagctcagaaggggatgttgagatgtga |
| | | gaatctctccattgggtagatggatttctctttttttcttttcttttctt |
| | | ttcttttcttttcttttcttttcttttcttttctttctttctttcttttc |
| | | tttttttttttttttttttttttttttgagacagagttttgctcttttgc |
| | | ccgggctggagtgcagtggcaccatctcggctcactggaacctccacctc |
| | | ccgggttcaagagattcccctgcctcagcctccctagtagctgaaactac |
| | | aggtgtgcaccaccacgccaggctaattttttgtattttagtagagacgg |
| | | gctttcaccatcttggccaggatggtctcaatctcctgaccttgtgatcc |
| | | acccatgttggcctcccaaagtgctgggattacaggcatgagccactgcg |
| | | cctggcttcttttttcttttttaaagagacaaggtcttgctctatgaccc |
| | | agtctgcagtacagtggcatgaccatagctcatggcagcctctccctcct |
| | | gggctcaagcgaccctcccacttcagcctcctgagtagctgggactacaa |
| | | gcacacaccactgtgcccagctaatttctaaaatttttaaaaagcttttg |
| | | tagagatggggtcttgatacattgcccaggttgatctcaaactcctgggc |
| | | tcaagcaagccaccacacccagctctaagatttcttttgtttcatttgtt |
| | | tgaaagtttttatctagaaaattatccctcaaaaggaaggtagctgagga |
| | | atcttgctctgtcgcccaggctggagtgcagtggcacaatctgggctcac |
| | | tgcaacctccgcctcccgggtccaagcgactctcctgcctcagtctcccc |
| | | agtagctgggattacaggtgtgtgccaccacacccagctaatttttgtat |
| | | ttttaatagagacagggttttgccatgttgaccaggctggtcttaaactc |
| | | ctgacctcaggtgatccacccgcctcagcctcccaaagggctgggattac |
| | | agggatgagccactgcgcccagccaaaatccgcattttcaaagtgcagta |
| | | aaatgatggctgctgggtccgggtgacacaggtctgtgggaccatgccac |
| | | ccagcgcggcctggagtctgcacctggctctccccccacctcccatactc |
| | | ccacttcccggacaaacctgctgggaatggaataggcagtgagcactgtc |
| | | ccggccctcaggaggcccgagcgtgggtgtctacagctggccatgtgacc |
| | | cagacagcccccttccttctgcaagcgcagcctcatttgccatttgtggg |
| | | cggctctgccagacctcacttctgtatcagaacttcttatcactgcaggg |
| | | tgcaggcctcggcctctgttcaaggggatgtaagcaaagcactgagttcc |
| | | ccacctgtggagaccactgccccaggcccccagagctcagcctcatccca |
| | | gtgggcatgcccgggctgcaccggccttccagcccctctgagggaagccc |
| | | ttgtggagggcatggagccccctgtcctcagtgtgccctctggtgccaag |
| | | ttggtttgactgtcacggtgacacattcatggctctaaatgcttagaaaa |
| | | tctcataaagccagtgtgccgtggggggcactgaagaagcaggcggcctc |
| | | gtcttaaagcacaggagggttggccgggcacggtggctcacgcctgtaat |
| | | cccagcactttgggagggcaaggcaggcagatcacttgaggccaggagtt |
| | | cgagaccagcctgaccaacgtggtgaaaccccatctctactgaaaataca |
| | | aaaataagccaggcgtggtggtgggcgcctgtaatcccagctactcagga |
| | | ggttgaggcagcagaattgcttgaacccaggaggtggaggttgcagtgag |
| | | ccaagatctccccactgcactccagcctgggtgacagagcgagactcagt |
| | | cccaaaaaaacaaacaaacaaaaaaagagagaaaagcacagcagatgaac |
| | | ttgaggcaagaaaaatacctgaaacagactgacgccaggagattcctgtg |
| | | actaaatggctacagaagaatagataccaagggctgttctgtctgtaatc |
| | | aaggacattgaattaccagatgaactttaatgaccagagagcatagttaa |
| | | cagaagttttcatattcactaccagcatttactgagcacgcacttgcact |
| | | tcgggctttggctgagccaagtgcagcagacgcttccacacctccagctt |
| | | cctttcctggtctaccggttgggccctgaaatggctggtgtgccccatca |
| | | agcaccaagccccccaccttagccacaccagagagttccctcaggcatct |
| | | tttaggcatgggtatcaggcacacctattttattcttctcttggtgttag |
| | | atttttttacatttaataagagcagtggtctcattggcaaatatatacat |
| | | atttatacatatatatatgtattttgagaatgggtttgctctgtcaccca |
| | | cgctggagtgcagtggcacaatcacagtgcactgcagccttgacttcctt |
| | | cgctgaagcaatcctcctgcctcagcctcccgagtggctgggaccacagg |
| | | cacgtgccatcaaggtcagctaattggttttatttttagtagagacaagg |
| | | tctccctatgttgcccaggctggtcttgaactcctggccctaagcagtcc |
| | | tcctgcttcggcctcccaaagttctgggattacaggtgtgagccagcaca |
| | | ccagcaaaatgtttagaagtttaactctgccagtcaataccaagaggtgc |
| | | tttcaaatagactcactgtatgttattgataactagaacattttacctct |
| | | gttgataatgtcagtttttaaagagctgtaagcaaatgttatctagatgc |
| | | cattatttgctcagatatgttaattaatgatttcgctagctgttttggca |
| | | tgtatattttcattttttgaatgcatacaaatgttcaaatttagcttgta |
| | | tgttacatcagattcccaaactatacaaccctgtagaatctttgtttctt |
| | | ttatttaatatagtacaggtctgcagtctaactttaaatacaacctgggt |
| | | catatctttgcccttaggtagtgacacttactttaaatttccaaaataat |
| | | atctggcctcccataatttatttcaatgttttacacttcttgctgcaagg |
| | | aaattgcttttgagtccaaaaaaaaaaaaaaatatatttcttgccacggt |
| | | ttgtcttcttcattggaagctgttagttgctttcttagatggttcctagg |
| | | ttacctggtcacattaataataataataagtttattacgggtatactgta |
| | | tgccaggaactgtgctaggctatatatatacttatttcatttaattatta |
| | | tttttttaagacagggtctttctcttttgccccaggctggagtgcagtgg |
| | | caccatcaaagctcactgcggccttgacctcccagactcaagcagccctc |
| | | ccacctcagccccacaagtagctgggactacaggcacatgccaccaggcc |
| | | aggctaatttttatgctttttgttgaggcagggttttgccatgttgccca |
| | | ggctggcctcaaattcctggactcaagccatcctcccaccccagcttccc |
| | | aaagtgctgggattacagccgccacacccagccaatttcatttaattctt |
| | | agcaacatatcttcactctttgtttatagatgagggaactgaggtaaagt |
| | | gatttgaacagagtctccaaactagtaagtacttgactagggtttgaatc |
| | | ctagtttatgagaaataaaggctttgctctttttagggtacctgtattaa |
| | | atcttgccttctcttttctaggtccattaattcctataaacttttaagta |
| | | cgcctagatctggaaatcgtgtacctttgattattcagcccatcttatga |
| | | gactagctcaccatcacacggagcttaagaaccttggatttgtgtcctcg |
| | | catagttggtggccagtttctttacttaataacagctttgttgagatata |
| | | attcacatactataaaatcgatctttttggccaggcacagtggctcatgc |
| | | ttgtaatcccagcactttgggaggccaagacgggtggatcacctgaggtc |
| | | aggagttcaagaccagcctggccaacatggcgaaaccccgtttctactaa |
| | | aaacacaaaacaagtagccgggcgtggtggcaggcacctgtaatcgcagc |
| | | tactcaggaggctgaggcaggagaatcgcttgaacccaggaggtggacgt |
| | | tgcagtgagccaagatcgtgacactgtactccagcctgggtgacaagagt |
| | | gaaactccatctcaaaaaatatttaaaaatcgatctttttaaagtgtaca |
| | | gttcagtggtttttagtatatttagcaaggttgtgcaaccatcaccgtga |
| | | tcaatttcagaacatttttatcatcccaaagagaaaccctgttgaaatac |
| | | cacaaaccaggtggcttttaaacaacagaaatgtatttctcagggttctg |
| | | gaggctgggaagtccgagatcaaggtcccggaagattcagtgtccagtga |
| | | ggacccgttcctgcttcacagatggtgccttctcccagggcgaagggacg |
| | | agggattcctctggggcctcttttatgagggcactaatcctattttgaag |
| | | gcttgtcccccatgacctaattacctcccaaggctttgccccccaatgcc |
| | | atccccttgggaattaggatttcagcgtctgacttttgagggggcacaaa |
| | | cattcgaatcgtagcaaaccccacgccccttagcagccactcccccttcc |
| | | tgcccccggtcccgcctggcagccaggaatctgccttctccctccaagga |
| | | ttcaccttttctgaatatttcacatagacgggatcacgtaatatctgtgc |
| | | tgtgtctggcttccctccccgagcataatgttttcgaggttcaccatgct |
| | | gtggcaggcgtcagtgcttccgtctttttcgtggctgagtaatatcccat |
| | | tgttcgaatagaccacagtttgttgatccattgttcagtcagtgaacatt |
| | | cccggttccatttttgggtcctgttgcgttcgtgcccgggtttctgtgtg |
| | | tttccactgtcactttgcccgttggctttcagctcatccccgagcggttc |
| | | tgtgtgccttccccttgctcaccaccatgctgcgtgacggcagatggcaa |
| | | cagcggacctgcctgaattcagggtggcagtcgcggtttctgggtgtgtc |
| | | cccacagtctgtgtttttcttctagACCTGCCCTTCGTGCGGAATCACAT |
| | | TTGCTCCCAAATCTGAAGCCGGCGCCGAGGGAGGAGCACCTCAAAACGGC |
| | | TGCCAAGACGAGCATAAAAACAGCACCAAGgtaacaagctcatggctgtt |
| | | gttcctgtcagttcctctcctggcgcatccacccaaggtgacattcctgt |
| | | cttgtcgacttcttagttctgagagtaactcctcttgggtaacagcactc |
| | | cgcacccgcgggtcctccgggtgttgaagagaccggcagggagctcgagg |
| | | gagggtcagcagcaacccacgtggaaaccagcgctcaggggctgagctgg |
| | | gcgggattcctttaaaggccaataagggtttaaaatgcgtggattcgagt |
| | | gccgggaggggatttgtggtactggaagctggactcagtttatgagagaa |
| | | tatagacgggcagagacaagagggggtcttcacggcgagaaaggctgggt |
| | | gaagagagaggttgggagttagggccggtggcaggaaggctttcaggaaa |
| | | accagacttggacaccagggccaggactcagcccacccgcgagagcctgt |
| | | cggcgggggctcatgcaggcgactcacctggggcgcgggctctcgtcctc |
| | | catctgctctccatctgccaaaggattgcaggtgcaggtcatcccatgcc |
| | | ttcccacagccacgggggacacccacgacacgagcccgtttccatccacc |
| | | ttgcctgtgccctgacagccctggacccctcgtcatacttctcctggacc |
| | | cccgacccatgcaccctgctcccagaaccgtggttctggcctaagcagac |
| | | tctgggggcctcggctcctcactttcctcccttcccctccacctccgcac |
| | | cgactcttcccatgtctcaatgtcctggcagcctgaacacccagcgttac |
| | | cactgggcactgttgggtaacaggaaggagcagacgggcagtcagggctg |
| | | ggattggagcaggtctgtaggcgcaggggggtgggggttcctcgcaggtc |
| | | agaaagtcccacgcccagtgcagcagcgccccgcagcggaggtcggggtg |
| | | ggacaggcgtgtgtgcccagtgcccggtgccacttcacttcccgtgtctg |
| | | cgtcctctttgtctcgaggcagGCTTCTGGAGGACCTAATCCCAAAACTC |
| | | AGAACGGGCTCCTCAGCCCTCCCCAAGAGGAGAAGCTCACCAACAGTCAG |
| | | ACTTCTCTGTGTGAGATCTTGCAGGAGAAGGGAAGGTGGGCAGGGGTGAG |
| | | CCTGGACCAGTCGGCTCTCCTTCCGCTGAGGTTCAAGAACATCCGGGAGA |
| | | AAACGGACGCCCACTTTGTGGACGTTATCAAAGAAGACAGgtacgactgg |
| | | ggctgtctgggaaaaagcaaaggttgcaggcagaagcctctctgcctttt |
| | | tcttttcttttctttaagactgagtctcgctctgtcatccaggctgcagt |
| | | gcagtggcgcgatcttggctcactgcagcctctatctcccaggttcaaac |
| | | gatgctcctgcctcagcctcccaagtagctggggttactggcacccacca |
| | | ccatgcctggctaggttttgtatttttagtagagatggagtttcatcatg |
| | | ttggccaggctggtctccatactcctgacctcgggtgatccacctacccc |
| | | accctcccagagtgctgggattataggcgtgagccaccacgcctggcttt |
| | | ttttttttttttttttttttttataaggactaggtcttgctgtgttgccc |
| | | aggctggagtgcagtggctcaatctcggctcactgcaacctccacctccc |
| | | aggttcaagtaattctcctgcctaggtctctcaagtagctgggactacag |
| | | gcatacacaaccatgcccagctaatttttgtgtttttagtagagatgggt |
| | | ttttaacatgttggccaggcgggtctcgaactcttggcctcaggtgatct |
| | | gcccgcctcggcctcctaaagtgctgggattacaggtgtgagcctgtgcc |
| | | tggcctaatggtaacatttttgaaaaatacaggtccagtccttttatcaa |
| | | atattcctgaagttggattggttagattcaggctctccctctggccaggc |
| | | agagactgcagaagggatgccgtgtcctcctcagggtgtcctctccaggg |
| | | acacgcagtgcccgtctgttcctcattagtgatactaattttgattaatt |
| | | aggcaaggtggtgccgggttgtctcatctagggttattatattcctctta |
| | | tgactaataagcaatctgtgagaaaatacttgaagatcttgtacatattt |
| | | cattccccatcagaattccctagatttaacattcattgatgattcttgcc |
| | | tctgtcttcactataatgcctcaaagggtgattttcagccaggcgctgtg |
| | | gctcacgcctataatcccaacactttgggaggccgaggcaggtagatcac |
| | | ctgaagtcaggggttcaagaccagcctggccaacaaggtgaaaccccgtc |
| | | tctactaaaagtacaaaaattagctgggcgtggtggcgggtgcctgtcat |
| | | cccagctactcaggaggctgaggtaggagaatcgcttgaacccaggaggc |
| | | agaggctgcaatgagctgagatcgcagcactgcaactccagcctgggcaa |
| | | cagagcgagtctccatctcaaaaaacaaataagaaataaatggagcagca |
| | | cagctaatagcagtgtcgcttctgccacttgactgctaaaatgtggcctt |
| | | gtgcacatcctctctgcagctgcgtggttgctcttccctcagtccacact |
| | | ccctgagtcaggggaggagagagggtcagtgtcctgggacagaattattt |
| | | ctcttctgagtctgagaatgttggaagttactggatacctgtcacagaaa |
| | | ggagagtgtccgccgtgctgcaaggacaggaaggaacacacgtgatgagc |
| | | cgtactttgtgtggggggcacatgaggcacttgacatatttcacctgatt |
| | | tactctttttttttttttttgagacatagtcttgctgtgtcacccaggct |
| | | gtaatgcagtggcgccatcttggctcattgcaacctccatctcccaggtt |
| | | caagtgattctcctgccttagccacccgagtagctgggattacaggcacc |
| | | tgccaccaggcctggctaatttttgtattttttgtagaaacgggggttca |
| | | ccatgttggccaggctggtcccgagctcctgacctcaggtgatccgcttg |
| | | cctcggcctcccaaaggtctggggattacaaggatgagccaccgcgccca |
| | | gcctcgtctaatttacttctgatagtcactgtatgagggagagaggctaa |
| | | cacagctatatttgctttctgaaatcatgttaacgttcccttagtttcta |
| | | gtaattttctgcagtatagaaaggatattactatttgctggcaattcata |
| | | aatgaatgaaagagtcatggaaagcggggttgatttccctgctattaaaa |
| | | tcactgagctttatcattctgcaggtttcacatgcaatctgcatatgtga |
| | | caatagcaccaaaattacgctaactataaacacaattttgtgataacaac |
| | | ctgttagcttttacggtggcaattactgtcagtgctctgaatgacaggac |
| | | cgccggttctgaattatgttttcgtttgaagcctccaaatccatgtaagt |
| | | actctcagcacagtgatctgcgtgttcttcacaaaagattgtttcaaaga |
| | | acagaaaagctgccttcatcttctcctaatcctagacattaggaatgtgt |
| | | tttcaaaaacaagcctcagcagctcacgctgtcatcccagcactttggga |
| | | ggccgagatgggtggatctcttgagcccaggagttcgagaccagcctgac |
| | | caacatggtgaaaccccgcgtctgctaaaaaatacaaaaattagctgggt |
| | | gcggtggcaggcgcctgtaatcccagctactcaggaggctgaggcaggag |
| | | aattgcttgaacctgggaggcggagattgcagtgagccaagatcgctgcg |
| | | ccattgcactccagcctgggcgacagagtgagaccttgcctcaaaaaaaa |
| | | aaaaccccaaaaaaccaaggttctgatgatgtgtgctataatcatggtgg |
| | | atggtcattctttaacgaaggtgaaacacaaaatatgattatgctttgag |
| | | gtgcgaaggactttccacagatctgttttaacaaggggcagctgagtggc |
| | | gtctgttccacaggcgcttttagacgcagctgttgtaatatacctaatga |
| | | gcagcgttttggtagctaattatgatctgaaaacattggcacagttatgg |
| | | tgctgggtattgatcgactgcatttgttgtgtcttctagCCTGATGAAAG |
| | | ATTACTTTTTTAAGCCGCCCATTAATCAGTTCAGCCTGAACTTCCTGGAT |
| | | CAGGAGCTGGAGCGATCCTACAGGACCAGCTATCAGGAAGAGgtgcgttc |
| | | atttctccttgcgcttttactaaagaatgtgtttgttgttctcacatggt |
| | | cattcactgttagttttgtgctttaccaagggatgggaacacgaaggcga |
| | | acattgatttcccacactctgtaaaccctaagtggcaactgtacatgaaa |
| | | tgtctctttgcaaaggaggaaaaaaaccctttctttagtcctttgtctta |
| | | aagcgcagcctcgccattactagcccctttcctctgccgtggcatctcca |
| | | ggctatccccagccttggaaggggcccccccaccacaacctgtcctgaaa |
| | | ggaaacccccaacctgccctggaaggaaacccccccacctgccttggaag |
| | | gggcccccacacctgccctggtaaaggaaccccccccacctgccatggaa |
| | | ggggtcccccacctgccctggaaaggaacccctccccacctgccctggaa |
| | | aggaaccccgacacctgccctggaagggaaccccccacacctgccctgga |
| | | agggaatcccccacacctgccctggaagggaaccccccacctgccctgga |
| | | agggaactccccaccgacctgccctggaagagcccccgtcccacctgtcc |
| | | tggagtcttgggtgagactcttctctgttgccctccctgctttcttcccc |
| | | tgagaagggcccaggaggccttcacctctctctctaactccggctccatg |
| | | ttccctctcccgcccccaccgccttaaccccttgtacttgacttctcagc |
| | | ccatcacccaatcccacttcctccaagaagctctccccgactgcagactg |
| | | gattgtacttcccttcatacacttccttctccgtgtgtgccccattttgg |
| | | aatctttccgtgtgtggttctggattcgtgttcctccctccctagatgct |
| | | cagctctgtgaggtaggacccccgtctctctcctccagcccctccttaca |
| | | gcctccagcacagtcccaacacccatctctctcctccagcccctcctcac |
| | | agcctccagcagagtcccagcgctcacaagtgctccttagggggttgctg |
| | | gaaggaataatacaagaacctagagcctggtttgaatcctggcggcttcc |
| | | agactgcattctctcctctgggcccaaacctaacctctgagtgttcccaa |
| | | gtcaagagtggatgccctccccacccggaacaaaagggatttctcactct |
| | | gattcccttggttcctccagcaggtcttcattaaactccgtggtgtcctg |
| | | gcttgggctctagagagaccctggtgagcccagtagagccagttcatgcc |
| | | tcgtggggtgggaggtgcccccttccgtcagcctccaagggacagtgaat |
| | | atccgttaatgtcccagacatggctcccggagtggacttgcaagttggca |
| | | gttggctggttggtggcccgtaaggcaaaatatcgtctcatttaatgctc |
| | | atagcaatgctgtagggaagctgatgattatcctccttttaccaaagagc |
| | | tgacgctgagcaaggctgaacaggtggctgtggtcccatagccggggtcg |
| | | gacacgagtggtctggctgccaaaccaggcgtccgggccgcttctcgccc |
| | | tgcactgctgagcctcccaaacagcagacacctttgccatgccttcctcc |
| | | aggcaccaaatgtacgctcagcctctgctctgccagggactgttcttggt |
| | | gctggggaagggggaaagacgtggcctgatggggagcaggaattagaaac |
| | | aacatccggagctggaggtctgcgagtgccctcccagcacacggcgaggc |
| | | accaagcccaggctgggggctccaaggggggaactcctgagcctgcctca |
| | | gtggggggactaggaattaatggaatgtgggatgagggaacattctggag |
| | | agtggggccagcacccacaaacgttagagataccaaagagcctggttcgt |
| | | ccagggagttgcaggcgcatccggggcagggtggggcaggagtggcagga |
| | | cgtggcaggggccaggtcacggctcccatgccatggactttatgccacag |
| | | gccatggggagctgttgaaggatctggaacaggttgtgttacaaagatca |
| | | cacagctcacacctgtaatcccaattactcaggaagctgaggcaggagga |
| | | tcacttgaggccagtagatagaccagcctgggcaacatagtgagaccctg |
| | | tctcttccaacaaaaagaagaaaaacatggccgggcgcggtggctcatgc |
| | | ctgtaatcccagcactttgggaggctgaggcgggcagatcacctgaggtc |
| | | aggagttcgagaccagcctggccaacatagtgaaaccccgtctctactaa |
| | | aaatacaaaacttagccaggtatggtggcgggcgcctgtaatcccagcta |
| | | cttgggaagctgagacaggaaaatcgcctgaacccgggaggcggaggttg |
| | | cagtgagccgagaccatgccgttgcactattgcactccggcctgggcaag |
| | | aagagtgaaactccgtctcaaaaaaagaaaaaaaacaaacatgtatcagc |
| | | tgagtttcggagggtgataagagtgtggacaagacaggaggccacatgac |
| | | aggtgggaggctgttaccgaaactcagggcaggtggccacagggtcccgg |
| | | tggggaggaaaggagagatgccaggacaagggagcctcccagctggcccg |
| | | cgtttggggactcattgtgctgggggctccacgtgggtgcgttcttagcc |
| | | gagcatcccaccgcccacaaggtgcaagtactggtttatccctggttttg |
| | | gataaagagactgaggcatggacgcgtgggtatcatagctaatcatggtg |
| | | gagtggccttgagcccagacagcccggctgcagagcctgtgctctgatga |
| | | tggtgggacggccgcacccggaggccctgggatgtgggtagtgagcaggg |
| | | cggagcctggcccacggtgccctggcagccagtggcactgtccctgggtt |
| | | tcgcccatggggagagagggcgaaagagtgtcatgatgtgggggagcgtc |
| | | gttctcagtgggactgggacaggtggcactcccgggatgcacaggggtgg |
| | | ggcagatgggcctgggagcctctggcatcagggccaggggtggcccagag |
| | | ggggaggctgtggcagggacaggtgtctctggcaagtagcagggcaggca |
| | | ggagagactgcaagggaggtggagggaggagcggggcctctggcagcgtc |
| | | catggatggggcagttctacagaggagatggcagccccaatcaggaaagg |
| | | ccctgtacccgagcccttggcttggaaggtgggaggtgtgggctctcaga |
| | | aggacggcagcccacgtggaaaccttccggcctcacatgtgggcaaggca |
| | | gtgatgtctcagggacatcacatgcatgcacccggccctgactcgaaccc |
| | | ctgacgggcactagatggagccccacagaaaggggacgtgcgtgggcctc |
| | | tgccaaggtgagtgggcatgggcagtgtcgcccctgaaattcctacacct |
| | | ggccgggtacagtggctcaggcctgtaatcccagcactttgggaggccga |
| | | ggcaggcagatcacgaggtcgagatagagaccatcctagccaacttggtg |
| | | aaatcccatctctactaatcatacaaaaattagctgggcgtggtggcggg |
| | | tgcctgtcctcccagctactccagaggctgaggcaggagaattgcttgaa |
| | | cctgggaggcggaggttgcagtgagccaagatcgcgccactgcactccag |
| | | cctggcgacagccagatcccatctcaaaaaaaaaaaaaaaaaaaaggaaa |
| | | agaaaagaaaaatgaaaaaaaagaaattcccacaccttctgaggggattc |
| | | cagggctgggacctgacttgcccttttgggcactacagagcctccagctt |
| | | ccttggagagaatttggaaccaaaccctaacccccaggttgccctggcag |
| | | ggtccagcctggcatcagggctgactggcagccttgctcccaccgtccaa |
| | | gtgcccctcctgccctcccacctagttcctgggagtggtcgaggcacccg |
| | | gagctggtttgtcccaaagaacgcacaccgcacccagggctgccaggaga |
| | | cctttccgctccgtgcgtctccagctcctgccctcagccccgagcctggc |
| | | cgggatgctgagtggacacccatggcctctcaggggttgccagaaggttg |
| | | caaggccaaatggaaagcagctgaactggctacatcccttcctctcccaa |
| | | ccacagcctctgcctttggatcctacaccttcctctaggttagggacaca |
| | | ctcaggagtcaaccggtccatgccaagtcctggcctggctccttgttcgc |
| | | tgtgtgccctaggggtagcccctggccttggcctcccaatctgtacaccg |
| | | agatgggtggtagttcctgcctgtggggtgccgtgagcgtcaatggagat |
| | | cgcacctccccagagccttgcagggcctgggcctggtcacctcttggacg |
| | | tgtcagcatcgttatttctccccctcaattgccgaacactagtgtgtgcc |
| | | tctctagtgtactggcccccagcacagagccaggccccgtaggttcccag |
| | | gcgatgatgtgccacttaaatccccgtcggccacttccgccacgaagccc |
| | | cgctttcgcacctgctgaacctaggactccgccatgcagcaggaccgccc |
| | | tgcacattgctctcgtccaactgccttacagatgcagggacctcacctgc |
| | | gccagtcagtaactcatcttgcagctaaatatataatgtgtctatgagaa |
| | | tgtgctgggtcataatcagttgctctgtgaccctggcagggttttttttg |
| | | tgtttttttgttttcggaaacggtataattctgcagtggtgtgatcatag |
| | | ttcactgcggcttcaaactcctgggctcaagcaatcctcccacctcagat |
| | | cccaagtacctgggactacaggcacaaaccactatgcccagctaattatt |
| | | ttattttttggtagacggggtctcgctatgttgcccaggctggagtgcag |
| | | tggcatgattatagctcactgcggcctgaaactcctgggctcacacaatc |
| | | ctcctgcgtcatcctcctgagtagctgggaccacactcacgcactgccat |
| | | gcctggctaattttaaagttttttgtagagatggagtctcactgtgttac |
| | | gctggccgggaactcctgggctccagcgaccctcctacgtcagcctccca |
| | | aagtgtcgggattacaggcgtgagccaccgtgcccagccccctgggcaag |
| | | tttaacttctctgtgccttggactccccagtgttaaatggggtagtagag |
| | | ggagacagccctccgagagccttcctgccctgtgcctggcccagagcagc |
| | | agtgctccgcacgtgttgcctgtttctccatctgaactgcgctcctggcc |
| | | gcccatggcctcccagtcattctgctccactcctgctccacacagGTCAT |
| | | AAAGAACTCCCCCGTGAAGACGTTTGCTAGTCCCACCTTCAGCTCCCTCC |
| | | TGGATGTGTTTCTGTCGACCACAGTGTTTCTGACGCTGTCCACCACCTGC |
| | | TTCCTGAAGTACGAGGCGGCCACCGTGCCTCCCCCGCCCGCCGCCCTGGC |
| | | GGTCTTCAGTGCAGCCCTGCTGCTGGAGGTGCTGTCCCTCGCGGTGTCCA |
| | | TCAGgtaagcgccgcgtggactgggtctccagctctgagccgttagctcc |
| | | ctcttctggctccatggcttatggctgagcggttggactggtccccagcc |
| | | agctgtccctgctggctgatttctggagatgggtccccgtgtcctccccg |
| | | agccctgtgccccagcatcctccctccctgacgttggcccagatgcgggg |
| | | ggaccacctcgctggagggggcggtgcctccacagccccaaccgctgcca |
| | | gcttgtccccagatttccaggttcagagactgcaaaggagaaggtgagaa |
| | | gataggaaacaatctggaggaaggaaaggaaaagacatgagcgggggtag |
| | | tgggcctgggacagcaggcgcctcttcagagtaccggagccaaacagcat |
| | | ctccacgcacttgcctcaggcagtggtctcttggatgtgacgccaacaga |
| | | agaaagaatagacaagcccaacttcctcagaagttaagctgttgcgctac |
| | | aaagaacaccatcaagaaagagaaagaacccacagaacgggagggaacac |
| | | ttgcagatcaggcctcggacaaggtctaacatctagaatggataaagagc |
| | | acacatagcctgataataaaaagacagctcattttaaaggggacagaggc |
| | | tccgaatagaccacaggtgcgtcttgggaaagctgggccctcgggaccag |
| | | cctggctccctgagggggctcgcagcggctctgtattcctgagcaggaag |
| | | gtggccctgtgtggtccccgtcctgggcaccacaagccccttgggtggtc |
| | | ctgtcaccctcaacaggatggctgccggcctagcagagtcccactagcaa |
| | | cggtgccccggagctgggcttcaaacagacatggggaggcagccctgacc |
| | | ttgagcgtctgctgcccttatccaacatcccagggcagaaatgaatggct |
| | | ttgagtctggcgtgaggcgccggaatgctggcttctcccatctggggtgg |
| | | gcaggctgggattcctgccccctccttcccacctgcactgtacccaggac |
| | | cctttcagcttcctccgaggccgccatgggccatgctgtctgtggcgtgt |
| | | gctcagtgtttcccagaactccctgtgggctcatgccccatcacccccag |
| | | cgagcagccctcttcctggcacagcctcccctcctgcctcagtgccctcc |
| | | tgtgcttctggggacccctcccaaaggagccgtcgcctgcaaaccctttc |
| | | agggtccacttccccagaacccaaaccagacatcctcaggcccctggttg |
| | | gtttgtttttcctctttaaaaagatcagctttaggctgggcatggtggct |
| | | catgcctgtaatcccagcactttgggaggccaaggcaggtggatcacctg |
| | | aggctgggagttcgagaccagcctggacagcatggtgaaacctcatctct |
| | | actaaaaaataccaaaaaaaaaaaaatagccattcttggttgtgggcacg |
| | | tgtaaccccagctactcgggaggctgaggcaggagaatcaacttgaaccc |
| | | aggaggtggagattgcagtgagctgagatcgcaccattgcactccattct |
| | | gggtgacgagcaaaactccatctcaaaaaagaaaaaaaagaagatctgct |
| | | ttattgtgacatgatttatatacaataaactcattcaagtggacaactca |
| | | gtgatttttagcaaatttgaagaattgtatgaatatcaccactatgtaac |
| | | tatcgggcacctccatcaccccaaaaagaaacccatcctcatgggcagtc |
| | | actcccccaggtctgccttccctggtccacccccaggttttcctctaatg |
| | | tgggtagctgtagcgtcttgagggctctgaggccagcattgtctccattc |
| | | ctttttactttcaccctttagctattattccctgctccttcagacccaat |
| | | tctaaagcattgagggcaaccccggtgtagactgccgagggctggggtga |
| | | ggtgcgctggaactgcctgcacagacatactccccaaggatctggtgaag |
| | | gtggtggctgctgcacccctgaacaggcctgtggtgggatttggttccat |
| | | ttaccaggaatcgtcaagcctgggagaggcccagaccctgaaaggcttcc |
| | | ctggcccctctgagggccgtgttgaggaccccaggttgacgggaacctgc |
| | | gctgtggggtatacgggcaccccatccctttcctgtacaggaggaaagcc |
| | | aggcgcctcctcagtgactggcccaggccccgcccgttgcctccaggagg |
| | | cttcctgcatcaccctctctgctctgccccagcaggcgtgaggtctcatt |
| | | ttcagatccctgcagtctcagggaccagcatgaaggccccctccaacctc |
| | | acatctttcccgctgctgactgggaacttggttccctctgtcccccctcc |
| | | tagggcctctgtattctgcatatttgtggttattcttggaagtattccaa |
| | | agtcttcattatcctttaaatatcaggaagccagggctgtccaggaccct |
| | | aaaagggtgaggctgtgcctgcgtccatgtggggaatggctggagagtga |
| | | gcgttccctcgcctggccacaccacgcagccaagagggagaggcatggta |
| | | ggtgttcaggcccgaaggctgggggcggggggctctgtgccagaggcttt |
| | | tttgctgttcagtcacaacagcggcccccgcccagcctctcctgaggcag |
| | | ggacagcaggcctatccccagctccctggcagcaagcttgagagaaggac |
| | | tgtctccccacgaggaccccacaccttctcttcacctccccatgtggccc |
| | | caccatggcccaggggtccttggactcagccaagacctgttcctcctcct |
| | | cacagtggttcttggcccgcacactcagctgcttctttatacagaaactt |
| | | ttattgtgtcaacatcacactcctgaaacgaagcccgctagacacacgcc |
| | | tgcaaaaggagtcagtgaatacagtcgctctgcttagaaggacaaagtct |
| | | tttaaaatgaaatacgtggtgctggacctcacgtacaacagagccccgct |
| | | gtctggagagtcctgaagccgccagctagaggtgaacatgcacgatgaac |
| | | atgacagcccagatgcagacggcacgcgtgtgcgggtttggtggtctaag |
| | | cgccacgagccaggttcagcctgtggtgtgattttctgaaacactgatga |
| | | tgatgccccaaacagcagagaccactgtcactcagtttccacaggagctg |
| | | cgctccaagaaaatccagggtgtgtcagcaccaccacagagcaccctgtt |
| | | tgtagctgggacacagcctctgggctaagatgctgtcagcagccttctgc |
| | | ctttgcaggtgcccaggggcttggctgctgtgtggggtgcagggcagaac |
| | | ctccctgggtggcacctgcccacatgcaggacagctctgtccctggcgcc |
| | | tgcccaccagatgccaggaccgcctcttctgctgcgacaactgcacgggc |
| | | tccacagaattccaggacgcctccaaagctgcccccgtcaagacctcctg |
| | | cctcagaagctgatgggagccggtgacggcaacgtggtggcccccaggag |
| | | actctcctcagggcctgtgatactctcacgtgttctgttaggcgagcgaa |
| | | gcatgtgcttttcacaagcatattttatcacaacgcaacatgtgacccta |
| | | gaatcaggtacaatgagctaagcctggcttccttgccttctaccaaaagt |
| | | gagggaggtagactcccaccccacgcctgctctcacgcccatcccagggg |
| | | caacacgggagagagcagagggcggcgctggctgcggcctcgcacctgtc |
| | | tcctggccgatgacccaccccgggccccgtcgctcctgctaacccccatg |
| | | cctctcgcagGATGGTGTTCTTCCTGGAGGACGTCATGGCCTGCACCAAG |
| | | CGCCTGCTGGAGTGGATCGCCGGCTGGCTACCACGTCACTGCATCGGGGC |
| | | CATCCTGGTGTCGCTTCCCGCACTGGCCGTCTACTCCCATGTCACCTCCG |
| | | AATATGAGACCAACATACACgtaagtaaaggctcagccgttttatttcca |
| | | cgctctcctggactccctaatctctcttttctttaaatcacttgggatag |
| | | caatttaaaaataaaccctttggccaggcatggtggctcacgcctgtaat |
| | | cccagcactgtgggaggtggaggtgggcagatcacaaggtcaagagatcg |
| | | agaccatcctggccaacatggtgaaaccccgtctctactaaaaatacaat |
| | | tagctggtcatggtggcaggcgcctttaatcccagctacttgggaggctg |
| | | aggcaggagaatcgcttgaacctgggaggcagaggttgcagtgagctgag |
| | | gttgcaccaccacactgcagcctgggtgatggagcgagactgcgtcacaa |
| | | aataaaataaaataaataaaataaaataaacccttgaagaggcacgaact |
| | | cttttactctcctgagccagcttttttagaagccgacttagcattggcct |
| | | gcccagatgctcacgcccgggcagtggccgggctggggagtccccggtgg |
| | | gcacgagtccctgccagtgtggcttcttcttcatcaagttccaaatcagt |
| | | tattttccttcacattttacaaagaagcgtctgtcccacctttcctaacc |
| | | atgcgagaagcatacctggcacacagaattgacttgtgcccgggtcggct |
| | | gctgctgatcaggaagggaactgtgctaaactccgtggagagggaaccag |
| | | cacggccgtgggagatgatccttctggtgcaagtgagcatgagcttccgg |
| | | gcgggctgatggtgtttgacactttagccccaaatcacatctcactgggc |
| | | cccaaggaaaagtgttttgaaggaaactgatcacacagcgtttctcaaac |
| | | agggaggcctccctttcctttccacgaagacacgtgccatttcccacacc |
| | | tgcagaagagtgtgatgccatgtgaacagggagcctcacgccgggtaggg |
| | | aacctagaggcttgatctgtccttgacactgcccactagcaaaagtaaga |
| | | ctgactgcagatagtgagcaattagctggtatcggtgatatttttaggtt |
| | | acacagaaacatggattaggccgggagcggtggctcatacctgtaatccc |
| | | aggcactttgagaggccgaggcaggtggatcacctgaggtcaggagttcg |
| | | agaccagcctggccaacatggtgaaacccagtctttactaaaaatacaaa |
| | | aattagcgaggtgtggtgccgggagcctgtcatcccagctactcatgagg |
| | | ctgaggtaggagaatcacttgaacccggaaggcagaggttgcagtgagct |
| | | gagattgcaccactgcactccagcctgggcgacagagtgagactcagtca |
| | | aaaaaaaaaggaaacatggattagtggcgaatgttttggcggccgggtat |
| | | agcaggggtggcggggtggccctgggcggctgtcctaacccttcgcctct |
| | | tgcagTTCCCAGTGTTCACAGGCTCGGCCGCGCTGATTGCCGTCGTGCAC |
| | | TACTGTAACTTCTGCCAGCTCAGCTCCTGGATGAGGTCCTCCCTCGCCAC |
| | | CGTCGTGGGGGCCGGGCCGCTGCTCCTGCTCTACGTCTCCCTGTGCCCAG |
| | | ACAGgtacgcggccagcactgcttgcgggcaccagggtgcgtggccacct |
| | | tgcccgaggttgcgtagggcctggctgcatttgcacattgcttctcacgc |
| | | cccgatagagatatttcccatcatgacccaaaatagctgtcagcacacat |
| | | cataccaaagagctaatgagatgccctcagagtaatatgcaaaagaaaga |
| | | aaaagaatatcatttagaacagcactctgggtagttgaatgtagacatcc |
| | | tggaaaacataatgctgccattgcattataccgaggtgacttctgatgaa |
| | | tgggctggaatctaagaaagtaggacctgaaggatttcatagaagatggt |
| | | caaggccatgaaagagcggagctgcagatggatgtgaaggtggagaggaa |
| | | gctggtgctgactttccttgagtggggtagggttcatgaaccgggacagg |
| | | tcacagcctggaaggaggaggtgaagacgtaggatgatcttgcaagtcac |
| | | ctggggcttatttcaaaatattatgtcaggctgggcacagtggctcatgc |
| | | ctgtaatcccagcactttgggaggccaaggcaggcagatcacctgaggtc |
| | | aggagttcaagaccagcctggccaacatggtgaaaccccgtctctacgaa |
| | | aaatacaaaaattagtcaggcatgggggcgtgcacctgtaatcgcagcta |
| | | cttgggaggctgaggtaggagaaccgcttgtacccgggaggcggaggttg |
| | | cagtgagctgagattgtgccattgcactccagcctgggtgacaagagtga |
| | | gattccatctcaaaaacaaaaaataataaataggtcaaaacagttcccca |
| | | tatgatgtcatgagacgtaggattaatgaccagtcactgaacataggtct |
| | | tttattttgaaatcctctgcatactgaggaggacgctggccttcagaacc |
| | | gtatcactggtaggtgctgaggaatgggatggcgcagacacagaaacagg |
| | | agtcagggaggccggtagacctctggggacaagcccagttctcatcattg |
| | | tactacaaaaaatgtaaaaacttacaggatggcagtgataggtgctatat |
| | | ttaaaatatttgccattagcgacatccagtaagatccctatctttcatgt |
| | | gttgcactaactgaataattcttgatttgttaaaaataagtcccagccgg |
| | | tcttggtgctcacgcctgtaatcctaccacttttgggaggctgaagcggg |
| | | tggattgtctgagctcaggagttcaagacagcctgggcaacacggtgaaa |
| | | ccctgtctctactaaaaatacaaaaaattagccaggcatggcggtgtgca |
| | | cctgtagtcccagctactcaggaggctgaggcaggagaattggttgaccc |
| | | tgggaggcggaggttgcagtgaaccgagatcgcgccactgcactccagcc |
| | | tgggtgacagagcaagactcagtctccaaaaataaaaataaaaaataagt |
| | | ctcaaaattgcctattacacatttgctactggtttcagggtattttaagc |
| | | cttttcaactaaatgtgttgttggaaatacagtaataccttgtataacag |
| | | aatgtaagcagtgaacaatttattcaaatatttcgtgatatcttaaaccc |
| | | attaagcttccattcatgtaataaatttctcccaaatattgggcttttcc |
| | | tataactgcttataaaaatgtttcactattagtgtgtattgaagaaccgc |
| | | ctgtttacaattacagagggtttaggtgcgcagaatgttttaaagtcaca |
| | | tgggacatttcttccttgtccttgcaggagatctaacattctggctccct |
| | | gagttattaagagaaccagagggacccctggcatttccgaaatgccccgt |
| | | tggagtggcattgctgttgccaaggacctctgcttgaattcattcaaaag |
| | | cacaaaatgattctatttgccgcagtattgtcagtatcagacggagtagg |
| | | cacggggtgtctttggttattagcaaagcaacagtccagccatgggcagt |
| | | gtgagcatccgtcctcagtttacttaagaaaagaatcacaatacaaaaga |
| | | catttaaattccctgcaaattaagatcaaaaagggaatttaaaataggac |
| | | tgtcattcagtacttgaaaggaaactttaatgaatagattaagcaaaata |
| | | aagcagacttggatttttctgaaggtatggccatctagaacgatggtcac |
| | | gatgagctctttctcgagagtatgaaatccagcaaactgctttttacctt |
| | | ttaaatccatcacaggtccctcatcgggcattcaccgtggcctttagtca |
| | | tggctgcggggcttttctacctggagctggagagccacagtgatggggga |
| | | cccctctgccgcctttgtgcccagagttcaaatagcagatctggggtagg |
| | | gatgatggttgaagttccactcggcccagaactcctccatccacttgagg |
| | | cctcagagcccctaatcccatcacggttgctaagcctcagcaggatagag |
| | | aggatgcgggatggtaaacaaagacgtaaactcagctttaccaagggaac |
| | | tgcccccagcattgggagacacagatgtgaagaaagaggtgggagcaggt |
| | | ggctttatgtggatgtacaaccacgtctaaaaaaggaacatagtttgttc |
| | | aagctcttctcaggttgccttgtggaatgccttctttgctctttatgata |
| | | atatttttctgcattttaatttcagTTCTGTATTAACTTCGCCCCTTGAC |
| | | GCAGTACAGAATTTCAGgtaagcttttaatattgcacttctgtctgaata |
| | | aacgagagtgactgtattaccctgcccatttcgaagcttcctgaaaacaa |
| | | tgggatcttgaataaaaggaacaatacgtgcattcttagaatagaaagtg |
| | | gaaaatatctcctcgttgaactgtatcatagttataatgtctaatgggga |
| | | aaataaaattagttttattattttaataagacattacagtcatgtaattt |
| | | tatgtaagactttcgcatgcaacaaaggaaatctcgcaaatcaaagaaaa |
| | | aagttattttgtaaaatattttaaaataaccgacttgaaatcagccaggc |
| | | acggtggctcacgcctgtaatcccagcattttgggaggccgaggcgggcg |
| | | gatcacgaggtcaggagatggagaccatcctggctaacacggtgaaaccc |
| | | cgtctctactaaacaaaatacaaaaaattagctgggcatggtggcgggcg |
| | | cctgtagtcccagctactcaggaggctgaggcgggagaatggcgtgaacc |
| | | cgggaggcggagcttgcagtgagccaagattgtgccactgcactccagcc |
| | | tgggtgacagaatgagactgtgtctcaaaaagtaataaaataactgactt |
| | | gaaatccaaaatactaaatgtttgacccttgctttactataaacagcaga |
| | | ataaattctaaataaattgaagagtcggaaataaaaattaaactatataa |
| | | aatctagaaaaagtaaaattcaatagttttcaagcctctaaaactgagat |
| | | aactttccaggcttacaaataataaatagaaccacaacgaaaagatggac |
| | | agatttgagaggaagggagagaaaatcaaagcacacagaaaaggaccggg |
| | | ctcggtgactcccgcctgtaatcccagcactttgggaggccaaggcagga |
| | | agattacttgaggcctggagttctagaccagcctgggcaacatagcgaaa |
| | | tcctgttttggcaaaaaattgtaaaaattagccaggtatggtggtatgca |
| | | cctgtagtcccagctactcgggaagctaaggtgggcttgaggccgggaat |
| | | ttgaggctgcattgagctgtgatggtgtccttgcaccccagcctggttgt |
| | | cacagcaagactctgtctcagaaaaaaaaggccaggcacgttggctcatg |
| | | cctgtaattccagcactttgggaggccaaggcaggtggatcacctgagat |
| | | caggagttcaagaccagcctggccaacatggcaaaaccccgtctctacta |
| | | aaaatacaaaaattagctggggtggtggtgggcacctgtaatcctagcca |
| | | cttgggaggctgaggcaggagaattgcttgaacctgggaagcggaggttg |
| | | cagtgagccaagatcggccactgcactccagcctgggcgacagagtgaga |
| | | ctctgtctcaaaaaataaaataaaataaaaaaaagattcattagcaactg |
| | | tagttgtcaataataaataaattaactgattatacatttactcaaataat |
| | | acaactgttaaaatgcagtaaatatggaaaacatttaagattttcattta |
| | | ctaggtgaaactatatatgtactataggtacaactaggtatttatatata |
| | | gtaaaaatatgtacaagatttatacttaggatataatgaatgagtgatga |
| | | ttacagctttagtacttaaaagtaaagcacctagcatctttttttaacct |
| | | tttattgatgtgtatgtcagtatgcacagaagagtacctgatcataaaag |
| | | gacagctcagtgcattttcaccagagaacacacccatttaactaccaccc |
| | | agaccaagaagtagagcattaccactaccccagaagccctccaaccccac |
| | | cgctgctgaccccctctcccccgacctgaccacctccctaggggcagatt |
| | | ttaaatggcactgaaatataccaaagtgtcaaccgaagccacgtttggct |
| | | ggttaggactgcaagtgatgtttgctttctatttttttcagtttttcaat |
| | | ccctcttctccatgataacggattacactgtattttactctaacttccat |
| | | ttatgacttataaattaactcctgcatttagtcacatgaaatgcctcttt |
| | | ttgagccaggtgtggtggctcacgcctgtaatcccaacactttgggaggc |
| | | tgaggcaggtggatcacctgaggtcaggagttcgagaccagcctgaccaa |
| | | catgtttgtgaaaccccatctctactaaaaatacaaaaattagctggacg |
| | | tggtggcgggcacctgtaatcccagctactcgggaggctgaggcaggaga |
| | | attgctagaacccgggaggcagaggttgcagtgagcctagatcgcaccac |
| | | tgcactccagcctgggggacagagcaagactccatctcaaaaaaaaaaaa |
| | | agaaaagaaaaagaaattcttctttttgaagtggatctttgctatttgaa |
| | | tttaggaataggatcggctcctttgttcctttggtattaaaataggaaac |
| | | acacattctagctgggaagctgtcctcgtacctgtttcttacagatgcct |
| | | tctgttgcgaggcagcccccttggtttctgagtgcacatgctgtgtgtga |
| | | tggcttttcttccttggaagcaaaggtggaaagcaaacgctgtgctctgc |
| | | cttcctctccctctggcactagcccatgtgctgctgtccctcctctgcct |
| | | ggaggggcctggcctgcgcccgtctcgagtcagctgtgctgctggctccg |
| | | gctcccgcagattagagtgagctgtacaagcgataactgattgagaagcc |
| | | cagatacagtctcccatttccaccacagagactggaaacaccccctgcac |
| | | ataatggggatgttagaccttggtgacacctgctcctaaaggggactgtt |
| | | cttggtgtcaccactttgagaaaccaaaagaaaggtcaactcctaaccca |
| | | acgagcggggctcacaaaaccttcccccagtccatattctcttcctgctc |
| | | tatttacaatcaaccagagggctcatggagctggacagaaatatgtttga |
| | | gtcatcatcgtaacaaatggttattttgcttagtacttgagatgtaggat |
| | | atgtaatgtcatctgtttcaatagcagttcacgaaatgagtcagtgaagc |
| | | acagggtgtcttcccactgaggactcttggttcctggtcataggcgttga |
| | | gtcacaaagcccgtatcagggctcccaagaccagccccaggtggactcag |
| | | gactccgcaatctgctctctcaccgctgtgattacagcgaaaagacataa |
| | | ggcagaatcagcaaagggaagggtgtgtgagttgacgtttggaggaagcc |
| | | ggcaccagcttccaagagtcctctcccggtagagtcacacgggatgggct |
| | | taattcctccagcagtgagtggtgacaacacttgtaacatgctgcctacc |
| | | agggaagctcattaaagtctcggtgtccaggcttgtattaggggctgttc |
| | | aggtctcccgcctctgcccagcacgtgcctaaattccagacttccagcag |
| | | gaaagcgggtattcagcgtaaacctccttatttagacagtgtaggcagtg |
| | | tgagccgctcttatcaaggaatggcaggaaccctccccaaatcccagctc |
| | | cctggtgccagccaccacctttgcaggcaggtctttccaaggatggcagc |
| | | ctcaggcctttcctgcacggaaccttaggctggaagggacgtagccatgc |
| | | tccagtccatcttcacagcttacaagcgaggaaactgagacccagaaaga |
| | | tggcatggcttacccaagattctgaaactcccccgcggtccagccaggat |
| | | ggggcccaggggttctgttccacccacggccagggcttttggcggttgtg |
| | | cttcgcagagggccgtgatggccacacaggtcaagctcattgttttttaa |
| | | tatcccaagaacagttagtgaaacttattagacactttgattaaaggact |
| | | gagcttttggccgggcatggtggctcgtgcctgtaatcccagcactttgg |
| | | gaggccaaggcgggtggatcacctgaggtcaggagttccagaccaacctg |
| | | gtcaacatggtgaaaccctgtctctactaaaaaatacaaaaattagccaa |
| | | gtatggtggcgcacacctgtagtcccagctactcgggaggctgaggcaag |
| | | agaatcacttgaacctggaagcggaggttgcagtgagccaaggtcgcgcc |
| | | attgcactccagccagggcaacagagcaagaaagaaaaaagaagaattga |
| | | gttttggccaggcgtggtggctcatccctgtaatcccagcacttggggag |
| | | gctaaggcaggcagattggttgagctcaggagttcaagaccagcctgggc |
| | | aagatggcgagaccctgtctctaaaagaaaaagaattgagctttccagtg |
| | | tgatggagctgtctaaactgtggatccgccagtcgcatatataagtatat |
| | | agtgaccaagcccattattgcatcaaatcaactggcctactctgtgcgcg |
| | | ggaggaatgcatcagggtacagcatttacatgaacagaacagtcacggtg |
| | | ttgtaaaaaagaaaaagactagaatgcagttcaacaaatgttaatagtga |
| | | ttgcctctggatggcaggacgatggacaaatcttttttgtctattttttt |
| | | ctacattttctatgatgagtatgtagtatttttaaatgggaaaaaatgta |
| | | ttccagcacactggggaggcagaagcagaacagtttgaacccaggagttt |
| | | gagaccagcctgggcaacatagtaagacctcatctctacaaaaagttcaa |
| | | aaaattagccaggtgtggtggtgtgtgcctgtagtcccagctaatctgga |
| | | ggctgaggcgggaggatcgctttagcctgggaggtcaaagctgcagtgag |
| | | ctaggatcgcaccactgcagtccagcttggatgacagagacagaccctgt |
| | | ctcttcttaaaaaaaaaaaaaaatacatatatatatatatatatatatat |
| | | atatatatatatatatttcaaacaaactttaagaaattgtgctactcagc |
| | | caggtgtggtggctcacacctgtaatcccagcactttgggaggtcgaggc |
| | | gggcagatcactttaggtcaggagtttgagaccagcccagccaacatggt |
| | | gaaaccccgtttctaccaaaaaaaaaatacaaaaattagctgggcctggt |
| | | ggcgcacactactcaggaggctgggccgcaagaatcgttgagcccaggag |
| | | gtggaggttgcagtgagctgagatcacaccactgcatgccagcccgggct |
| | | agagagtgagactctgtctcaaaaaagaaaaaagaaaaagaaaaaaagaa |
| | | attgtgctactcaccactcatctgcagccccctgaccatggctgtctcca |
| | | tcagtgccactgaagtggagacagccccgctgaaagcaggctcagttatc |
| | | aaagggagcagagactgggcgcggtggctcacgcctgtgatcccagcact |
| | | ttgggaggccgaggcgggtggatcacgaggtcacgagtttgagaccaacc |
| | | tgtctctagtaaaagtacaaaaataatctgggcatggtggcgtgcacctg |
| | | tagtccgagctacacagaaggctgaagcaggagaattgcttgaacccagg |
| | | aggcggagtttgtggtgaggcgagatcacgccactgcactttaacctggg |
| | | caacaaagcgagactccttctcaaaaaaaagaaaaaaaaaagcagaaact |
| | | ggttcagccttgtttagaagggcaatttggaaatttctaaggaaattcta |
| | | aatgtgcaatatttaaaatgactcagcacttccatacgcacaaagatagg |
| | | ggtaggggaatattccctgtgacactgtttgtggtaagaaaagacagact |
| | | cgggcccagttccatcagggggaatctggctaaattacagaatttccgag |
| | | tgctggaacactgcacagccatgagaaaataatggcactggttcagatgt |
| | | tagcattgtcctgagtgatttcggcaaaaagaactaacccagaatcaggg |
| | | gagatggcgtcttgtcaaacttttttatacaggtagagagtatagaactc |
| | | ataaaagctatttgtaaaatgataagtttattggattagaaatctaagaa |
| | | ccaaggccaggcgcggtggctcacacctgtaatcccagcactttgggagg |
| | | ctgaggtgggcggatcacctgaggtcgggagtttgagacaagcctgacca |
| | | acatggagaaaccccgtctctactaaaaatacaaaattagctgggtgtgg |
| | | tggcacatgtctgtgaacccagctactggggaggctgaggcaggagaatc |
| | | acttgaacccgggaggcggaggttgtagtgagccaagatctcaccagcaa |
| | | caagagcaaaactccgtctcaaaaaaaaagaaatctaagaaccaaatata |
| | | tcagagaatgttaaatttcttgggtatgatgatgatattgtagttacata |
| | | gggaaatatccttattcttaggagatacatgctgaaatatttagagctga |
| | | agtgccatgtcagcaacttactttcaaaaagttcattcagcaaaaagccg |
| | | ggtgcagtggctcacacctgtaatcccagcactttaaggcaagctgatca |
| | | ctttgagatcaggagtttgagaccagcctggccaacatgatgaaaccctg |
| | | tctctactaaaaatacaaaaattagccaggcatggtggcacacacctata |
| | | gtcccagctattcaggaggctgaggcaggaggatcacttgaactcaggaa |
| | | gcagagggtgcagtgagccaagatcgcaccactgcactctagcctgggca |
| | | acagagcaagactcaatcttaaaaaaaaaaaaaaaaaaagatgttaggcc |
| | | aagcacagttcaagcctgtaatcccagcactttggaaggccaaggtggga |
| | | ggatggcttgaagttacgagtttaagaccagccttggcaaccagcaagac |
| | | cctgtctcttcaaaaaaaaaattttttaattagctaggtgtggtggtaca |
| | | tgcgtgtaatcccagctacttaggaggctgggcaggaggatcgtttgagc |
| | | ccaggagtttaaggctgcagtgagccatgattgtgccattgcactccagc |
| | | ctggacaacaaccatgtctctaaaaaaacaaaggagagagagaaaagaaa |
| | | agatttttgaaagaagctaaagacttctaaaaaaaaagaaaaagaaaaag |
| | | aaaagaaaaacgattaccaagcacttactaaagatgttagttacagggct |
| | | gggcacagcagctcacgcctgtaattccagcactttgggaggccaaggca |
| | | ggaaaattgcttgagcccaggagtgcaagaccagcctgggcaacacagtg |
| | | agaccccacctctatttaacaaaatgaaatcaatcagtttcaaaagttag |
| | | tctttatccctccataccttcagccagcccaaaccaggaatgggcagctt |
| | | gacagcttcaggttgttgaaggcctggactcaacaaagctttgcgtagct |
| | | agcggggctgttctgagcttggaagcgccgagcagtaacctctccctttc |
| | | ccgtgtctttgctctccagTTCCGAGAGGAACCCGTGCAATAGTTCGGTG |
| | | CCGCGTGACCTCCGGCGGCCCGCCAGCCTCATCGGCCAGGAGGTGGTTCT |
| | | CGTCTTCTTTCTCCTGCTCTTGTTGGTCTGGTTCCTGAATCGCGAATTTG |
| | | AAGTCAGCTACCGCCTCCACTACCACGGAGACGTGGAAGCGGATCTTCAC |
| | | CGCACCAAGATCCAGAGCATGCGGGACCAGGCAGACTGGCTGCTGAGGAA |
| | | CATCATCCCCTACCACGTGGCTGAGCAGCTGAAGGTGTCCCAGACCTACT |
| | | CCAAGAACCATGACAGCGGAGGGGTGATCTTCGCCAGCATCGTCAACTTC |
| | | AGCGAGTTCTACGAGGAGAACTACGAGGGCGGCAAGGAGTGCTACCGGGT |
| | | CCTCAACGAGCTCATCGGGGACTTTGACGAGCTCCTAAGCAAGCCGGACT |
| | | ACAGCAGCATCGAGAAGATCAAGACCATCGGAGCCACGTACATGGCGGCG |
| | | TCAGGGCTGAACACCGCGCAGGCCCAGGACGGCAGCCACCCGCAGGAGCA |
| | | CCTGCAGATCCTGTTCGAGTTCGCCAAGGAGATGATGCGCGTGGTGGACG |
| | | ACTTCAACAACAACATGCTGTGGTTCAACTTCAAGCTCCGCGTCGGCTTC |
| | | AACCATGGGCCCCTCACGGCCGGGGTCATCGGCACCACCAAGCTGCTGTA |
| | | CGACATCTGGGGAGACACCGTCAACATCGCCAGCAGGATGGACACCACCG |
| | | GCGTGGAGTGCCGCATCCAGGTGAGCGAAGAGAGCTACCGCGTCTTGAGC |
| | | AAGATGGGCTATGACTTCGACTACAGAGGGACCGTGAATGTCAAGGGGAA |
| | | AGGCCAGATGAAGACCTACCTGTACCCAAAGTGCACGGATCACAGGGTCA |
| | | TCCCACAGCACCAGCTGTCCATCTCCCCAGACATCCGCGTCCAGGTGGAT |
| | | GGCAGCATCGGACGGTCTCCCACAGACGAGATTGCCAACCTGGTGCCTTC |
| | | TGTCCAGTATGTGGACAAGACATCTCTGGGTTCTGACAGCAGCACGCAGG |
| | | CCAAGGATGCCCACCTGTCCCCCAAGAGACCGTGGAAGGAGCCCGTCAAA |
| | | GCCGAAGAAAGGGGTCGATTTGGCAAAGCCATAGAGAAAGACGACTGTGA |
| | | CGAAACAGGAATAGAAGAAGCCAACGAACTCACCAAGCTCAACGTTTCAA |
| | | AGAGTGTGTGAGGCGGCGCCCACCCGCTGCCCGAGGTGCTCTGTTTGTCG |
| | | AAACACAGTAATATTTGTATTTGGCTGTTGTGCTTTCCAAGCGCCACAGT |
| | | TGCCCTCCCCGGACGTGGTGTTATGTGGTCATTTCAGCCCTAACTTCTGT |
| | | GTGGATCACAGTTATTCAGGGTTCATTTTCATCCATTCTTCCCTTTCGCT |
| | | CCCTTCCCTGGAAACCCCGCTGCCTCTGGGTCATCCGTTCAGCACGTGGT |
| | | GGAGAACAAGTGCCTTCAGGGCTGGCCTCGGCCTCGAGTCTCGGGACAGA |
| | | GGCCGCCAGTGGAGATCATGGCTTTGGGTATTATTTGACTTTTAGAACAA |
| | | AAGCTGTGGTTAAGATCTCATTTTTATTGCTTTTTCCCACGTCCCACGAG |
| | | ACACTATTTTCGGTTCTCTGGCTAATACCCTGTTTTTGAGTTTATTTTGT |
| | | TTCTGTCTATGTCACAGTGTTCCTCTACGACCCGACCTCTCTATGTAAGC |
| | | ACACATGCGCACACACACTTGCATTCATGAATCTGATATAAAGTGCCAGT |
| | | AATCCGCCAAGAGGGGGTGCGAAGGGGGCATGTCACGACAGCTCCGCCAC |
| | | CCCCCATTGCCCACCCGCACTTTCCCGAGCACCGCGCCCCGTGGGCTGTG |
| | | GGTGAGCCGCGCTCCCTGCACTGAGCGGGTTTAGGGGCTCGCCCACATGC |
| | | ATGCAGGCCAAGACAGCAAATGCCAGCCGGGCACGACGCCCGTGTGCCCA |
| | | GGCCTCGGGGGTCTCAGAGCCGCCTCTCACCCCCGACCCTCCACCCAGGG |
| | | GTCTCCCCATCGGGAGTGGAGGTGTTGGTCCTGGAAGCTGACTCATCGGA |
| | | GAGGGAAATACCAAATAAACATCCGAGGTTGCATTTATTTATTTATTTAT |
| | | TTACCCGGAAGCATGTGCGCTCCCGGGAGGGGTGCTGGGTCCTCAGGGTG |
| | | GGGGGTCATAGCCACAGGGGAGGGCAGTTCCCAGGAAGGTAACCAGATGT |
| | | CTCTTTTGTGTTTTGGCCACCTGGGGCCGCCCCCAACCTCCCCACCCCAA |
| | | GAGGGGTCTGAGTGGGACCTCAAAGCATGGCCAGTGCAATTCTGAGGCTA |
| | | GGGGAATAGGAGAAGCCCACCAGCTGCCCCTGCCACCCACGGGGGCTCCT |
| | | CCCTGCCGGGGTCCAGCTCAGAACCACGGGGGTCTGTGCCGGCAGCACCG |
| | | GTGCCATCTTGGCTTCCACAAATTCCAGAAGTTTCTTGGCTGCCTCCCGC |
| | | TCTCAGATGGCACAGGGCCTATCAGTGGCAGGATTTAATCTGGGGAGCAA |
| | | ATTCCAGCTCTGAAATGGAGTTCTCACACCGTATCTAGGGACAGAGGCAT |
| | | CTGGATGCAAACGCGGGGTTTGTTAGGGGCGGAATCCCGAGGGTGGTGTG |
| | | GGGGAGCTCCGTCCGGGCAGTTGGCTGCTGGTCCTGTCTGATTATGTTTT |
| | | GCCATATTGAGTCTGAAGTTTGTGTGGAAGTGAGCTCGCCCGAGTTGTCC |
| | | GGTCGGAGTTGTAGCCAGTCTTTCAGGGGACCCCGGGACGCTGATGAGGC |
| | | CCAGGGTGCACTCCTTGTCAGTCTTGCCCGTTGTCCCTGTGCATTTTGCT |
| | | TTCCGCTGTCAAATCTCACTGTCTCGTTTTCACACCGCATCAGTCCCCAT |
| | | CATCACTACTCCTCCCCAGAGACATACAGATTGCCCTCCCCGGCATGAAG |
| | | AAGTCACAATTCTGACCCGCCACCTCTCCAGGGGCGGTGCCTGGGATGGC |
| | | CACCCATCAGGACAGCCAGAGCCAGGGTCCCGGCGTCCTGAGAGTCCAGG |
| | | GCACCTCCCCCCATTAGCTGTAAGGTTTCCTGTCAGGTGTGACTGTCAGT |
| | | TTGCTGGTCTTCCAATACCGAAGAAAGGGTGGTTGTGACAATACCTCTTG |
| | | CTTCTAAAGAATGTATTATAAAACACCGCAGATTTTTTTTTTTCCTTAAA |
| | | AAACACTACCTGATGCTTTCCTTGTTCGTGGGGATTGTGGTCACATGAAG |
| | | CTCTTTCTGCATCAGTATTAAGGTGTATATTTGAATGTCCTCCCCTCCCC |
| | | TTTCCCCTCCAGGCTGTGTAGCTTTGAGGGGCTGGGCGTTTGCTCACGAC |
| | | CTTGCTGTCTCGCTCAGAACATGCTCCGCAAAGTTCTCCGCACACACTTC |
| | | TTCCCCATCAAGCCCATTTCCTTCCCCAACCACAAAGGTGTTTGTGATTC |
| | | CTCACCCCGGGAAACCAAGGAGCTGCAAAGTGGAGTCTGGTTCAGCCCCG |
| | | TGCAGACTCACCCAGAGCTTAACGTTGTCTTTCAAACACCCTGAGCCTTC |
| | | CTAAACAGCCAGTGCAGACGTTCTCTCTGGGCCACGAAGCCCCTCGGGTC |
| | | CTCCCCGTCCCCTGCTCCGATGCATACCTCAGTGCAGAACCACAGAATCT |
| | | CTGCAGCGGAAACGCCGTGCATCTCTTGTCTGTTGGCAGCGAGCACATCG |
| | | TGCTGGAGACACGAGTTTCTAAGCAGCTGGCACGAGGGCTGCTGACGGCA |
| | | TGGGTCGTGCTTCAGGGTGGCAATACCTCTTAGGAACTTAGGGCAGGAAG |
| | | CAATACTTCAGCATTGAATGTGTGTAAATAGTTGCTTTGAGTTGCAATTG |
| | | CTATTTTCTTCTCAGTCCCAGCTCAGATCGAATTATATATCCATATATAT |
| | | ATATATATATATATATATATATATATATATATATATATGGTAAACAAGCA |
| | | CACACAATTTTATCCAATGCAAACAAATGTAGAGCATCAGTTACAAAACC |
| | | CTCGAATAGCTTGAGAGCCCCACAGGCTCTGCCACACCCGTGACTTCATC |
| | | CACACTGACGTCACCCGCGGGGGCTCCCCCTGCACATTTGCACACGATCC |
| | | GGAGAGCCGAAGGCCGCGTGCTTCCTGTCACATGGGCTGTAATCATTTGT |
| | | AGTTTCCAAAGACACGTCTGCATTTGAATTTCTAGATTTTCGAGGTAAGG |
| | | AGTTTTTTTTAATTGGTTGTTTGGAAAATCACATCATGCCTAGAATCTGA |
| | | AATTGAATTAGCAAGAACCGACTGTTTGCATTTTCCATATATCCTTTTAT |
| | | CTGCTCTTTTTAAATTGTTAATTCTAATAATTTCAAAATGCATTCACTGA |
| | | AGAAATGGACATTAAAATATTCTAAAATTTAAATATA |

Double-Stranded RNA (dsRNA)

dsRNAs of the disclosure are ds nucleic acid molecules made of DNA, RNA, or both DNA and RNA (e.g., a chimeric) that are complementary to a target gene of interest and prevent translation of the target's mRNA into a protein. Typically, dsRNAs are longer than an siRNA and are processed within a cell to form an siRNA molecule. The siRNA is then incorporated into an RNA-induced silencing complex (RISC). Upon siRNA hybridization to a target mRNA, the RISC complex will cleave the target mRNA, thereby inactivating the target mRNA, resulting in reduced mRNA and protein levels of the target.

In some embodiments, dsRNAs of the disclosure may include a sense strand and an antisense strand, each containing a nucleotide sequence of about 25 to about 5000 nucleotides in length, or longer (e.g., 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 210, about 220, about 230, about 240, about 250, about 260, about 270, about 280, about 290, about 300, about 310, about 320, about 330, about 340, about 350, about 360, about 370, about 380, about 380, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1600, about 1700, about 1800, about 1900, about 2000, about 2200, about 2400, about 2600, about 2800, about 3000, about 3250, about 3500, about 3750, about 4000, about 4250, about 4500, about 4750, about 5000, about 6000, about 7000, about 8000, about 9000, or about 10000 nucleotides in length).

In some embodiments, dsRNAs of the disclosure may include a sense strand and an antisense strand, each containing a nucleotide sequence of 25 to 5000 nucleotides in length, or longer (e.g., 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 380, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2200, 2400, 2600, 2800, 3000, 3250, 3500, 3750, 4000, 4250, 4500, 4750, 5000, 6000, 7000, 8000, 9000 or 10000 nucleotides in length).

It is within the scope of the disclosure that any length, known and previously unknown in the art, may be employed for the current invention.

In some embodiments, the sense and antisense strands of an dsRNA molecule of the disclosure are completely complementary. In some embodiments, the sense and antisense strands of an dsRNA molecule of the disclosure are completely complementary to the extent that their lengths overlap with one another. Depending on the sequence of the first and second strand, complementarity need not be complete or perfect, which means that the first and second strand are not 100% base-paired due to mismatches. One or more mismatches may be present within the ds dsRNA without impacting the dsRNA's ability to reduced expression of a target gene of interest.

The nucleotide sequence of an dsRNA of the disclosure may contain sufficient complementary to a portion of a target gene of interest (e.g., AC9 mRNA) such that the dsRNA can hybridize with the target gene of interest. In some embodiments, the dsRNA is at least 70%, at least 75%, at least 80%, at least 85%, at least 90%, at least 95%, at least 96%, at least 97%, at least 98%, or at least 99% complementary to the target gene of interest (e.g., AC9 mRNA), or a portion thereof. In some embodiments, the dsRNA is complementary to the target gene of interest (e.g., AC9 mRNA), or a portion thereof.

In some embodiments, the nucleotide sequence of the dsRNA may contain sufficient complementary to an exon sequence of a target gene of interest (e.g., an exon of AC9). In some embodiments, the nucleotide sequence of the dsRNA may contain sufficient complementary to an intron sequence of a target gene of interest (e.g., an intron of AC9). In some embodiments, the dsRNA of the disclosure may contain sufficient complementarity to a pre-mRNA transcript or an mRNA transcript encoding AC9. The target gene of interest (e.g., AC9) may be any one of SEQ ID NOs: 16-17 (e.g., see Table 3).

Different dsRNAs can be combined for decreasing the protein expression of a target gene of interest (e.g., AC9). A combination of two dsRNAs may be used in a method of the invention, such as two different dsRNAs, three different dsRNAs, four different dsRNAs, or five different dsRNAs targeting the same gene of interest (e.g., AC9, or variants thereof).

### Anti-Sense Oligonucleotide (ASO)

ASOs of the disclosure are single (ss) nucleic acid molecules made of DNA, RNA, or both DNA and RNA (e.g., a chimeric) that are complementary to a target gene of interest and prevent translation of the target's mRNA into a protein. Upon hybridization to a target mRNA, RNase H will degrade the mRNA by hydrolyzation, resulting in reduced mRNA and protein levels of the target.

In some embodiments, ASOs of the disclosure may include a nucleotide sequence of 12 to 50 nucleotides in length (e.g., 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51 nucleotides in length).

In some embodiments, ASOs of the disclosure may include a nucleotide sequence of 12 to 50 nucleotides in length (e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length).

It is within the scope of the disclosure that any length, known and previously unknown in the art, may be employed for the current invention.

The nucleotide sequence of the ASO may contain sufficient complementary to a portion of a target gene of interest (e.g., AC9 mRNA) such that the ASO can hybridize with the target gene of interest. In some embodiments, the ASO is at least 70%, at least 75%, at least 80%, at least 85%, at least 90%, at least 95%, at least 96%, at least 97%, at least 98%, or at least 99% complementary to the target gene of interest (e.g., AC9 mRNA), or a portion thereof. In some embodiments, the ASO is complementary to the target gene of interest (e.g., AC9 mRNA), or a portion thereof.

In some embodiments, the nucleotide sequence of the ASO may contain sufficient complementary to an exon sequence of a target gene of interest (e.g., an exon of AC9). In some embodiments, the nucleotide sequence of the ASO may contain sufficient complementary to an intron sequence of a target gene of interest (e.g., an intron of AC9). In some embodiments, the ASO of the disclosure may contain sufficient complementarity to a pre-mRNA transcript or an mRNA transcript encoding AC9. The target gene of interest (e.g., AC9) may be any one of SEQ ID NOs: 16-17 (e.g., see Table 3).

Different ASOs can be combined for decreasing the protein expression of a target gene of interest (e.g., AC9). A combination of two ASOs may be used in a method of the invention, such as two different ASOs, different three ASOs, four different ASOs, or five different ASOs targeting the same gene of interest (e.g., AC9, or variants thereof)

Micro RNA (miRNA)

miRNAs of the disclosure are single stranded (ss) nucleic acid molecules made of DNA, RNA, or both DNA and RNA (e.g., a chimeric) that are complementary to a target gene of interest and prevent translation of the target's mRNA into a protein. Once a miRNA molecule enters a cell, it is incorporated into a RNA-induced silencing complex (RISC). Upon miRNA hybridization to a target mRNA, the RISC complex will cleave the target mRNA, thereby inactivating the target mRNA, resulting in reduced mRNA and protein levels of the target.

In some embodiments, miRNAs of the disclosure may include a nucleotide sequence of 6 to 30 nucleotides in length (e.g., 5, 6, 7, 8, 9, 10, 11, 12, 13, 14, 15, 16, 17, 18, 19, 20, 21, 22, 23, 24, 25, 26, 27, 28, 29, 30, or 31 nucleotides in length).

In some embodiments, miRNAs of the disclosure may include a nucleotide sequence of 6 to 30 nucleotides in length (e.g., 6, 7, 8, 9, 10, 11, 12, 13, 14, 15, 16, 17, 18, 19, 20, 21, 22, 23, 24, 25, 26, 27, 28, 29, or 30 nucleotides in length).

The nucleotide sequence of the miRNA may contain sufficient complementary to a portion of a target gene of interest (e.g., AC9 mRNA) such that the miRNA can hybridize with the target gene of interest. In some embodiments, the miRNA is at least 70%, at least 75%, at least 80%, at least 85%, at least 90%, at least 95%, at least 96%, at least 97%, at least 98%, or at least 99% complementary to the target gene of interest (e.g., AC9 mRNA), or a portion thereof. In some embodiments, the miRNA is complementary to the target gene of interest (e.g., AC9 mRNA), or a portion thereof.

In some embodiments, the nucleotide sequence of the miRNA may contain sufficient complementary to an exon sequence of a target gene of interest (e.g., an exon of AC9). In some embodiments, the nucleotide sequence of the miRNA may contain sufficient complementary to an intron sequence of a target gene of interest (e.g., an intron of AC9). In some embodiments, the miRNA of the disclosure may contain sufficient complementarity to a pre-mRNA transcript or an mRNA transcript encoding AC9. The target gene of interest (e.g., AC9) may be any one of SEQ ID NOs: 16-17 (e.g., see Table 3).

Different miRNAs can be combined for decreasing the protein expression of a target gene of interest (e.g., AC9). A combination of two or more miRNAs may be used in a method of the invention, such as two different miRNAs, three different miRNAs, four different miRNAs, or five different miRNAs targeting the same gene of interest (e.g., AC9, or variants thereof)

Short Hairpin RNA (shRNA)

shRNAs of the disclosure are ss or ds nucleic acid molecules made of DNA, RNA, or both DNA and RNA (e.g., a chimeric) that are complementary to a target gene of interest and prevent translation of the target's mRNA into a protein. Once a shRNA molecule enters a cell, it is incorporated into a RNA-induced silencing complex (RISC). Upon shRNA hybridization to a target mRNA, the RISC complex will cleave the target mRNA, thereby inactivating the target mRNA, resulting in reduced mRNA and protein levels of the target.

In some embodiments, shRNAs of the disclosure may include a nucleotide sequence of 60 to 100 nucleotides in length (e.g., 50, 55, 60, 65, 70, 75, 80, 85, 90, 95, 100, 105, or 110 nucleotides in length).

In some embodiments, shRNAs of the disclosure may include a nucleotide sequence of 60 to 100 nucleotides in length (e.g., 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length).

shRNAs of the disclosure contain a variable hairpin loop structure and a stem sequence. In some embodiments the stem sequence may be 10 to 50 nucleotides in length (e.g., 18, 19, 20, 21, 22, 23, 24, 25, 26, 27, 28, 29, or 30 nucleotides in length). In some embodiments, the hairpin size is between 4 to 50 nucleotides in length, although the loop size may be larger without significantly affecting silencing activity. shRNA molecules of the disclosure may contain mismatches, for example G-U mismatches between two strands of the shRNA stem without decreasing potency. In some embodiments, shRNAs are designed to include one or several G-U pairings in the hairpin stem to stabilize hairpins during propagation in bacteria, for example.

The nucleotide sequence of the shRNA may contain sufficient complementary to a portion of a target gene of interest (e.g., AC9 mRNA) such that the shRNA can hybridize with the target gene of interest. In some embodiments, the shRNA is at least 70%, at least 75%, at least 80%, at least 85%, at least 90%, at least 95%, at least 96%, at least 97%, at least 98%, or at least 99% complementary to the target gene of interest (e.g., AC9 mRNA), or a portion thereof. In some embodiments, the shRNA is complementary to the target gene of interest (e.g., AC9 mRNA), or a portion thereof.

In some embodiments, the nucleotide sequence of the shRNA may contain sufficient complementary to an exon sequence of a target gene of interest (e.g., an exon of AC9). In some embodiments, the nucleotide sequence of the shRNA may contain sufficient complementary to an intron sequence of a target gene of interest (e.g., an intron of AC9). In some embodiments, the shRNA of the disclosure may contain sufficient complementarity to a pre-mRNA transcript or an mRNA transcript encoding AC9. The target gene of interest (e.g., AC9) may be any one of SEQ ID NOs: 16-17 (e.g., see Table 3).

Different shRNAs can be combined for decreasing the protein expression of a target gene of interest (e.g., AC9). A combination of two or more shRNAs may be used in a method of the invention, such as two different shRNAs, three different shRNAs, four different shRNAs, or five different shRNAs targeting the same gene of interest (e.g., AC9, or variants thereof).

### Modifications to the Inhibitory Nucleic Acid Molecules

It is contemplated that any of the inhibitory nucleic acid molecules disclosed herein may be used in the methods disclosed herein in an unmodified or in a modified form. Unmodified inhibitory nucleic acid molecules contain nucleobases that include the purine bases adenine (A) and guanine (G), and the pyrimidine bases thymine (T), cytosine (C), and uracil (U). Modified nucleic acid molecules are described in more detail below.

Modifications may be achieved by systematically adding or removing linked nucleosides to generate longer or shorter sequences.

Modifications may be achieved by incorporating, for example, one or more alternative nucleosides, alternative 2′ sugar moieties, and/or alternative internucleoside linkages, which are described further below. Typically, these types of modifications are introduced to optimize the molecule's efficacy or biophysical properties (e.g., increasing serum stability or circulating half-life, increasing thermal stability, enhancing transmembrane delivery, reduce immunogenicity, and/or targeting to a particular location or cell type).

Modification may further be achieved by covalently or non-covalently conjugating a moiety (e.g., a targeting moiety, a hydrophobic moiety, a cell penetrating peptide, or a polymer) to the 5′ end and/or 3′ end of the inhibitory nucleic acid molecule, as described in more detail below.

### Nucleoside Modifications

Modification of the inhibitory nucleic acid molecules described herein include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The inhibitory nucleic acid molecules may also include nucleobases in which the purine or pyrimidine base is replaced with other heterocycles, for example 7-deaza-adenine, 7-deazaguanosine, 2-aminopyridine, and/or 2-pyridone. Further modification of the inhibitory nucleic acid molecules described herein may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

### Sugar Modifications

Modifications of the inhibitory nucleic acid molecules described herein may also include one or more of the following 2′ sugar modifications: 2′-O-methyl (2′-O-Me), 2′-methoxyethoxy (2′-O—CH2CH2OCH3, also known as 2′-O-(2-methoxyethyl) or 2′-MOE), 2′-dimethylaminooxyethoxy, i.e., a O(CH2)2ON(CH3)2 group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH2OCH2N(CH3)2. Other possible 2′-modifications that can modify the inhibitory nucleic acid molecules described herein include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH2CH2CH2NH2), allyl (—CH2-CH═CH2), —O-allyl (—O—CH2-CH═CH2) and fluoro (F). 2′-sugar substituent groups may be in the arabino (up) position or ribo (down) position. In some embodiments, the 2′-arabino modification is 2′-F. Similar modifications may also be made at other positions on the interfering RNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Internucleoside Linkage Modifications

Modifications of the inhibitory nucleic acid molecules described herein may include one or more of the following internucleoside modifications: phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

### Conjugates

Any of the inhibitory nucleic acid molecules described herein may be modified via the addition of an auxiliary moiety, e.g., a cell penetrating peptide (CPP), a polymer, a hydrophobic moiety, or a targeting moiety. The auxiliary moiety may be present as a 5′ terminal modification (e.g., covalently bonded to a 5′-terminal nucleoside), a 3′ terminal modification (e.g., covalently bonded to a 3-terminal nucleoside), or an internucleoside linkage (e.g., covalently bonded to phosphate or phosphorothioate in an internucleoside linkage).

CPPs are known in the art (e.g., TAT or Arg8) (Snyder and Dowdy, 2005, Expert Opin. Drug Deliv. 2, 43-51). Specific examples of CPPs are provided in WO2011157713, which is incorporated herein by reference in its entirety.

Inhibitory nucleic acid molecules of the disclosure may include covalently attached neutral polymer-based auxiliary moieties. Neutral polymers include poly(C1-6 alkylene oxide), e.g., poly(ethylene glycol) and poly(propylene glycol) and copolymers thereof, e.g., di- and triblock copolymers.

An inhibitory nucleic acid molecule containing a hydrophobic moiety may exhibit superior cellular uptake, as compared to an inhibitory nucleic acid molecule lacking the hydrophobic moiety. A hydrophobic moiety is a monovalent group (e.g., a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen) covalently linked to the nucleic acid backbone (e.g., 5′-terminus) of the inhibitory nucleic acid molecule.

A targeting moiety is selected based on its ability to target oligonucleotides of the invention to a desired or selected cell population that expresses the corresponding binding partner (e.g., either the corresponding receptor or ligand) for the selected targeting moiety. For example, an oligonucleotide of the invention could be targeted to hepatocytes expressing asialoglycoprotein receptor (ASGP-R) by selecting a targeting moiety containing N-acetylgalactosamine (GalNAc).

A targeting moiety may include one or more ligands (e.g., 1 to 9 ligands, 1 to 6 ligands, 1 to 3 ligands, 3 ligands, or 1 ligand). The ligand may target a cell expressing asialoglycoprotein receptor (ASGP-R), IgA receptor, HDL receptor, LDL receptor, or transferrin receptor. Non-limiting examples of the ligands include N-acetylgalactosamine (e.g., a triantennary N-acetylgalactosamine), glycyrrhetinic acid, glycyrrhizin, lactobionic acid, lactoferrin, IgA, or a bile acid (e.g., lithocholyltaurine or taurocholic acid).

The ligand may be a small molecule, e.g., a small molecule targeting a cell expressing asialoglycoprotein receptor (ASGP-R). A non-limiting example of a small molecule targeting an asialoglycoprotein receptor is N-acetylgalactosamine. Alternatively, the ligand can be an antibody or an antigen-binding fragment or an engineered derivative thereof (e.g., Fcab or a fusion protein (e.g., scFv)).

### Preparation of Inhibitory Nucleic Acid Molecules

Inhibitory nucleic acid molecules of the disclosure may be prepared using techniques and methods known in the art for the oligonucleotide synthesis. For example, inhibitory nucleic acid molecules of the disclosure may be prepared using a phosphoramidite-based synthesis cycle. This synthesis cycle includes the steps of (1) de-blocking a 5′-protected nucleotide to produce a 5′-deblocked nucleotide, (2) coupling the 5′-deblocked nucleotide with a 5′-protected nucleoside phosphoramidite to produce nucleosides linked through a phosphite, (3) repeating steps (1) and (2) one or more times as needed, (4) capping the 5′-terminus, and (5) oxidation or sulfurization of internucleoside phosphites. The reagents and reaction conditions useful for the oligonucleotide synthesis are known in the art.

The inhibitory nucleic acid molecules disclosed herein may be linked to solid support as a result of solid-phase synthesis. Cleavable solid supports that may be used are known in the art. Non-limiting examples of the solid support include, e.g., controlled pore glass or macroporous polystyrene bonded to a strand through a cleavable linker (e.g., succinate-based linker) known in the art (e.g., UnyLinker™). A nucleic acid linked to solid support may be removed from the solid support by cleaving the linker connecting a nucleic acid and solid support.

### Compositions

The inhibitory nucleic acid molecules described herein may be formulated into various compositions (e.g., a pharmaceutical composition) for administration to a subject in a biologically compatible form suitable for administration in vivo. For example, the inhibitory nucleic acid molecules described herein (e.g., the siRNA molecules of SEQ ID NOs: 1-10, or variants thereof) may be administered in a suitable diluent, carrier, or excipient, and may further contain a preservative, e.g., to prevent the growth of microorganisms. Conventional procedures and ingredients for the selection and preparation of suitable compositions are described, for example, in Remington, J. P. The Science and Practice of Pharmacy, Easton, PA. Mack Publishers, 2012, 22^{nd} ed. And in The United States Pharmacopeial Convention, The National Formulary, United States Pharmacopeial, 2015, USP 38 NF 33).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates and mammals.

Compositions containing the inhibitory nucleic acids described herein may further include a second therapeutic agent (e.g., a nucleic acid molecule to be expressed within a cell, a polypeptide, or a drug). For example, a second therapeutic agent may be a blood pressure medication, an anti-inflammatory medication (e.g., a steroid or colchicine), or immunosuppressive agent. In some embodiments, the second therapeutic agent is a statin. Non-limiting examples of second therapeutic agents are a statin (e.g., atorvastatin), a proprotein convertase subtilisin/kexin type 9 (PCSK9) inhibitor (e.g., an siRNA or monoclonal antibody targeting PCSK9), an ATP Citrate Lyase (ACL) inhibitor (e.g., bempedoic acid), a lipoprotein(a) (Lp(a)) inhibitor (e.g., an siRNA targeting Lp(a)), an angiopoietin-like 3 (ANGPTL3) inhibitor (e.g., an siRNA targeting ANGPTL3), a cholesterylester transfer protein (CETP) inhibitor, a microsomal triglyceride transfer protein (MTP) inhibitor (e.g., lomitapide), an apolipoprotein B (ApoB) inhibitor (e.g., mipomersen), a bile acid binding resin, and an anti-inflammatory medication (e.g., colchicine).

In some embodiments, the second therapeutic agent (e.g., statin) is administered in combination with an inhibitory nucleic acid molecule of the disclosure. In some embodiments, the subject is orally administered a statin. In some embodiments, the subject is administered a statin daily.

### Methods of Treatment

The disclosure provides methods of decreasing LDL in the serum of a subject. In some embodiments, the method contains the steps of administering to a subject an inhibitory nucleic acid molecule described herein, wherein the inhibitory nucleic acid molecule targets AC9 (e.g., SEQ ID NO: 16 or 17). In some embodiments, the method contains the steps of administering to a subject an siRNA molecule described herein (e.g., SEQ ID NOs: 1-10, or a variant thereof), wherein the siRNA molecule targets AC9 (e.g., SEQ ID NO: 16 or 17). In some embodiments, the method contains the steps of administering to a subject an dsRNA molecule described herein, wherein the dsRNA molecule targets AC9 (e.g., SEQ ID NO: 16 or 17). In some embodiments, the method contains the steps of administering to a subject an ASO molecule described herein, wherein the ASO molecule targets AC9 (e.g., SEQ ID NO: 16 or 17). In some embodiments, the method contains the steps of administering to a subject an miRNA molecule described herein, wherein the miRNA molecule targets AC9 (e.g., SEQ ID NO: 16 or 17). In some embodiments, the method contains the steps of administering to a subject an shRNA molecule described herein, wherein the shRNA molecule targets AC9 (e.g., SEQ ID NO: 16 or 17).

The disclosure provides methods of increasing LDLr in a subject. In some embodiments, the method contains the steps of administering to a subject an inhibitory nucleic acid molecule described herein, wherein the inhibitory nucleic acid molecule targets AC9 (e.g., SEQ ID NO: 16 or 17). In some embodiments, the method contains the steps of administering to a subject an siRNA molecule described herein (e.g., SEQ ID NOs: 1-10, or a variant thereof), wherein the siRNA molecule targets AC9 (e.g., SEQ ID NO: 16 or 17). In some embodiments, the method contains the steps of administering to a subject an dsRNA molecule described herein, wherein the dsRNA molecule targets AC9 (e.g., SEQ ID NO: 16 or 17). In some embodiments, the method contains the steps of administering to a subject an ASO molecule described herein, wherein the ASO molecule targets AC9 (e.g., SEQ ID NO: 16 or 17). In some embodiments, the method contains the steps of administering to a subject an miRNA molecule described herein, wherein the miRNA molecule targets AC9 (e.g., SEQ ID NO: 16 or 17). In some embodiments, the method contains the steps of administering to a subject an shRNA molecule described herein, wherein the shRNA molecule targets AC9 (e.g., SEQ ID NO: 16 or 17).

Any of the methods can administer a composition (e.g., a pharmaceutical composition) or delivery vehicle (e.g., a vector or nanoparticle) that contains or expresses any of the inhibitory nucleic acid molecules described herein (e.g., siRNA, dsRNA, ASO, miRNA, or shRNA).

### Delivery Vehicle

The inhibitory nucleic acid molecule of the disclosure may be delivered to a subject (e.g., a human) using any suitable delivery vehicle. For example, a delivery vehicle for any of the inhibitory nucleic acid molecules described herein may be a vector, plasmid, or nano particle, (e.g., a micelle, a liposome, an exosome, or a lipid nano particle (LNP)).

The inhibitory nucleic acid molecule of the disclosure and compositions thereof may be delivered to a subject via a vector (e.g., a viral vector). Any suitable viral vector system can be used including, e.g., adenoviruses (e.g., Ad2, Ad5, Ad9, Ad15, Ad17, Ad19, Ad20, Ad22, Ad26, Ad27, Ad28, Ad30, or Ad39), rhabdoviruses (e.g., vesicular stomatitis virus), retroviruses, adeno-associated vectors, poxviruses, herpes viral vectors, and Sindbis viral vectors.

The inhibitory nucleic acid molecule of the disclosure and compositions thereof may be delivered to a subject via liposomes. Liposomes are artificially-prepared vesicles which may primarily be composed of a lipid bilayer and may be used as a delivery vehicle for the administration of the inhibitory nucleic acids described herein, and compositions thereof. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical composition.

The inhibitory nucleic acid molecule of the disclosure and compositions thereof may be delivered to a subject via exosomes. Exosomes produced from cells can be collected from cell culture medium by any suitable method. Typically, a preparation of exosomes can be prepared from cell culture or tissue supernatant by centrifugation, filtration or combinations of these methods. For example, using standard methods, exosomes can be prepared by differential centrifugation, that is low speed (<20000 g) centrifugation to pellet larger particles followed by high speed (>100000 g) centrifugation to pellet exosomes, size filtration with appropriate filters (for example, 0.22 micrometer filter), gradient ultracentrifugation (for example, with sucrose gradient) or a combination of these methods.

The inhibitory nucleic acid molecules of the disclosure, and compositions thereof, may be delivered to a subject via LNPs. For example, the inhibitory nucleic acid molecules (e.g., siRNA, dsRNA, ASO, miRNA, or shRNA) may be formulated in a lipid nanoparticle such as those described in International Publication No. WO2012170930, herein incorporated by reference in its entirety. As a non-limiting example, LNP formulations may contain cationic lipids, distearoylphosphatidylcholine (DSPC), cholesterol, polyethylene glycol (PEG), R-3-[(w-methoxy poly(ethylene glycol)2000)carbamoyl)]-1,2-dimyristyloxl-propyl-3-amine (PEG-c-DOMG), distearoyl-rac-glycerol (DSG) and/or dimethylaminobutanoate (DMA). As a non-limiting example, 1-5% of the lipid molar ratio of PEG-c-DOMG as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypoly ethylene glycol) or PEG-DPG (1,2-Dipalmitoyl-sn-glycerol, methoxypolyethylene glycol). The cationic lipid may be selected from any lipid known in the art such as, but not limited to, (6Z,9Z,28Z,31Z)-heptatriacont-6,9,28,31-tetraene-19-yl 4-(dimethylamino)butanoate (DLin-MC3-DMA), 1,2-dilinoleyloxy-n,n-dimethyl-3-aminopropane (DLin-DMA), C 12-200, and N,N-dimethyl-2,2-di-(9Z,12Z)-9,12-octadecadien-1-yl-1,3-dioxolane-4-ethanamine (DLin-KC2-DMA).

Exemplary commercial reagents useful for lipid-based delivery of inhibitory nucleic acid molecules including, but not limited to, TransIT-TKO™ (Mirus, Catalog No. MIR 2150), Transmessenger™ (Qiagen, Catalog No. 301525), Oligofectamine™ and Lipofectamine™ (Invitrogen, Catalog No. MIR 12252-011 and Catalog No. 13778-075), siPORT™ (Ambion, Catalog No. 1631), and DharmaFECT™ (Fisher Scientific, Catalog No. T-2001-01).

### Dosage

The actual dosage amount of a composition of the present disclosure administered to a subject can be determined by physical and physiological factors such as body weight, severity of condition, previous or concurrent therapeutic interventions, idiopathy of the patient and on the route of administration. Depending upon the dosage (e.g., mg/kg) and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Routes of Administration

The compositions utilized in the methods described herein can be administered to a subject by any suitable route of administration. For example, a composition containing an inhibitory nucleic acid of the disclosure may be administered intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions.

In some embodiments, the compositions utilized in the methods described herein can be administered to the subject intravenously. In some embodiments, the compositions utilized in the methods described herein can be administered to the subject subcutaneously.

### EXAMPLES

### Example 1. Effects of siRNA Targeting of Human AC9 In Vitro

The following Example describes the materials and methods that were utilized for obtaining the results described herein.

### Material and Methods

### Cell Culture

HepG2 (hepatocellular carcinoma) cells were growth in Eagle's minimum essential medium (EMEM). The medium was supplemented with 10% FBS, 100 units/ml penicillin, and 100 μg/ml streptomycin. Cells were cultured in 5% CO₂ at 37° C. and were harvested once a week with trypsin-EDTA. For experiments, cells were trypsinized, seeded, and cultured at least for 3 days prior to the assays.

siRNA Transfection

HepG2 cells were transfected with ADCY9, SREBP2, LDLr or scramble siRNA in the presence of Lipofectamine RNAiMAX in Opti-MEM for 72 h unless otherwise stated.

### Cellular Cholesterol Efflux

Cells were labeled in EMEM containing 2 μCi/ml [1,2-³H]cholesterol plus 1% FBS for 24 h at 37° C. Then, cells were equilibrated with EMEM containing 0.2% BSA for 18 h at 37° C. with or without H89 or GSK-2033. An efflux assay was performed in the absence or presence of 10 μg/ml of apo A-I. At the end of the incubation, the medium was harvested and cells were solubilized. Medium and cells were counted for radioactivity in a p-counter. The percentage of efflux was calculated by subtracting the radioactive counts in the medium in the absence of cholesterol acceptors from the radioactive counts in the presence of acceptor and then dividing by the sum of the radioactive counts in the medium plus the cell fraction.

### Isolation and Radiolabeling of Lipoproteins

Lipoproteins were isolated from human plasma obtained from BioIVT (Westbury, NY). Before the isolation, the plasma was adjusted to 0.01% ethylenediamine tetraacetate (EDTA), 0.02% sodium azide, and 10 μM phenylmethylsulfonyl fluoride (PMSF). Human LDL (d=1.025-1.063 g/ml) was prepared by ultracentrifugation as described by Brissette et al. (doi: 10.1042/bj3180841). LDL was labeled with 1,2-[³H]cholesteryl oleate essentially as described by Roberts et al. (doi: 10.1042/bj2260319). Thereafter the labeled LDL were reisolated by ultracentrifugation. The specific activity of LDL labeled in CE ranged from 7000 to 12,000 cpm/μg protein.

### Lipoprotein Cell Association Assays

Cell association of [³H]CE-lipoprotein (20 μg of protein/ml) was measured at 37° C. for 4 h in 12-well plates. Cells were washed twice with 1 ml of phosphate-buffered saline (PBS) and incubated in a total volume of 250 μl containing 125 μl of culture medium (2×), 4% bovine serum albumin, pH 7.4 (total binding). Nonspecific association was assessed by the addition of 1.5 mg of protein/ml of unlabeled lipoproteins. At the end of the incubation the cells were washed twice with 1 ml of PBS containing 0.2% BSA (PBS-BSA) followed by two washes with 1 ml of PBS. The cells were then solubilized in 1.5 ml of 0.2 N NaOH. Radioactivity counts in the homogenates were obtained with a beta-counter. To compare the association of lipoproteins labeled in CE (³H), the association data were estimated as micrograms of protein per milligram of cell protein (apparent uptake). To achieve this, the specific activity of [³H]CE-lipoprotein was expressed in counts per microgram of lipoprotein protein. The specific association was calculated by subtracting the nonspecific association from the total association.

### Immunoblotting

For Western blotting, cells were washed with cold PBS and then scraped and lysed in ice-cold lysis buffer (50 mM Tris, pH 7.5, 150 mM NaCl, 1% Triton X-100, 0.1% SDS, 0.5% sodium deoxycholate, 1 mM PMSF, and protease inhibitor cocktail). Lysate was microcentrifugated for 20 min at 4° C. and supernatant was assessed for cell proteins. Proteins (30-50 mg) were separated by sodium dodecyl sulfate-polyacrylamide gel electrophoresis (SDS-PAGE) followed by Western blotting to PVDF membrane.

Quantification of mRNA Expression by Reverse Transcription-Quantitative PCR

HepG2 cells total RNA were extracted using RNeasy isolation kits according to the manufacturer's protocol. cDNA was synthesized with components from High-Capacity cDNA Reverse Transcription kits and with the use of MultiScribe Reverse Transcriptase, according to the manufacturer's procedures. RNA was quantified by the Quant-it RiboGreen RNA Assay Kit according to the manufacturer's procedures and RNA quality was assessed using Agilent RNA 6000 Nano Kit for Bioanalyzer 2100 System. Primers were designed using the Beacon designer software v.8 and obtained from IDT. The reference genes for normalization, PPIA and TBP, were selected by using the Bio-Rad CFX Maestro software which uses the GeNorm method. The qPCR was performed with SYBR-Green reaction mix. The qPCR conditions consisted of an initial denaturation at 95° C. for 5 minutes, followed by 40 cycles of amplification, with each cycle consisting of 95° C. for 15 seconds, and 60° C. for 60 seconds. Results were analysed with the delta-delta Ct method with the Bio-Rad CFX Maestro software.

### SREBP-2 Transcriptional Activity

SREBP-2 transcriptional activity was estimated by using a kit in which SREBP-2 contained in a nuclear extract obtained from transfected HepG2 cells, following the protocol of kit supplier, binds specifically to immobilized SREBP-response element oligonucleotides and is detected by addition of a specific primary antibody against SREBP-2.

### Proteomic Analysis by Tandem Mass Tags (TMT)

HepG2 cells were transfected and 3 days after cells were harvested and used for untargeted proteomic analysis. Briefly, 8 tandem mass tags (TMT) served to label two different conditions (siScramble and siAC9) of four different assays. Once labeled, all samples were mixed and analyzed in a single liquid chromatography-mass spectrometry (LC-MS) experiment.

### Other Methods

Protein content was determined by the method of Lowry using BSA as standard.

### Results

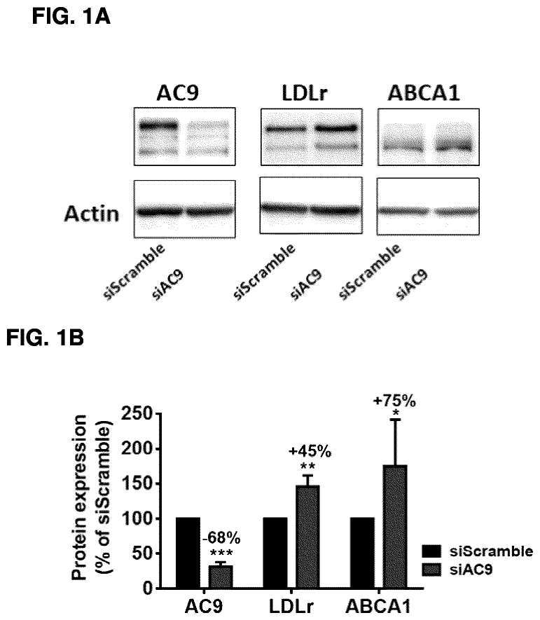

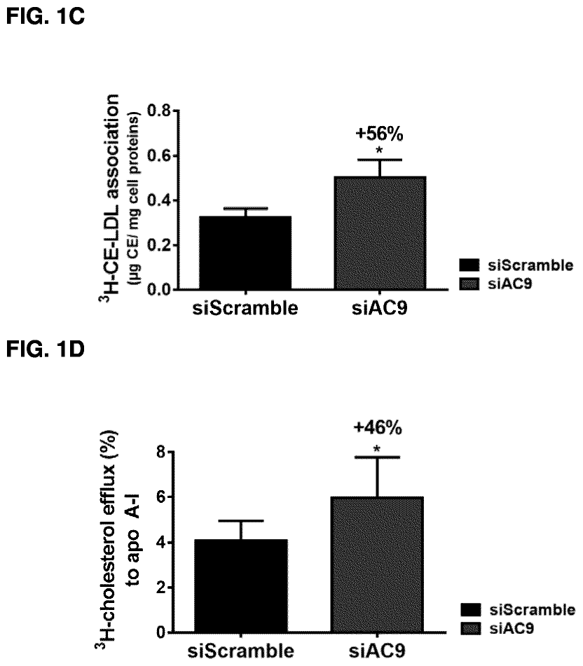

Protein expression of adenylate cyclase type 9 (AC9), low density lipoprotein receptor (LDLr), and ATP binding cassette subfamily A member 1 (ABCA1) was analyzed in HepG2 cells after siRNA-mediated knockdown (KD) of AC9 (FIG. 1A). The protein expression of AC9 was significantly reduced, while protein expression of LDLr and ABCA1 was significantly increased (FIG. 1B). A quantification of ³H-CE-LDL association (FIG. 1C) and ³H-CE-LDL cholesterol efflux (FIG. 1D) showed a significant increase after siRNA-mediated KD of AC9.

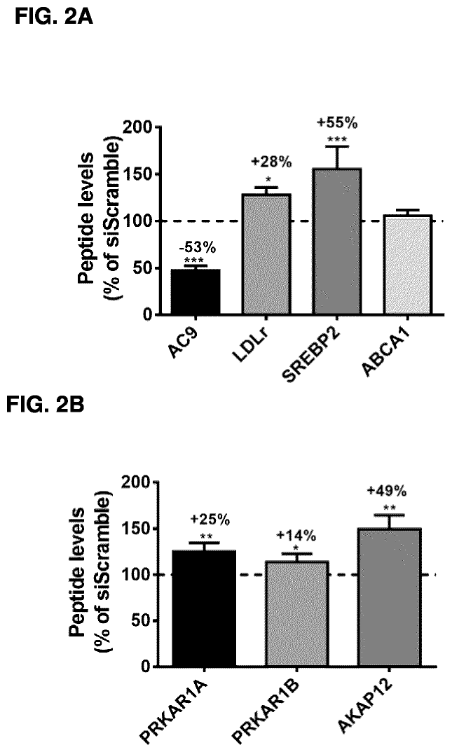

Protein expression of AC9, LDLr, sterol regulatory element-binding protein-2 (SREBP-2), and ABCA1 was analyzed by untargeted relative proteomics in HepG2 cells after siRNA-mediated KD of AC9. The peptide levels of AC9 were significantly reduced while the peptide levels of LDLr and SREBP2 were significantly increased (FIG. 2A).

Protein expression of protein kinase c-AMP-dependent type I regulatory alpha (PRKAR1A), protein kinase c-AMP-dependent type I regulatory beta (PRKAR1B), and A-kinase anchoring protein 12 (AKAP12) was analyzed by untargeted relative proteomics in HepG2 cells after siRNA-mediated KD of AC9. The peptide levels of PRKAR1A, PRKAR1B, and AKAP12 were significantly increased (FIG. 2B).

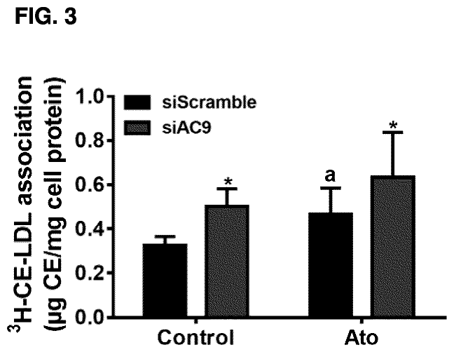

³H-CE-LDL association was analyzed in HepG2 cells after siRNA-mediated KD of AC9 (siAC9), with and without co-treatment of 5 μM of atorvastatin (Ato). Co-treatment with siAC9 and Ato resulted in additive effects on LDL cholesterol uptake by LDLr (FIG. 3).

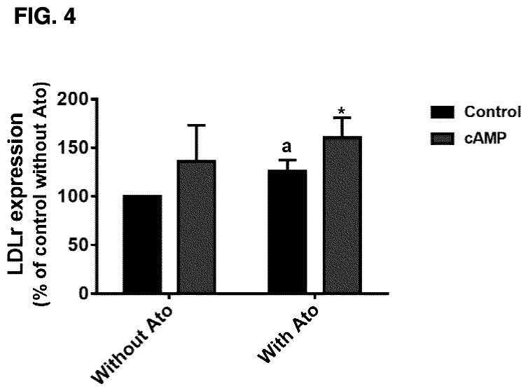

LDLr expression was analyzed in HepG2 cells after treatment with 300 μM of exogenous cAMP (to replicate the AC9 knock-down effect), 5 μM of Ato, or both cAMP and Ato. The combined effect of exogenous cAMP and atorvastatin increased LDLr protein expression (FIG. 4).

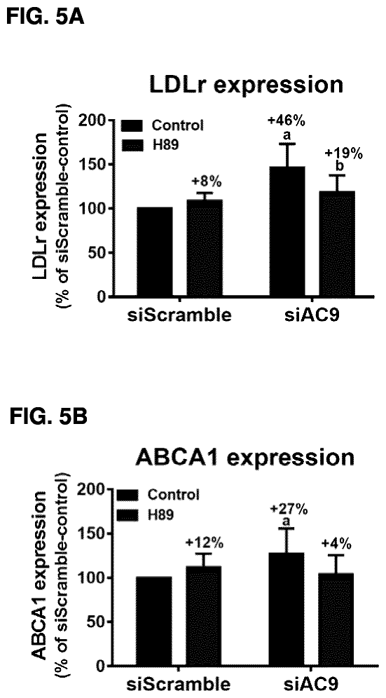

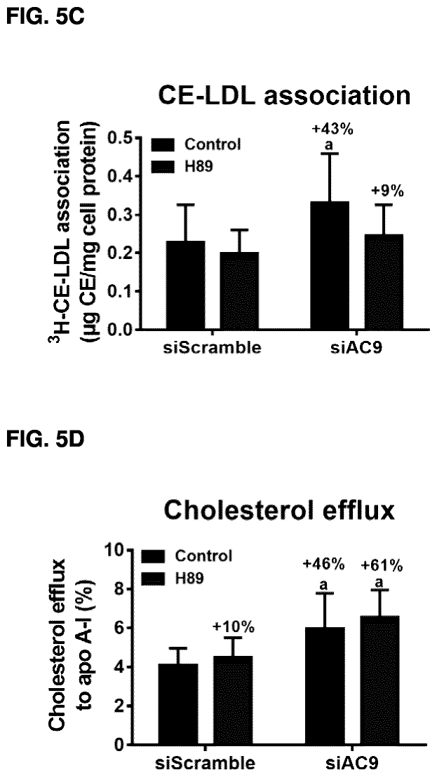

LDLr and ABCA1 expression was analyzed in HepG2 cells after siRNA-mediated KD of AC9, with and without co-treatment of 2 μM of H89 (a PKA inhibitor). siRNA-mediated KD of AC9 resulted in increased LDLr and ABCA1 expression; however, this increased expression was reversed upon co-treatment with the PKA inhibitor, H89 (FIG. 5A-B). Further, a reduction in ³H-CE-LDL association but not cholesterol efflux was observed in HepG2 cells treated with siAC9 and H89 (FIG. 5C-D). Taken together, these results indicate that the LDLr and ABCA1 increase observed upon KD of AC9 is mostly PKA-dependent.

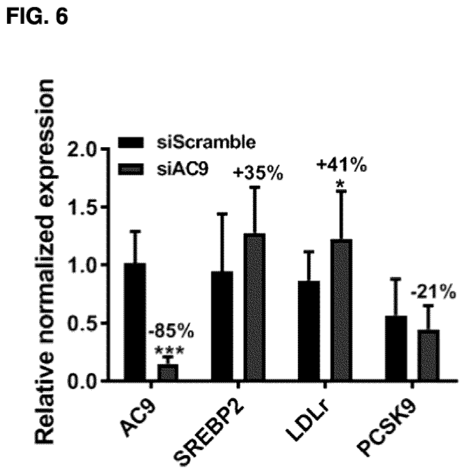

Gene expression of AC9, SREBP2, proprotein convertase subtilisin/kexin type 9 (PCSK9), and LDLr was analyzed in HepG2 cells after siRNA-mediated KD of AC9 (FIG. 6). These results illustrate that AC9 siRNA increases mRNA expression of SREBP2 and LDLR, but not PCSK9.

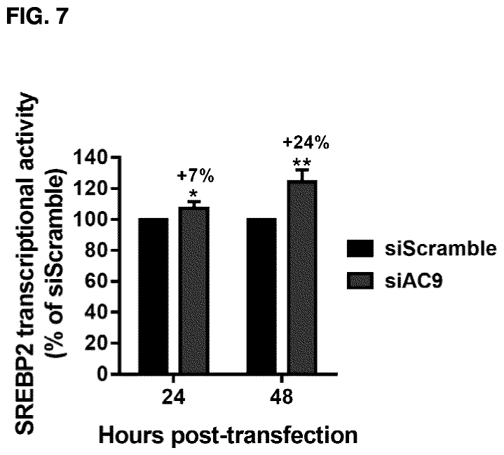

SREBP2 transcriptional activity was analyzed in HepG2 cells 24-hours and 48-hours after siRNA-mediated KD of AC9. These results show that AC9 siRNA increases SREBP2 transcriptional activity (FIG. 7).

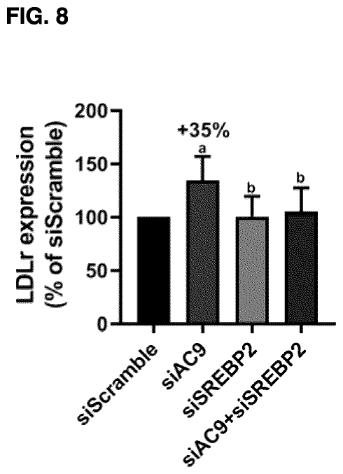

Expression of LDLr protein was analyzed in HepG2 cells after siRNA-mediated KD of AC9, SREBP2, or both AC9 and SREBP2 (FIG. 8). KD of AC9 results in an increase in LDLr protein expression. These results indicate the SREBP2 siRNA can block the siAC9-mediated increase of LDLr protein.

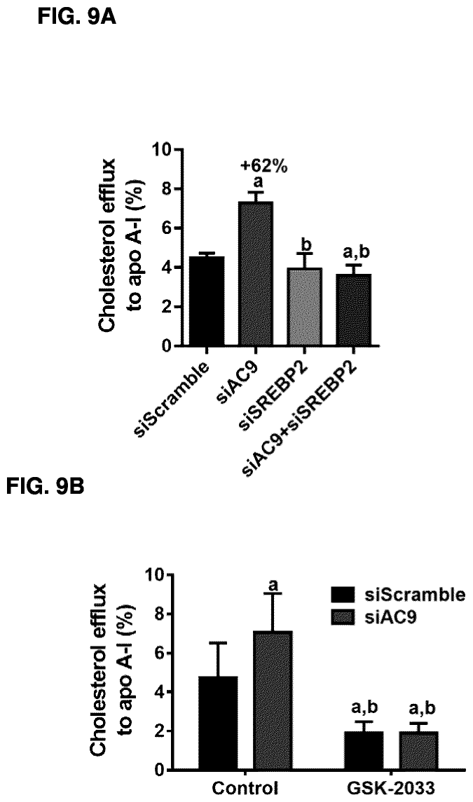

Cholesterol efflux to apo A-I in HepG2 cells was analyzed after siRNA-mediated KD of AC9, SREBP2, or both AC9 and SREBP2, revealing a significant increase in cholesterol efflux only upon the KD of AC9 (FIG. 9A). Next, cholesterol efflux was investigated in HepG2 cells after siRNA-mediated KD of AC9, with and without co-treatment with 5 μM of GSK-2033 (a LXR inhibitor). The LXR inhibitor was able to significantly reduce the siAC9-mediated increase of ABCA1-mediated cholesterol efflux (FIG. 9B). These results illustrate that LXR is involved in the effects of AC9 siRNA on cholesterol efflux via ABCA1.

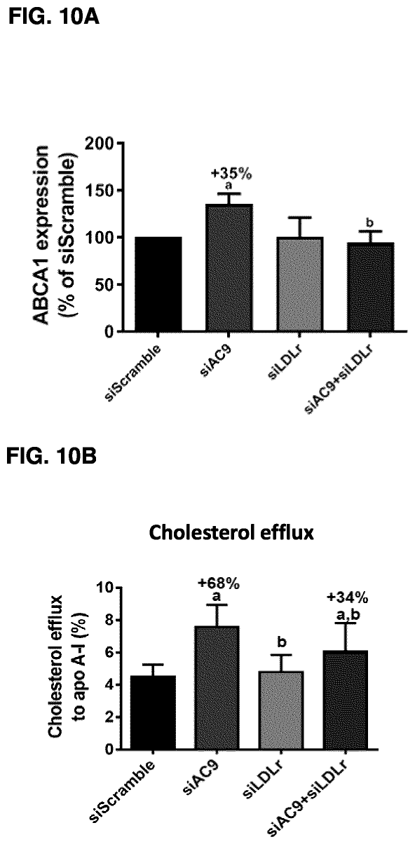

ABCA1 protein expression was analyzed in HepG2 cells after siRNA-mediated KD of AC9, LDLr, or both AC9 and LDLr (FIG. 10A). Only the KD of AC9 alone increased ABCA1 expression. Cholesterol efflux also increased upon KD of AC9 (FIG. 10B). Upon co-treatment with AC9 and LDLr siRNA, cholesterol efflux also increased, albeit significantly less so compared to AC9 siRNA alone. These results indicate that LDLr siRNA partially blocks the siAC9-mediated increase of ABCA1 protein.

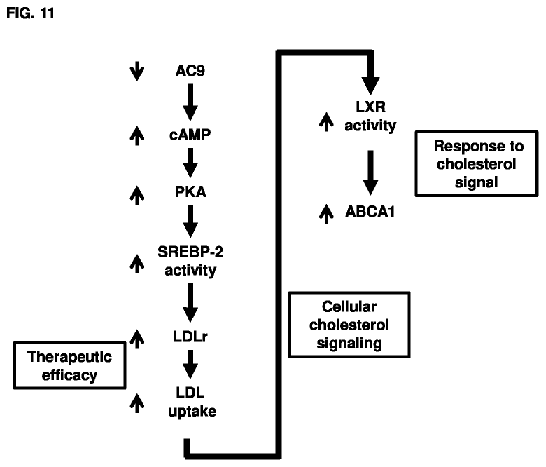

The above results illustrate AC9 KD effects on LDLr and ABCA1 expression and function (e.g., see FIG. 11).

Protein expression of AC9 and LDLr was analyzed in HepG2 cells transfected with different siRNA sequences targeting ADCY9 mRNA at different exons. Protein expression of AC9 was significantly reduced, while protein expression of LDLr was significantly increased by all siRNA sequences used (Table 4).

TABLE 4
Impact of various siRNA sequences targeting ADCY9 mRNA
on AC9 and LDLr protein expression in HepG2 cells

| | Protein expression in human HepG2 cells | |
|---|---|---|
| | (% of siScramble) | |
| siRNA sequences | AC9 | LDLr |
| ADCY9-1 (Exon 2) | 24% *** | 131% ** |
| ADCY9-2 (Exon 10) | 16% *** | 122% ** |
| ADCY9-3 (Exon 5-6) | 17% *** | 126% * |
| ADCY9-4 (Exon 10) | 15% *** | 150% ** |
| ADCY9-ID1039 (Exon 5) | 15% *** | 123% * |

Table 4 shows AC9 and LDLr protein levels following transfection of HepG2 cells with different siRNA sequences targeting ADCY9 mRNA at different exon, relative to an siScramble control. Results were obtained by Western blotting and actin was used as a loading control. Protein expression is represented as a percentage of the control, siScramble. n=8. Paired t-test: *=p≤0.05; **=p≤0.01; and ***=p≤0.001, relative to siScramble.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations following, in general, the principles and including such departures from the invention that come within known or customary practice within the art to which the invention pertains and may be applied to the essential features hereinbefore set forth, and follows in the scope of the claims.

Other embodiments are within the claims.

What is claimed is:

1. A method of decreasing serum low density lipoprotein (LDL) level in a subject, the method comprising inhibiting expression or function of an adenylate cyclase in the subject, wherein the inhibiting comprises administration of an inhibitory nucleic acid molecule to the subject.

2. A method of increasing LDL receptor expression in a subject, the method comprising inhibiting expression or function of an adenylate cyclase in the subject, wherein the inhibiting comprises administration of an inhibitory nucleic acid molecule to the subject.

3. The method of claim 1 or 2, wherein the adenylate cyclase is adenylate cyclase type 9 (AC9).

4. The method of claim 3, wherein the AC9 comprises:
(i) an mRNA sequence of SEQ ID NO: 16; and/or
(ii) a DNA sequence of SEQ ID NO: 17.

5. The method of any one of claims 1-4, wherein the inhibitory nucleic acid molecule is an anti-sense oligonucleotide (ASO), a small interfering RNA (siRNA), a short hairpin RNA (shRNA), a double-stranded RNA (dsRNA), or a microRNA (miRNA).

6. The method of claim 4, wherein the inhibitory nucleic acid molecule is an siRNA.

7. The method of claim 5, wherein the siRNA comprises a sequence complementary to at least 15 contiguous nucleotides set forth within any one of SEQ ID NOs: 1-10, 16, and 17.

8. The method of claim 6, wherein the siRNA comprises a sequence complementary to at least 19 contiguous nucleotides set forth within any one of SEQ ID NOs: 1-10, 16, and 17.

9. The method of claim 7, wherein the siRNA comprises a sequence complementary to at least 21 contiguous nucleotides set forth within any one of SEQ ID NOs: 1-10, 16, and 17.

10. The method of claim 8, wherein the siRNA comprises a sequence complementary to at least 25 contiguous nucleotides set forth within any one of SEQ ID NOs: 16 and 17.

11. The method of any one of claims 5-9, wherein the siRNA molecule contains 3′ overhangs selected from the group consisting of:
(i) a single uracil overhang at one or more 3′ ends of the siRNA;
(ii) a double uracil overhang at one or more 3′ ends of the siRNA;
(iii) a single thymine overhang at one or more 3′ ends of the siRNA;
(iv) a double thymine overhang at one or more 3′ ends of the siRNA; or
(v) a single cytosine and single thymine overhang at one or more 3′ ends of the siRNA.

12. The method of any one of claims 5-11, wherein the siRNA comprises a nucleotide sequence of any one or more of SEQ ID NOs: 1-10.

13. The method of claim 12, wherein the siRNA comprises:
(i) a sense strand comprising the sequence of SEQ ID NO: 1 and an antisense strand comprising the sequence of SEQ ID NO: 2;
(ii) a sense strand comprising the sequence of SEQ ID NO: 3 and an antisense strand comprising the sequence of SEQ ID NO: 4;
(iii) a sense strand comprising the sequence of SEQ ID NO: 5 and an antisense strand comprising the sequence of SEQ ID NO: 6;
(iv) a sense strand comprising the sequence of SEQ ID NO: 7 and an antisense strand comprising the sequence of SEQ ID NO: 8; or
(v) a sense strand comprising the sequence of SEQ ID NO: 9 and an antisense strand comprising the sequence of SEQ ID NO: 10.

14. The method of any one of claims 5-13, wherein the siRNA comprises a non-natural or modified nucleoside or nucleotide.

15. The method of claim 11, wherein the wherein the modification is chosen from a 2′-O-methyl (2′-O-Me) modified nucleoside, a phosphorothioate (PS) bond between nucleosides, and a 2′-fluoro (2′-F) modified nucleoside.

16. The method of any one of claims 5-12, wherein the siRNA molecule targets the sequence of any one of SEQ ID NOs: 11-15.

17. The method of any one of claims 1-16, wherein the method further comprises administering a second therapeutic agent to the subject.

18. The method of claim 17, wherein the second therapeutic agent is selected from the group consisting of a statin, a proprotein convertase subtilisin/kexin type 9 (PCSK9) inhibitor, an ATP Citrate Lyase (ACL) inhibitor, a lipoprotein(a) (Lp(a)) inhibitor, an angiopoietin-like 3 (ANGPTL3) inhibitor, a cholesterylester transfer protein (CETP) inhibitor, a microsomal triglyceride transfer protein (MTP) inhibitor, an apolipoprotein B (ApoB) inhibitor, a bile acid binding resin, and colchicine.

19. The method of claim 18, wherein the statin is atorvastatin.

20. The method of claim 18, wherein the PCSK9 inhibitor is an siRNA molecule targeting PCSK9 or a monoclonal antibody.

21. The method of claim 18, wherein the ACL inhibitor is bempedoic acid.

22. The method of claim 18, wherein the Lp(a) inhibitor is an siRNA molecule targeting Lp(a).

23. The method of claim 18, wherein the MTP inhibitor is lomitapide.

24. The method of claim 18, wherein the ApoB inhibitor is mipomersen.

25. An siRNA molecule comprising:
(i) a sense strand comprising the sequence of SEQ ID NO: 3 and an antisense strand comprising the sequence of SEQ ID NO: 4;
(ii) a sense strand comprising the sequence of SEQ ID NO: 5 and an antisense strand comprising the sequence of SEQ ID NO: 6;
(iii) a sense strand comprising the sequence of SEQ ID NO: 7 and an antisense strand comprising the sequence of SEQ ID NO: 8; or
(iv) a sense strand comprising the sequence of SEQ ID NO: 9 and an antisense strand comprising the sequence of SEQ ID NO: 10.

26. The siRNA molecule of claim 20, wherein the siRNA comprises a non-natural or modified nucleoside or nucleotide.

27. The siRNA molecule of claim 21, wherein the wherein the modification is chosen from a 2′-O-methyl (2′-O-Me) modified nucleoside, a phosphorothioate (PS) bond between nucleosides, and a 2′-fluoro (2′-F) modified nucleoside.

28. The siRNA molecule of claim 22, wherein the siRNA molecule targets the sequence of any one of SEQ ID NOs: 11-15.

29. Use of an inhibitory nucleic acid molecule to decrease serum low density lipoprotein (LDL) level in a subject, wherein expression or function of an adenylate cyclase is inhibited in the subject by administration of an inhibitory nucleic acid molecule.

30. Use of an inhibitory nucleic acid molecule to decrease LDL receptor expression in a subject, wherein expression or function of an adenylate cyclase is inhibited in the subject by administration of an inhibitory nucleic acid molecule.

31. The use of claim 29 or 30, wherein the adenylate cyclase is adenylate cyclase type 9 (AC9).
